(19) United States
(12) Patent Application Publication
(10) Pub. No.: US 2025/0215489 A1
(43) Pub. Date: Jul. 3, 2025

(54) Method of Sequencing L-Polynucleotide

(71) Applicant: Dxome Co., Ltd., Seongnam-si (KR)

(72) Inventors: Dae Hyun Kim, Irvine, CA (US); Jun Hyuk Chung, Irvine, CA (US); Bo Shun Huang, Chino, CA (US)

(21) Appl. No.: 18/848,478

(22) PCT Filed: Mar. 21, 2023
(86) PCT No.: PCT/US2023/015834

§ 371 Date: Sep. 18, 2024

Related U.S. Application Data
(60) Provisional application No. 63/322,451, filed on Mar. 22, 2022.

(51) Int. Cl. C12Q 1/6869; C07H 19/20; C07H 19/24; C12Q 1/48

(52) U.S. Cl. CPC C12Q 1/6869; C07H 19/20; C07H 19/24; C12Q 1/485

(57) ABSTRACT

The present disclosure provides a novel method of amplifying and sequencing L-form polynucleotide. Further provided include L-form nucleotides and D-form enzymes that can be used in the method. The method of making the L-form nucleotides and D-form enzymes are also provided.

### 1. SEQUENCE LISTING

The instant application contains a Sequence Listing.

### 2. BACKGROUND

Chirality is a geometric property of molecules. A molecule is chiral if it cannot be superposed on its mirror image by combination of rotations, translations, and some conformational changes.

Many natural biomolecules, such as proteins, DNA and RNA, are chiral. Amino acids except glycine have a chiral carbon atom adjacent to the carboxyl group. This chiral center allows the amino acids to exist as stereoisomers, also referred to as enantiomers, that can be characterized as either L or D based on optical activity. However, most amino acids found in nature have L-form, except glycine which has no chirality. Nucleic acids have their chiral centers on their backbones. Unlike amino acids, nucleic acids are dominantly in D-form in nature. Due to chiral specificity, biomolecules can interact and make use of other molecules or substrates only when they have certain chirality. L-form polymerase, for example, can synthesize a polynucleotide using D-form nucleotide, but not L-form nucleotide.

Recently, researchers have generated mirror-image forms of some biomolecules as an attempt to create a mirror-image artificial system based on D-form amino acids, L-form nucleic acids and D-form polymerases. Further, mirror-image therapeutic proteins or polynucleotides have been developed, e.g., with advantageous such slower biodegradation in the body. However, development of mirror-image biomolecules and systems faces a crucial barrier by lacking an efficient and reliable technology for sequencing L-polynucleotide.

### 3. SUMMARY

The present disclosure provides a novel method of sequencing L-form polynucleotide. Compositions for the sequencing methods, such as novel L-form nucleotides and D-form enzymes, are also provided. The sequencing method is expected to enable high-throughput sequencing of L-polynucleotides.

Accordingly, in a first aspect, the present disclosure provides a mirror image nucleotide comprising:
a. a pentose sugar selected from a (3R,4S)-3,4,5-trihydroxypentanal and a (4R)-4,5-dihydroxypentanal; wherein the H of the 5′ hydroxyl is substituted by one or more phosphate group; and wherein H of the 3′ hydroxyl group, if present, is optionally substituted by a cleavable protecting group;
b. a nitrogenous base, and
c. optionally a cleavable label comprising a cleavable linker and a label;
   wherein at least one of a cleavable protecting group and a cleavable label is present.

In certain embodiments, the cleavable label is linked to the nitrogenous base, the 3′ O, or the 5′ phosphate group.

In certain embodiments the mirror image nucleotide has a structure according to Formula I:

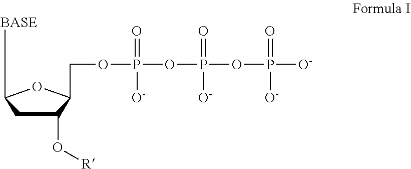

wherein BASE is a nitrogenous base, and R′ is a cleavable protecting group.

In certain embodiments the mirror image nucleotide has a structure according to Formula II

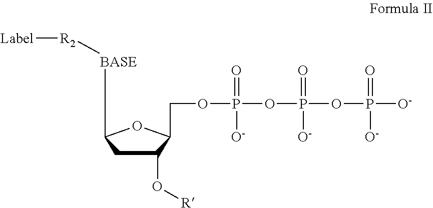

wherein BASE is a nitrogenous base; R′ is a cleavable protecting group or H; R₂—Label is a cleavable label comprising a cleavable linker R₂ and a label.

In certain embodiments the mirror image nucleotide has structure according to Formula III:

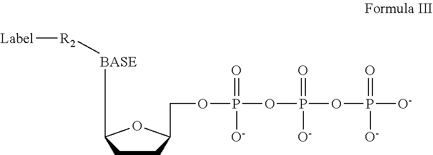

wherein BASE is a nitrogenous base; R₂—Label is a cleavable label comprising a cleavable linker R₂ and a label.

In certain embodiments of the mirror image nucleotide of Formula II or III, the label is selected from the group consisting of:

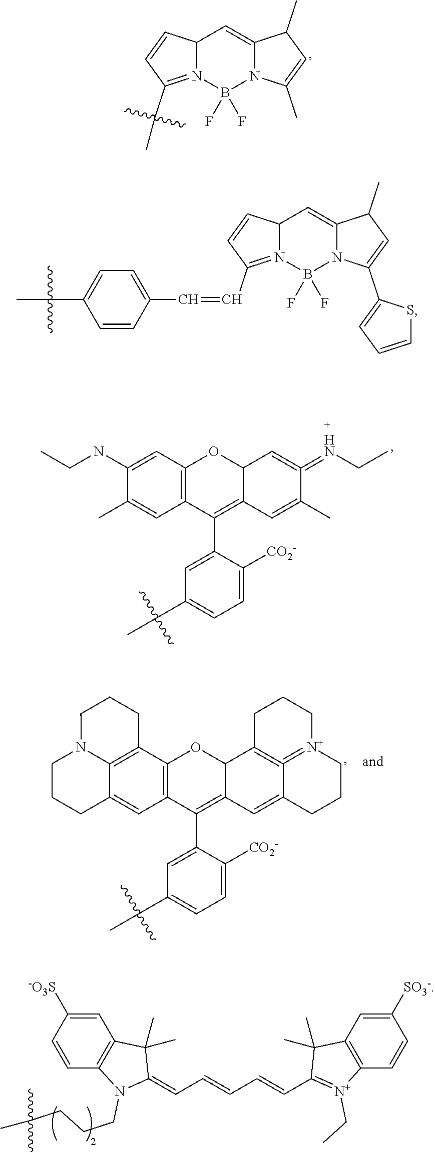

In certain embodiments of the mirror image nucleotide of Formula I or II, the cleavable protecting group is selected from an allyl, a dimethyl disulfide, a nitrobenzyl, and an azido protecting group.

In certain embodiments of the mirror image nucleotide of Formula I or II, the cleavable protecting group is selected from:

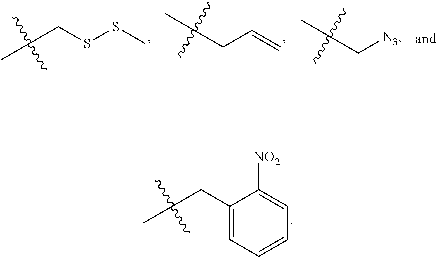

In certain embodiments of the mirror image nucleotide of Formula II or III, wherein the cleavable linker is photocleavable, is cleaved by contact with water-soluble phosphines, or is cleaved by water-soluble transition metal-containing catalysts.

In certain embodiments of the mirror image nucleotide of Formula II or III, wherein the cleavable linker comprises an allyl, a disulfide, or an azido group.

In certain embodiments of the mirror image nucleotide of Formula II or III, wherein the cleavable linker comprises:

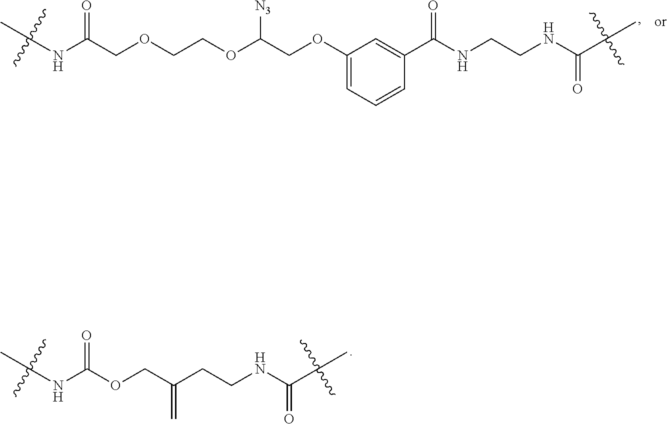 [SMILES: *NC(=O)COCCOC(COc1cccc(C(=O)NCCNC(*)=O)c1)N=[N+]=[N-] ; *NC(=O)OCC(=C)CCNC(*)=O]

In certain embodiments of the mirror image nucleotide of Formula I, the compound has a structure selected from:

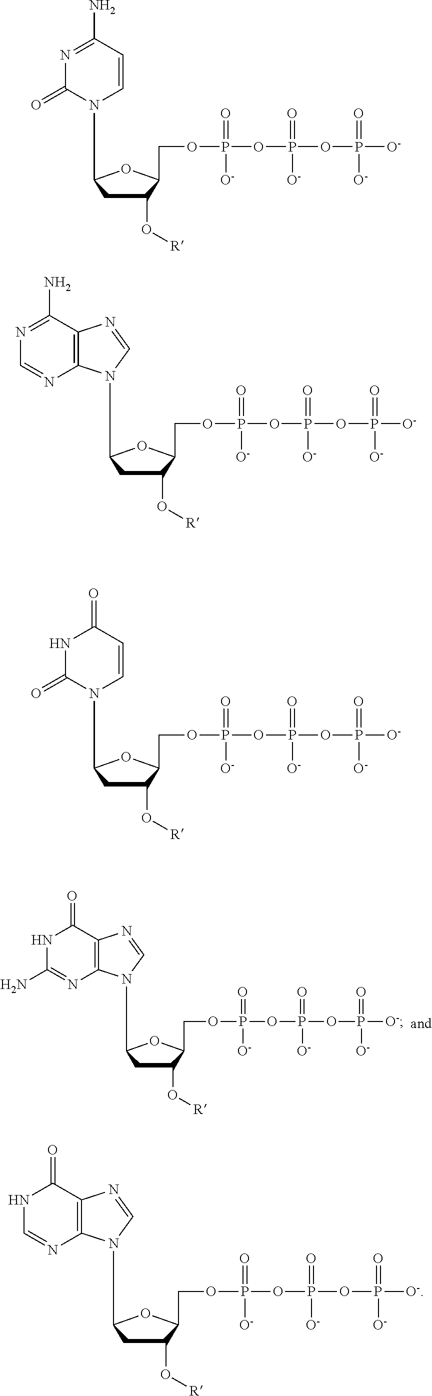

In certain embodiments, R′ comprises a methyl disulfide, an allyl, an azide, or nitrobenzyl moiety.

In certain embodiments, R′ is selected from:

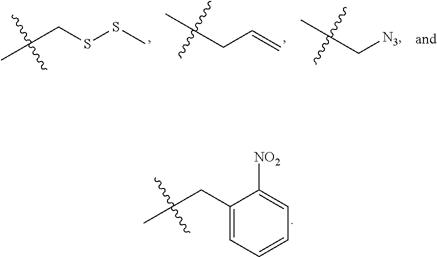

In certain embodiments of the mirror image nucleotide of Formula II, the compound has a structure selected from:

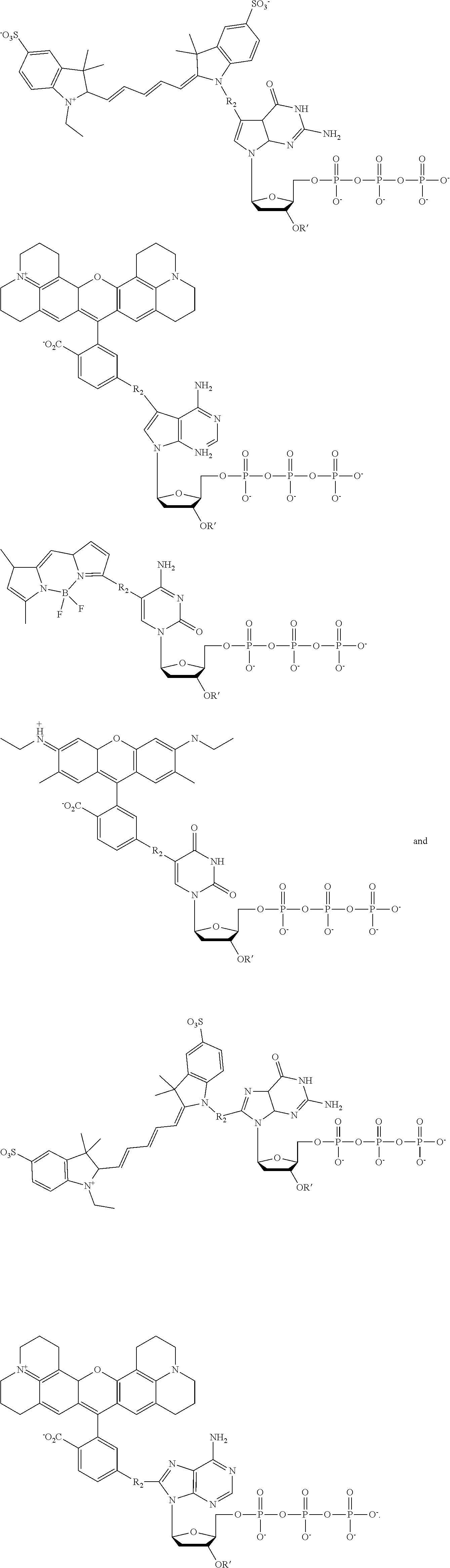

In certain embodiments, R′ comprises a methyl disulfide, an allyl, an azide, or nitrobenzyl moiety.

In certain embodiments, R′ is selected from:

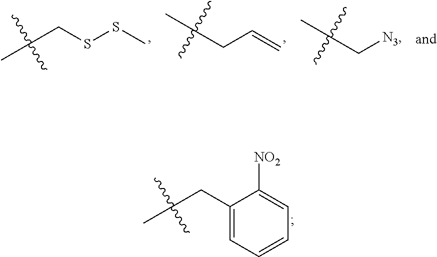

and

In certain embodiments, R₂ comprises:

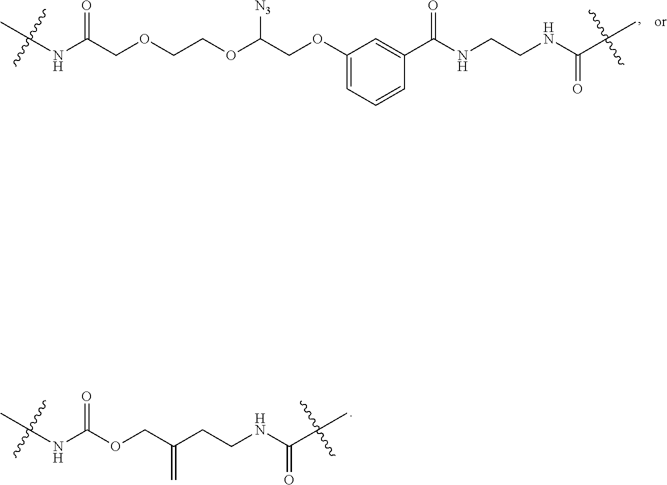 [SMILES: *NC(=O)COCCOC(COc1cccc(C(=O)NCCNC(*)=O)c1)N=[N+]=[N-] ; *NC(=O)OCC(=C)CCNC(*)=O]

In certain embodiments of the mirror image nucleotide of Formula III, the compound has a structure selected from:

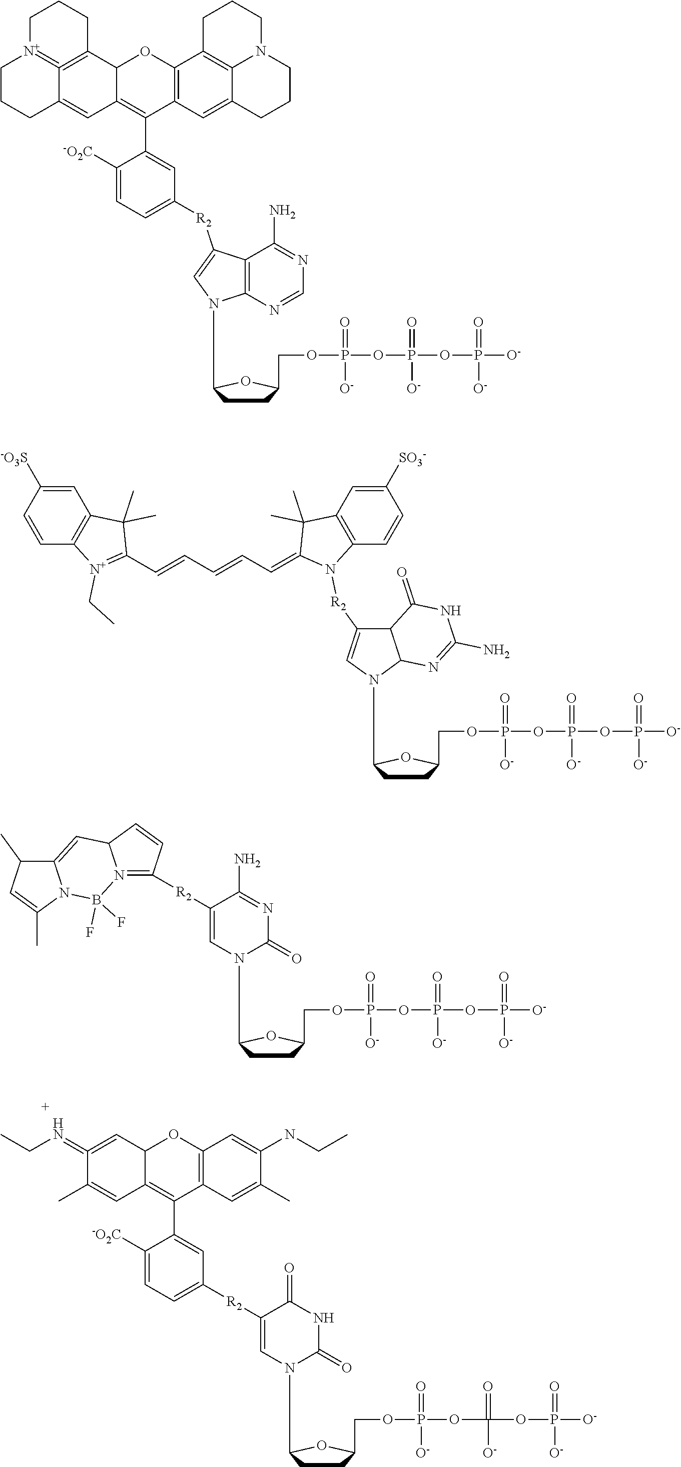

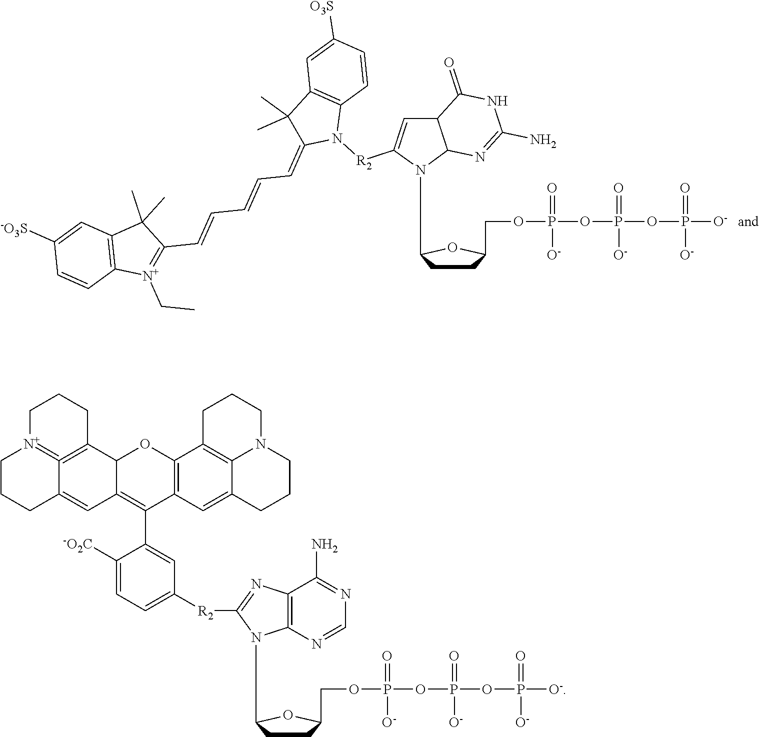

In certain embodiments R₂ comprises:

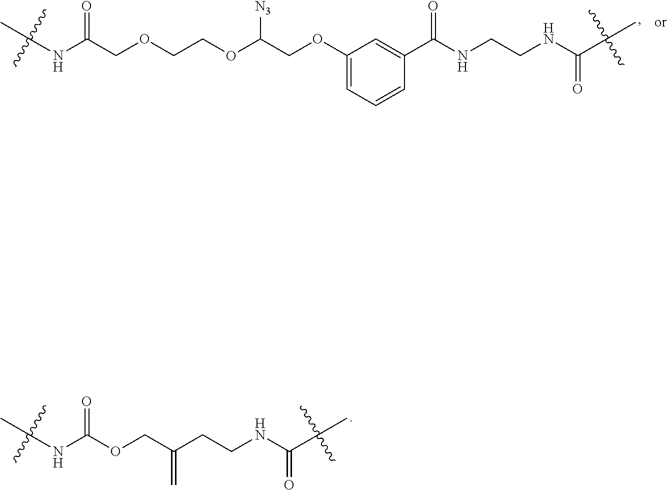 [SMILES: *NC(=O)COCCOC(COc1cccc(C(=O)NCCNC(*)=O)c1)N=[N+]=[N-] ; *NC(=O)OCC(=C)CCNC(*)=O]

In certain embodiments of the mirror image nucleotide of Formula II, the compound has a structure selected from:

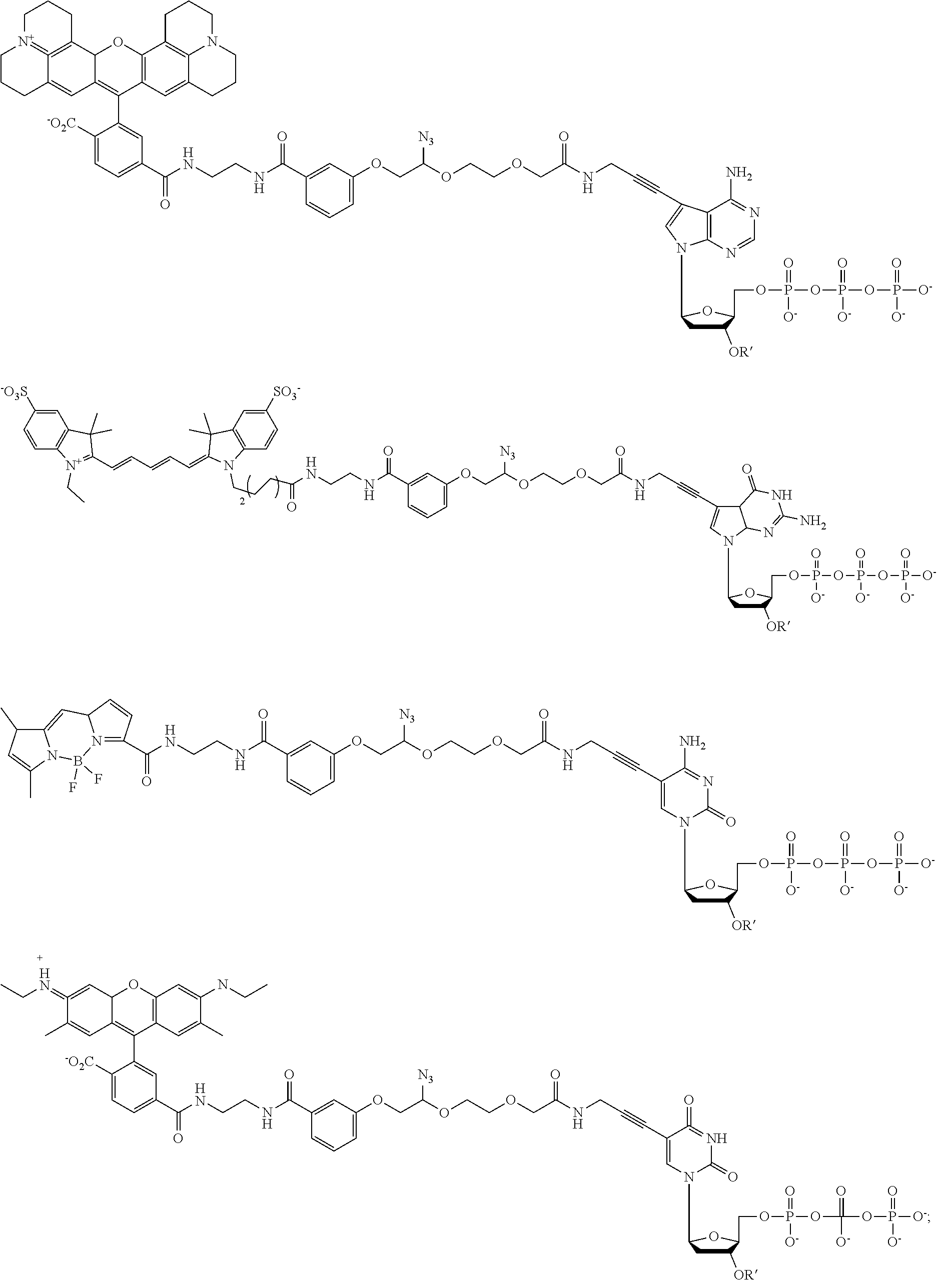

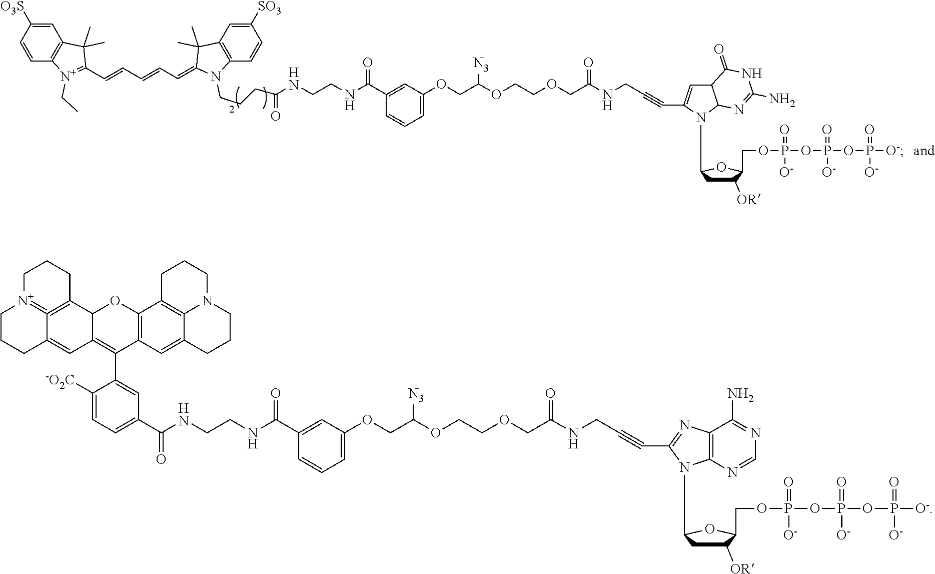

wherein the R′ is selected from:
H,

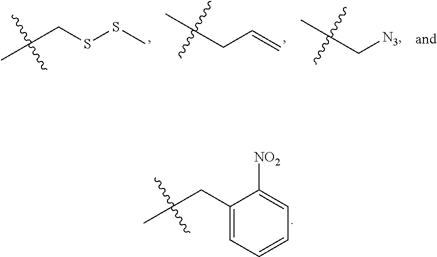

In certain embodiments of the mirror image nucleotide of Formula III, the compound has a structure selected from:

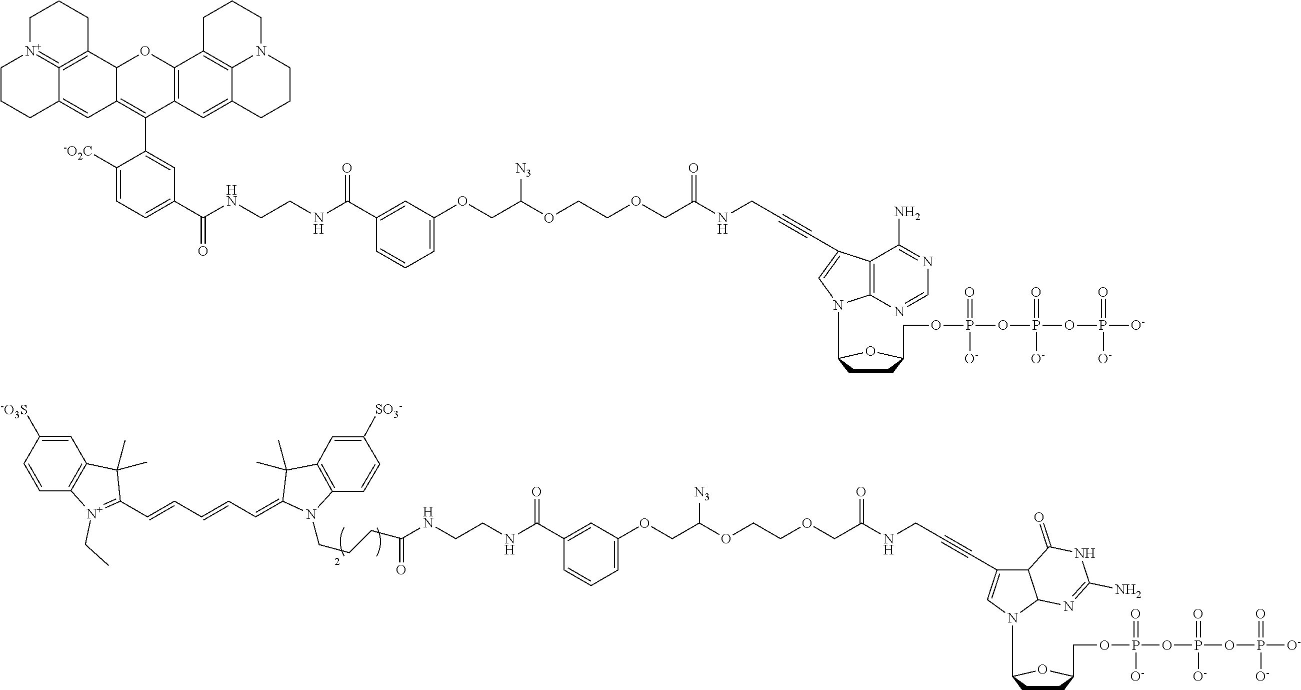

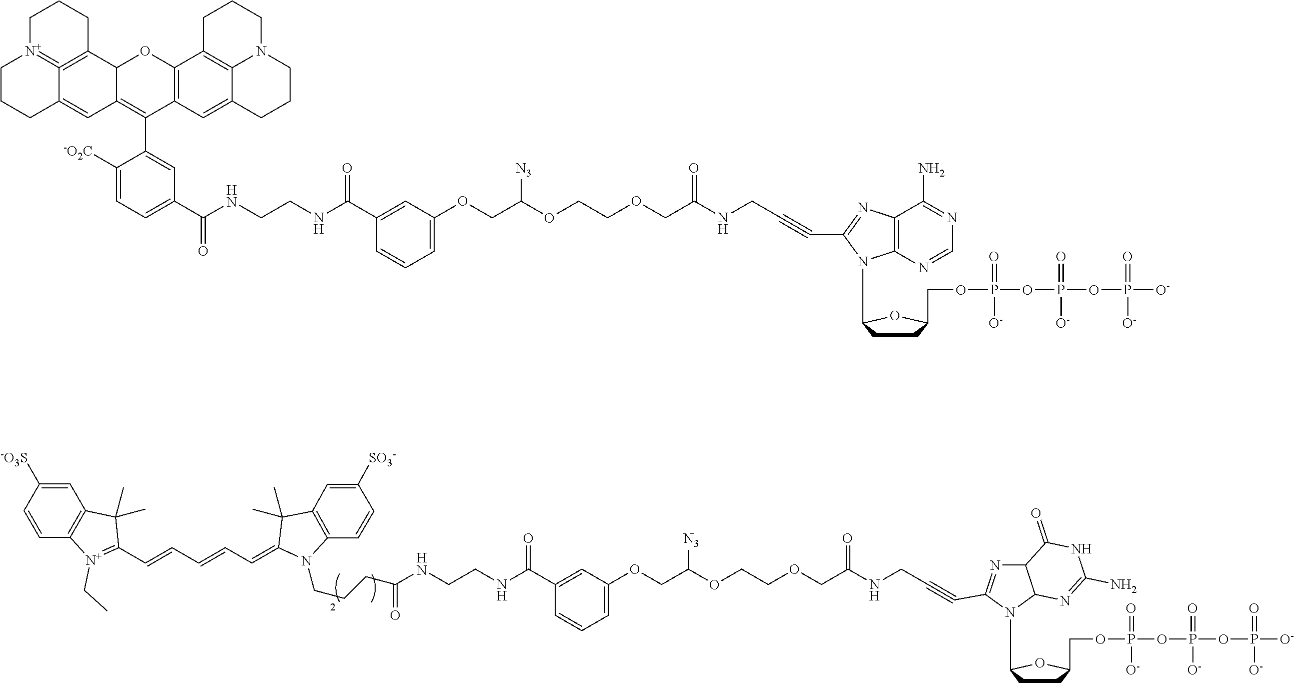

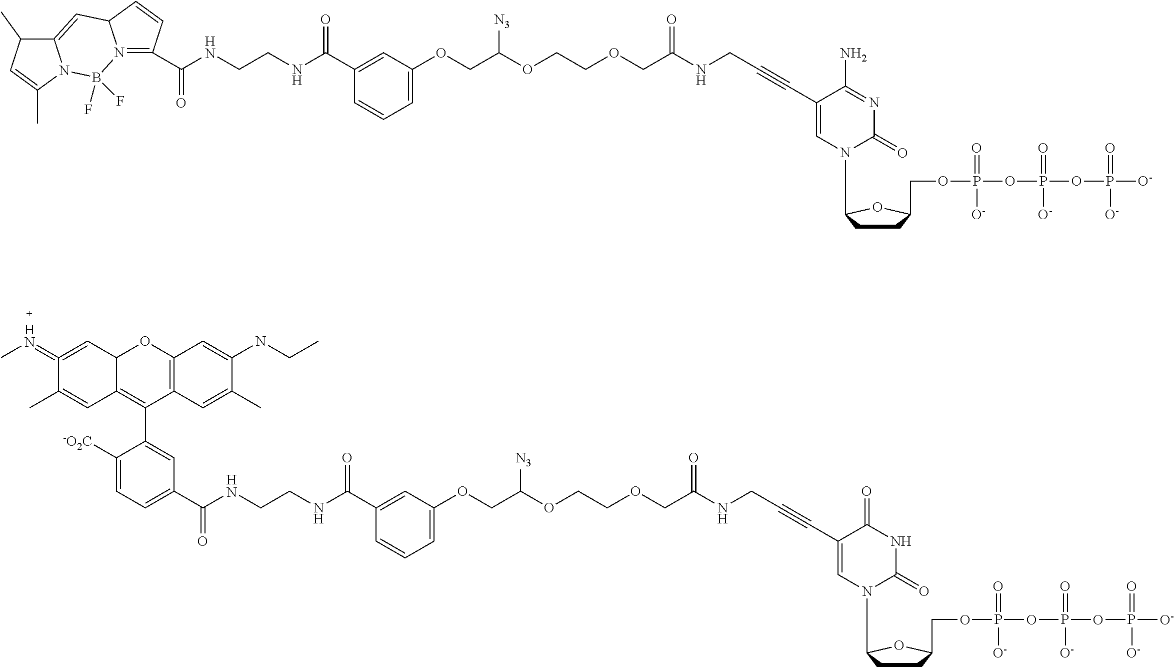

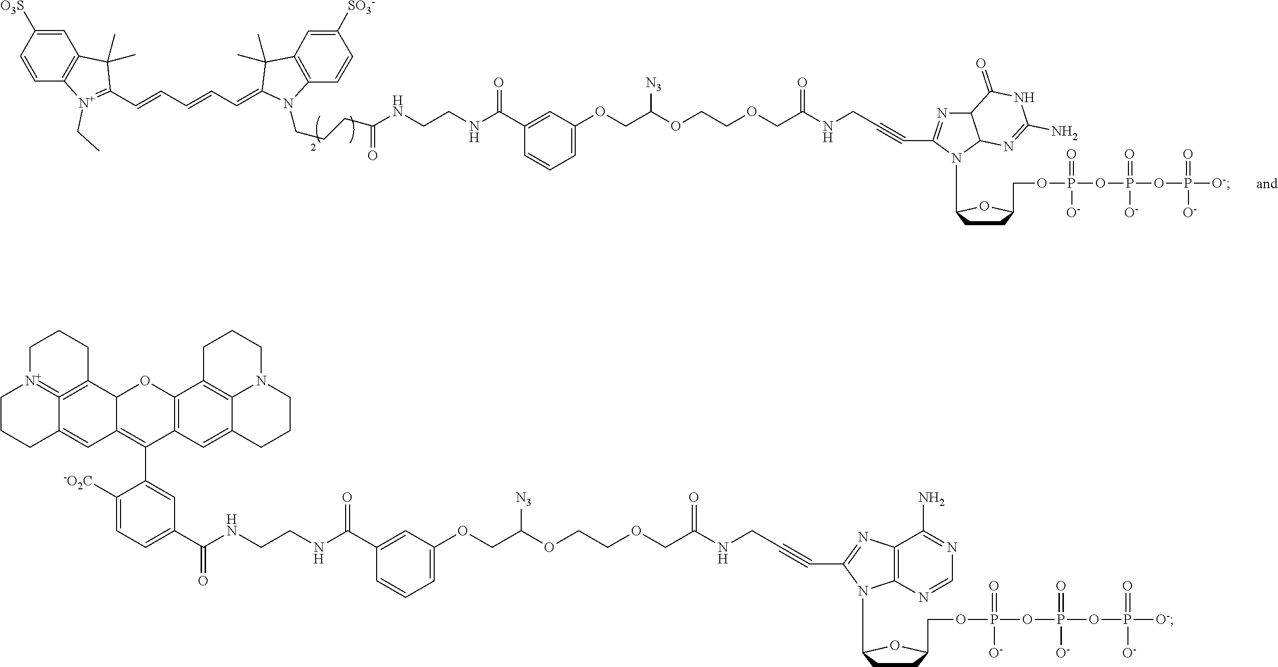

In certain embodiments, R′ comprises a methyl disulfide, an allyl, an azide, or nitrobenzyl moiety.

In certain embodiments, R′ is selected from:
H,

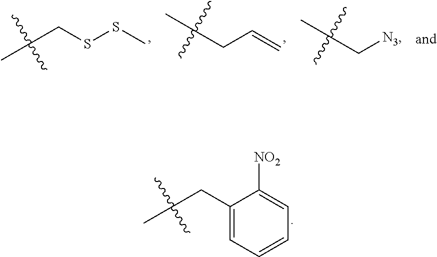

In a second aspect, the present disclosure provides a mirror-image nucleic acid polymerase comprising a sequence having at least 90% sequence identity to SEQ ID NO: 1, wherein the polymerase comprises D-form amino acids.

In certain embodiments, the polymerase consists of D-form amino acids.

In certain embodiments, the polymerase comprises a sequence having at least 95% sequence identity to SEQ ID NO: 1. In further embodiments, the polymerase comprises a sequence having at least 96%, 97%, 98% or 99% sequence identity to SEQ ID NO: 1.

In certain embodiments, the polymerase comprises one or more modifications at one or more amino acid sites selected from E276, K317, N424, and S651 compared to SEQ ID NO. 1.

In certain embodiments, the polymerase comprises one or more substitutions selected from E276A, K317G, N424A, and S651A compared to SEQ ID NO: 1.

In certain embodiments, the polymerase comprises E276A, K317G, N424A, and S651A substitutions compared to SEQ ID NO: 1.

In certain embodiments, the polymerase comprises one or more modifications at one or more amino acid sites selected from I80, I127, I171, I176, I191, I228, I256, I264, I268, I400, I597, I610, I618, I630, I642, I715, I733, and I744 compared to SEQ ID NO: 1.

In certain embodiments, the polymerase comprises one or more substitutions from Ile to Ala, Val, Leu, or Tyr at one or more amino acid sites selected from I80, I127, I171, I176, I191, I228, I256, I264, I268, I400, I597, I610, I618, I630, I642, I715, I733, and I744 compared to SEQ ID NO: 1.

In certain embodiments, the polymerase comprises one or more substitutions selected from I80V, I127V, I171A, I176V, I191V, I228V, I256V, I264A, I268L, I400V, I597V, 1610V, I618A, I630L, I642V, I715Y, I733V, and I744V compared to SEQ ID NO: 1.

In certain embodiments, the polymerase comprises substitutions of 180V, I127V, I171A, I176V, I191V, I228V, I256V, I264A, I268L, I400V, I597V, I610V, I618A, I630L, 1642V, I715Y, I733V, and 1744V compared to SEQ ID NO: 1.

In certain embodiments, the polymerase comprises one or more modifications at one or more amino acid sites selected from M129, I130, G131, D141, E143, L408, Y409, P410, A485, T514, and I521 compared to SEQ ID NO: 1.

In certain embodiments, the polymerase comprises one or more modifications at one or more amino acid sites selected from D141, E143, Y409, and A485 compared to SEQ ID NO: 1.

In certain embodiments, the polymerase comprises one or more substitutions selected from D141A, E143A, Y409V, and A485L compared to SEQ ID NO: 1.

In certain embodiments, the polymerase comprises substitutions of D141A, E143A, Y409V, and A485L compared to SEQ ID NO: 1.

In certain embodiments, the polymerase comprises one or more modifications at one or more amino acid sites selected from D141, E143, L408, Y409, P410, A485, T514, and I521 compared to SEQ ID NO: 1.

In certain embodiments, the polymerase comprises one or more substitutions selected from D141A, E143A, L408A, Y409A, P410I, A485V, T514S, and I521L compared to SEQ ID NO: 1.

In certain embodiments, the polymerase comprises substitutions of D141A, E143A, L408A, Y409A, P410I, A485V, T514S, and I521L compared to SEQ ID NO: 1.

In certain embodiments, the polymerase comprises one or more modifications selected from substitution of M129L, D141A, E143A, L408A, Y409A, P410I, A485V, T514S, I521L or addition of D between I130 and G131 compared to SEQ ID NO: 1.

In certain embodiments, the polymerase comprises substitutions of M129L, D141A, E143A, L408A, Y409A, P410I, A485V, T514S, and I521L and addition of D between I130 and G131 compared to SEQ ID NO: 1.

In certain embodiments, the polymerase comprises the sequence selected from SEQ ID NOs: 2-7.

In certain embodiments, the polymerase consists of the sequence selected from SEQ ID NOs: 2-7.

In a third aspect of the present disclosure, a method of replicating an L-polynucleotide is provided, the method comprising the step of: incubating a mixture comprising (i) the L-polynucleotide, (ii) an L-primer, (iii) L-dNTP, (iv) the polymerase of any one of claims 1-38, and (v) a buffer, thereby inducing replication of the L-polymerase.

In certain embodiments, the L-polynucleotide is DNA or RNA.

In certain embodiments, wherein the mixture comprises L-dATP, L-dGTP, L-dCTP, and L-dTTP.

In certain embodiments, the buffer comprises 50 mM Tris-HCl, pH 7.5, 20 mM MgCl₂, 1 mM DTT, and 50 mM KCl.

In certain embodiments, the incubation step comprises PCR.

In a fourth aspect of the present disclosure, a method of sequencing an L-polynucleotide is provided, the method comprising the cycle of:
a. incubating a mixture comprising (i) the L-polynucleotide, (ii) an L-primer, (iii) L-3′-O—R′-dNTP-R₂—Label, (iv) the polymerase of any one of claims 17-38, and (v) a buffer, thereby obtaining a replication product;
b. detecting a signal from the L-3′-O—R′-dNTP-R₂—Label incorporated into the replication product; and
c. inducing cleavage of the R′ group and R₂ group of the L-3′-O—R′-dNTP-R₂-Label incorporated into the replication product.

In certain embodiments, the cycle is repeated at least 3, 5, 10, 50, 100, 150, 200, 250, 300, 400, 500, or 1000 times.

In certain embodiments, the L-3′-O—R′-dNTP-R₂-Label comprises L-3′-O—R′-dATP-R₂-Label, L-3′-O—R′-dTTP-R₂-Label, L-3′-O—R′-dGTP-R₂-Label, and L-3′-O—R′-dCTP-R₂-Label, wherein each label is different. In certain embodiments, the L-3′-O—R′-dNTP-R₂-Label has a structure according to Formula II as defined in Section 5.2.2.1.2. In certain embodiments, the signal is a fluorescent signal.

In a fifth aspect of the present disclosure, a method of sequencing an L-polynucleotide is provided, the method comprising the steps of:
a. incubating a mixture comprising (i) the L-polynucleotide, (ii) an L-primer, (iii) L-dNTP, (iv) an L-ddNTP-R₂-Label or L-3′-O—R′-dNTP-R₂-Label, (v) the polymerase of any one of claims 17-38, and (vi) a buffer, thereby obtaining a replication product;
b. separating the replication product; and
c. detecting a signal from the L-ddNTP-R₂-Label or L-3′-O—R′-dNTP-R₂-Label incorporated into the replication product.

In certain embodiments, the L-dNTP comprises L-dATP, L-dTTP, L-dGTP, and L-dCTP. In certain embodiments, the L-ddNTP-R₂-Label comprises L-ddATP-R₂-Label, L-ddTTP-R₂-Label, L-ddGTP-R₂-Label, and L-ddCTP-R₂-Label, wherein each label is different.

In certain embodiments, the L-ddNTP-R₂-Label has Formula III as defined section 5.2.2.2.1. In certain embodiments, the L-3′-O—R′-dNTP-R₂-Label comprises L-3′-O—R′-dATP-R₂-Label, L-3′-O—R′-dTTP-R₂-Label, L-3′-O—R′-dGTP-R₂-Label, and L-3′-O—R′-dCTP-R₂-Label, wherein each label is different. In certain embodiments, the L-3′-O—R′-dNTP-R₂-Label has Formula II as defined in section 5.2.2.1.2. In certain of these embodiments, the signal is a fluorescent signal.

In certain of these embodiments, the incubation step comprises PCR. In certain of these embodiments, the separation step comprises separating the replication product by size.

In a sixth aspect of the present disclosure, a method of sequencing an L-polynucleotide is provided, the method comprising the cycle of:
a. incubating a mixture comprising (i) the L-polynucleotide, (ii) an L-primer, (iii) L-3′-O—R′-dNTP, (iv) L-ddNTPs-R₂-Label or L-dNTPs-R₂-Label, (v) the polymerase of any one of claims 17-38, and (vi) a buffer, thereby obtaining a replication product;
b. detecting a signal from the L-ddNTP-R₂-Label or L-3′-O—R′-dNTP-R₂-Label incorporated into the replication product, and
c. inducing cleavage of i) the R′ group of the L-3′-O—R′-dNTP and R₂ group of L-ddNTPs-R₂-Label incorporated into the replication product; or ii) the R′ group of the L-3′-O—R′-dNTP, R′ and R₂ group of the L-3′-O—R′-dNTP-R₂-Label incorporated into the replication product.

In certain embodiments, the cycle is repeated at least 3, 5, 10, 50, 100, 150, 200, 250, 300, 400, 500, or 1000 times.

In certain embodiments, the L-3′-O—R′-dNTP comprises L-3′-O—R′-dATP, L-3′-O—R′-dTTP, L-3′-O—R′-dGTP, and L-3′-O—R′-dCTP.

In certain embodiments, the L-3′-O—R′-dNTP has a structure according to Formula I as defined in Section 5.2.2.1.1.

In certain embodiments, the L-ddNTP-R₂-Label comprises L-ddATP-R₂-Label, L-ddTTP-R₂-Label, L-ddGTP-R₂-Label, and L-ddCTP-R₂-Label, wherein each label is different.

In certain embodiments, the L-ddNTP-R₂-Label has a structure according to Formula III as defined in Section 5.2.2.2.1. In certain embodiments, the L-3′-O—R′-dNTP-R₂-Label comprises L-3′-O—R′-dATP-R₂-Label, L-3′-O—R′-dTTP-R₂-Label, L-3′-O—R′-dGTP-R₂-Label, and L-3′-O—R′-dCTP-R₂-Label, wherein each label is different. In certain embodiments, the L-3′-O—R′-dNTP-R₂-Label has a structure according to has Formula II as defined in Section 5.2.2.1.2. In certain embodiments, the signal is a fluorescent signal.

In a sixth aspect of the present disclosure, a kit is provided for replication of an L-polynucleotide, the kit comprising (i) the polymerase of any one of the preceding embodiments and (ii) optionally, a buffer.

In certain embodiments, the kit comprises L-dNTP. In certain embodiments, the L-dNTP comprises L-dATP, L-dGTP, L-dCTP, L-dTTP or L-UTP.

In certain embodiments, the kit comprises L-3′-O—R′-dNTP-R₂-Label. In certain embodiments, the kit comprises L-3′-O—R′-dNTP-R₂-Label comprises L-3′-O—R′-dATP-R₂-Label, L-3′-O—R′-dTTP-R₂-Label, L-3′-O—R′-dGTP-R₂-Label, L-3′-O—R′-dCTP-R₂-Label or L-3′-O—R′-dUTP-R₂-Label. In certain embodiments, the kit comprises the L-3′-O—R′-dNTP-R₂-Label has Formula II as defined in section 5.2.2.1.2.

In certain embodiments, the kit comprises L-ddNTP-R₂-Label. In certain embodiments, the kit comprises the L-ddNTP-R₂-Label comprises L-ddATP-R₂-Label, L-ddTTP-R₂-Label, L-ddGTP-R₂-Label, L-ddCTP-R₂-Label or L-ddUTP-R₂-Label. In certain embodiments, the L-ddNTP-R₂-Label has Formula III as defined in Section 5.2.2.2.1.

In certain embodiments, the kit comprises L-3′-O—R′-dNTP. In certain embodiments, the L-3′-O—R′-dNTP comprises L-3′-O—R′-dATP, L-3′-O—R′-dTTP, L-3′-O—R′-dGTP, and L-3′-O—R′-dCTP. In certain embodiments, the L-3′-O—R′-dNTP has Formula I as defined in Section 5.2.2.1.1.

### 4. BRIEF DESCRIPTION OF THE DRAWINGS

These and other features, aspects, and advantages of the present invention will become better understood with regard to the following description, and accompanying drawings, where:

FIG. 1 provides the amino-acid sequence of (D)-form mutant 9°N DNA polymerase (candidate #1-1) (SEQ ID NO: 8). All the amino acids are D-form amino acids. Diamond=mutation introduced for NCL; Circle=NCL site (start of the ‘second’ strand); Square=potential substitution sites for isoleucines; Triangle=mutation introduced for modified nucleotides.

FIG. 2A shows the sequence of D-form 9° N DNA polymerase mutant 1-I, 9° N—N fragment (SEQ ID NO: 9), where sequences of nine synthetic peptides are presented in separate lines. FIG. 2B shows synthetic route for the D-form 9° N DNA polymerase mutant 1-I, 9° N—N fragment.

FIG. 3A shows the sequence of D-form 9° N DNA polymerase mutant 1-I, 9° N—C fragment (SEQ ID NO: 10), where sequences of six synthetic peptides are presented in separate lines. FIG. 3B shows synthetic route for the D-form 9° N DNA polymerase mutant 1-I, 9° N—C fragment.

FIG. 4A provides TLC staining of Tfa-(D)-Thz-OH, i.e. (S)-3-(2,2,2-trifluoroacetyl)thiazolidine-4-carboxylic acid). FIG. 4B provides ¹H NMR (500 MHz, CDCl₃) result of Tfa-(D)-Thz-OH, i.e. (S)-3-(2,2,2-trifluoroacetyl)thiazolidine-4-carboxylic acid).

FIG. 5 illustrates synthesis of 2-Cl-(Trt)-NHNH₂ as detailed in Example 13.

FIG. 6A is the peptide sequence of D-9° N—N-6@5-mer (SEQ ID NO: 11). FIG. 6B provides analytical HPLC chromatogram of the crude D-9° N—N-6@5-mer (λ=214 nm). Column: Welch C4. Gradient: 0-20 min 100-80% A in B, 20-40 min 80-50% A in B, 40-60 min 50-20% A in B, and 60-70 min 20-0% A in B. Where A=H₂O (0.1% TFA) and B=CH₃CN (0.1% TFA). FIG. 6C provides analytical HPLC chromatogram of the pure D-9° N—N-6@5-mer. Gradient: 95-5% CH₃CN (0.1% TFA) in H₂O (0.1% TFA) over 30 min. Purification conditions: Column: Welch C4 semipreparative column. Gradient: 1-20 min 100-80% A in B, 20-40 min 80-50% A in B, 40-50 min 50-20% A in B, and 50-55 min 20-0% A in B. Where A=H₂O (0.1% TFA) and B=CH₃CN (0.1% TFA).

FIG. 7A is the peptide sequence of D-9° N—N-6@0.11-mer (SEQ ID NO: 12); FIG. 7B provides analytical HPLC chromatogram of the crude D-9° N—N-6@11-mer (λ=214 nm). Column: Welch C4. Gradient: 95-5% CH₃CN (0.1% TFA) in H₂O (0.1% TFA) over 30 min; FIG. 7C provides analytical HPLC chromatogram of the pure D-Pfu-9° N—N-6@11-mer. Gradient: 95-5% CH₃CN (0.1% TFA) in H₂O (0.1% TFA) over 30 min. Purification conditions: Column: Welch C4 semipreparative column. Gradient: 1-20 min 100-80% A in B, 20-40 min 80-50% A in B, 40-50 min 50-20% A in B, and 50-55 min 20-0% A in B. Where A=H₂O (0.1% TFA) and B=CH₃CN (0.1% TFA); FIG. 7D provides MALDI-TOF mass spectrum of D-9° N—N-6@11-mer.

FIG. 8A is the peptide sequence of D-9° N—N-6@24-mer (SEQ ID NO: 13); FIG. 8B provides analytical HPLC chromatogram of the crude D-9° N—N-6@24-mer (λ=214 nm). Column: Welch C4. Gradient: 0-20 min 100-80% A in B, 20-40 min 80-50% A in B, 40-60 min 50-20% A in B, and 60-70 min 20-0% A in B. Where A=H₂O (0.1% TFA) and B=CH₃CN (0.1% TFA); FIG. 8C provides analytical HPLC chromatogram of the pure D-Pfu-9° N—N-6@5-mer. Gradient: 95-5% CH₃CN (0.1% TFA) in H₂O (0.1% TFA) over 30 min. Purification conditions: Column: Welch C4 semipreparative column. Gradient: 1-20 min 100-80% A in B, 20-40 min 80-50% A in B, 40-50 min 50-20% A in B, and 50-55 min 20-0% A in B. Where A=H₂O (0.1% TFA) and B=CH₃CN (0.1% TFA).

FIG. 9A is the peptide sequence of D-9° N—C-3@9-mer (SEQ ID NO: 14); FIG. 9B provides analytical HPLC chromatogram of the crude D-9° N—C-3@9-mer (λ=214 nm). Column: Welch C4. Gradient: 0-20 min 100-80% A in B, 20-40 min 80-50% A in B, 40-60 min 50-20% A in B, and 60-70 min 20-0% A in B. Where A=H₂O (0.1% TFA) and B=CH₃CN (0.1% TFA). Purification conditions: Column: Polaris C18-A semipreparative column. Gradient: 1-20 min 100-80% A in B, 20-40 min 80-50% A in B, 40-60 min 50-20% A in B, and 60-70 min 20-0% A in B. Where A=H₂O (0.1% TFA) and B=CH₃CN (0.1% TFA); FIG. 9C provides HRMS (ESI) spectrum of D-9° N—C-3@9-mer: [M+H]⁺ Calcd for C₃₆H₅₉N₁₃O₁₅S 946.4053. found 946.4064.

FIG. 10A provides analytical HPLC chromatogram of the purified D-9° N—C-1@33-mer (λ=214 nm). Column: Welch C4. Gradient: 0-20 min 75-35% A in B, 20-23 min 35-0% A in B. Where A=H₂O (0.1% TFA) and B=CH₃CN (0.1% TFA). b) ESI-MS spectrum of D-9° N—C-1@33-mer: Observed 3985.5, calculated 3985.6. FIG. 10B provides ESI-MS spectrum of D-9° N—C-1@33-mer.

FIG. 11A provides analytical HPLC chromatogram of the purified D-9° N—C-2@39-mer (λ=214 nm). Column: Welch C4. Gradient: 0-20 min 75-35% A in B, 20-23 min 35-0% A in B. Where A=H₂O (0.1% TFA) and B=CH₃CN (0.1% TFA). FIG. 11B provides ESI-MS spectrum of D-9° N—C-2@39-mer: Observed 4921.5, calculated 4921.6.

FIG. 12A provides peptide sequence of D-9° N—C-3@21-mer (SEQ ID NO: 15); FIG. 12B provides analytical HPLC chromatogram of the crude D-9° N—C-3@21-mer (λ=214 nm). Column: Welch C4. Gradient: 0-30 min 90-50% A in B, 30-40 min 50-5% A in B. Where A=H₂O (0.1% TFA) and B=CH₃CN (0.1% TFA). Purification conditions: Column: Polaris C18-A semipreparative column. Gradient: 0-30 min 90-50% A in B, 30-40 min 50-5% A in B. Where A=H₂O (0.1% TFA) and B=CH₃CN (0.1% TFA). FIG. 12C provides ESI-MS spectrum of D-9° N—C-3@21-mer. FIG. 12D provides the MS spectrum and observed mass 2196.1 of D-9° N—C-3@21-mer without TFA protection.

FIG. 13A is the peptide sequence of D-9° N—C-3@Cys35-mer (SEQ ID NO: 16); FIG. 13B provides analytical HPLC chromatogram of the crude D-9° N—C-3@Cys35-mer (λ=214 nm). Column: Welch C4. Gradient: 0-30 min 70-20% A in B, 30-40 min 20-5% A in B. Where A=H₂O (0.1% TFA) and B=CH₃CN (0.1% TFA). Purification conditions: Column: Polaris C18-A semipreparative column. Gradient: 0-30 min 70-20% A in B, 30-40 min 20-5% A in B. Where A=H₂O (0.1% TFA) and B=CH₃CN (0.1% TFA). FIG. 13C provides ESI-MS spectrum of D-9° N—C-3@21-mer; FIG. 13D provides observed mass of 4276 of D-9° N—C-3@Cys35-mer.

FIG. 14A is the peptide sequence of D-9° N—C-3@56-mer (SEQ ID NO: 17); FIG. 14B provides analytical HPLC chromatogram of the crude D-9° N—C-3@56-mer (λ=214 nm). Column: Welch C4. Gradient: 0-30 min 80-30% A in B, 30-40 min 30-5% A in B. Where A=H₂O (0.1% TFA) and B=CH₃CN (0.10% TFA). Purification conditions: Column: Polaris C18-A semipreparative column. Gradient: 0-30 min 80-30% A in B, 30-40 min 30-5% A in B. Where A=H₂O (0.1% TFA) and B=CH₃CN (0.10% TFA).

FIG. 15A is the peptide sequence of D-Pfu-9° N—C-3@35-mer (SEQ ID NO: 18); FIG. 15B provides analytical HPLC chromatogram of the crude D-Pfu-9° N—C-3@35-mer (λ=214 nm). Column: Welch C4. Gradient: 0-30 min 80-30% A in B, 30-40 min 30-5% A in B. Where A=H₂O (0.1% TFA) and B=CH₃CN (0.1% TFA). Purification conditions: Column: Polaris C18-A semipreparative column. Gradient: 0-30 min 80-30% A in B, 30-40 min 30-5% A in B. Where A=H₂O (0.1% TFA) and B=CH₃CN (0.1% TFA).

FIG. 16A is the peptide sequence of D-9° N—N-6@5-mer and D-9° N—N-6@0.11-mer (SEQ ID NO 19 and SEQ ID NO: 20); FIG. 16B provides synthetic route for the preparation of D-9° N—N-6@16-mer. D-9° N—N-6@5-mer (4 mg, 1 equiv.) was dissolved in 0.4 mL acidified ligation buffer (aqueous solution of 6 M Gn·HCl and 0.1 M NaH₂PO₄, pH 3.0). The mixture was cooled in ice-salt bath (−15° C.), and 40 μl 0.5 M NaNO₂ (in acidified ligation buffer) was added. The reaction was kept in ice-salt bath under stirring for 15 min, after which 0.4 ml 0.2 M MPAA (in 6 M Gn·HCl and 0.1 M Na₂HPO₄, pH 5.7) was added. After the addition of D-Pfu-9° N—N-6@11-mer (7.5 mg), the pH of the reaction mixture was adjusted to 6.6-6.8 with NaOH solution at room temperature. After 12 h, the reaction mixture was reduced by TCEP and purified by HPLC (purification conditions: 5-95% CH₃CN (with 0.1% TFA) gradient in H₂O (with 0.1% TFA) over 30 min on a Welch C4 column). The ligation product was obtained with a yield of 80% (9.0 mg); FIG. 16C provides analytical HPLC chromatogram of the peaks transformation in progressing the NCL reaction (λ=214 nm). Column: Welch C4. Gradient: 5-95% CH₃CN (with 0.1% TFA) in H₂O (with 0.1% TFA) over 30 min.

FIG. 17A is the peptide sequence of D-9° N—C-1 and D-9° N—C-2 (SEQ ID NO: 21, 22, 23); FIG. 17B provides the synthetic route for the preparation of D-9° N—C-7@72-mer. D-9° N—C-1@33-mer (5.4 mg) was dissolved in 0.27 mL acidified ligation buffer (aqueous solution of 6 M Gn·HCl and 0.1 M NaH₂PO₄, pH 3.0). The mixture was cooled in ice-salt bath (−15° C.), and 27 μl 0.5 M NaNO₂ (in acidified ligation buffer) was added. The reaction was kept in ice-salt bath under stirring for 20 min, after which 0.14 ml 0.4 M MPAA (in 8 M Gn·HCl and 0.1 M Na₂HPO₄, pH 5.7) was added. After the addition of D-9° N—C-2@39-mer (5.8 mg), the pH of the reaction mixture was adjusted to 6.6-6.8 with NaOH solution at room temperature. After 16 h the mixture was purified by HPLC (purification conditions: 20-70% CH₃CN (with 0.1% TFA) gradient in H₂O (with 0.1% TFA) over 30 min on a Polaris C18 column. The ligation product was obtained. FIG. 17C is the analytical HPLC chromatogram of the peaks transformation in progressing the NCL reaction (λ=214 nm). Column: Welch C4. Gradient: 20-70% CH₃CN (with 0.1% TFA) in H₂O (with 0.1% TFA) over 30 min. FIG. 17D is ESI-MS of D-9° N—C-7@72-mer with MPAA attachment; FIG. 17E shows MPAA-attached ligated product (Observed mass: 9042; calculated mass: 9042).

### 5. DETAILED DESCRIPTION

### 5.1. Definitions

“L-Nucleic acid” or “L-nucleotide” shall mean any nucleic acid or nucleotide molecule having L chirality, including, without limitation, L-DNA, L-RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, New Jersey, USA).

The term “D-polymerase” as used herein refers to a protein having a mirror form of an original polymerase having L-chirality. The original polymerase can be a polymerase obtained or modified from nature or synthetically created. The original polymerase can be a DNA or RNA polymerase.

Although the base is usually referred to as a purine or pyrimidine, the skilled person will appreciate that derivatives and analogs are available which do not alter the capability of the nucleotide or nucleoside to undergo Watson-Crick base pairing. As used herein, the term “base” includes a compound or molecule whose core structure is the same as, or closely resembles that of, a natural base, but which has a chemical or physical modification, such as a different or additional side groups, which allows the derivative nucleotide or nucleoside to be linked to another molecule. For example, the base can be a deazapurine.

“Hybridize” shall mean the annealing of one single-stranded nucleic acid to another nucleic acid based on sequence complementarity. The propensity for hybridization between nucleic acids depends on the temperature and ionic strength of their milieu, the length of the nucleic acids and the degree of complementarity. The effect of these parameters on hybridization is well known in the art (see Sambrook J, Fritsch E F, Maniatis T. 1989. Molecular cloning: a laboratory manual. Cold Spring Harbor Laboratory Press, New York.)

Dehybridize is understood by those skilled in the art to mean to disassociate the hybridized primer (or extended strand thereof) from the target nucleic acid without destroying the target nucleic acid and thus permitting further hybridization of a second primer to the target nucleic acid. Hybridization as used herein in one embodiment means stringent hybridization, for examples as described in Sambrook, J., Russell, D. W., (2000) Molecular Cloning: A Laboratory Manual: Third Edition.

As used herein, hybridization of a primer sequence shall mean annealing sufficient such that the primer is extendable by creation of a phosphodiester bond.

Where a range of values is provided, it is understood that each intervening value, to the tenth of the unit (if appropriate) of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

### 5.2. Mirror Image Nucleotides for Use in Sequencing

### 5.2.1. L-Nucleotide and L-Nucleosides

In one aspect, the disclosure provides mirror image nucleosides or nucleotides that are modified to include at least one of (i) a cleavable protecting group on a 3′ O of the pentose ring (if present) and (ii) a cleavable label. The mirror image nucleosides or nucleotides can be used for sequencing reactions, polynucleotide synthesis, nucleic acid amplification, nucleic acid hybridization assays, single nucleotide polymorphism studies, techniques using enzymes such as polymerase, reverse transcriptase, terminal transferase, techniques that use Labelled dNTPs (e.g., nick translation, random primer labeling, end-labeling (e.g., with terminal deoxynucleotidyltransferase), reverse transcription, or nucleic acid amplification.

In embodiments of the invention, a chemically modified mirror image nucleoside or nucleotide is provided that comprises at least one of (i) a cleavable protecting group on a 3′ O of the pentose sugar if present and (ii) a cleavable label. In embodiments where the mirror image nucleotide comprises a cleavable label, the label is not particularly limited, provided that the label provides means for detection and R′ includes H.

### 5.2.1.1 Base

In certain embodiments, the nitrogenous base is a purine, or a pyrimidine. In certain embodiments, the nitrogenous base is a deazapurine. In certain embodiments, the nitrogenous base is selected from thymine (5-methyl-2,4-dioxipyrimidine), cytosine (2-oxo-4-aminopyrimidine), 5-methyl-cytosine (2-oxo-5-methyl-4-aminopyrimidine), and uracil (2,4-dioxoypyrimidine), adenine (6-aminopurine), 7-deaza adenine (7H-Pyrrolo[2,3-d]pyrimidin-4-amine: 6-Amino-7-deazapurine), guanine (2-amino-6-oxypurine), 7-deaza guanine (2-Amino-4-hydroxy-pyrrolo-[2,3-d]-pyrimidine; 2-amino-7deaza-6-oxypurine), hypoxanthine (1,9-Dihydro-6H-purin-6-one), Xanthine (3,7-Dihydro-1H-purine-2,6-dione) and 5-nitroindole. In certain embodiments, the nitrogenous base is thymine, uracil, cytosine, adenine, guanine, 7-deaza adenine or 7-deaza guanine.

### 5.2.1.2 R′—Cleavable Protecting Groups

In certain embodiments, R′ is selected from H and a cleavable protecting group. The cleavable protecting groups of the invention are not particularly limited, as long as the resulting nucleotides are efficient substrates for the mirror image DNA polymerase.

The skilled person will appreciate how to attach a suitable protecting group to the pentose ring to block interactions with the 3′-OH. The protecting group can be attached directly at the 3′ position or can be attached at the 2′ position (the protecting group being of sufficient size or charge to block interactions at the 3′ position). Alternatively, the protecting group can be attached at both the 3′ and 2′ positions and can be cleaved to expose the 3′ OH group.

Suitable protecting groups will be apparent to the skilled person and can be formed from any suitable protecting group disclosed in Greene & Wuts, Protective Groups in Organic Synthesis, John Wiley & Sons. The protecting group should be removable (or modifiable) to produce a 3′ OH group. The process used to obtain the 3′ OH group can be any suitable chemical or enzymic reaction.

In some embodiments, a cleavable protecting group comprises an allyl, nitrobenzyl, 1-methyl-2-alkyl disulfide or methyl azido moiety.

In particular embodiments, the cleavable protecting group is selected from:

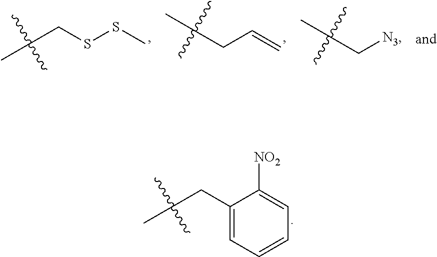

where the squiggly line demarks the point of attachment to the 3′O.

### 5.2.1.3 Cleavable Label Constructs (R₂-Label)

### 5.2.1.3.1 Labels

The present invention can make use of conventional detectable labels that can be modified to covalently attach to a nucleotide via a cleavable linker.

Detection can be carried out by any suitable method, including fluorescence spectroscopy or by other optical means. A particularly contemplated label is a fluorophore, which, after absorption of energy, emits radiation at a defined wavelength. Many suitable fluorescent labels are known. For example, Welch et al. (Chem. Eur. J. 5(3):951-960, 1999) discloses dansyl-functionalised fluorescent moieties that can be used in the present invention. Zhu et al. (Cytometry 28:206-211, 1997) describes the use of the fluorescent labels Cy3 and Cy5, which can also be used in the present invention. Labels suitable for use are also disclosed in Prober et al. (Science 238:336-341, 1987); Connell et al. (BioTechniques 5(4):342-384, 1987), Ansorge et al. (Nucl. Acids Res. 15(11):4593-4602, 1987) and Smith et al. (Nature 321:674, 1986). Other commercially available labels include, but are not limited to: ATTO-dyes (e.g., Atto 655 and Atto 647N), Quasar-dyes, CF-dyes, fluorescein, rhodamine (including TMR, texas red and Rox), Alexa Fluor®647, 488, 532, 594, 633, Dyomics-dyes, bodipy, acridine, R6G, Cy3, Cy3.5, Cy5, Cy5.5, coumarin, pyrene, benzanthracene and the cyanins.

Although fluorescent labels are particularly contemplated, other forms of detectable labels will be apparent as useful to those of ordinary skill. For example, microparticles, including quantum dots (Empodocles, et al., Nature 399:126-130, 1999), gold nanoparticles (Reichert et al., Anal. Chem. 72:6025-6029, 2000), microbeads (Lacoste et al., Proc. Natl. Acad. Sci USA 97(17):9461-9466, 2000), and tags detectable by changes in pH and voltage, spectrometry such as mass spectroscopy, Raman spectroscopy and surface plasmon resonance (SPR) sensing can all be used.

i. Fluorophores

In certain embodiments of the L-nucleotides of the present disclosure, the label is a fluorophore selected from: Rox, bodipy, bodipy-FL-510, R6G and Cy5 and functional derivatives thereof.

In particular embodiments, the fluorophore is selected from:

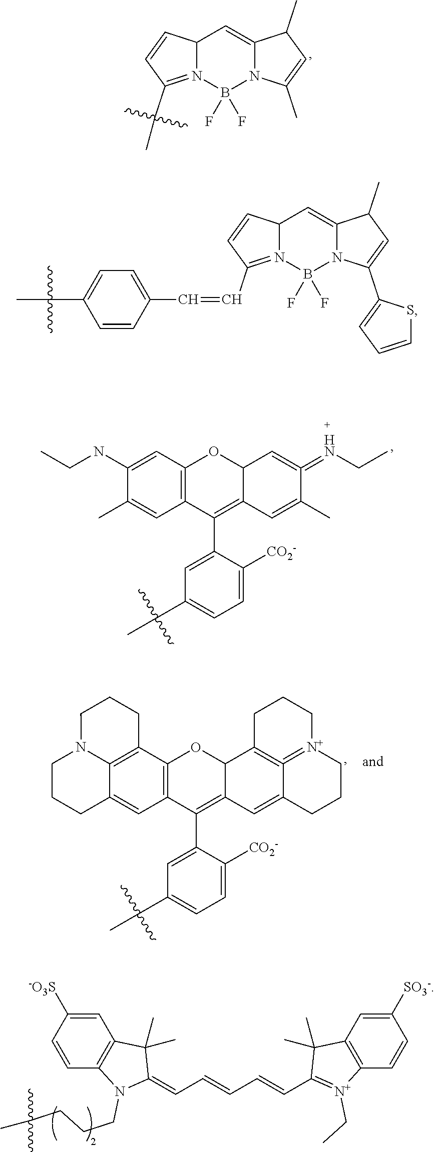

### 5.2.1.3.2 Cleavable Linkers (R₂)

Certain embodiments of the L-nucleotides of the present disclosure comprise a cleavable label linked to, e.g., the nitrogenous base, the 3′ O, or the 5′ phosphate group through a cleavable linker. In certain embodiments, the linker is attached to the nitrogenous base. In certain embodiments, the linker is attached to C8 of a purine base, the C7 of a 7-deaza purine base, or to the C5 of a pyrimidine base.

In certain embodiments, the cleavable linker is photocleavable, is cleaved by contact with water-soluble phosphines, or is cleaved by water-soluble transition metal-containing catalysts.

Suitable linkers known to those of skill in the art include, but are not limited to, disulfide linkers, acid labile linkers (including dialkoxybenzyl linkers, Sieber linkers, indole linkers, t-butyl Sieber linkers, electrophilically cleavable linkers, nucleophilically cleavable linkers, photocleavable linkers, cleavage under reductive conditions, oxidative conditions, cleavage via use of safety-catch linkers, and cleavage by elimination mechanisms.

i. Electrophilically Cleaved Linkers.

Electrophilically cleaved linkers are typically cleaved by protons and include cleavages sensitive to acids. Suitable linkers include the modified benzylic systems such as trityl, p-alkoxybenzyl esters and p-alkoxybenzyl amides. Other suitable linkers include tert-butyloxycarbonyl (Boc) groups and the acetal system.

The use of thiophilic metals, such as nickel, silver or mercury, in the cleavage of thioacetal or other sulphur-containing protecting groups can also be considered for the preparation of suitable linker molecules.

ii. Nucleophilically Cleaved Linkers.

Nucleophilic cleavage is also a well-recognized method in the preparation of linker molecules. Groups such as esters that are labile in water (i.e., can be cleaved simply at basic pH) and groups that are labile to non-aqueous nucleophiles, can be used. Fluoride ions can be used to cleave silicon-oxygen bonds in groups such as triisopropyl silane (TIPS) or t-butyldimethyl silane (TBDMS).

ii. Photocleavable Linkers.

Photocleavable linkers have been used widely in carbohydrate chemistry. It is preferable that the light required to activate cleavage does not affect the other components of the modified nucleotides. For example, if a fluorophore is used as the label, it is preferable if this absorbs light of a different wavelength to that required to cleave the linker molecule. Suitable linkers include those based on O-nitrobenzyl compounds and nitroveratryl compounds. Linkers based on benzoin chemistry can also be used (Lee et al., J. Org. Chem. 64:3454-3460, 1999).

iv. Cleavage Under Reductive Conditions

There are many linkers known that are susceptible to reductive cleavage. Catalytic hydrogenation using palladium-based catalysts has been used to cleave benzyl and benzyloxycarbonyl groups. Disulphide bond reduction is also known in the art.

v. Cleavage Under Oxidative Conditions

Oxidation-based approaches are well known in the art. These include oxidation of p-alkoxybenzyl groups and the oxidation of sulphur and selenium linkers. The use of aqueous iodine to cleave disulphides and other sulphur or selenium-based linkers is also within the scope of the invention.

vi. Safety-Catch Linkers

Safety-catch linkers are those that cleave in two steps. In a preferred system the first step is the generation of a reactive nucleophilic center followed by a second step involving an intra-molecular cyclization that results in cleavage. For example, levulinic ester linkages can be treated with hydrazine or photochemistry to release an active amine, which can then be cyclized to cleave an ester elsewhere in the molecule (Burgess et al., J. Org. Chem. 62:5165-5168, 1997).

vii. Cleavage by Elimination Mechanisms

Elimination reactions can also be used. For example, the base-catalyzed elimination of groups such as Fmoc and cyanoethyl, and palladium-catalyzed reductive elimination of allylic systems, can be used.

### 5.2.1.3.3 Spacers

As well as the cleavage site, the linker can comprise one or more a spacer moiety. A spacers (linkers and bridges) can be any typically used in, e.g., the synthesis of bioconjugates. The spacer distances the nucleotide base from the cleavage site or label. The length of the linker is unimportant provided that the label is held a sufficient distance from the nucleotide so as not to interfere with any interaction between the nucleotide and an enzyme.

In particular embodiments, the cleavable linker comprises an allyl, disulfide or azido group along with additional spacers. In certain embodiments, the cleavable linker comprises:

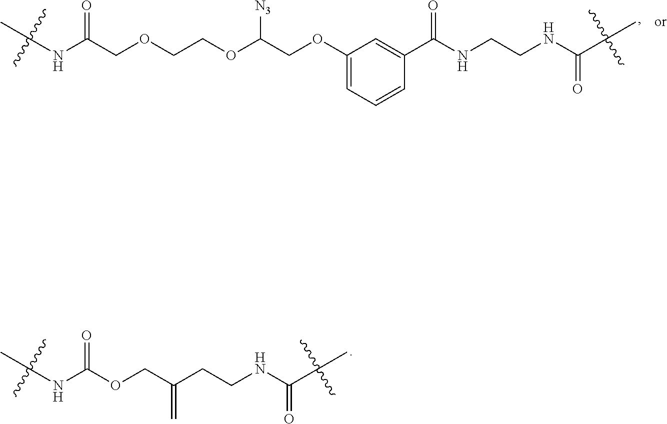 [SMILES: *NC(=O)COCCOC(COc1cccc(C(=O)NCCNC(*)=O)c1)N=[N+]=[N-] ; *NC(=O)OCC(=C)CCNC(*)=O]

### 5.2.1.3.4 Cleavable Label Constructs

In certain embodiments of the mirror image nucleosides or nucleotides provided herein, the cleavable label comprises a label as described in section 5.2.1.3.1 and a cleavable linker in more detail in section 5.2.1.3.2.

In particular embodiments, the cleavable label construct is selected from:

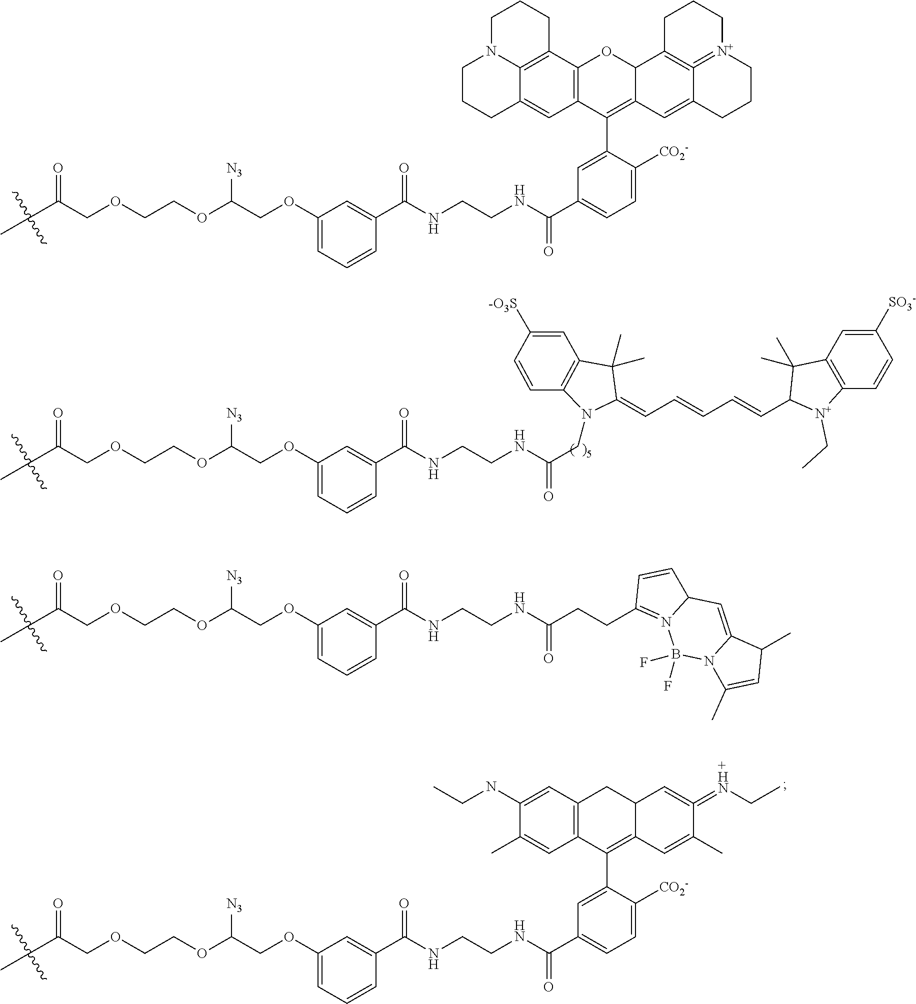

wherein the squiggly line demarks the point of attachment to the mirror image nucleotide, including attachment to a linker/spacer attached to a nucleotide for purposes of enabling attachment, e.g., an amine moiety.

### 5.2.2. Embodiments of the Present Disclosure

In certain embodiments, an L-nucleoside or L-nucleotide are provided comprising:
a pentose sugar selected from a (3R,4S)-3,4,5-trihydroxypentanal and a (4R)-4,5-dihydroxypentanal;
   wherein the C-5 of the sugar is substituted by a hydroxyl, monophosphate, diphosphate or triphosphate; and
   the C-3 of the sugar is substituted by H, or O—R′ where R′ is H or a cleavable protecting group;
a nitrogenous base; and
optionally a cleavable label;
   wherein the L-nucleoside or L-nucleotide comprises at least one selected from (i) a cleavable protecting group and (ii) a cleavable label.

Embodiments provided herein will be further described with reference to nucleotides. However, unless indicated otherwise, the reference to nucleotides is also intended to be applicable to nucleosides.

In certain embodiments, an L-nucleotide is provided having a structure according to Formula A:

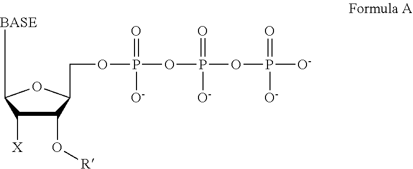

wherein R′ is H or a cleavable protecting group; X is H or hydroxyl, BASE is a nitrogenous base, and wherein the nucleotide optionally comprises a cleavable label, and wherein at least one of a cleavable protecting group or a cleavable label is present.

In certain embodiments, the nitrogenous base is as described in Section 5.2.1.1 herein. In certain embodiments, the cleavable protecting group is as described in Section 5.2.1.2 herein. In certain embodiments, the cleavable label is as described in Section 5.2.1.3 herein.

In any of the described embodiments, the location of the cleavable label is not particularly limited and may be linked to the nucleotide through any position chemically feasible and/or commonly known in the art. In certain embodiments, the cleavable label is attached to the nitrogenous base, through a terminal phosphate, or at the 3′ O in which case the cleavable protecting group is a cleavable label.

### 5.2.2.1 (L)—Nucleotide Reversible Terminator (L-NRT)

### 5.2.2.1.1 [(L)-3′-O—R′-dNTPs]—Formula I

In an aspect of the present disclosure, an L-nucleotide is provided having a structure according to Formula I:

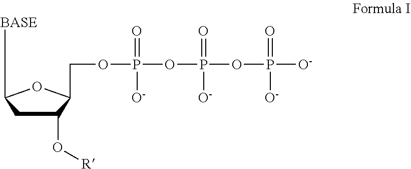

wherein Base is a nitrogenous base, and R′ is a cleavable protecting group.

In certain embodiments, the nitrogenous base is as described in Section 5.2.1.1 herein. In certain embodiments, the cleavable protecting group is as described in Section 5.2.1.2 herein.

In certain embodiments, an L-nucleotide is provided having a structure according to:

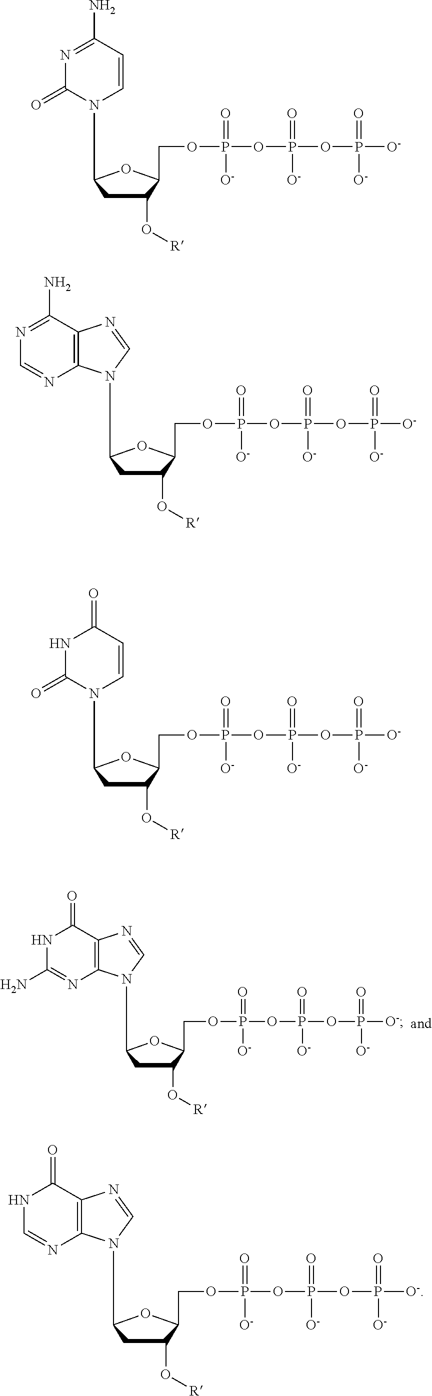

In certain embodiments, R′ comprises a cleavable protecting group as described in Section 5.2.1.2 herein. In further embodiments, the cleavable protecting group, R′, is selected from:

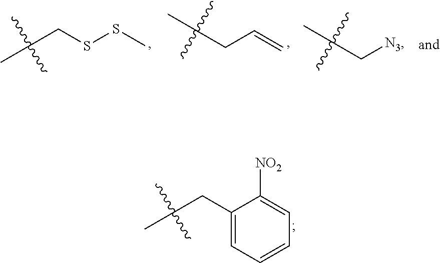

where the squiggly line demarks the point of attachment to the 3′O.

### 5.2.2.1.2 [(L)-3′-O—R′-dNTP-R₂-Label]—Formula II

In an aspect of the present disclosure, an L-nucleotide is provided having a structure according to Formula II:

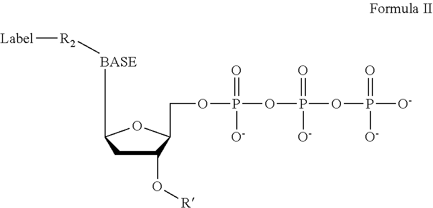

wherein Base is a nitrogenous base; R′ is a cleavable protecting group or H; and Label-R₂ together comprise a cleavable label construct.

In certain embodiments, the nitrogenous base is as described in Section 5.2.1.1 herein. In certain embodiments, the cleavable protecting group is as described in Section 5.2.1.2 herein. In certain embodiments, the cleavable label construct is as described in Section 5.2.1.3 herein.

In certain embodiments, an L-nucleotide is provided having a structure selected from:

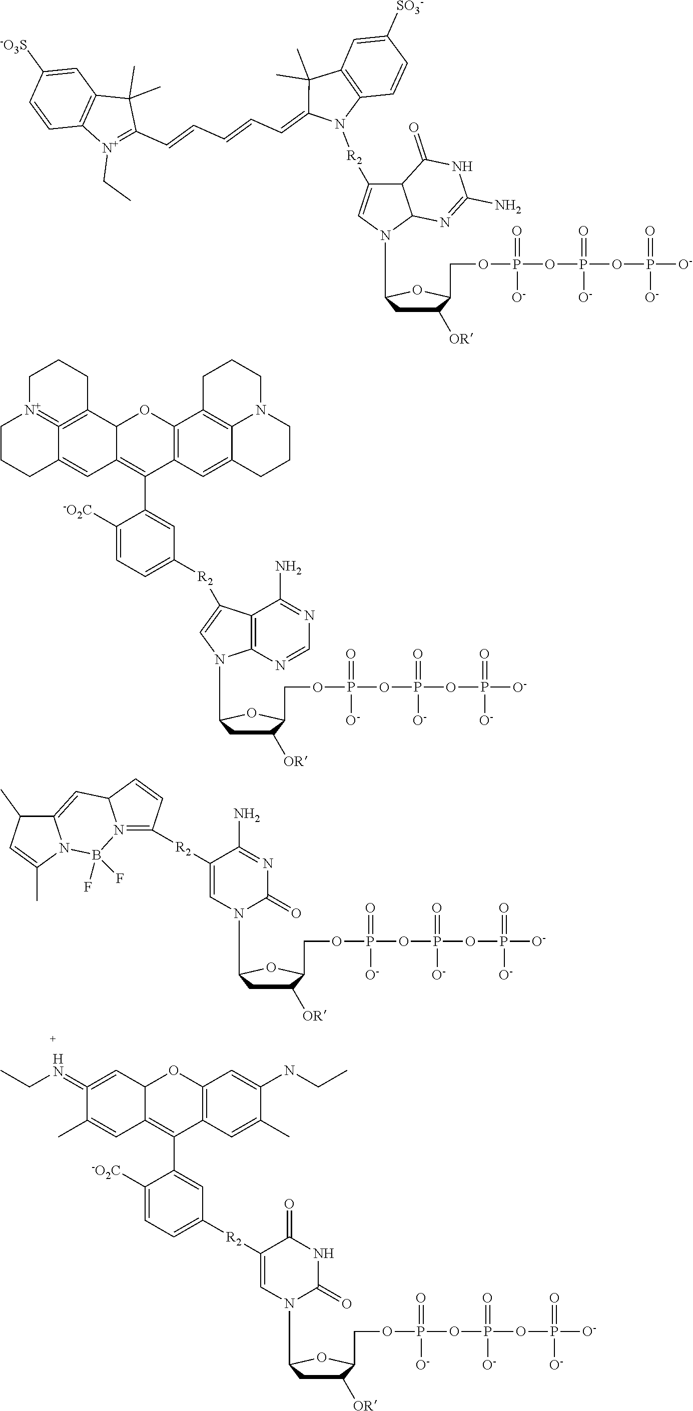

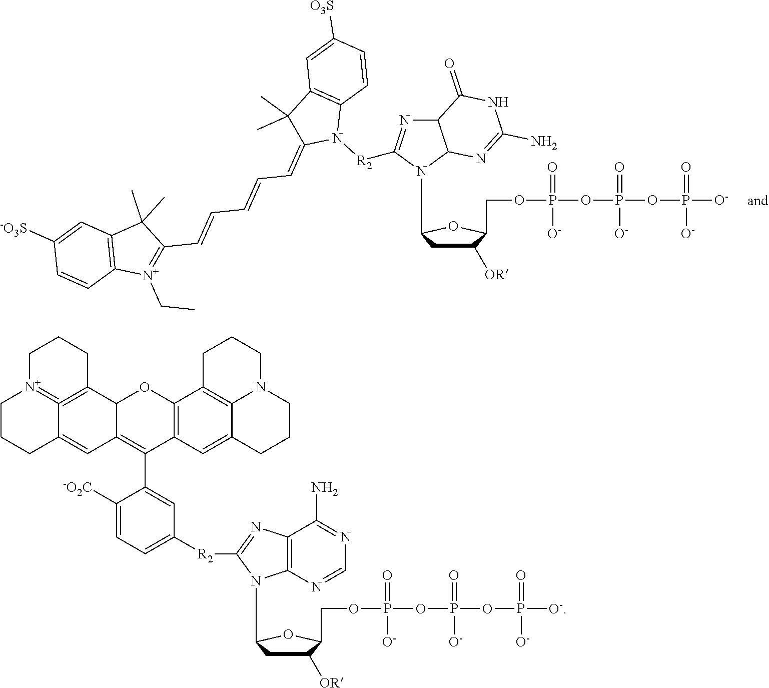

In certain embodiments, R′, the cleavable protecting group is as described in Section 5.2.1.2 herein. In certain embodiments, R₂, the cleavable linker, is as described in Section 5.2.1.3.2 herein.

In particular embodiments, R′ is selected from:

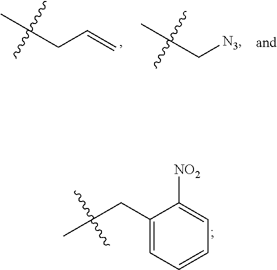

where the squiggly line demarks the point of attachment to the 3′O.

In particular embodiments, R₂ comprises:

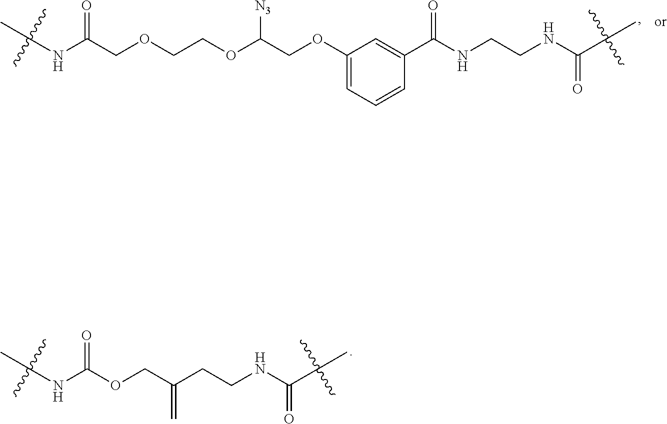 [SMILES: *NC(=O)COCCOC(COc1cccc(C(=O)NCCNC(*)=O)c1)N=[N+]=[N-] ; *NC(=O)OCC(=C)CCNC(*)=O]

In particular embodiments, an L-nucleotide is provided having a structure selected from:

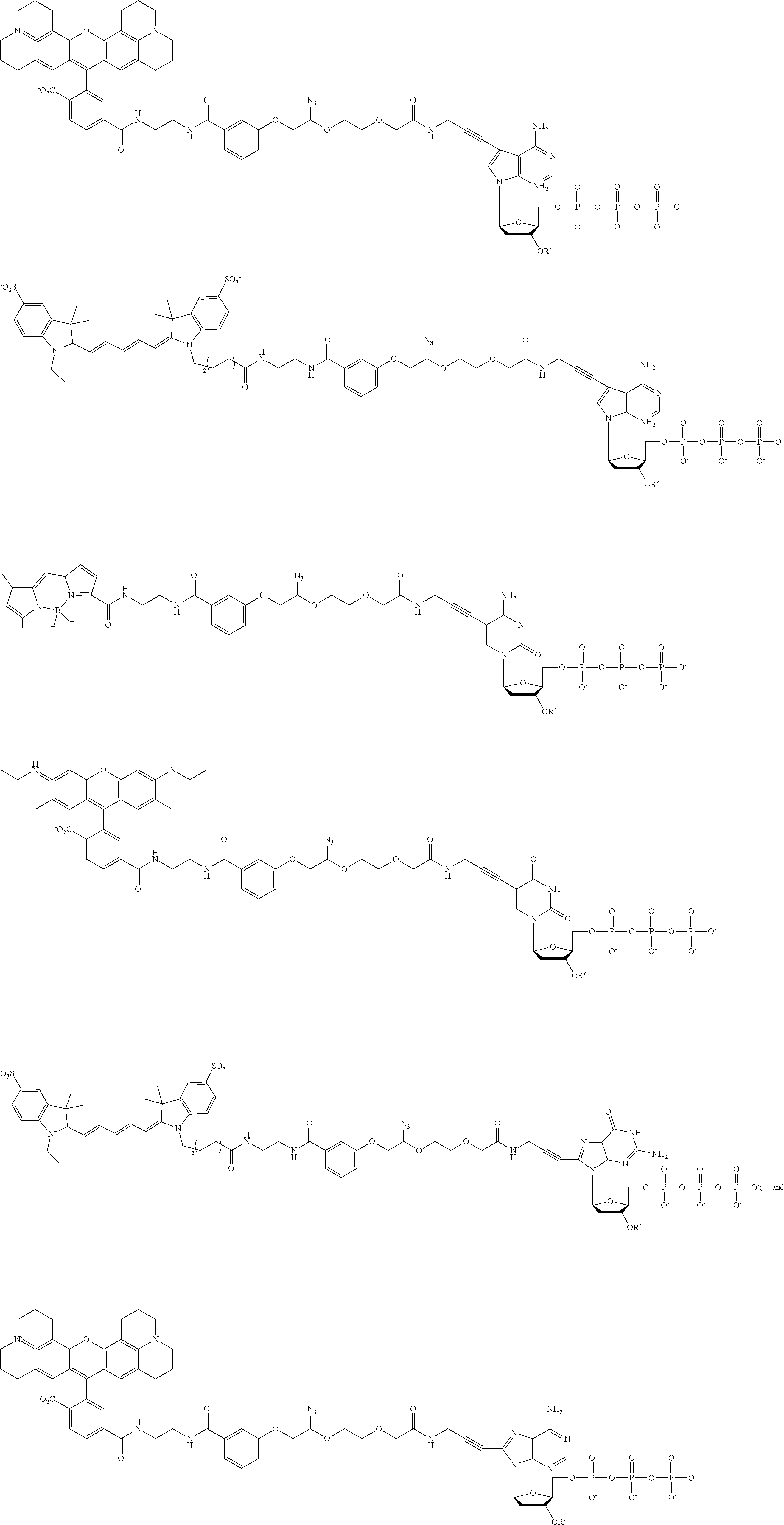

wherein R′ is H or a cleavable protecting group is as described in Section 5.2.1.2 herein.

In certain embodiments, R′ is selected from: H,

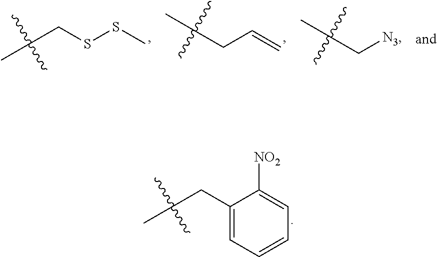

where the squiggly line demarks the point of attachment to the 3′O.

### 5.2.2.2 Labeled Terminator

### 5.2.2.2.1 [(L)-ddNTP-R₂-Label] Formula III

In an aspect of the present disclosure, an L-nucleotide is provided having a structure according to Formula III:

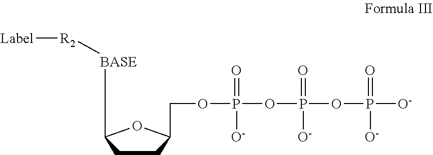

wherein Base is a nitrogenous base; R₂ is a cleavable linker; and Label-R₂ together comprise a cleavable label construct.

In certain embodiments, the nitrogenous base is as described in Section 5.2.1.1 herein. In certain embodiments, Label-R₂, the cleavable label construct, is as described in Section 5.2.1.3 herein.

In certain embodiments, an L-nucleotide is provided having a structure selected from:

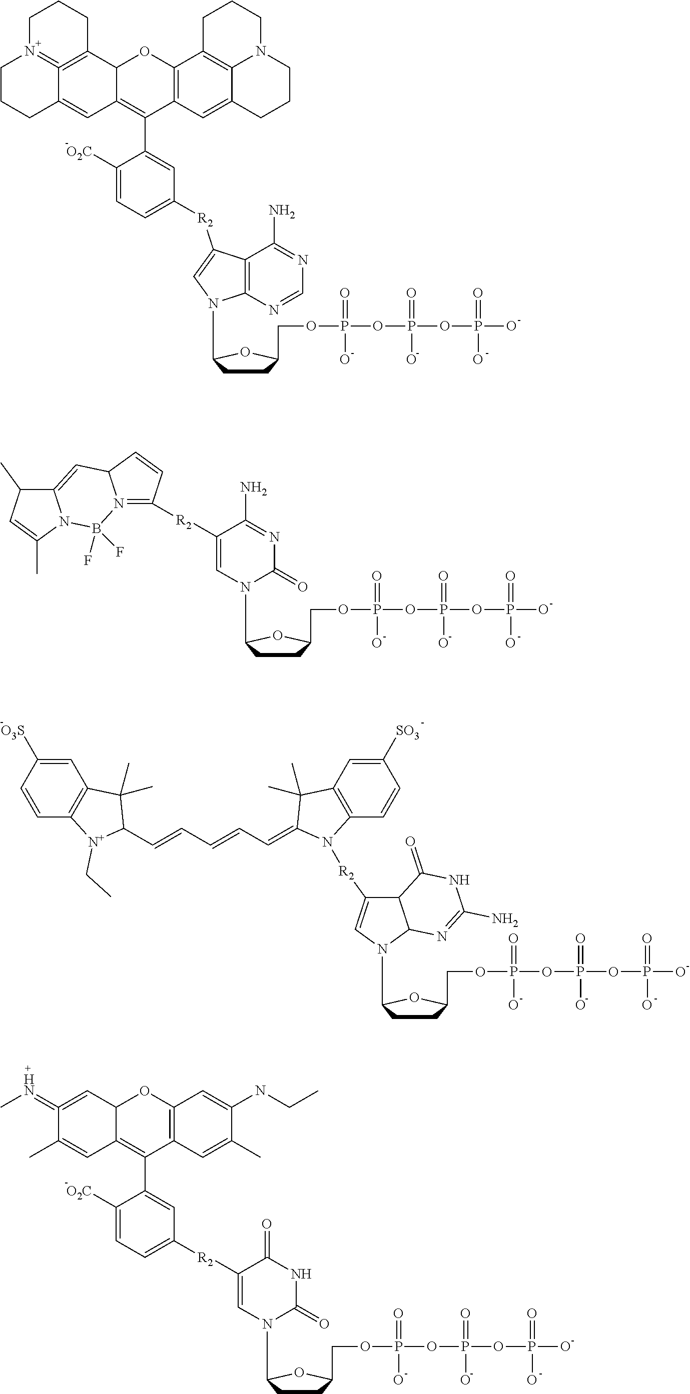

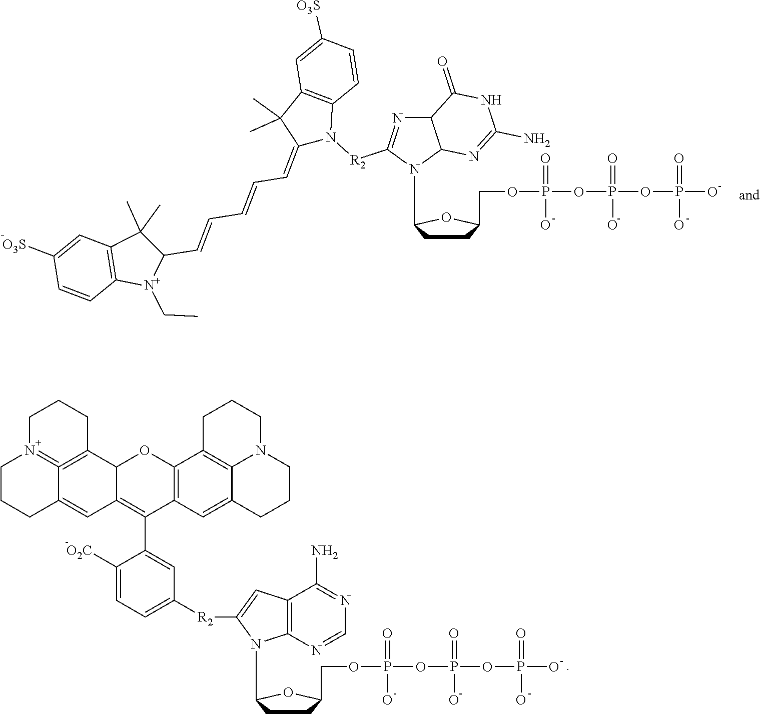

In certain embodiments, R₂, the cleavable linker is as described in Section 5.2.1.3.2 herein.

In particular embodiments, R₂ comprises:

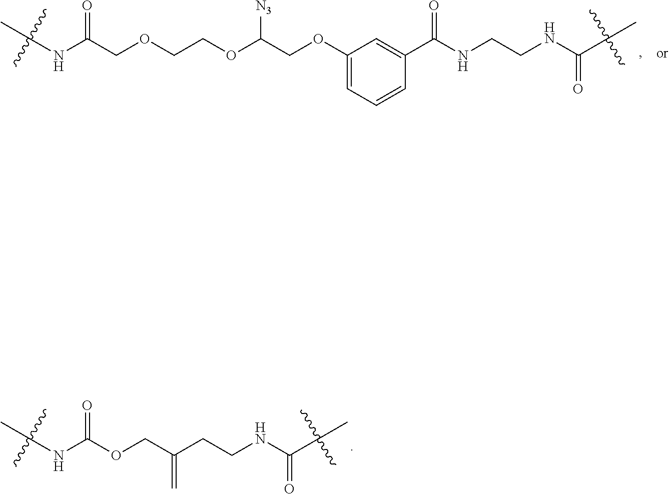 [SMILES: *NC(=O)COCCOC(COc1cccc(C(=O)NCCNC(*)=O)c1)N=[N+]=[N-] ; *NC(=O)OCC(=C)CCNC(*)=O]

In particular embodiments, an L-nucleotide is provided having a structure selected from:

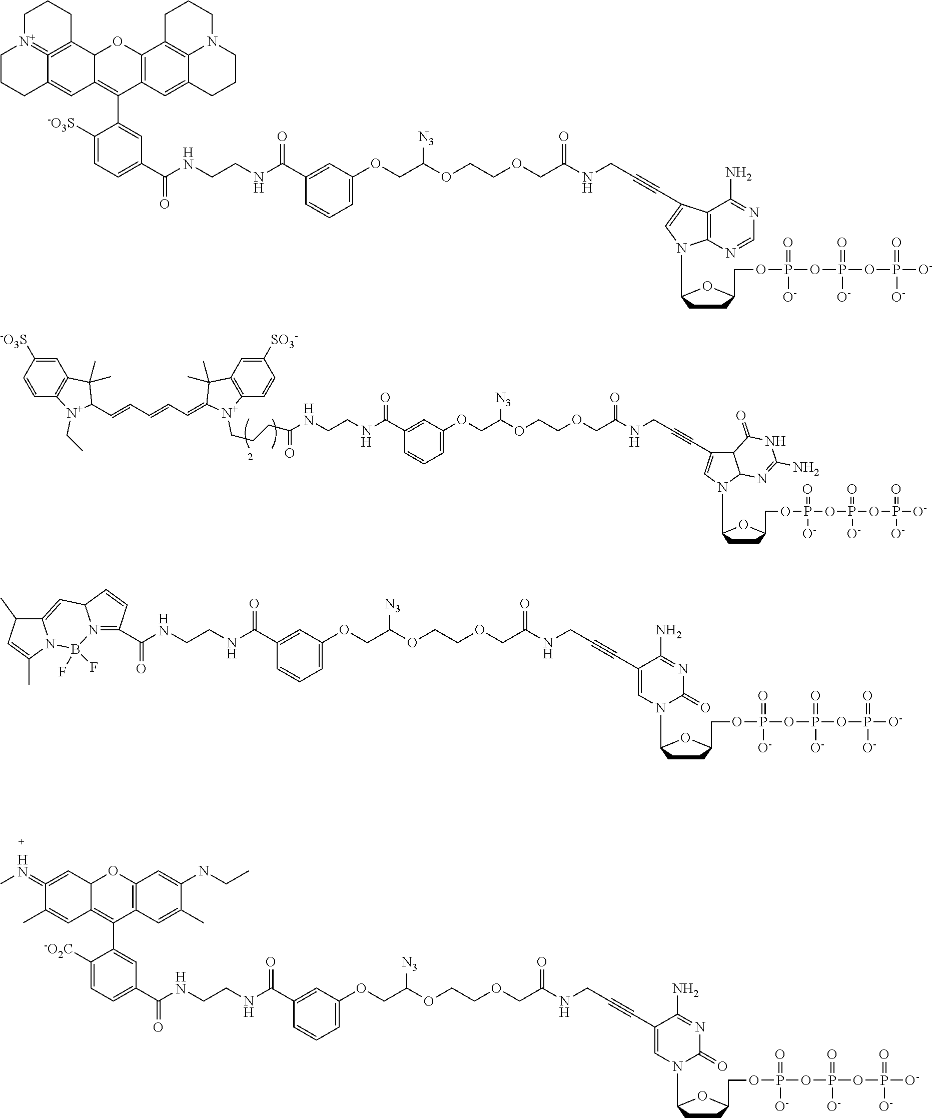

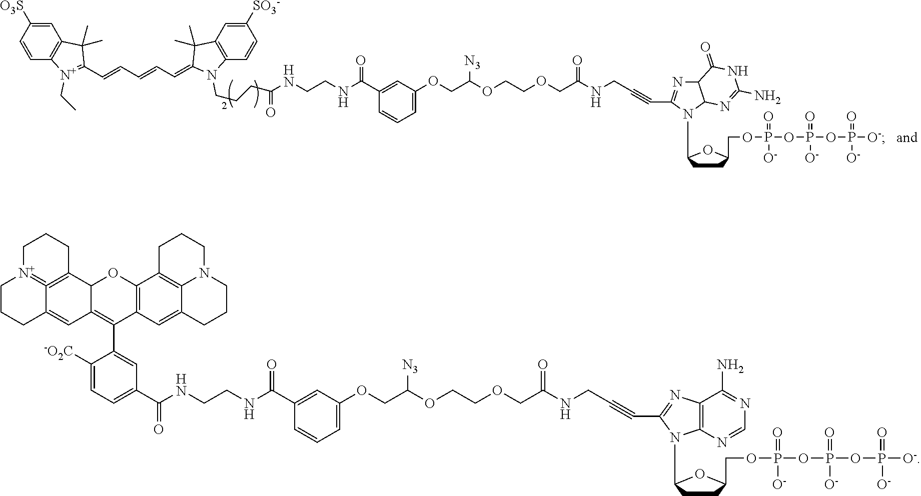

### 5.3. Mirror-Image Polymerase

In another aspect, the present disclosure provides a mirror-image polymerase. Specifically, the mirror-image polymerase comprises D-form amino acids. In some embodiments, the mirror-image polymerase consists of D-form amino acids. In some embodiments, the mirror-image polymerase comprises both D-form and L-form amino acids. In some embodiments, the mirror-image polymerase does not comprise L-form amino acids.

In some embodiments, the mirror-image nucleic acid is a mirror image of a DNA polymerase or an RNA polymerase. In some embodiments, the mirror-image nucleic acid is a mirror image of 9° N DNA polymerase or a modification thereof.

In some embodiments, the mirror-image polymerase comprises a sequence having at least 90% sequence identity to SEQ ID NO: 1. In some embodiments, the mirror-image polymerase comprises a sequence having at least 96% sequence identity to SEQ ID NO: 1. In some embodiments, the mirror-image polymerase comprises a sequence having at least 96% sequence identity to SEQ ID NO: 1. In some embodiments, the mirror-image polymerase comprises a sequence having at least 97% sequence identity to SEQ ID NO: 1. In some embodiments, the mirror-image polymerase comprises a sequence having at least 98% sequence identity to SEQ ID NO: 1. In some embodiments, the mirror-image polymerase comprises a sequence having at least 99% sequence identity to SEQ ID NO: 1. In some embodiments, the mirror-image polymerase comprises the sequence of SEQ ID NO: 1.

In some embodiments, the mirror-image polymerase has a sequence having at least 90% sequence identity to SEQ ID NO: 1. In some embodiments, the mirror-image polymerase has a sequence having at least 96% sequence identity to SEQ ID NO: 1. In some embodiments, the mirror-image polymerase has a sequence having at least 96% sequence identity to SEQ ID NO: 1. In some embodiments, the mirror-image polymerase has a sequence having at least 97% sequence identity to SEQ ID NO: 1. In some embodiments, the mirror-image polymerase has a sequence having at least 98% sequence identity to SEQ ID NO: 1. In some embodiments, the mirror-image polymerase has a sequence having at least 99% sequence identity to SEQ ID NO: 1. In some embodiments, the mirror-image polymerase has the sequence of SEQ ID NO: 1.

In some embodiments, the mirror-image polymerase comprises one or more modifications at one or more amino acid positions disclosed in Table 1 compared to SEQ ID NO: 1. In some embodiments, the mirror-image polymerase comprises one or more amino acid substitutions disclosed in Table 1 compared to SEQ ID NO: 1. In some embodiments, the mirror-image polymerase comprises all the amino acid substitutions disclosed in Table 1.

TABLE 1

| | Amino acid positions | |
|---|---|---|
| | (in reference to SEQ ID NO: 1) | modifications |
| Modification | E276, K317, N424, S651 | E276A, K317G, N424A, S651A |
| for NCL | | |
| (native | | |
| chemical | | |
| ligation) sites | | |
| Isoleucine for | I80, I127, I171, I176, I191, I228, | I80V, I127V, I171A, I176V, I191V, |
| substitution | I256, I264, I268, I400, I597, I610, | I228V, I256V, I264A, I268L, I400V, |
| | I618, I630, I642, I715, I733, I744 | IS97V, I610V, I618A, I630L, I642V, |
| | | I715Y, I733V, I744V |
| Modification | M129, 130-131, D141, E143, L408, | M129L, 130-131D, D141A, E143A, |
| for interaction | Y409, P410, A485, T514, I521 | L408A, Y409A, P410I, A485V, |
| with modified | | T514S, I521L |
| nucleotides | | |

In some embodiments, the mirror-image polymerase comprises one or more modifications compared to SEQ ID NO: 1 for native chemical ligation (NCL). In some embodiments, the mirror-image polymerase comprises one or more modifications at one or more amino acid sites selected from E276, K317, N424, and S651 compared to SEQ ID NO: 1. In some embodiments, the mirror-image polymerase comprises one or more substitutions selected from E276A, K317G, N424A, and S651A compared to SEQ ID NO: 1. In some embodiments, the mirror-image polymerase comprises E276A, K317G, N424A, and S651A substitutions compared to SEQ ID NO: 1.

In some embodiments, the mirror-image polymerase comprises one or more substitution of isoleucine compared to SEQ ID NO: 1. In some embodiments, the mirror-image polymerase comprises one or more modifications at one or more amino acid sites selected from I80, I127, I171, I176, I191, I228, I256, I264, I268, I400, I597, I610, I618, I630, I642, I715, I733, and I744 compared to SEQ ID NO: 1. In some embodiments, the mirror-image polymerase comprises one or more substitutions from Ile to Ala, Val, Leu, or Tyr at one or more amino acid sites selected from I80, I127, I171, I176, I191, I228, I256, I264, I268, I400, I597, I610, I618, I630, I642, I715, I733, and I744 compared to SEQ ID NO: 1. In some embodiments, the mirror-image polymerase comprises one or more substitutions selected from I80V, I127V, I171A, I176V, I191V, I228V, I256V, I264A, I268L, I400V, I597V, I610V, I618A, I630L, I642V, I715Y, I733V, and I744V compared to SEQ ID NO: 1.

In some embodiments, the mirror-image polymerase comprises one or more modifications compared to SEQ ID NO: 1 to improve its interaction with an L-nucleotide or a modification thereof disclosed herein.

In some embodiments, the mirror-image polymerase comprises one or more modifications at one or more amino acid sites selected from M129, I130, G131, D141, E143, L408, Y409, P410, A485, T514, and I521 compared to SEQ ID NO: 1. In some embodiments, the mirror-image polymerase comprises one or more modifications at one or more amino acid sites selected from D141, E143, Y409, and A485 compared to SEQ ID NO: 1. In some embodiments, the mirror-image polymerase comprises one or more substitutions selected from D141A, E143A, Y409V, and A485L compared to SEQ ID NO: 1. In some embodiments, the mirror-image polymerase comprises substitutions of D141A, E143A, Y409V, and A485L compared to SEQ ID NO: 1.

In some embodiments, the mirror-image polymerase comprises one or more modifications at one or more amino acid sites selected from D141, E143, L408, Y409, P410, A485, T514, and I521 compared to SEQ ID NO: 1. In some embodiments, the mirror-image polymerase comprises one or more substitutions selected from D141A, E143A, L408A, Y409A, P410I, A485V, T514S, and I521L compared to SEQ ID NO: 1. In some embodiments, the mirror-image polymerase comprises substitutions of D141A, E143A, L408A, Y409A, P410I, A485V, T514S, and I521L compared to SEQ ID NO: 1.

In some embodiments, the mirror-image polymerase comprises one or more modifications selected from substitution of M129L, D141A, E143A, L408A, Y409A, P410I, A485V, T514S, 1521L or addition of D between I130 and G131 compared to SEQ ID NO: 1. In some embodiments, the mirror-image polymerase comprises substitutions of M129L, D141A, E143A, L408A, Y409A, P410I, A485V, T514S, and I521L and addition of D between I130 and G131 compared to SEQ ID NO: 1.

In some embodiments, the mirror-image polymerase comprises a sequence selected from SEQ ID Nos 1-7. In some embodiments, the polymerase has a sequence selected from SEQ ID Nos: 1-7.

The mirror-image polymerase can be obtained by chemical synthesis. In some embodiments, polypeptides or small peptides are synthesized by solid phase peptide synthesis and then ligated by chemical ligation to obtain the mirror-image polymerase. In some embodiments, the mirror-image polymerase has the same enzymatic activity as the original polymerase but acts on D-form nucleotides.

### 5.4. Method of Use

In one aspect, the present disclosure provides methods of using mirror-image nucleotides and mirror-image polymerase disclosed herein. The mirror-image compositions can be used for replicating or sequencing L-polynucleotide. In some embodiments, the compositions are used for a sequencing method adopted from sequencing-by-synthesis method, Sanger method, or a combination thereof.

### 5.4.1. Method of Replicating L-Form Template

The present disclosure provides a method of replicating an L-polynucleotide. The method can comprise the step of: incubating a mixture comprising (i) the L-polynucleotide, (ii) an L-primer, (iii) L-dNTP, (iv) the mirror-form polymerase, and (v) a buffer, thereby inducing replication of the L-polymerase. In some embodiments, the L-polymerase is L-form DNA or RNA. In some embodiments, the L-polymerase comprises at least 10, 20, 30, 50, 100, 200, 500, or more nucleotides.

In some embodiments, the mixture comprises two or more L-dNTPs. In some embodiments, the mixture comprises L-dATP, L-dGTP, L-dCTP, L-dTTP, or a modification thereof. In some embodiments, the mixture comprises L-dATP, L-dGTP, L-dCTP, and L-dTTP.

In some embodiments, the mixture comprises a buffer developed for use with 9° N DNA polymerase. In some embodiments the buffer comprises Tris-HCl at a concentration of 1 mM to 200 mM, 5 mM to 150 mM, 10 mM to 100 mM, 20 mM to 100 mM or 30 mM to 80 mM. In some embodiments, the buffer comprises Tris-HCl at a concentration of about 10 mM, 20 mM, 30 mM, 40 mM, 50 mM, 60 mM, 70 mM, 80 mM, 90 mM or 100 mM. In some embodiments, the buffer comprises MgCl₂ at a concentration of 1 mM to 100 mM, 5 mM to 100 mM, 10 mM to 100 mM, 15 mM to 50 mM, or 15 mM to 30 mM. In some embodiments, the buffer comprises MgCl₂ at a concentration of about 10 mM, 20 mM, 30 mM, 40 mM or 50 mM. In some embodiments, the buffer comprises MnCl₂ at a concentration of 1 mM to 100 mM, 5 mM to 100 mM, 10 mM to 100 mM, 15 mM to 50 mM, or 15 mM to 30 mM. In some embodiments, the buffer comprises MnCl₂ at a concentration of about 10 mM, 20 mM, 30 mM, 40 mM or 50 mM. In some embodiments, the buffer comprises DTT at a concentration of 0.1 mM to 10 mM, 0.5 mM to 5 mM, 0.5 mM to 3 mM or 0.5 mM to 2 mM. In some embodiments, the buffer comprises DTT at a concentration of about 0.5 mM, 1 mM, 1.5 mM or 2 mM. In some embodiments, the buffer comprises KCl at a concentration of 10 mM to 100 mM, 20 mM to 80 mM, 25 mM to 75 mM or 30 mM to 70 mM. In some embodiments, the buffer comprises KCl at a concentration of about 25 mM, 30 mM, 40 mM, 50 mM, 60 mM, 70 mM or 100 mM. In some embodiments, the buffer has a pH between 7 and 8. In some embodiments, the buffer has a pH at about 7, 7.5 or 8.

In some embodiments, the buffer comprises Tris-HCl, MgCl₂, DTT and KCl. In some embodiments, the buffer comprises Tris-HCl, MnCl₂, DTT and KCl. In some embodiments, the buffer comprises Tris-HCl, MnCl₂, MgCl₂, DTT and KCl. In some embodiments, the buffer comprises 50 mM Tris-HCl, pH 7.5, 20 mM MgCl₂, 1 mM DTT, and 50 mM KCl.

### 5.4.2. Method of Sequencing

The present disclosure provides a method of sequencing an L-polynucleotide. The L-polynucleotide comprises L-form nucleotides. The L-polynucleotide can be DNA or RNA.

In some embodiments, the sequencing method comprises a cycle of: (a) incubating a mixture comprising (i) an L-polynucleotide, (ii) an L-primer, (iii) L-3′-O—R′-dNTP-R₂-Label, (iv) a mirror-form polymerase, and (v) a buffer, thereby obtaining a replication product; (b) detecting a signal from the L-3′-O—R′-dNTP-R₂-Label incorporated into the replication product; and (iii) inducing cleavage of the R′ group and R₂ group of the L-3′-O—R′-dNTP-R₂-Label incorporated into the replication product. The method involves (i) incorporation of L-3′-O—R′-dNTP-R₂-Label, (ii) identification of the incorporated nucleotide by signals from the incorporated L-3′-O—R′-dNTP-R₂-Label, and (iii) cleavage of the Label, along with the reinitiation of the polymerase reaction for continuing sequence determination. The L-3′-O—R′-dNTP-R₂-Label includes a chemical moiety (R′) capping the 3′-OH and a Label tethered to the base through a chemically cleavable linker (R₂). The 3′-capping moiety (R′) and the Label on the reaction product are cleaved to reinitiate the polymerase reaction.

In some embodiments, the method comprises multiple cycles. In some embodiments, the cycle is repeated at least 3, 5, 10, 50, 100, 150, 200, 250, 300, 400, 500, or 1000 times. In some embodiments, the cycle is repeated less than 5, 10, 50, 100, 150, 200, 250, 300, 400, 500, 1000, 2000, 3000, or 5000 times.

In some embodiments, the L-polynucleotide comprises more than 3, 5, 10, 50, 100, 150, 200, 250, 300, 400, 500, or 1000 nucleotides. In some embodiments, the L-polynucleotide comprises less than 5, 10, 50, 100, 150, 200, 250, 300, 400, 500, 1000, 2000, 3000, or 5000 nucleotides.

In some embodiments, the L-3′-O—R′-dNTP-R₂-Label comprises L-3′-O—R′-dATP-R₂-Label, L-3′-O—R′-dTTP-R₂-Label, L-3′-O—R′-dGTP-R₂-Label, or L-3′-O—R′-dCTP-R₂-Label. In some embodiments, the L-3′-O—R′-dNTP-R₂-Label comprises two or more of L-3′-O—R′-dATP-R₂-Label, L-3′-O—R′-dTTP-R₂-Label, L-3′-O—R′-dGTP-R₂-Label, and L-3′-O—R′-dCTP-R₂-Label. In some embodiments, the L-3′-O—R′-dNTP-R₂-Label comprises L-3′-O—R′-dATP-R₂-Label, L-3′-O—R′-dTTP-R₂-Label, L-3′-O—R′-dGTP-R₂-Label, and L-3′-O—R′-dCTP-R₂-Label. Labels attached to each kind of dNTP can be unique and identifiable.

In some embodiments, the L-3′-O—R′-dNTP-R₂-Label has a structure according to Formula I as described in 5.2.2.1.2. In some embodiments, the L-3′-O—R′-dNTP-R₂-Label has a structure according to Formula II as described in 5.2.2.1.2.

In some embodiments, the L-3′-O—R′-dNTP-R₂-Label includes a fluorescent label. In the case, the signal is a fluorescent signal. In some embodiments, each type of L-3′-O—R′-dNTP-R₂-Label (e.g., dATP, dGTP, dTTP, dCTP) is tagged to a unique label. In some embodiments, each type of L-3′-O—R′-dNTP-R₂-Label (e.g., dATP, dGTP, dTTP, dCTP) provides a unique fluorescent signal. The L-3′-O—R′-dNTP-R₂-Label can include any of the labels disclosed in 5.2.1.3.1. In some embodiments, the L-3′-O—R′-dNTP-R₂-Label includes a non-fluorescent label.

In some embodiments, the sequencing method comprises the steps of (a) incubating a mixture comprising (i) the L-polynucleotide, (ii) an L-primer, (iii) L-dNTP, (iv) an L-ddNTP-R₂-Label, (v) a mirror-image polymerase disclosed herein, and (vi) a buffer, thereby obtaining a replication product; (b) separating the replication product; and (c) detecting a signal from the L-ddNTP-R₂-Label incorporated into the replication product. The incubation step can comprise PCR reaction.

In preferred embodiments of the method, a low ratio of L-ddNTP-R₂-Label is added to the mixture compared to L-dNTP. During incubation (e.g., PCR reaction), L-ddNTP-R₂-Label lacking 3′—OH group can be incorporated at random by the mirror-image polymerase to the replication product. Incorporation of L-ddNTP-R₂-Label terminates the replication process. The reaction can induce production of oligonucleotide copies of the replication products terminated at a random length by L-ddNTP-R₂-Label.

In the method, the replication product can be separated by size. In some embodiments, the replication product is separated by size via gel electrophoresis. By detecting a signal from the L-ddNTP-R₂-Label incorporated into the replication product, the identity of the terminal L-ddNTP-R₂-Label (e.g., ddATP, ddGTP, ddTTP, or ddCTP) can be determined for the replication product. The data can show types of nucleotides along the length of the L-polynucleotide. This process can allow determination of the sequence of the L-polynucleotide.

In some embodiments, the L-dNTP comprises L-dATP, L-dTTP, L-dGTP, or L-dCTP. In some embodiments, the L-dNTP comprises one or more of L-dATP, L-dTTP, L-dGTP, and L-dCTP. In some embodiments, the L-dNTP comprises L-dATP, L-dTTP, L-dGTP, and L-dCTP.

In some embodiments, the L-ddNTP-R₂-Label comprises L-ddATP-R₂-Label, L-ddTTP-R₂-Label, L-ddGTP-R₂-Label, or L-ddCTP-R₂-Label. In some embodiments, the L-ddNTP-R₂-Label comprises one or more of L-ddATP-R₂-Label, L-ddTTP-R₂-Label, L-ddGTP-R₂-Label, and L-ddCTP-R₂-Label. In some embodiments, the L-ddNTP-R₂-Label comprises L-ddATP-R₂-Label, L-ddTTP-R₂-Label, L-ddGTP-R₂-Label, and L-ddCTP-R₂-Label. Labels attached to each kind of ddNTP can be unique and identifiable.

In some embodiments, the L-ddNTP-R₂-Label has a structure disclosed in 5.2.2.2.1. In some embodiments, the L-ddNTP-R₂-Label has Formula III as defined in 5.2.2.2.1.

In some embodiments, the L-ddNTP-R₂-Label includes a fluorescent label. In the case, the signal is a fluorescent signal. In some embodiments, each type of L-ddNTP-R₂-Label (e.g., ddATP, ddGTP, ddTTP, ddCTP) is tagged to a unique label. In some embodiments, each type of L-ddNTP-R₂-Label (e.g., ddATP, ddGTP, ddTTP, ddCTP) provides a unique fluorescent signal. The L-ddNTP-R₂-Label can include any of the labels disclosed in 5.2.1.3.1. In some embodiments, the L-ddNTP-R₂-Label includes a non-fluorescent label.

In some embodiments, the sequencing method comprises a cycle of (a) incubating a mixture comprising (i) an L-polynucleotide, (ii) an L-primer, (iii) L-3′-O—R′-dNTP, (iv) L-ddNTPs-R₂-Label, (v) a mirror-image polymerase provided herein, and (vi) a buffer, thereby obtaining a replication product: (b) detecting a signal from the L-ddNTP-R₂-Label incorporated into the replication product; and (c) inducing cleavage of the R′ group of the L-3′-O—R′-dNTP and R₂ group of L-ddNTPs-R₂-Label incorporated into the replication product. In this method, the polymerase reaction is performed with the combination of 3′-capped nucleotide reversible terminators (L-3′-O—R′-dNTP) and cleavable fluorescent dideoxynucleotides (L-ddNTPs-R₂-Label). In this method, sequences are determined by the signal of each Label on the reaction products terminated by ddNTPs (L-ddNTPs-R₂-Label). Upon removing the 3′-OH capping group (R′ group) from the incorporated nucleotide reversible terminators (L-3′-O—R′-dNTP) and the Label from the DNA products terminated by ddNTPs (L-ddNTPs-R₂-Label), the polymerase reaction reinitiates to continue the sequence determination.

In some embodiments, the method comprises multiple cycles. In some embodiments, the cycle is repeated at least 3, 5, 10, 50, 100, 150, 200, 250, 300, 400, 500, or 1000 times. In some embodiments, the cycle is repeated less than 5, 10, 50, 100, 150, 200, 250, 300, 400, 500, 1000, 2000, 3000, or 5000 times.

In some embodiments, the L-polynucleotide comprises more than 3, 5, 10, 50, 100, 150, 200, 250, 300, 400, 500, or 1000 nucleotides. In some embodiments, the L-polynucleotide comprises less than 5, 10, 50, 100, 150, 200, 250, 300, 400, 500, 1000, 2000, 3000, or 5000 nucleotides.

In some embodiments, the L-3′-O—R′-dNTP comprises L-3′-O—R′-dATP, L-3′-O—R′-dTTP, L-3′-O—R′-dGTP, or L-3′-O—R′-dCTP. In some embodiments, the L-3′-O—R′-dNTP comprises one or more of L-3′-O—R′-dATP, L-3′-O—R′-dTTP, L-3′-O—R′-dGTP, and L-3′-O—R′-dCTP. In some embodiments, the L-3′-O—R′-dNTP comprises L-3′-O—R′-dATP, L-3′-O—R′-dTTP, L-3′-O—R′-dGTP, and L-3′-O—R′-dCTP.

In some embodiments, the L-3′-O—R′-dNTP has any of the structure provided in 5.2.2.1.1. In some embodiments, the L-3′-O—R′-dNTP has the Formula I as defined in 5.2.2.1.1

In some embodiments, the L-ddNTP-R₂-Label comprises L-ddATP-R₂-Label, L-ddTTP-R₂-Label, L-ddGTP-R₂-Label, or L-ddCTP-R₂-Label. In some embodiments, the L-ddNTP-R₂-Label comprises one or more of L-ddATP-R₂-Label, L-ddTTP-R₂-Label, L-ddGTP-R₂-Label, and L-ddCTP-R₂-Label. In some embodiments, the L-ddNTP-R₂-Label comprises L-ddATP-R₂-Label, L-ddTTP-R₂-Label, L-ddGTP-R₂-Label, and L-ddCTP-R₂-Label. Labels attached to each kind of dNTP can be unique and identifiable.

In some embodiments, the L-ddNTP-R₂-Label has any of the structure provided in 5.2.2.2.1. In some embodiments, the L-ddNTP-R₂-Label has a formula III as defined in 5.2.2.2.1. In some embodiments, each type of L-3′-O—R′-dNTP-R₂-Label (e.g., dATP, dGTP, dTTP, dCTP) provides a unique fluorescent signal. The L-ddNTP-R₂-Label can include any of the labels disclosed in 5.2.1.3.1. In some embodiments, the L-ddNTP-R₂-Label includes a non-fluorescent label.

In some embodiments, the sequencing method comprises a cycle of: (a) incubating a mixture comprising (i) an L-polynucleotide, (ii) an L-primer, (iii) L-dNTP, (iv) a mirror-form polymerase, and (v) a buffer, thereby obtaining a reaction product; and (b) detecting release of PPi molecule in the reaction product. In some embodiments, the sequencing method comprises a plurality of the cycles, wherein in each cycle, the mixture comprises a single type of L-dNTP selected from L-dATP, L-dTTP, L-dGTP and L-dCTP. In some embodiments, the sequencing method comprises a plurality of cycles, wherein each cycle is performed by changing the type of L-dNTP to one of L-dATP, L-dTTP, L-dGTP and L-dCTP stepwise.

In some embodiments, the step of detecting release of PPi molecule comprises the steps of (i) processing the reaction product to convert PPi molecule ATP if present and to generate a detectable signal from the ATP if present; and (ii) detecting presence or absence of the detectable signal. In some embodiments, the ATP is processed with luciferase to generate the detectable signal. In some embodiments, the detectable signal is light. In some embodiments, the PPi molecule is converted into ATP by ATP-sulfurylase.

### 5.5. Kit

The present disclosure provides a kit for replicating or sequencing L-polymerase. The kit comprises a mirror-image polymerase disclosed herein and optionally, a buffer.

In some embodiments, the kit further comprises L-dNTP. In some embodiments, the L-NTP comprises L-dATP, L-dGTP, L-dCTP, L-dTTP or L-UTP. In some embodiments, the L-NTP comprises one or more of L-dATP, L-dGTP, L-dCTP, L-dTTP and L-UTP. In some embodiments, the L-NTP comprises L-dATP, L-dGTP, L-dCTP, and L-dTTP. In some embodiments, the L-NTP comprises L-dATP, L-dGTP, L-dCTP, and L-UTP. In some embodiments, the L-NTP comprises L-dATP, L-dGTP, L-dCTP, L-dTTP and L-UTP.

In some embodiments, the kit comprises L-3′-O—R′-dNTP-R₂-Label. In some embodiments, the L-3′-O—R′-dNTP-R₂-Label comprises L-3′-O—R′-dATP-R₂-Label, L-3′-O—R′-dTTP-R₂-Label, L-3′-O—R′-dGTP-R₂-Label, L-3′-O—R′-dCTP-R₂-Label or L-3′-O—R′-dUTP-R₂-Label. In some embodiments, the L-3′-O—R′-dNTP-R₂-Label comprises one or more of L-3′-O—R′-dATP-R₂-Label, L-3′-O—R′-dTTP-R₂-Label, L-3′-O—R′-dGTP-R₂-Label, L-3′-O—R′-dCTP-R₂-Label and L-3′-O—R′-dUTP-R₂-Label. In some embodiments, the L-3′-O—R′-dNTP-R₂-Label comprises one or more of L-3′-O—R′-dATP-R₂-Label, L-3′-O—R′-dTTP-R₂-Label, L-3′-O—R′-dGTP-R₂-Label, and L-3′-O—R′-dCTP-R₂-Label. In some embodiments, the L-3′-O—R′-dNTP-R₂-Label comprises one or more of L-3′-O—R′-dATP-R₂-Label, L-3′-O—R′-dGTP-R₂-Label, L-3′-O—R′-dCTP-R₂-Label and L-3′-O—R′-dUTP-R₂-Label. In some embodiments, each type of L-3′-O—R′-dNTP-R₂-Label (e.g., dATP, dGTP, dTTP, dCTP) provides a unique fluorescent signal.

In some embodiments, the L-3′-O—R′-dNTP-R₂-Label has Formula II as defined in 5.2.2.1.2. In some embodiments, the L-3′-O—R′-dNTP-R₂-Label has any of the structures disclosed in 5.2.2.1.2.

In some embodiments, the kit comprises L-ddNTP-R₂-Label. In some embodiments, the L-ddNTP-R₂-Label comprises L-ddATP-R₂-Label, L-ddTTP-R₂-Label, L-ddGTP-R₂-Label, L-ddCTP-R₂-Label or L-ddUTP-R₂-Label. L-ddNTP-R₂-Label. In some embodiments, the L-ddNTP-R₂-Label comprises one or more of L-ddATP-R₂-Label, L-ddTTP-R₂-Label, L-ddGTP-R₂-Label, L-ddCTP-R₂-Label and L-ddUTP-R₂-Label. In some embodiments, the L-ddNTP-R₂-Label comprises L-ddATP-R₂-Label, L-ddTTP-R₂-Label, L-ddGTP-R₂-Label, and L-ddCTP-R₂-Label. In some embodiments, the L-ddNTP-R₂-Label comprises L-ddATP-R₂-Label, L-ddGTP-R₂-Label, L-ddCTP-R₂-Label and L-ddUTP-R₂-Label.

In some embodiments, the L-ddNTP-R₂-Label has Formula III as defined in 5.2.2.2.1. In some embodiments, the L-ddNTP-R₂-Label has any of the structures disclosed in 5.2.2.2.1.

In some embodiments, the kit further comprises L-3′-O—R′-dNTP. The L-3′-O—R′-dNTP can comprise L-3′-O—R′-dATP, L-3′-O—R′-dTTP, L-3′-O—R′-dGTP, or L-3′-O—R′-dCTP. In some embodiments, the L-3′-O—R′-dNTP comprises one or more of L-3′-O—R′-dATP, L-3′-O—R′-dTTP, L-3′-O—R′-dGTP, and L-3′-O—R′-dCTP. In some embodiments, the L-3′-O—R′-dNTP comprises L-3′-O—R′-dATP, L-3′-O—R′-dTTP, L-3′-O—R′-dGTP, and L-3′-O—R′-dCTP.

In some embodiments, the L-3′-O—R′-dNTP has Formula I as defined in 5.2.2.1.1. In some embodiments, the L-3′-O—R′-dNTP has any of the structures disclosed in 5.2.2.1.1.

In some embodiments, each of the L-nucleotides-Label in the kit has a unique label. In some embodiments, some of the L-nucleotides-Label in the kit has the same label. In some embodiment, the label is a fluorescent label. In some embodiments, each of the L-nucleotides-Label in the kit provides a unique fluorescent signal.

### 6. EXAMPLES

### 6.1. Example 1. Synthesis of (L)-3′-O-Azidomethyl-dNTPs

### Materials and Methods

Starting material beta-L-deoxy adenosine, L-deoxy guanosine, L-deoxy cytidine, L-deoxy thymidine are available for purchase, e.g., at Chemgenes. All solvents and reagents are reagent grades, purchased commercially, and used without further purification unless specified.

### 6.1.1. Synthesis of (L) 3′-O-Azidomethyl-dATP

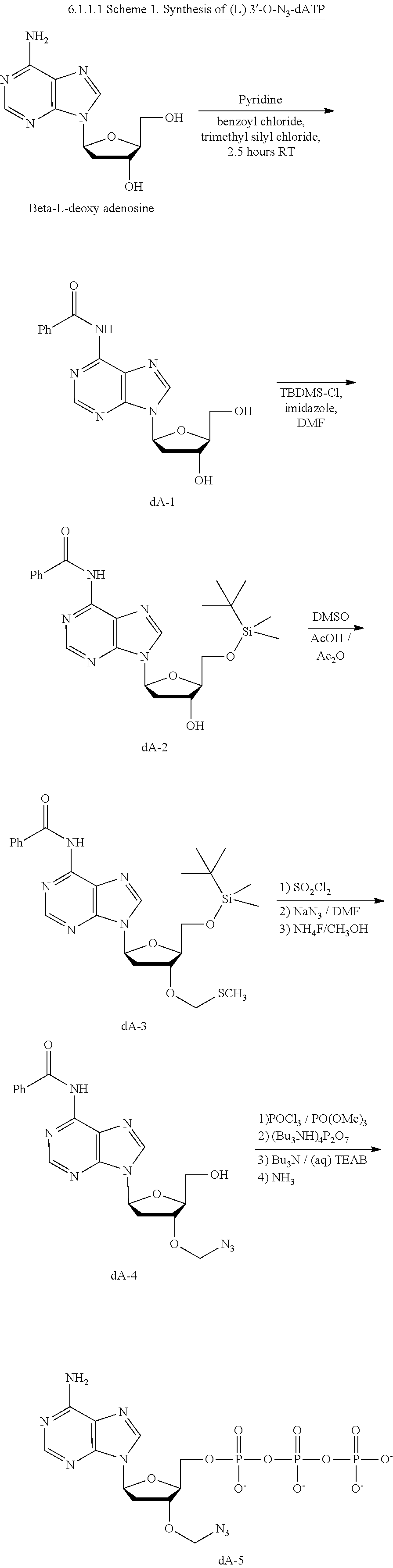 [SMILES: Nc1ncnc2c1ncn2[C@@H]1C[C@H](O)[C@H](CO)O1 ; O=C(Nc1ncnc2c1ncn2[C@@H]1C[C@H](O)[C@H](CO)O1)c1ccccc1 ; CC(C)(C)[Si](C)(C)OC[C@@H]1O[C@H](n2cnc3c(NC(=O)c4ccccc4)ncnc32)C[C@@H]1O ; CSCO[C@H]1C[C@@H](n2cnc3c(NC(=O)c4ccccc4)ncnc32)O[C@H]1CO[Si](C)(C)C(C)(C)C ; [N-]=[N+]=NCO[C@H]1C[C@@H](n2cnc3c(NC(=O)c4ccccc4)ncnc32)O[C@H]1CO ; [N-]=[N+]=NCO[C@H]1C[C@@H](n2cnc3c(N)ncnc32)O[C@H]1COP(=O)([O-])OP(=O)([O-])OP(=O)([O-])[O-]]

To a stirred solution of the ‘starting material’ (beta-L-deoxy adenosine) (1.0 eq, 2.00 g, 7.48 mmol) is co-evaporated with anhydrous pyridine, dissolved in 15 ml of anhydrous pyridine and sealed with septum. After stirring for 5 minutes under argon, trimethylsilyl chloride (TMS-Cl, 5.0 eq, 4.06 g, 37.4 mmol) is added via syringe. Benzoyl chloride (1.2 eq, 1.26 g, 8.98 mmol) is added dropwise via syringe over a period of 20 minutes and after 30 minutes and stirring is continued for 2.5 h, while a clear yellow solution is formed. Then, 4 ml H₂O is added at once and after 5 minutes, 8 ml of aqueous ammonia solution (28-30/6) is added at once stirring for additional 15 minutes. The mixture is evaporated to dryness and the oily residue co-evaporated twice with toluene to give a yellow solid (compound dA-1). To a stirred mixture of compound dA-1 (1.50 g; 3.96 mmol) and imidazole (693 mg; 9.51 mmol) in anhydrous DMF (21.0 mL), tert-butyldimethylsilyl chloride (TBDMSCl) (765 mg; 4.92 mmol) is added. The reaction mixture is stirred at room temperature for 20 h. After evaporation, the residue is purified by flash column chromatography using CH₃OH—CH₂Cl₂ (1:20) as the eluent to afford compound dA-2 as white solid. To a stirred solution of compound dA-2 (3.0 g; 6.38 mmol) in DMSO (12 ml), acetic acid (5.5 ml) and acetic anhydride (17.6 ml) are added. The reaction mixture is stirred at room temperature for 48 h. A saturated NaHCO₃ solution (100 ml) is added and the aqueous layer is extracted with CH₂Cl₂ (3×100 ml). The combined organic extract is washed with saturated NaHCO₃ solution (100 ml) and dried over Na₂SO₄. After concentration, the residue is purified by flash column chromatography (hexane/ethyl acetate, 1:1 to 1:4) to afford compound dA-3 as a white powder. To a stirred solution of compound dA-3 (400 mg; 0.76 mmol) in dry CH₂Cl₂ (7 ml) under nitrogen, cyclohexene (400 μl), and SO₂Cl₂ (155 μl; 1.91 mmol, redistilled) are added. The reaction mixture is stirred at 0° C. for 2 h. The solvent is first removed under reduced pressure and then under a high-vacuum pump for 10 min. The residue is dissolved in dry DMF (5 ml) and reacted with NaN₃ (400 mg; 6.6 mmol) at room temperature for 3 h. The reaction mixture is dispersed in distilled water (50 ml) and extracted with CH₂Cl₂ (3×50 ml). The combined organic layer is dried over Na₂SO₄ and concentrated under reduced pressure. The residue is dissolved in MeOH (5 ml) and stirred with NH₄F (300 mg; 8.1 mmol) at room temperature for 24 h. The solvent is removed under reduced pressure. The reaction mixture is concentrated under reduced pressure and partitioned between H₂O and CH₂Cl₂. The organic layer is separated and dried over Na₂SO₄. After concentration, the crude product is purified by flash column chromatography (ethyl acetate/methanol, 100:0 to 98:2) to afford compound dA-4 as a white powder. Compound dA-4 (123 mg; 0.3 mmol) and proton sponge (75.8 mg; 0.35 mmol) are dried in a vacuum desiccator over P₂O₅ overnight before dissolving in trimethyl phosphate (600 μl). Then freshly distilled POCl₃ (40 μl; 0.35 mmol) is added dropwise at 0° C. and the mixture is stirred at 0° C. for 2 h. Subsequently, a well-vortexed mixture of tributylammonium pyrophosphate (552 mg) and tributylamine (0.55 ml; 2.31 mmol) in anhydrous DMF (2.33 ml) is added in one portion at room temperature and stirred for 30 min. Triethyl ammonium bicarbonate solution (TEAB) (0.1 M; pH 8.0; 15 ml) is then added and the mixture is stirred for 1 h at room temperature. Then concentrated NH₄OH (15 ml) is added and stirred overnight at room temperature. The resulting mixture is concentrated under vacuum and the residue is diluted with 5 ml of water. The crude mixture is then purified with anion exchange chromatography on DEAE-Sephadex A-25 at 4° C. using a gradient of TEAB (pH 8.0; 0.1-1.0 M). The crude product is further purified by reverse-phase HPLC to afford (L) 3′-O-azidomethyl-dATP (compound dA-5).

### 6.1.2. Synthesis of (L) 3′-O-azidomethyl-dGTP

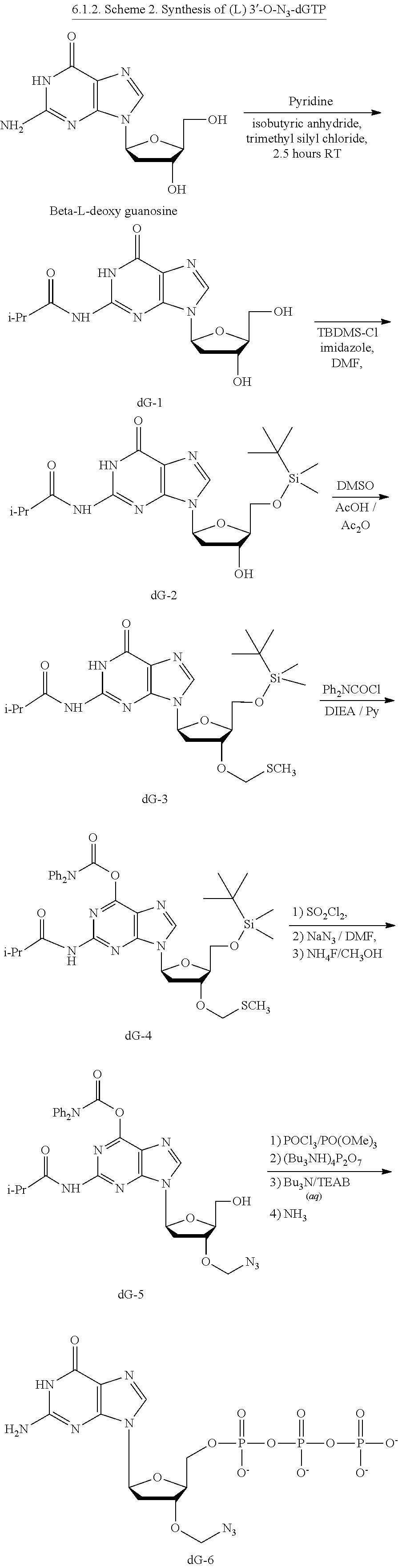

To a stirred solution of the ‘starting material’ (beta-L-deoxy guanosine) (1.0 eq, 2.00 g, 7.13 mmol) is co-evaporated with anhydrous pyridine (3×4 mL) and then dissolved in anhydrous pyridine (2 mL). The resulting solution is protected from moisture (drying tube), purged with argon and placed on ice. To the ice cold solution, TMS-Cl (4.51 mL, 58.4 mmol, 8.2 eq.) is added dropwise via a syringe. The ice bath is then removed and the mixture is stirred for 2 hours. The solution is cooled on ice and isobutyric anhydride (0.29 mL, 15.69 mmol, 2.2 eq.) is added dropwise via a syringe and the ice bath is removed. After stirring for another 2 hours at room temperature, the reaction is placed again on ice and ice cold water (20 mL) is slowly added, followed after 15 minutes by concentrated ammonia solution (1.5 mL) to get a final 2.5 M concentration of ammonia. The mixture is kept on ice for 30 minutes, and then evaporated to dryness. The residue is co-evaporated with toluene (3×5 mL) to remove traces of water, resuspended in MeOH and filtered to remove the precipitate. The filtrate is then concentrated, dissolved in a small amount of MeOH, absorbed on silica gel and purified by column chromatography (DCM/MeOH 95:5 to 91:9 (v/v)) to give compound dG-1 as a yellow solid. Compound dG-1 (495 mg, 1.07 mmol) is co-evaporated three times with dry pyridine, dried under high vacuum, and dissolved in 2 cm³ dry N,N-dimethylformamide in an ice bath. Di-tert-butylsilyl bis(trifluoromethanesulfonate) (590 mg, 1.34 mmol) is added dropwise over a period of 15 min and the reaction mixture is stirred at 0° C. for 30 min. Imidazole (419 mg, 6.15 mmol) is added and the mixture is stirred for 15 min at 0° C. and for 15 min at room temperature. Then, 241 mg tert-butyldimethylsilyl chloride (1.59 mmol) is added and the solution is stirred at 60° C. for another 2 h. The mixture is diluted with dichloromethane, washed with brine, dried over sodium sulfate, and evaporated. The crude product is purified by column chromatography on silica gel (methanol:dichloromethane 0:100-2:98) as white foam (compound dG-2). To a stirred solution of compound dG-2 (3.0 g; 6.08 mmol) in DMSO (12 ml), acetic acid (5.5 ml) and acetic anhydride (17.6 ml) are added. The reaction mixture is stirred at room temperature for 48 h. A saturated NaHCO₃ solution (100 ml) is added and the aqueous layer is extracted with CH₂Cl₂ (3×100 ml). The combined organic extract is washed with saturated NaHCO₃ solution (100 ml) and dried over Na₂SO₄. After concentration, the residue is purified by flash column chromatography (hexane/ethyl acetate, 1:1 to 1:4) to afford compound dG-3 as a white powder. To a stirred solution of compound dG-3 (1.0 g; 2.0 mmol) in dry pyridine (22 ml), diphenylcarbamoyl chloride (677 mg; 2.92 mmol) and DIEA (N, N-diisopropylethylamine) (1.02 ml; 5.9 mmol) are added. The reaction mixture is stirred under nitrogen atmosphere at room temperature for 3 h. The solvent is removed under high vacuum. The crude product is purified by flash column chromatography (ethyl acetate/hexane, 1:1 to 7:3) to afford compound dG-4 as a yellowish powder. To a stirred solution of compound dG-4 (400 mg; 0.71 mmol) in dry CH₂Cl₂ (7 ml) under nitrogen, cyclohexene (400 μl), and SO₂Cl₂ (155 μl; 1.91 mmol, redistilled) are added. The reaction mixture is stirred at 0° C. for 2 h. The solvent is first removed under reduced pressure and then under a high-vacuum pump for 10 min. The residue is dissolved in dry DMF (5 ml) and reacted with NaN₃ (400 mg; 6.6 mmol) at room temperature for 3 h. The reaction mixture is dispersed in distilled water (50 ml) and extracted with CH₂Cl₂ (3×50 ml). The combined organic layer is dried over Na₂SO₄ and concentrated under reduced pressure. The residue is dissolved in MeOH (5 ml) and stirred with NH₄F (300 mg; 8.1 mmol) at room temperature for 24 h. The solvent is removed under reduced pressure. The reaction mixture is concentrated under reduced pressure and partitioned between H₂O and CH₂Cl₂. The organic layer is separated and dried over Na₂SO₄. After concentration, the crude product is purified by flash column chromatography (ethyl acetate/methanol, 100:0 to 98:2) to afford compound dG-5 as a white powder. Compound dG-5 (123 mg; 0.28 mmol) and proton sponge (75.8 mg; 0.35 mmol) are dried in a vacuum desiccator over P₂O₅ overnight before dissolving in trimethyl phosphate (600 μl). Then freshly distilled POCl₃ (40 μl; 0.35 mmol) is added dropwise at 0° C. and the mixture is stirred at 0° C. for 2 h. Subsequently, a well-vortexed mixture of tributylammonium pyrophosphate (552 mg) and tributylamine (0.55 ml; 2.31 mmol) in anhydrous DMF (2.33 ml) is added in one portion at room temperature and stirred for 30 min. Triethyl ammonium bicarbonate solution (TEAB) (0.1 M; pH 8.0; 15 ml) is then added and the mixture is stirred for 1 h at room temperature. Then concentrated NH₄OH (15 ml) is added and stirred overnight at room temperature. The resulting mixture is concentrated under vacuum and the residue is diluted with 5 ml of water. The crude mixture is then purified with anion exchange chromatography on DEAE-Sephadex A-25 at 4° C. using a gradient of TEAB (pH 8.0; 0.1-1.0 M). The crude product is further purified by reverse-phase HPLC to afford (L) 3′-O-azidomethyl-dGTP (compound dG-6).

### 6.1.3. Synthesis of (L) 3′-O-azidomethyl-dCTP

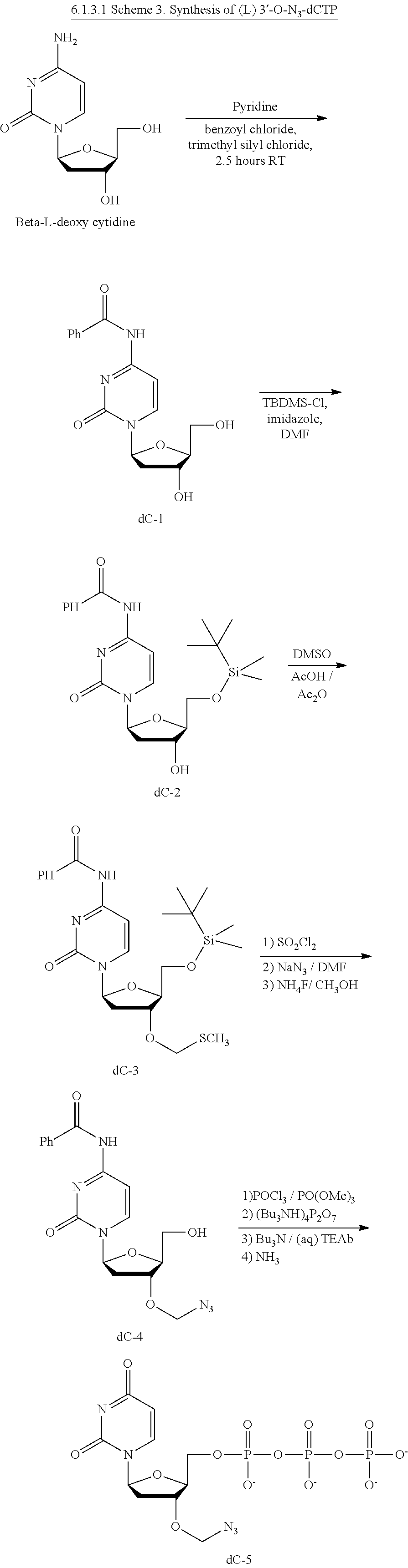

The preparation procedure for (L) 3′-O-azidomethyl-dCTP (compound dC-5) is similar to the synthesis protocol (5 steps) outlined above for (L) 3′-O-azidomethyl-dATP. Starting material for synthesis of (L) 3′-O-azidomethyl-dCTP is beta-L-deoxy cytidine (Chemgenes).

### 6.1.4. Prophetic Synthesis of (L) 3′-O-Azidomethyl-dTTP

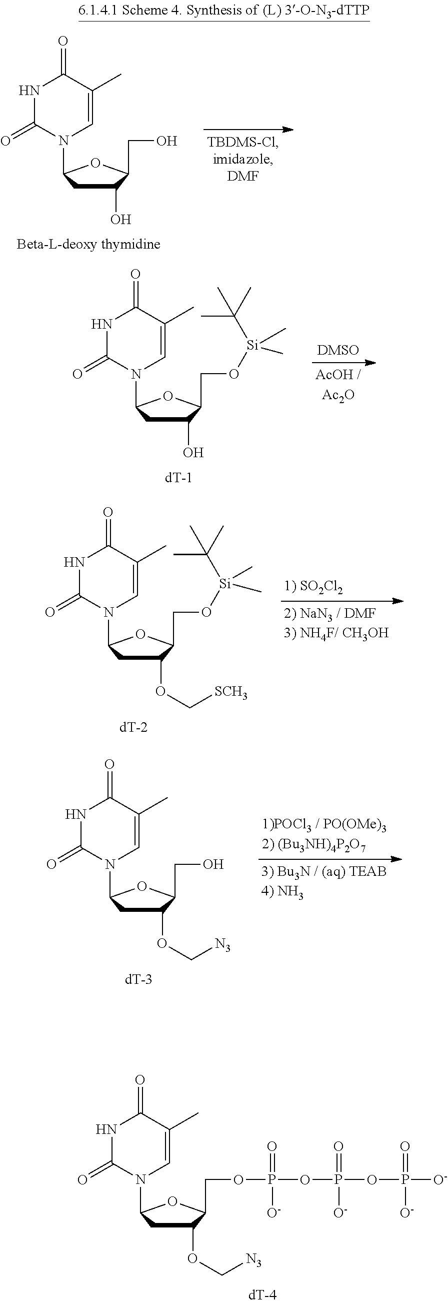 [SMILES: Cc1cn([C@@H]2C[C@H](O)[C@H](CO)O2)c(=O)[nH]c1=O ; Cc1cn([C@@H]2C[C@H](O)[C@H](CO[Si](C)(C)C(C)(C)C)O2)c(=O)[nH]c1=O ; CSCO[C@H]1C[C@@H](n2cc(C)c(=O)[nH]c2=O)O[C@H]1CO[Si](C)(C)C(C)(C)C ; Cc1cn([C@@H]2C[C@H](OCN=[N+]=[N-])[C@H](CO)O2)c(=O)[nH]c1=O ; Cc1cn([C@@H]2C[C@H](OCN=[N+]=[N-])[C@H](COP(=O)([O-])OP(=O)([O-])OP(=O)([O-])[O-])O2)c(=O)[nH]c1=O]

To a stirred mixture of the ‘starting material’ (beta-L-deoxy thymidine) (1.3 g; 3.87 mmol) and imidazole (693 mg; 9.51 mmol) in anhydrous DMF (21.0 mL), tert-butyldimethylsilyl chloride (TBDMSCl) (765 mg; 4.92 mmol) is added. The reaction mixture is stirred at room temperature for 20 h. After evaporation, the residue is purified by flash column chromatography using CH₃OH—CH₂Cl₂ (1:20) as the eluent to afford compound dT-1 as white solid. To a stirred solution of compound dT-1 (3.0 g; 6.38 mmol) in DMSO (12 ml), acetic acid (5.5 ml) and acetic anhydride (17.6 ml) are added. The reaction mixture is stirred at room temperature for 48 h. A saturated NaHCO₃ solution (100 ml) is added and the aqueous layer is extracted with CH₂Cl₂ (3×100 ml). The combined organic extract is washed with saturated NaHCO₃ solution (100 ml) and dried over Na₂SO₄. After concentration, the residue is purified by flash column chromatography (hexane/ethyl acetate, 1:1 to 1:4) to afford compound dT-2 as a white powder. To a stirred solution of compound dT-2 (400 mg, 0.76 mmol) in dry CH₂Cl₂ (7 ml) under nitrogen, cyclohexene (400 μl), and SO₂Cl₂ (155 μl, 1.91 mmol, redistilled) are added. The reaction mixture is stirred at 0° C. for 2 h. The solvent is first removed under reduced pressure and then under a high-vacuum pump for 10 min. The residue is dissolved in dry DMF (5 ml) and reacted with NaN₃ (400 mg; 6.6 mmol) at room temperature for 3 h. The reaction mixture is dispersed in distilled water (50 ml) and extracted with CH₂Cl₂ (3×50 ml). The combined organic layer is dried over Na₂SO₄ and concentrated under reduced pressure. The residue is dissolved in MeOH (5 ml) and stirred with NH₄F (300 mg; 8.1 mmol) at room temperature for 24 h. The solvent is removed under reduced pressure. The reaction mixture is concentrated under reduced pressure and partitioned between H₂O and CH₂Cl₂. The organic layer is separated and dried over Na₂SO₄. After concentration, the crude product is purified by flash column chromatography (ethyl acetate/methanol, 100:0 to 98:2) to afford compound dT-3 as a white powder. Compound dT-3 (123 mg; 0.3 mmol) and proton sponge (75.8 mg; 0.35 mmol) are dried in a vacuum desiccator over P₂O₅ overnight before dissolving in trimethyl phosphate (600 μl). Then freshly distilled POCl₃ (40 μl; 0.35 mmol) is added dropwise at 0° C. and the mixture is stirred at 0° C. for 2 h. Subsequently, a well-vortexed mixture of tributylammonium pyrophosphate (552 mg) and tributylamine (0.55 ml; 2.31 mmol) in anhydrous DMF (2.33 ml) is added in one portion at room temperature and stirred for 30 min. Triethyl ammonium bicarbonate solution (TEAB) (0.1 M; pH 8.0; 15 ml) is then added and the mixture is stirred for 1 h at room temperature. Then concentrated NH₄OH (15 ml) is added and stirred overnight at room temperature. The resulting mixture is concentrated under vacuum and the residue is diluted with 5 ml of water. The crude mixture is then purified with anion exchange chromatography on DEAE-Sephadex A-25 at 4° C. using a gradient of TEAB (pH 8.0; 0.1-1.0 M). The crude product is further purified by reverse-phase HPLC to afford (L) 3′-O-azidomethyl-dTTP (compound dT-4).

### 6.2. Example 2. Synthesis of (L) 3′-O—R′-dNTP

A person of skill in the art, having reviewed the exemplified methods provided herein would readily be able to adapt these methods to produce compounds with various R′ groups protecting the 3′O. Specifically, the allyl and the nitrobenzyl analogs of the described (L)-3′-O-azidomethyl-dNTPs are contemplated utilizing allyl bromide and 2-nitrobenzyl bromide respectively, instead of the acetic acid/acetic anyhydride step in the above reaction schemes 1-4. Specific reference is made to the methods provided in Ju J. et al. 2006, PNAS, vol. 103, No. 52 and Wu J. et al. 2007, PNAS, vol. 104, No. 42 respectively, the entire contents of each of which, including the supporting information, are incorporated herein by reference.

### 6.3. Example 3. Synthesis of (L) 3′-O-Azidomethyl-dNTP-Label

### 6.3.1. Synthesis of (L) 3′-O-azidomethyl-dATP-ROX

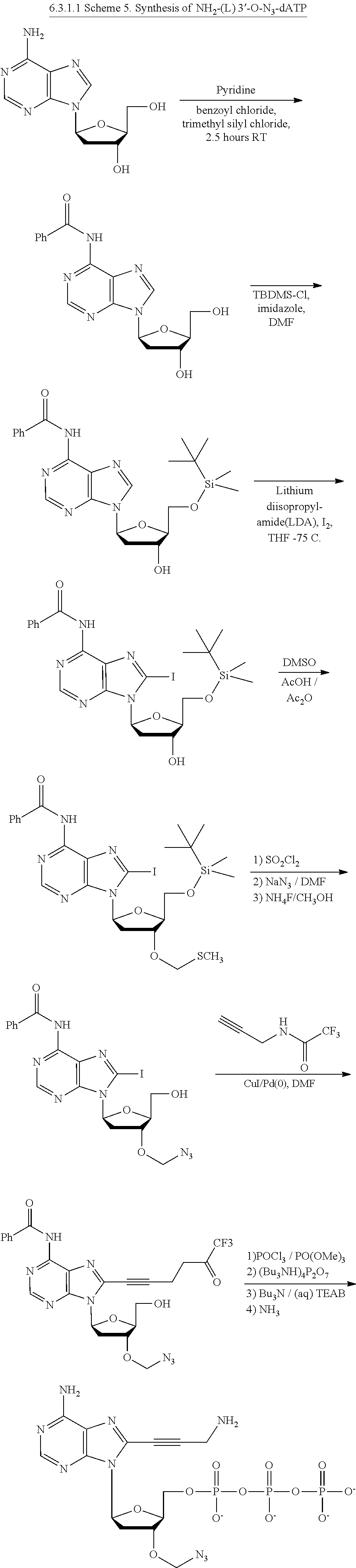

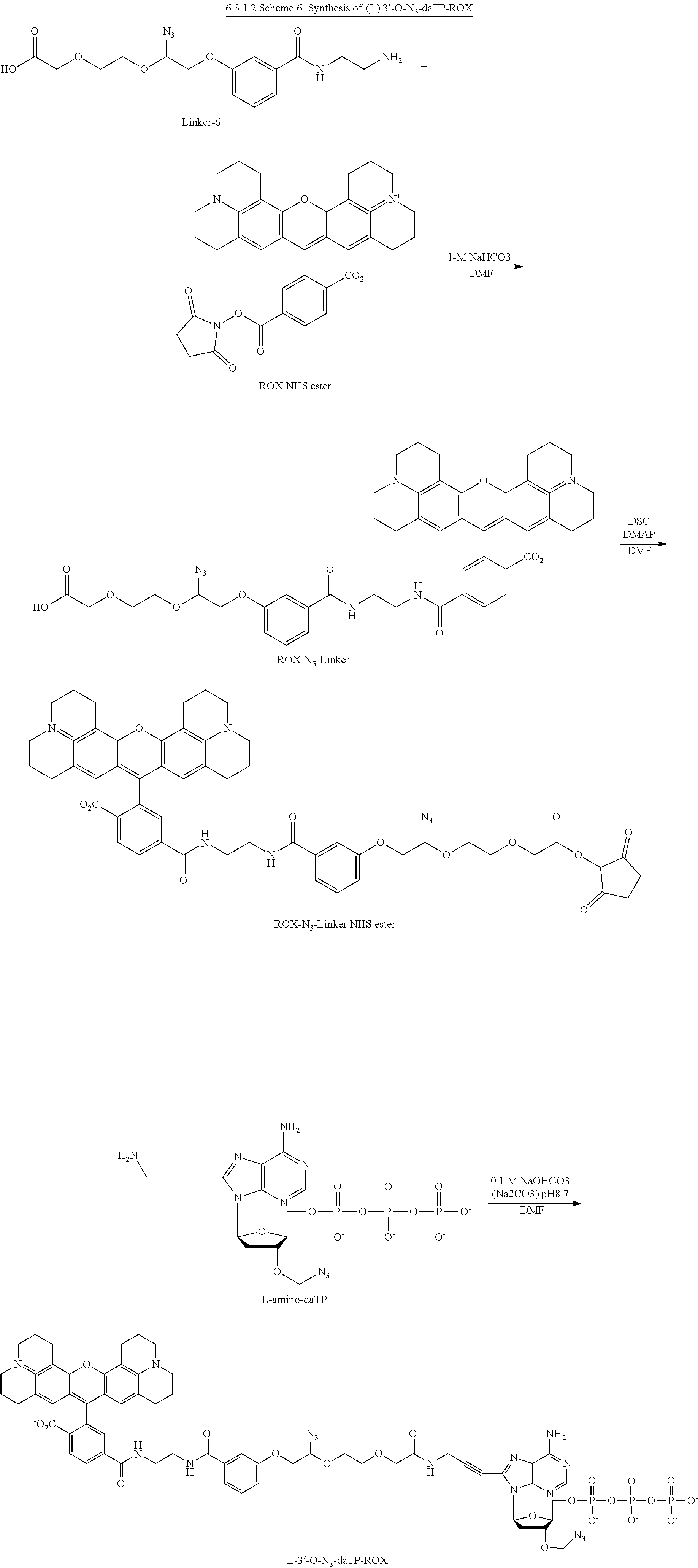

Azido-ROX compound (ROX-N₃-Linker). (2-{2-[3-(2-Amino-ethylcarbamoyl)-phenoxy]-1-azido-ethoxy}-ethoxy)-acetic acid Linker-6 (7.0 mg, 0.019 mmol) prepared according to the literature (Milton J, Ruediger S, Liu X (2006) US Patent Appl US20060160081A1) is dissolved in DMF (300 μl) and 1 M NaHCO₃ aqueous solution (100 μl). A solution of ROX NHS (N-hydroxysuccinimide) ester (Invitrogen) (0.013 mmol) in DMF (400 μl) is added slowly to the above reaction mixture and then stirred at room temperature for 5 h with exclusion of light. The crude product is purified on a preparative silica gel TLC plate (CHCl₃/CH₃OH, 1:4).

L-3′-O-azidomethyl-dATP-ROX compound (L-3′-O′—N₃-dATP-ROX). To a stirred solution of ROX-N3-Linker in dry DMF (2 ml), DSC (N,N′-disuccinimidyl carbonate) (3.4 mg, 13.2 μmol) and DMAP (4-dimethylaminopyridine) (1.6 mg, 13.2 μmol) is added. The reaction mixture is stirred at room temperature for 2 h. TLC indicated that ROX-N₃-Linker is completely converted to compound ROX-N₃-Linker NHS ester, which is directly used to couple with L-amino-dATP (13 μmol) in NaHCO₃/Na₂CO₃ buffer (pH 8.7, 0.1 M) (300 μl). The reaction mixture is stirred at room temperature for 3 h with exclusion of light. The reaction mixture is purified by a preparative silica gel TLC plate (CH₃OH/CH₂Cl₂, 1:1). The crude product is further purified on reverse-phase HPLC to afford L-3′-O-azidomethyl-dATP-ROX (L-3′-O—N₃-dATP-ROX).

### 6.3.2. Synthesis of (L) 3′-O-Azidomethyl-dGTP-Cy5

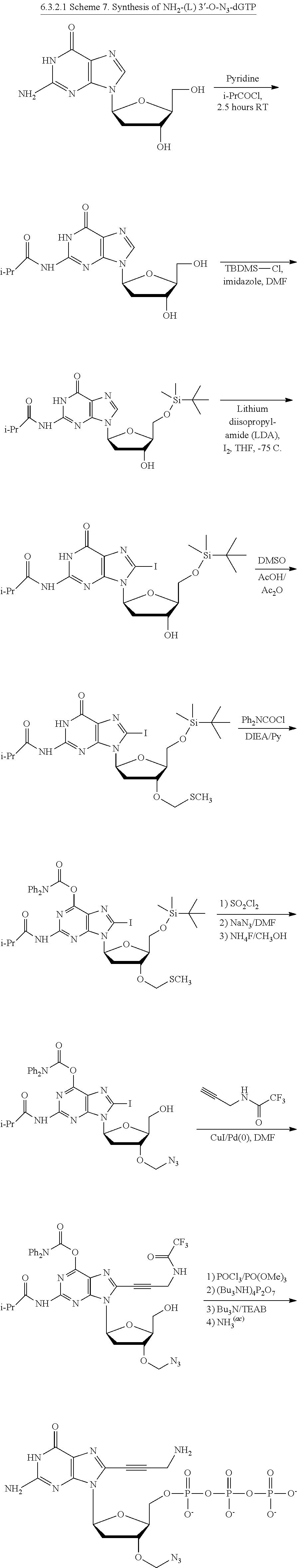

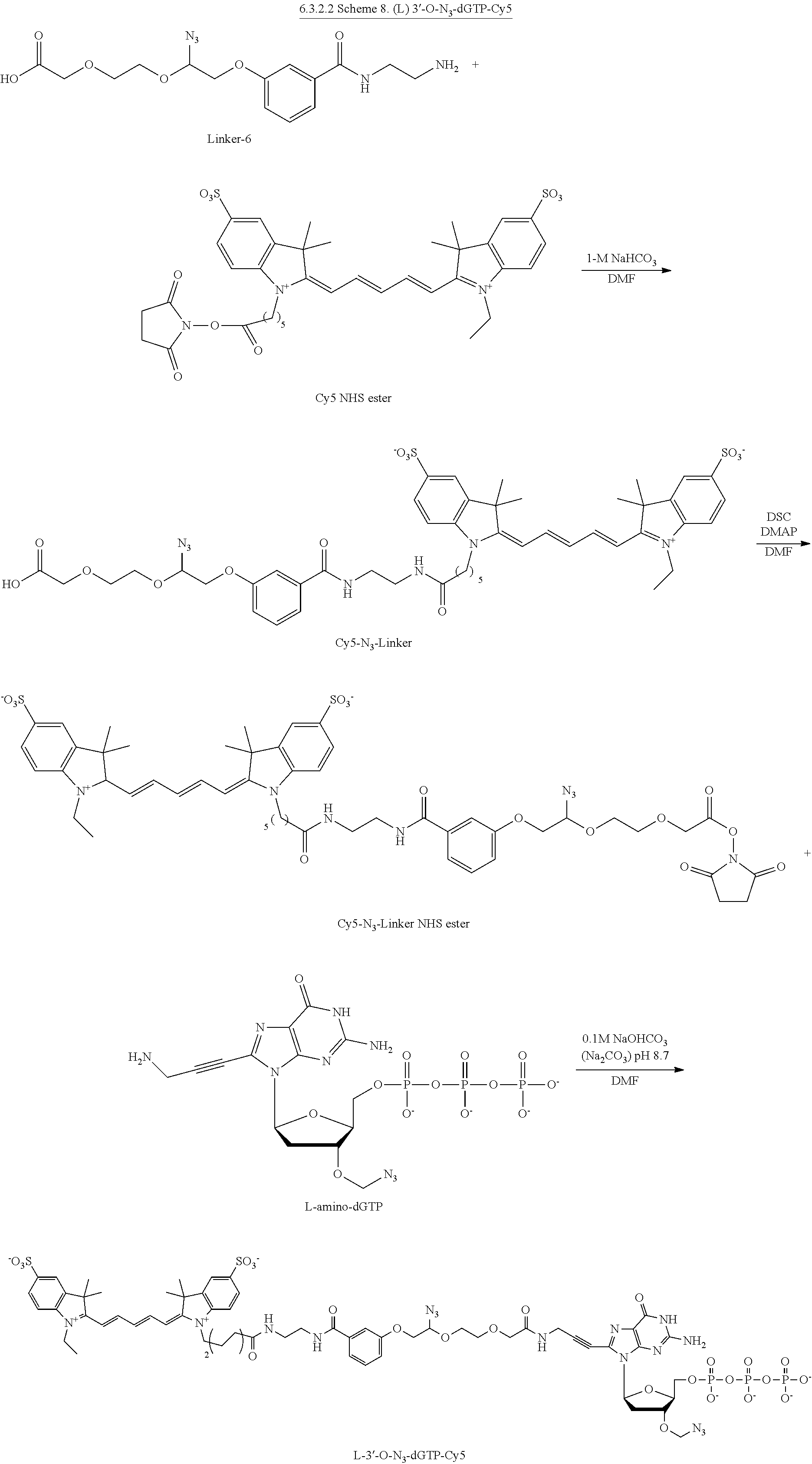

Azido-Cy5 compound (Cy5-N₃-Linker). (2-12-[3-(2-Amino-ethylcarbamoyl)-phenoxy]-1-azido-ethoxy)-ethoxy)-acetic acid Linker-6 (7.0 mg, 0.019 mmol) prepared according to the literature (Milton J, Ruediger S, Liu X (2006) US Patent Appl US20060160081A 1) is dissolved in DMF (300 μl) and 1 M NaHCO₃ aqueous solution (100 μl). A solution of Cy5 NHS (N-hydroxysuccinimide) ester (Invitrogen) (0.013 mmol) in DMF (400 μl) is added slowly to the above reaction mixture and then stirred at room temperature for 5 h with exclusion of light. The crude product is purified on a preparative silica gel TLC plate (CHCl₃/CH₃OH, 1:4).

L-3′-O-azidomethyl-dGTP-Cy5 compound (L-3′-O—N₃-dGTP-Cy5). To a stirred solution of Cy5-N₃-Linker in dry DMF (2 ml), DSC (N,N′-disuccinimidyl carbonate) (3.4 mg, 13.2 μmol) and DMAP (4-dimethylaminopyridine) (1.6 mg, 13.2 μmol) is added. The reaction mixture is stirred at room temperature for 2 h. TLC indicated that Cy5-N3-Linker is completely converted to compound Cy5-N3-Linker NHS ester, which is directly used to couple with L-amino-dGTP (13 μmol) in NaHCO₃/Na₂CO₃ buffer (pH 8.7, 0.1 M) (300 μl). The reaction mixture is stirred at room temperature for 3 h with exclusion of light. The reaction mixture is purified by a preparative silica gel TLC plate (CH₃OH/CH₂Cl₂, 1:1). The crude product is further purified on reverse-phase HPLC to afford L-3′-O-azidomethyl-dGTP-Cy5 (L-3′-O—N₃-dGTP-Cy5).

### 6.3.3. Synthesis of (L) 3′-O-Azidomethyl-dCTP-Bodipy-FL-510

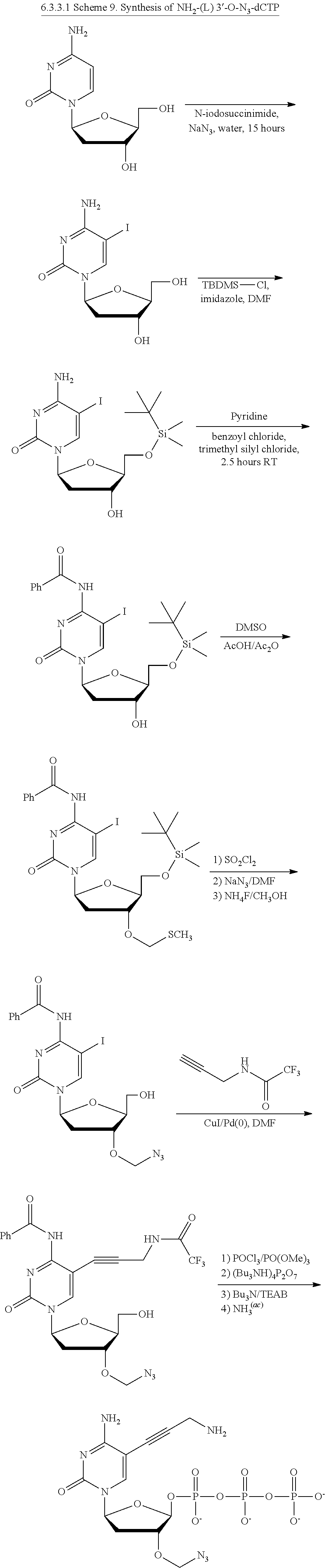

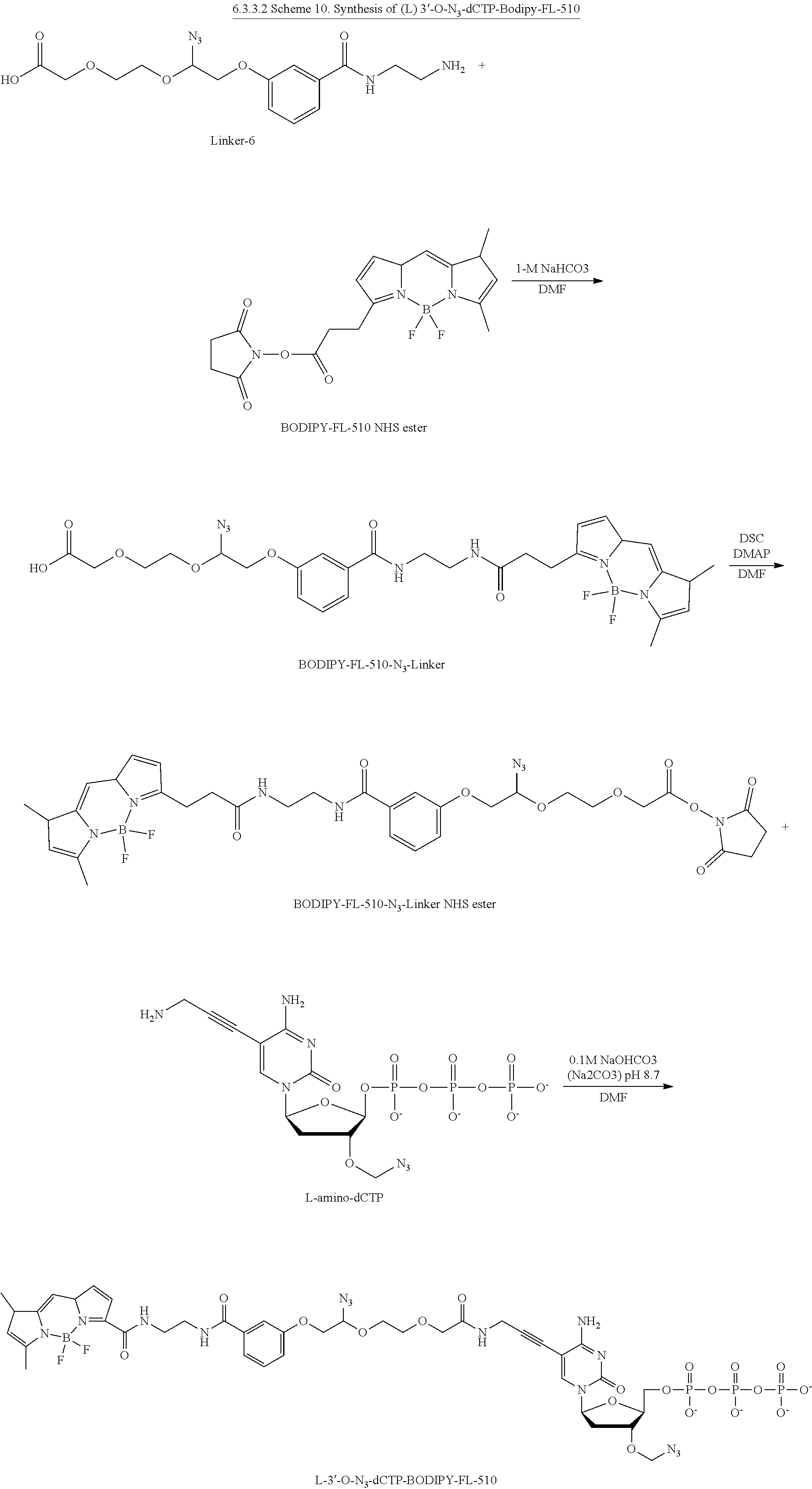

Azido-Bodipy-FL-510 (Compound BODIPY-FL-510-N3-Linker). (2-(2-[3-(2-Amino-ethylcarbamoyl)-phenoxy]-1-azido-ethoxy)-ethoxy)-acetic acid Linker-6 (7.0 mg, 0.019 mmol) prepared according to the literature (Milton J, Ruediger S, Liu X (2006) US Patent Appl US20060160081A1) is dissolved in DMF (300 μl) and 1 M NaHCO₃ aqueous solution (100 μl). A solution of Bodipy-FL-510 NHS (N-hydroxysuccinimide) ester (Invitrogen) (5.0 mg, 0.013 mmol) in DMF (400 μl) is added slowly to the above reaction mixture and then stirred at room temperature for 5 h with exclusion of light. The crude product is purified on a preparative silica gel TLC plate (CHCl₃/CH₃OH, 1:4) to afford BODIPY-FL-510-N₃-Linker.

L-3′-O-azidomethyl-dCTP-Bodipy-FL-510 (compound L-3′-O—N₃-dCTP-Bodipy-FL-510). To a stirred solution of BODIPY-FL-510-N₃-Linker in dry DMF (2 ml), DSC (N,N′-disuccinimidyl carbonate) (3.4 mg, 13.2 μmol) and DMAP (4-dimethylaminopyridine) (1.6 mg, 13.2 μmol) is added. The reaction mixture is stirred at room temperature for 2 h. TLC indicated that BODIPY-FL-510-N₃-Linker is completely converted to compound BODIPY-FL-510-N₃-Linker NHS ester, which is directly used to couple with L-amino-dCTP (13 μmol) in NaHCO₃/Na₂CO₃ buffer (pH 8.7, 0.1 M) (300 μl). The reaction mixture is stirred at room temperature for 3 h with exclusion of light. The reaction mixture is purified by a preparative silica gel TLC plate (CH₃OH/CH₂Cl₂, 1:1). The crude product is further purified on reverse-phase HPLC to afford L-3′-O-azidomethyl-dCTP-Bodipy-FL-510.

### 6.3.4. Synthesis of (L) 3′-O-Azidomethyl-dUTP-R6G

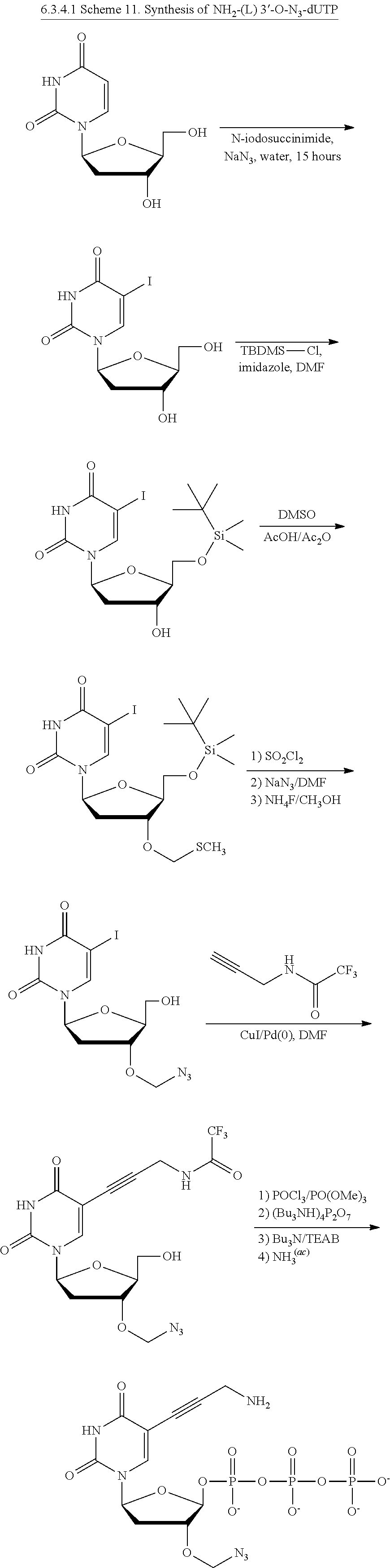

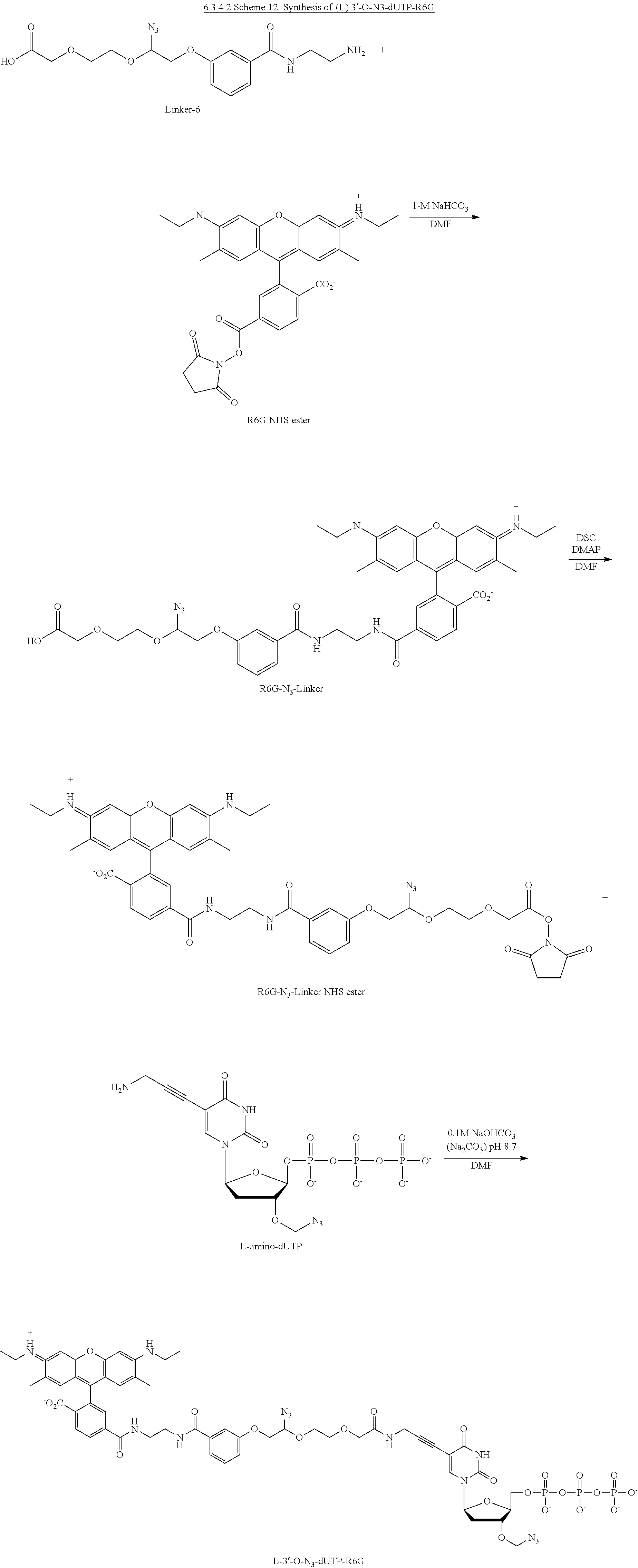

Azido-R6G (Compound R6G-N₃-Linker). (2-{2-[3-(2-Amino-ethylcarbamoyl)-phenoxy]-1-azido-ethoxy}-ethoxy)-acetic acid (7.0 mg, 0.019 mmol) prepared according to the literature (Milton J, Ruediger S, Liu X (2006) US Patent Appl US20060160081A1) is dissolved in DMF (300 μl) and 1 M NaHCO₃ aqueous solution (100 μl). A solution of R6G NHS (N-hydroxysuccinimide) ester (Invitrogen) (0.013 mmol) in DMF (400 μl) is added slowly to the above reaction mixture and then stirred at room temperature for 5 h with exclusion of light. The crude product is purified on a preparative silica gel TLC plate (CHCl₃/CH₃OH, 1:4).

L-3′-O-azidomethyl-dUTP-R6G. To a stirred solution of R6G-N3-Linker in dry DMF (2 ml), DSC (N,N′-disuccinimidyl carbonate) (3.4 mg, 13.2 μmol) and DMAP (4-dimethylaminopyridine) (1.6 mg, 13.2 μmol) is added. The reaction mixture is stirred at room temperature for 2 h. TLC indicated that R6G-N₃-Linker is completely converted to compound R6G-N₃-Linker NHS ester, which is directly used to couple with L-amino-dUTP (13 μmol) in NaHCO₃/Na₂CO₃ buffer (pH 8.7, 0.1 M) (300 μl). The reaction mixture is stirred at room temperature for 3 h with exclusion of light. The reaction mixture is purified by a preparative silica gel TLC plate (CH₃OH/CH₂Cl₂, 1:1). The crude product is further purified on reverse-phase HPLC to afford L-3′-O-azidomethyl-dUTP-R6G (L-3′-O—N₃-dUTP-R6G).

### 6.4. Example 4. Synthesis of (L) 3′-O-allyl-dNTP-allyl-Label

A person of skill in the art, having reviewed the exemplified methods provided herein would readily be able to adapt the provided methods to produce compounds with various R′ groups protecting the 3′O and alternative cleavable linkers, for example incorporating the allyl-fluorophore linkers reported in Ju J. et al. 2006, PNAS, vol. 103, No. 52, in accordance with the schemes below. Specifically, the allyl analogs of the described (L)-3′-O-azidomethyl-dNTP-NH₂ compounds are contemplated utilizing allyl bromide instead of the acetic acid/acetic anyhydride step in the above reaction schemes 5, 7, 9, and 11. Specific reference is made to the methods provided in Ju J. et al. 2006, PNAS, vol. 103, No. 52, the entire contents of which, including the supporting information, are incorporated herein by reference.

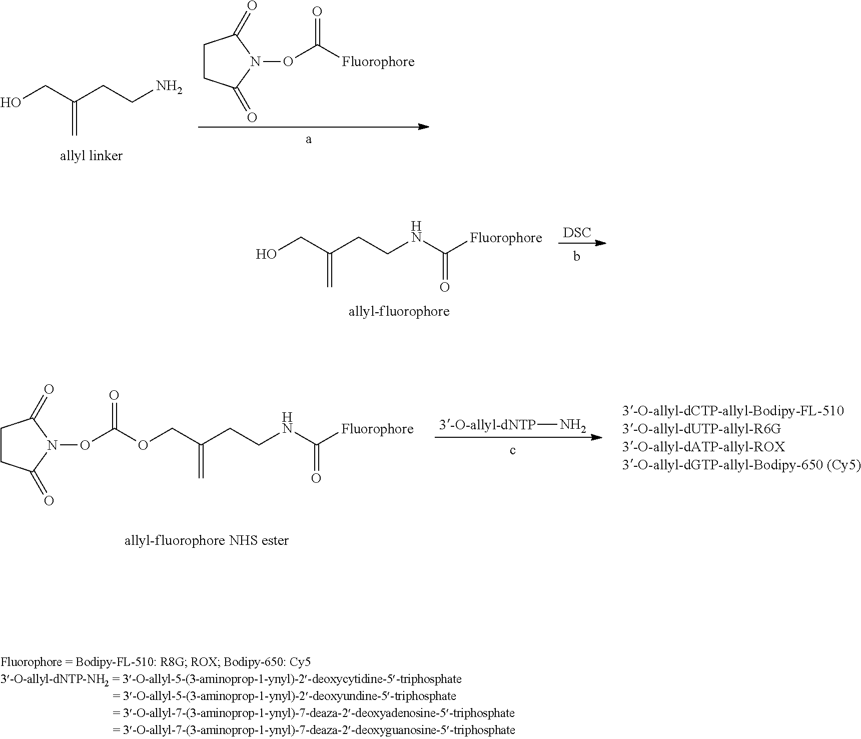

### 1. Synthesis of Allyl-Linker

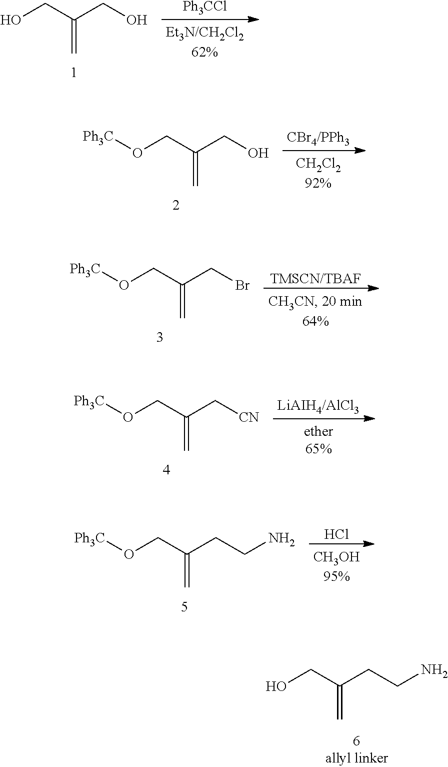

### 6.5. Example 5. Synthesis of Mirror-Image Polymerase

The amino acid sequence of 9° N polymerase is divided into multiple segments. Each polypeptide segment is synthesized using a solid phase peptide synthesis method (Fmoc-SPPS) based on a strategy of using 9-fluorenylmethoxycarbonyl (Fmoc) as a protecting group. Once polypeptide segments are synthesized, the polypeptide segments are separated and purified using a semi-preparative grade reversed-phase high performance liquid chromatography (RP-HPLC). The separated polypeptide segments are ligated using a natural chemical ligation method from the C-terminus to the N-terminus. After the chemical ligation reaction is completed, the target product is isolated using semi-preparative grade RP-HPLC.

The synthesized mirror-image polymerase is processed for folding renaturation. Using circular dichroism and mass spectrometry, it is confirmed that the mirror-image polymerase is correctly folded.

### 6.6. Example 6. Activity of Mirror-Image Polymerase

The mirror-image polymerase is mixed with (i) a polymerase reaction buffer; (ii) L-primer; (iii) L-polynucleotide template and (iv) four kinds of 0.4 mM (L) dNTPs. The reaction mixture is placed at 37° C. for 4 hours, and the reaction is terminated by adding 1 μl of 0.5 M EDTA. Activity of the mirror-image polymerase is measured based on yields of a polynucleotide fragment complementary to the template or using double stranded DNA intercalating dyes (e.g. EvaGreen dyes) and measuring the increase in fluorescence.

### 6.7. Example 7. Mirror-Image Polymerase Chain Reaction

The mirror-image polymerase is mixed with (i) a polymerase reaction buffer; (ii) 2.5 μM L-primer; (iii) 2.5 μM L-polynucleotide template and (iv) four kinds of 0.4 mM (L) dNTPs. The initial cycle consisted of 5 min at 95° C., 5 min at 50° C. (during which polymerase and BSA additions are made) and 5 min at 70° C. The segments of each subsequent PCR cycle are the following: 1 min at 93° C., 1 min at 50° C. and 5 min at 70° C. After 0, 13, 23 and 40 cycles, 20 μl amounts of 100 μl volumes are removed and subjected to agarose gel electrophoresis with ethidium bromide present to quantitate the amplification of the template sequence. The reaction yields polynucleotide fragments complementary to the template.

### 6.8. Example 8. A General Procedure of Specific Single Base Extension, Deprotection and Re-Initiation of Extension of L-Primer Using (L)-3′-O—R-dNTPs, (L)-3′-O—R-dNTPs-R-Label, (L)-ddNTPs-R₂-Label and -D-Polymerase to Demonstrate Sequencing by Synthesis (SBS)

To obtain de novo DNA sequencing data on a L-Primer/L-DNA template immobilized on a solid surface, first, verification of accurate and specific single base extension is performed using a combination mixture of solution A consisting of 3′-O-N3-dCTP (3 μM), 3′-O—N₃-dTTP (3 μM), 3′-O—N₃-dATP (3 μM) and 3′-O—N₃-dGTP (0.5 μM) and solution B consisting of ddCTP-N3-Bodipy-FL-510 (50 nM), ddUTP-N₃-R6G (100 nM), ddATP-N₃-ROX (200 nM), and ddGTP-N3-Cy5 (100 nM) in each polymerase extension reaction. For example, along with the modified L-nucleotide reversible terminators, 60 pmol of the self-priming DNA template, 1× Thermopol II reaction buffer, 40 nmol of MnCl₂ and 1 unit of D-polymerase is added together in a total reaction volume of 20 μl. The reaction consisted of incubations at 94° C. for 5 min, 4° C. for 5 min, and 65° C. for 20 min. Subsequently, the extension product is analyzed by fluorescence based gel electrophoresis (and additionally, MALDI-TOF MS can be used as an alternative option) to confirm specific incorporation of the correct nucleotide. For cleavage of the DNA extension product bearing the 3′-O—N₃-dNTP and ddNTP-N₃-fluorophores, the DNA product is resuspended in 50 μl of 100 mM TCEP solution (pH 9.0) at 65° C. for 15 min and then analyzed by either gel electrophoresis or MALDI-TOF MS to confirm cleavage of the R-label/protection group, thereby allowing re-initiation of extension of the next base. The above describes a complete single cycle of SBS (extension, label detection, and cleavage).

Generally, separate solutions, ‘Solution A’ consisting of four kinds of L-3′-O—R′-dNTP (each with dATP, dTTP, dGTP or dCTP) and ‘Solution B’ consisting of four kinds of L-ddNTP-R₂-Label (each with ddATP, ddTTP, ddGTP or ddCTP) are used in the polymerase extension reaction. Solution A and Solution B are mixed in a specific ratio (i.e. 7:3 v/v, 9:1 v/v) with a mirror-image polymerase, a L-primer, buffer and L-polynucleotide template and the mixture is incubated over multiple cycles of sequence by synthesis (SBS). During the SBS cycles, the mirror-image polymerase synthesizes a complementary sequence to the L-polynucleotide using the combination of 3′-capped nucleotide reversible terminators (L-3′-O—R′-dNTP) and cleavable fluorescent dideoxynucleotides (L-ddNTPs-R₂-Label). Replication products terminated by ddNTPs (L-ddNTPs-R₂-Label) are detected using the signal from the Label specific to ddATP, ddTTP, ddGTP or ddCTP. After detection of the signal, the 3′-OH capping group (R′ group) from the incorporated nucleotide reversible terminators (L-3′-O—R′-dNTP) and the Label from the DNA products terminated by ddNTPs (L-ddNTPs-R₂-Label) are cleaved and the polymerase reaction reinitiates. Since each Label conjugated to dATP, dTTP, dGTP or dCTP is unique, the fluorescent signals from the Label indicate the nucleotide corresponding to the termination site. Based on the signals, the sequence of the L-polynucleotide template is determined.

The L-polynucleotide template is also sequenced using four kinds of L-3′-O—R′-dNTP-R₂-Label (each with dATP, dTTP, dGTP or dCTP). The L-polynucleotide template is mixed with the four kinds of L-3′-O—R′-dNTP-R₂-Label (each with dATP, dTTP, dGTP or dCTP), L-primer, D-polymerase and buffer in a similar reaction condition described above. The mixture is incubated over multiple cycles of sequence by synthesis (SBS). During the SBS step, L-3′-O—R′-dNTP-R₂-Label is incorporated to the synthesized product. The L-3′-O—R′-dNTP-R₂-Label includes a chemical moiety (R′) capping the 3′-OH and a Label tethered to the base through a chemically cleavable linker (R₂). Following the incorporation, fluorescent signals from the Label is detected and then the 3′-capping moiety (R′) and the Label on the reaction product are cleaved to reinitiate the polymerase reaction. Since each Label conjugated to dATP, dTTP, dGTP or dCTP is unique, the fluorescent signals from the Label indicate the nucleotide corresponding to the termination site. Based on the signals, the sequence of the L-polynucleotide template is determined.

### 6.9. Example 9. Sequencing L-Polynucleotide by a Method Analogous to Sanger Sequencing

The L-polynucleotide template is sequenced (analogous to sanger sequencing) using four kinds of L-ddNTP-R₂-Label (each with ddATP, ddTTP, ddGTP or ddCTP). The four kinds of L-ddNTP-R₂-Label are mixed with an L-polynucleotide template, an L-primer, four kinds of L-dNTP (L-dATP, L-dGTP, L-dCTP and L-dTTP), D-polymerase and buffer in a similar reaction condition described above. In the mixture, L-ddNTP-R₂-Label is added to the mixture in a much smaller amount compared to L-dNTP. PCR (cycle sequencing—generation of DNA fragment ladder) is performed with the mixture. The replication product is separated by size using gel electrophoresis and signals from the L-ddNTPs-R₂-Label incorporated into the replication product are detected. Since each Label conjugated to dATP, dTTP, dGTP or dCTP is unique, the fluorescent signals from the Label indicate the nucleotide corresponding to the termination site. Based on the fluorescence signals and the fragment mobility (based on size), the sequence of the L-polynucleotide template is determined.

### 6.10. Example 10. Completed Synthesis of (L)-3′-O-Azidomethyl-dNTPs

Four target molecules and their synthetic routes were designed as shown in Scheme 13-16. The L-3′-O-Azidomethyl-dTTP (dT-4) and L-3′-O-Azidomethyl-dCTP (dC-5) were prepared and characterized by ¹H, ³¹P NMR and HRMS. For the synthesis of L-3′-O-Azidomethyl-dATP (dA-5) and L-3′-O-Azidomethyl-dGTP (dG-6), we have synthesized the intermediates dA-3 and dG-5, respectively.

### Materials and Methods

All solvents and reagents were reagent grades, purchased commercially, and used without further purification unless specified. All chemicals were purchased from Sigma-Aldrich, Fisher Scientific, TCI etc. ¹H NMR spectra were recorded on a Bruker Ascend™ (400 MHz) spectrometer from Chapman University and reported in parts per million (ppm) from a CDCl₃ (7.26 ppm) or D₂O. Data were reported as follows: (s=singlet, d=doublet, t!triplet, q=quartet, m=multiplet, dd=doublet of doublets, J=coupling constant in Hz, integration). Proton-decoupled ³¹P NMR spectra were recorded on a Bruker Ascend™ (121.4 MHz) spectrometer from Chapman University. High-resolution mass spectra (HRMS) were obtained from School of Pharmacy, Chapman University and Analytical Chemistry Instrumentation Facility at University California of Riverside. Starting materials beta-L-deoxythymidine, beta-L-deoxycytidine, beta-L-deoxyadenosine, and beta-L-deoxyguanosine were purchased from Chemgenes. Analytical (Polaris 180A C18-A, 4.6×250 mm, 5 um) and semi-prep (Polaris 180A C18-A, 4.6×250 mm, 5 um) HPLC columns were purchased from Agilent. The 3′-O-modified nucleotides were purified with reverse-phase HPLC on a 4.6×250 mm C18 column (Polaris), mobile phase: A, 25 mM TEAB buffer in water; B, 25 mM TEAB buffer in acetonitrile. Elution was performed isocratic conditions as described in each procedure.

### 6.10.1. Synthesis of (L) 3′-O-Azidomethyl-dTTP

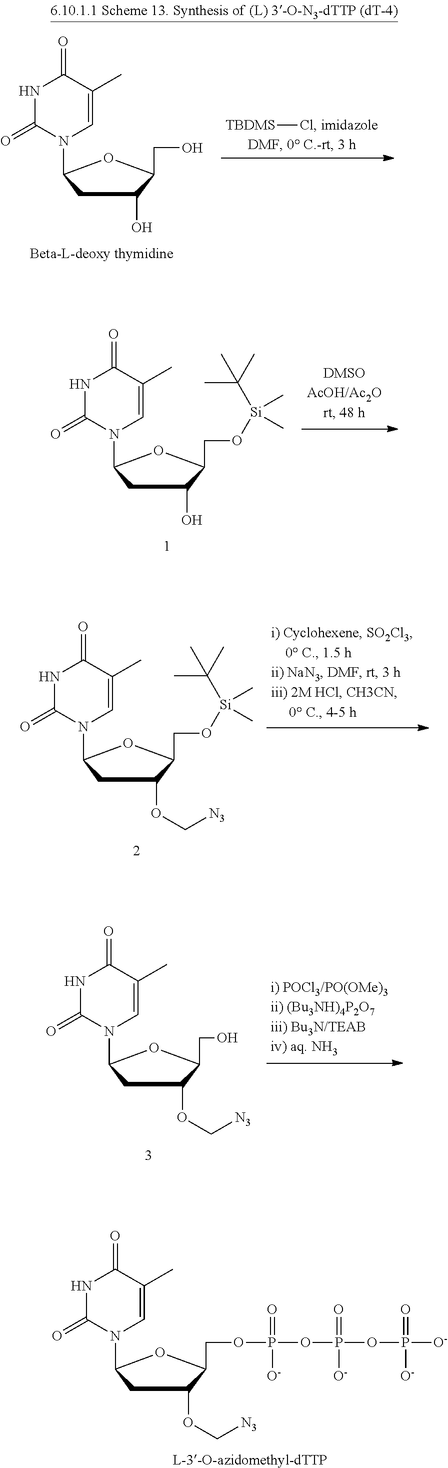

### Experimental Procedure:

dT-1 synthesis: To a solution of β-L-deoxy Thymidine (1.5 g, 6.18 mmol) in anhydrous N,N-dimethylformamide (DMF) (37.5 mL) was added imidazole (633 mg, 9.30 mmol) and tert-butyldimethylsilyl chloride (1.02 g, 6.81 mmol) were added at 0° C. under nitrogen. After stirred at room temperature for 3 h, the solution was added iced clod water and extracted with EtOAc (2×50 mL). The combined organic layers were dried over Na₂SO₄, filtered and concentrated. The resulting residue was dissolved in CH₃OH and added silica gel. The solution was dried under reduced pressure until dryness. The resulting residue was purified by column chromatography (2:98 to 5:95, CH₃OH—CH₂Cl₂) to afford dT-1 (1.8 g, 82%) as a white solid. R_{f} 0.35 (1:19 CH₃OH—CH₂Cl₂). Product was confirmed by TLC.

dT-2 synthesis: To a stirred solution of dT-1 (1.9 g; 5.33 mmol) in DMSO (18 ml) was added acetic acid (9 ml) and acetic anhydride (27 ml) at room temperature. The reaction mixture was stirred at room temperature for 48 h. A saturated NaHCO₃ solution was added at 0° C. and stirred for 30 min, and the aqueous layer was extracted with CH₂Cl₂ (2×100 ml). The combined organic layers were dried over Na₂SO₄, filtered and concentrated. The resulting residue was purified by flash column chromatography (1:1, Hexanes-EtOAc) to afford dT-2 as a yellowish syrup (2.0 g, 90%). R_{f} 0.55 (1:1 hexanes-EtOAc); ¹H NMR (400 MHz, CDCL₃): δ 9.36 (s, 1H, NH), 7.48 (d, 1H, ⁴J=1.1 Hz, H-6), 6.31 (dd, 1H, J_{1′,2′a}=5.6 Hz, J_{1′,2′b}8.6 Hz, H-1′), 4.68 (d, 1H, J_{gem}=11.8 Hz, CH₂S), 4.61 (d, 1H, J_{gem}=11.8 Hz, CH₂S), 4.47 (app dt, 1H, J_{3′,2′b}5.9 Hz, J_{3′,2′a}1.9 Hz, H-3′), 4.12-4.08 (m, 1H, H-4′), 3.89 (dd, 1H, J_{5′a,4}=2.6 Hz, J_{gem}=11.3 Hz, H-5′a), 3.80 (dd, 1H, J_{5′a,4}=2.9 Hz, J_{gem}=11.3 Hz, H-5′b), 2.41 (ddd, 1H, J_{2′a,3′}=1.9 Hz, J_{2′a,1′}=5.6 Hz, J_{gem}=13.6 Hz, H-2′a), 2.16 (s, 3H, SCH₃), 1.99 (ddd, 1H, J_{2′b,3′}=5.9 Hz, J_{2′b,1}=8.6 Hz, J_{gem}=13.6 Hz, H-2′b), 1.92 (d, 3H, ⁴J=1.1 Hz, CH₃), 0.93 (s, 9H, (CH₃)₃CSi), 0.12 (s, 6H, (CH₃)₂Si)); HRMS (ESI) m/z [M+H]⁺ calcd for C₁₈H₃₃N₂O₅SSi 417.1879. Found 417.1881.

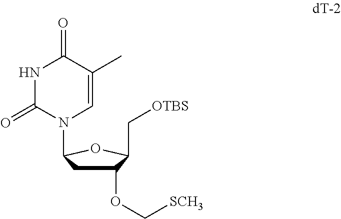 [SMILES: CSCO[C@H]1C[C@@H](n2cc(C)c(=O)[nH]c2=O)O[C@H]1CO[Si](C)(C)C(C)(C)C]

dT-3 synthesis: To a stirred solution of dT-2 (2.0 g, 4.80 mmol) in dry CH₂Cl₂ (45 mL), cyclohexene (2.1 mL) and SO₂C2 (1.0 M in DCM) (6.48 mL, 6.48 mmol) were added. The reaction mixture was stirred at 0° C. for 3 h. The volatiles were removed under reduced pressure. The residue was dissolved in dry DMF (30 mL) and reacted with NaN₃ (1.88 g, 28.8 mmol) at room temperature for 2 h. The reaction mixture was dispersed in cold distilled water (100 mL) and extracted with EtOAc (2×200 mL). The combined organic extract was dried over Na₂SO₄ and concentrated under reduced pressure. The resulting residue was dissolved in CH₃CN (10 mL) and reacted with 2M HCl (2-3 mL) at 0° C. for 5 h. Saturated Na₂CO₃ solution was added and extracted with CH₂Cl₂ (2×50 mL), dried on Na₂SO₄, and concentrated. The organic layer was washed with water and the organic layer was dried on Na₂SO₄, and concentrated. The resulting residue was purified by flash column chromatography (hexane/ethyl acetate, 3:7 to 1:4) to afford 3 as a white powder. R_{f} 0.20 (1:4 hexanes-EtOAc); ¹H NMR (400 MHz, CDCL₃): δ 8.63 (s, 1H, NH), 7.38 (d, 1H, ⁴J=1.1 Hz, H-6), 6.13 (app t, 1H, J_{1′,2′a}=J_{1′,2′b}=7.0 Hz, H-1′), 4.77 (d, 1H, J_{gem}=9.0 Hz, CH₂N₃), 4.70 (d, 1H, J_{gem}=9.0 Hz, CH₂N₃), 4.50 (app dt, 1H, J_{3′,2′b}=6.0 Hz, J_{3′,2′a}=3.5 Hz, H-3′), 4.16-4.12 (m, 1H, H-4′), 3.98 (dd, 1H, J_{5′a,4′}=2.7 Hz, J_{gem}=12.0 Hz, H-5′a), 3.84 (dd, 1H, J_{5′a,4′}=2.8 Hz, J_{gem}=12.0 Hz, H-5′b), 2.51-2.38 (m, 3H, H-2′a, H-2′b, 5′-OH), 1.94 (d, 3H, ⁴J=1.1 Hz, CH₃); HRMS (ESI) m/z [M+H]⁺ calcd for C₁₁H₁₆N₅O₅S 298.1151. Found 298.1144

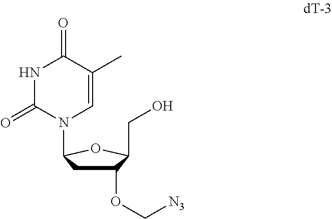 [SMILES: Cc1cn([C@@H]2C[C@H](OCN=[N+]=[N-])[C@H](CO)O2)c(=O)[nH]c1=O]

dT-4 synthesis: dT-3 (120 mg, 0.403 mmol) was dried in a vacuum desiccator over P₂O₅ overnight. To a solution of dT-3 in trimethyl phosphate (5 mL) was added POCl₃ (94.3 uL, 1.00 mmol) dropwisely at 0° C. The mixture was stirred at 0° C. for 2 hours and then was added a well-vortexed mixture of tributylammonium pyrophosphate (552 mg) and tributylamine (738 uL, 3.10 mmol) in anhydrous DMF (2 mL). The mixture was stirred for 1 hour at room temperature and 0.1M triethylammonium bicarbonate buffer (TEAB buffer, pH 8.5, 20 ml) was then added and the mixture was stirred overnight at room temperature. The resulting mixture was concentrated under reduced pressure and the residue was diluted with 10 ml of water. The crude mixture was extracted with CH₂Cl₂ (2×10 mL) and the aqueous layer was concentrated under reduced pressure. The residue was then purified with anion exchange chromatography on DEAE-Sephadex A-25 using a gradient of TEAB (pH 8.5; 0.1-0.8 M). The fractions with products (0.3M-0.4M) was collected and concentrated under reduced pressure. The residue was diluted with ddH₂O and subjected to C18 HPLC (4% isocratic, 25 mM TEAB in acetonitrile:25 mM TEAB in water). The product (retention time: 19 min) was collected and concentrated under reduced pressure to afford dT-4 (14 mg, 6.5%) as syrup. R_{f} 0.28 (1.2 TEAB-ACN); ¹H NMR (400 MHz, D₂O): δ 7.61 (d, 1H, ⁴J=1.0 Hz, H-6), 6.19 (dd, 1H, J_{1′,2′a}=5.8 Hz, J_{1′,2′b}=8.6 Hz, H-1′), 4.74 (d, 1H, J_{gem}=8.8 Hz, CH₂N₃), 4.69 (d, 1H, J_{gem}=8.8 Hz, CH₂N₃), 4.53-4.48 (m, 1H, H-3′), 4.25-4.20 (m, 1H, H-4′), 4.12-4.00 (m, 2H, H-5′a, H-5′b), 2.37 (ddd, 1H, J_{2′a,1′}=5.8 Hz, J_{2′a,3′}=2.0 Hz, J_{gem}=14.4 Hz, H-2′a), 2.25 (ddd, 1H, J_{2′a,1′}=8.8 Hz, J_{2′a,3′}=5.9 Hz, J_{gem}=14.4 Hz, H-2′b), 1.78 (d, 3H, ⁴J=1.0 Hz, CH₃); ³¹P NMR (121.4 MHz, D₂O): δ −10.95 (bs, 1P), −11.75 (d, J=20.0 Hz, 1P), −23.36 (bs, 1P); HRMS (ESI) m/z [M−H]⁻ calcd for C₁₁H₁₇N₅O₁₄P₃⁻ 535.9990. Found 535.9993.

dT-4:

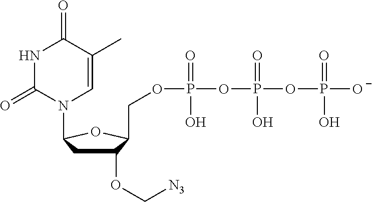 [SMILES: Cc1cn([C@@H]2C[C@H](OCN=[N+]=[N-])[C@H](COP(=O)(O)OP(=O)(O)OP(=O)([O-])O)O2)c(=O)[nH]c1=O]

### 6.10.2. Synthesis of L-3′-O-Azidomethy-dCTP

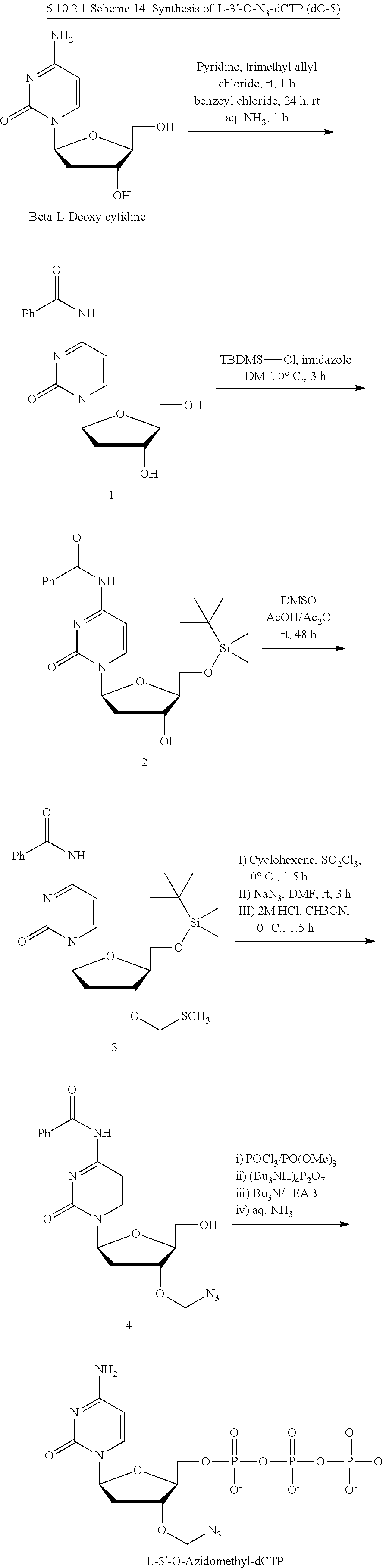

dC-1 synthesis: O-L-Deoxycytidine (1 g, 4.40 mmol) was co-evaporated with anhydrous pyridine (2×10 mL) and then dissolved in anhydrous pyridine (15 mL) under nitrogen. Trimethylsilyl chloride (TMSCl, 2.79 mL, 22.0 mmol) was slowly added and the mixture was stirred at room temperature for 1 hour, after which benzoyl chloride (2.56 mL, 22.0 mmol) was added and the solution was stirred at room temperature for another 24 h. After cooling to 0° C., water (10 mL) was added and the mixture was stirred at 0° C. for 20 min. Then, concentrated ammonia solution (15 mL) was added and the solution was stirred for a further 1 hour while warming to rt. The solvent was evaporated under reduced pressure and the resultant crude product was purified by column chromatography (19:1, CH₂Cl₂—CH₃OH) to afford dC-1 (520 mg, 36%) as white solid. R_{f} 0.35 (1:19 CH₃OH—CH₂Cl₂).

dC-2 synthesis: To a solution of dC-1 (280 mg, 0.84 mmol) in anhydrous DMF (5.0 mL) with imidazole (172 mg, 2.53 mmol) and tert-butyldimethylsilyl chloride (204 mg, 1.35 mmol) at 0° C. The solution was stirred under nitrogen at 0° C. for 3 hours. After completion of the reaction (TLC monitoring), cold water (10 mL) was added and extracted with EtOAc (3×20 mL). The combined organic layers were dried over Na₂SO₄, filtered and concentrated. The resulting residue was purified by column chromatography (19:1 to 9:1, CH₂Cl₂—CH₃OH) afforded dC-2 (280 mg, 74%) as colorless oil. R_{f}0.32 (1:19 CH₃OH—CH₂Cl₂).

dC-3 synthesis. To a stirred solution of dC-2 (240 mg, 0.538 mmol) in DMSO (6 ml) was added acetic acid (3 ml) and acetic anhydride (9 ml) at room temperature. The reaction mixture was stirred at room temperature for 72 h. A saturated NaHCO₃ solution was added at 0° C. and stirred for 30 min, and the aqueous layer was extracted with CH₂C2 (2×30 ml). The combined organic extract was dried over Na₂SO₄, filtered and concentrated. The crude product was purified by flash column chromatography (1:1, hexanes-EtOAc) to afford dC-3 (163 mg, 60/6) as a white powder. R_{f}0.22 (1:1 hexanes-EtOAc); ¹H NMR (400 MHz, CDCL₃): δ 8.42 (d, 1H, J=7.5 Hz), 7.92 (d, 2H, J=7.5 Hz, ArH), 7.67-7.58 (m, 1H, ArH), 7.56-7.38 (m, 3H, ArH), 6.29 (app t, 1H, J_{1′,2′a}=J_{1′,2′b}=6.0 Hz, H-1′), 4.69 (d, 1H, J_{gem}=11.7 Hz, CH₂S), 4.61 (d, 1H, J_{gem}=11.7 Hz, CH₂S), 4.50 (app dt, 1H, J_{3′,2′a}=J_{3′,4′}=4.0 Hz, J_{3′,2′b}=6.0 Hz, H-3′), 4.21-4.16 (m, 1H, H-4′), 4.00 (dd, 1H, J_{5′a,4′}=3.2 Hz, J_{gem}=11.8 Hz, H-5′a), 3.84 (dd, 1H, J_{5′a,4′}=2.6 Hz, J_{gem}=11.8 Hz, H-5′b), 2.79 (ddd, 1H, J_{2′a,1′}=6.0 Hz, J_{2′a,3′}=4.0 Hz, J_{gem}=13.7 Hz, H-2′a), 2.21-2.13 (m, 4H, H-2′b, SCH₃), 0.95 (s, 9H, (CH₃)₃CSi); 0.15 (s, 3H, CH₃Si), 0.14 (s, 3H, CH₃Si); HRMS (ESI) m/z [M+H]⁺ calcd for C₂₄H₃₆N₃O₅SSi 506.2145. Found 506.2150.

dC-4 synthesis. To a stirred solution of dC-3 (220 mg, 0.67 mmol) in dry CH₂Cl₂ (6 mL) was added cyclohexene (200 uL) and SO₂Cl₂ in CH₂Cl₂ (1.0 M, 0.6 mL) were added. After stirred at 0° C. for 1.5 hours, the volatiles were removed under reduced pressure. To a solution of the residue in dry DMF (3 mL) was added NaN₃ (169 mg, 2.61 mmol) at room temperature for 3 hours. The reaction mixture was dispersed in cold distilled water (30 mL) and extracted with EtOAc (2×30 mL). The combined organic extract was dried over Na₂SO₄ and concentrated under reduced pressure. The resulting residue was performed de-TBDMS reaction by 2M HCl, the reaction was over 1-2 h at room temperature. The mixture was neutralized by saturated NaHCO₃ solution and diluted with EtOAc. The organic layer was dried over Na₂SO₄ and concentrated under reduced pressure. The residue was purified by flash column chromatography (1:2, Hexanes-EtOAc to 100% EtOAc) to afford dC-4 (73 mg, 56%) as a colorless oil. R_{f} 0.3 (1:4 hexanes-EtOAc); ¹H NMR (400 MHz, CDCL₃): δ 8.99 (bs, 1H, NH), 8.32 (d, 1H, J=7.5 Hz), 7.87 (d, 2H, J=7.5 Hz, ArH), 7.65-7.53 (m, 2H), 7.52-7.45 (m, 2H, ArH), 6.18 (app t, 1H, J_{1′,2′a}=J_{1′,2′b}=6.3 Hz, H-1′), 4.79 (d, 1H, J_{gem}=9.2 Hz, CH₂N₃), 4.68 (d, 1H, J_{gem}=9.2 Hz, CH₂N₃), 4.52 (app dt, 1H, J_{3′,2′a}=J_{3′,4′}=3.9 Hz, J_{3′,2′b}=6.3 Hz, H-3′), 4.26-4.22 (m, 1H, H-4′), 4.04 (dd, 1H, J_{5′a,4′}=2.8 Hz, J_{gem}=12.0 Hz, H-5′a), 3.89 (dd, 1H, J_{5′a,4′}=2.8 Hz, J_{gem}=12.0 Hz, H-5′b), 3.45 (bs, 1H, 5′-OH), 2.68 (ddd, 1H, J_{2′a,1′}=6.3 Hz, J_{2′a,3′}=3.9 Hz, J_{gem}=13.5 Hz, H-2′a), 2.44 (app dt, 1H, J_{2′b,1′}=J_{2′a,3′}=6.3 Hz, J_{gem}=13.5 Hz, H-2′b); HRMS (ESI) m/z [M+H]⁺ calcd for C₁₇H₁₉N₆O₅ 387.1417. Found 387.1426.

dC-5 synthesis: dC-4 (100 mg, 0.259 mmol) was dried in a vacuum desiccator over P₂O₅ overnight. To a solution of dC-4 in trimethyl phosphate (5 mL) was added POCl₃ (60.5 uL, 0.647 mmol) dropwisely at 0° C. The mixture was stirred at 0° C. for 2 hours and then was added a well-vortexed mixture of tributylammonium pyrophosphate (355 mg, 0.647 mmol) and tributylamine (473.8 uL, 1.99 mmol) in anhydrous DMF (2 mL). The mixture was stirred for 1.5 hours at room temperature and 0.1M triethylammonium bicarbonate buffer (TEAB buffer, pH 8.5, 0.1 M, 15 ml) was then added and the mixture was stirred for 1 hour at room temperature. The mixture was then added concentrated ammonium hydroxide (10 mL) and stirred overnight at room temperature. The resulting mixture was concentrated under reduced pressure and the residue was diluted with 30 ml of water. The crude mixture was extracted with CH₂Cl₂ (2×20 mL) and the aqueous layer was concentrated under reduced pressure. The residue was then purified with anion exchange chromatography on DEAE-Sephadex A-25 using a gradient of TEAB (pH 8.5, 0.2-0.8 M). The fractions with products (0.3M-0.4M) was collected and concentrated under reduced pressure. The residue was diluted with ddH₂O and subjected to C18 HPLC (2% isocratic, 25 mM TEAB in ACN:25 mM TEAB in water). The product (retention time: 13.2 min) was collected and concentrated under reduced pressure to afford dC-5 (18 mg, 13.4%) as syrup. R_{f} 0.43 (1:2 TEAB-ACN); ¹H NMR (400 MHz, D₂O): δ 8.00-7.88 (m, 1H), 6.23 (dd, 1H, J_{1′,2′a}=5.6 Hz, J_{1′,2′b}=7.9 Hz, H-1′), 6.18-6.06 (m, 1H) 4.85-4.74 (m, 2H, CH₂N₃), 4.56-4.51 (m, 1H, H-3′), 4.35-4.29 (m, 1H, H-4′), 4.18-4.09 (m, 2H, H-5′a, H-5′b), 2.55-2.46 (m, 1H, H-2′a), 2.31-2.19 (m, 1H, H-2′b); HRMS (ESI) m/z [M−H]⁻ calcd for C₁₀H₁₆N₅O₁₃P₃⁻ 520.9994. Found 520.9990.

### 6.10.3. L-3′-O-Azidomethyl-dATP Synthesis

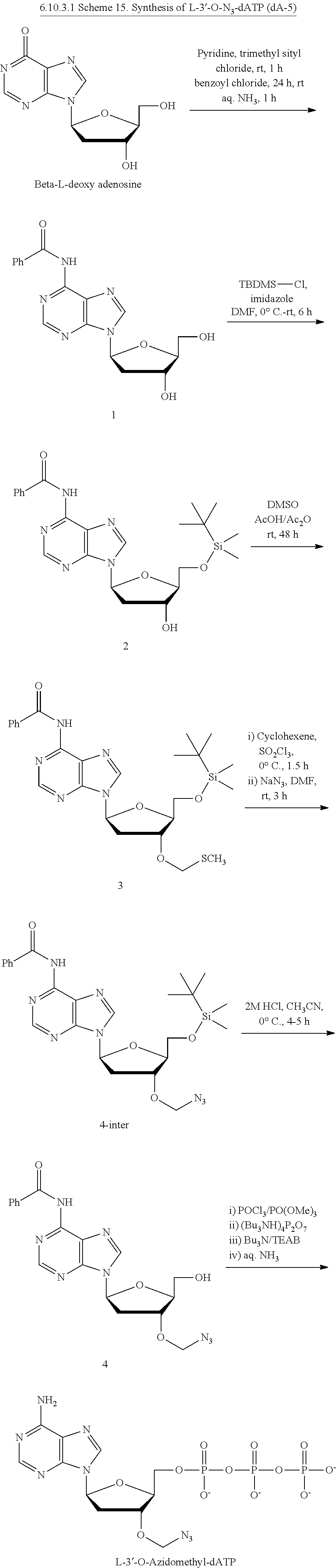

### Experimental Procedure:

dA-1 synthesis: O-L-deoxy Adenosine (2.0 g, 7.96 mmol) was co-evaporated with anhydrous pyridine (2 times: 10 mL+10 mL) and dissolved in anhydrous pyridine (20 mL). The resulting solution was cooled to 0° C. and added chlorotrimethylsilane (TMSCl, 5.06 mL, 39.8 mmol) dropwisely via a syringe. The mixture was stirred for 1 hours at 0° C. The solution was added benzoyl chloride (4.62 mL, 39.8 mmol) dropwisely via a syringe and ice bath was removed. After stirred at room temperature for 3 hours, the flask was placed on ice bath and concentrated ammonia solution (15 mL) was added. The solution was stirred for 30 min at 0° C., and then evaporated to dryness. The residue was dissolved in CH₃OH and silica gel was added. The mixture was dried under reduced pressure. The residue was purified by column chromatography (49:1 to 19:1, CH₂Cl₂—CH₃OH) to afford dA-1 (2.12 g, 75%) as white solid.

dA-2 synthesis: To a solution of dA-1 (2.12 g, 5.98 mmol) in anhydrous DMF (30 mL) was added imidazole (608 mg, 8.95 mmol) and tert-butyldimethylsilyl chloride (989 mg, 6.56 mmol) at 0° C. under nitrogen. The reaction mixture was stirred for 3 hours at 0° C. After the completion of reaction, cold water (50 mL) was added and the mixture was extracted with EtOAc (3×50 mL). The combined organic layers were dried over anhydrous Na₂SO₄, filtered and concentrated The resulting residue was purified by column chromatography (1:2, Hexanes-EtOAc to 100% EtOAc) to afford compound dA-2 (1.92 g, 69/6) as colorless oil.

dA-3 synthesis: To a stirred solution of dA-2 (1.8 g; 3.83 mmol) in DMSO (24 ml) was added acetic acid (12 ml) and acetic anhydride (36 ml) at room temperature. After stirred at room temperature for 72 hours. The solution was added saturated NaHCO₃ solution at 0° C. and stirred for 30 min. The mixture was extracted with CH₂Cl₂ (2×100 ml). The combined organic layers were dried over Na₂SO₄, filtered and concentrated. The residue was purified by flash column chromatography (1:1, Hexanes-EtOAc) to afford dA-3 (1.27 g, 63%) as a white foam. R_{f}0.28 (1:7 hexanes-EtOAc); ¹H NMR (400 MHz, CDCL₃): δ 9.28 (bs, 1H, NH), 8.77 (s, 1H), 8.33 (s, 1H), 8.02 (d, 2H, J=7.5 Hz, ArH), 7.62-7.55 (m, 1H, ArH), 7.53-7.47 (m, 2H, ArH), 6.51 (dd, 1H, J_{1′,2′a}=7.2 Hz, J_{1′,2′b}=6.0 Hz, H-1′), 4.74-4.64 (m, 3H, CH₂S, H-3′), 4.25-4.18 (m, 1H, H-4′), 3.89 (dd, 1H, J_{5′a,4′}=4.4 Hz, J_{gem}=11.0 Hz, H-5′a), 3.82 (dd, 1H, J_{5′a,4′}=3.3 Hz, J_{gem}=11.0 Hz, H-5′b), 2.79 (ddd, 1H, J_{2′a,1′}=7.2 Hz, J_{2′a,3′}=6.0 Hz, J_{gem}=13.7 Hz, H-2′a), 2.63 (ddd, 1H, J_{2′b,1′}=6.0 Hz, J_{2′b,3′}=3.0 Hz, J_{gem}=13.7 Hz, H-2′b), 2.18 (s, 3H, SCH₃), 0.91 (s, 9H, (CH₃)₃CSi); 0.10 (s, 6H, (CH₃)₂Si); HRMS (ESI) m/z [M+H]⁺ calcd for C₂₅H₃₆N₅O₄SSi 530.2157. Found 530.2132.

### 6.10.4. L-3′-O-Azidomethy-dGTP Synthesis

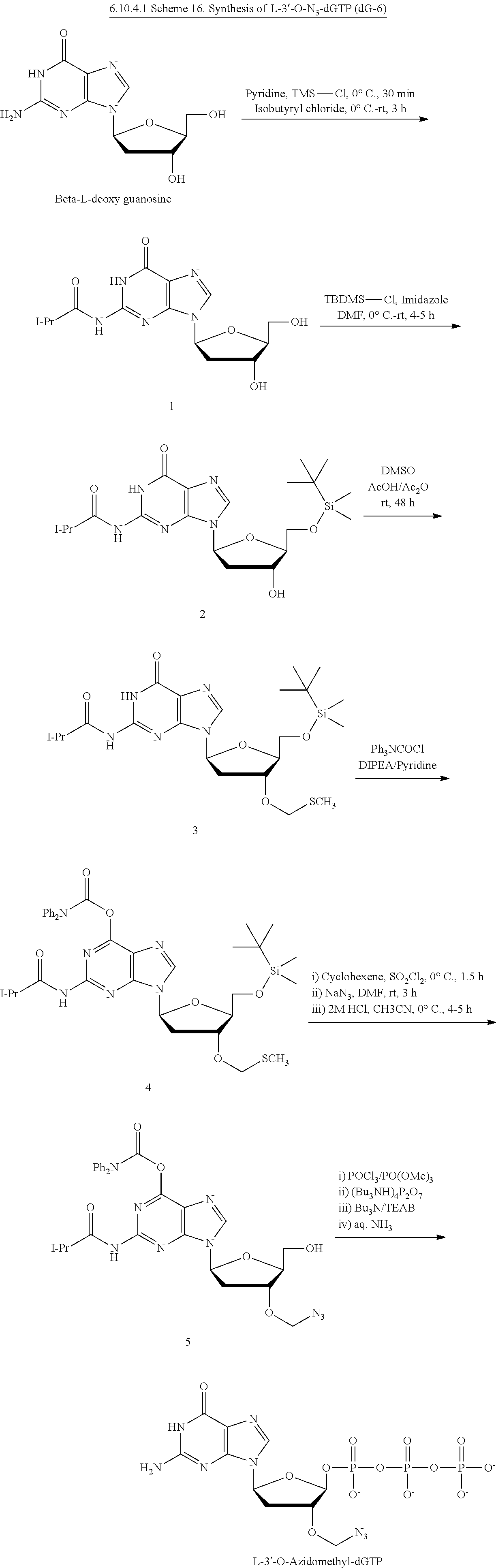

### Experimental Procedure:

dG-1 synthesis: O-L-deoxyguanosine (2 g, 7.48 mmol) was co-evaporated (2×12 mL), and then added pyridine (30 mL) and cool the suspension to 0° C. Trimethylsilyl chloride (4.8 mL, 37.4 mmol) was added dropwise via syringe. The ice bath was then removed and the mixture was stirred for 1 hour. The solution was cooled to 0° C. and isobutyric anhydride (6.2 mL, 37.4 mmol) was added dropwise via a syringe. The ice bath was removed and the resulting solution was stirred at room temperature for 3 hours. After stirred for 3 hours, the mixture was cooled to 0° C. and ice cold water (5 mL) was slowly added and stirred for 15 min, followed by concentrated ammonia solution (10 mL) to a final 2.5 M concentration of ammonia. The mixture was stirred on ice bath for 1 hour and then evaporated to dryness. The residue was redissolved in CH₃OH and was added silica gel and concentrated until dryness. The mixture was concentrated until dryness. The resulting residue was purified by column chromatography (19:1 to 9:1, CH₂Cl₂—CH₃OH) to give dG-1 (1.85 g, 73%) as a white solid.

dG-2 synthesis: To a solution of dG-1 (1.8 g, 5.33 mmol) in anhydrous DMF (30 mL) was added imidazole (544 mg, 8.0 mmol) and tert-butyldimethylsilyl chloride (885 mg, 5.87 mmo) at 0° C. under nitrogen, and warmed to room temperature. After stirred for 8 h at room temperature, the solution was added iced cold water and extracted with EtOAc (3×100 mL). The combined organic layers were dried over anhydrous Na₂SO₄, filtered and concentrated. The resulting residue was purified by column chromatography (19:1 to 9:1, CH₂Cl₂—CH₃OH) to afford compound dG-2 (1.9 g, 79%) as a white foam.

dG-3 synthesis: To a stirred solution of dG-2 (1.7 g; 3.76 mmol) in DMSO (12 ml) was added acetic acid (6 ml) and acetic anhydride (18 ml). After stirred at room temperature for 48 h, the solution was added saturated NaHCO₃ solution at 0° C. and stirred for 30 min, and the aqueous layer was extracted with EtOAc (2×100 ml). The combined organic layers were dried over Na₂SO₄, filtered and concentrated. The crude product was purified by flash column chromatography (1:1 to 1:3, Hexanes-EtOAc) to afford dG-3 (1.2 g; 65%) as a light yellowish foam R_{f} 0.26 (1:4 hexanes-EtOAc); ¹H NMR (400 MHz, CDCL₃): δ 12.1 (s, 1H, NH), 9.40 (s, 1H, NH), 7.98 (s, 1H), 6.18 (app t, 1H, J_{1′,2′a}=J_{1′,2′b}=6.9 Hz, H-1′), 4.71-4.61 (m, 3H, CH₂S, H-3′), 4.15-4.09 (m, 1H, H-4′), 3.78 (d, 2H, J=3.9 Hz, H-5′a, H-5′b), 2.74 (sep, 1H, J=7.0 Hz, COCH(CH₃)₂), 2.57-2.46 (m, 2H, H-2′a, H-2′b), 2.15 (s, 3H, SCH₃), 1.25 (app t, 6H, J=7.0 Hz, COCH(CH₃)₂), 0.89 (s, 9H, (CH₃)₃CSi); 0.08 (s, 3H, (CH₃)₂Si), 0.07 (s, 3H, (CH₃)₂Si); HRMS (ESI) m/z [M+H]⁺ calcd for C₂₂H₃₈N₅O₅SSi 512.2363. Found 512.2360.

dG-4 synthesis. To a stirred solution of dG-3 (1.20 g; 2.35 mmol) in dry pyridine (25 ml) was added diphenylcarbamoyl chloride (814 mg; 3.52 mmol) and DIPEA (N,N-diisopropylethylamine) (1.23 ml; 7.03 mmol) at room temperature. After stirred at room temperature for 3 hours, the solvent was removed under high vacuum. The residue was diluted with EtOAc and washed with 2M HCl and saturated NaHCO₃. The organic layer was dried with Na₂SO₄, filtered and concentrated. The residue was purified by flash column chromatography (1:1 to 1:3, Hexanes-EtOAc) to afford dG-4 (1.5 g, 90%) as a foam-type yellowish powder. R_{f}0.5 (1:1 hexanes-EtOAc); ¹H NMR (400 MHz, CDCL₃): δ 8.25 (s, 1H, NH), 8.05 (s, 1H), 7.50-7.33 (m, 8H, ArH), 7.29-7.21 (m, 2H, ArH), 6.41 (app t, 1H, J_{1′,2′a}=J_{1′,2′b}=6.7 Hz, H-1′), 4.76-4.66 (m, 3H, CH₂S, H-3′), 4.19-4.13 (m, 1H, H-4′), 3.88 (dd, 1H, J_{5′a,4′}=4.5 Hz, J_{gem}=11.1 Hz, H-5′a), 3.81 (dd, 1H, J_{5′b,4′}=3.6 Hz, J_{gem}=11.1 Hz, H-5′b), 2.96 (bs, 1H, COCH(CH₃)₂), 2.75 (ddd, 1H, J_{2′b,1′}=6.7 Hz, J_{2′a,3′}=7.2 Hz, J_{gem}=13.6 Hz, H-2′a), 2.57 (ddd, 1H, J_{a′a,1′}=6.7 Hz, J_{2′a,3′}=3.2 Hz, J_{gem}=13.6 Hz, H-2′b), 2.18 (s, 3H, SCH), 1.28 (d, 6H, J=6.8 Hz, COCH(CH₃)₂), 0.92 (s, 9H, (CH₃)₃CSi); 0.11 (s, 3H, (CH₃)₂Si), 0.10 (s, 3H, (CH₃)₂Si); HRMS (ESI) m/z [M+H]⁺ calcd for C₃₅H₄₇N₆O₆SSi 707.3047. Found 707.3048.

dG-5 synthesis: To a stirred solution of dG-4 (490 mg, 0.694 mmol) in dry CH₂Cl₂ (14 mL) was added cyclohexene (2.1 mL) and 1.0 M SO₂Cl₂ in CH₂Cl₂ (1.39 mL, 1.39 mmol) at 0° C. After stirred at 0° C. for 1 hour, the volatiles were removed under reduced pressure. To a solution of the residue in dry DMF (14 mL) was added NaN; (271 mg, 4.61 mmol) at room temperature. After stirred at room temperature for 2 h, the reaction mixture was dispersed in distilled water (200 mL) and extracted with EtOAc (2×200 mL). The combined organic layer was dried over Na₂SO₄, filtered and concentrated under reduced pressure. To a solution of the residue win acetonitrile (14 mL) was added 2M HCl (7 mL) at 0° C.). The reaction mixture was stirred for 30 min. The solution was diluted with EtOAc (100 mL) and washed with saturated NaHCO₃. The organic layer was washed with water (2×50 mL) and the organic layers were dried over Na₂SO₄, filtered and concentrated under reduced pressure. The resulting residue was purified by flash column chromatography (1:2 to 0:1, Hexanes-EtOAc) to afford dG-4 as colorless oil. R_{f} 0.32 (1:4 hexanes-EtOAc); ¹H NMR (400 MHz, CDCL₃): δ 8.38 (s, 1H, NH), 8.08 (s, 1H), 7.46-7.39 (m, 4H, ArH), 7.37-7.32 (m, 4H, ArH), 7.27-7.20 (m, 2H, ArH), 6.22 (app t, 1H, J_{1′,2′a}=J_{1′,2′b}=6.6 Hz, H-1′), 5.04-4.98 (m, 1H, H-3′), 4.77 (s, 2H, CH₂N₃), 4.19-4.14 (m, 1H, H-4′), 3.88 (dd, 1H, J_{5′a,4′}=2.9 Hz, J_{gem}=12.6 Hz, H-5′a), 3.80 (dd, 1H, J_{5′b,4′}=2.9 Hz, J_{gem}=12.6 Hz, H-5′b), 2.99 (app dt, J_{2′b,1}=J_{2′a,3′}=6.6 Hz, J_{gem}=13.7 Hz, H-2′a), 2.79-2.66 (m, 1H, COCH(CH₃)₂), 2.48 (ddd, 1H, J_{2′a,1}=6.6 Hz, J_{2′a,3′}=3.8 Hz, J_{gem}=13.7 Hz, H-2′b); HRMS (ESI) m/z [M+H]⁺ calcd for C₂₈H₃₀N₉O₆ 588.2219. Found 588.2177.

### 6.11. Example 11. Completed Synthesis of (L) 3′-O-Azidomethy-dNTP-Label Intermediates

The synthesis of L-3′-O-azidomethy-dNTP-FLs was designed and split into three two parts, synthesis of linker (Scheme 17), synthesis of the NH₂-L-3′-O-azidomethyl-dNTP intermediates (Schemes 18-23) and then coupling of the intermediate with the fluorophore, as described in Example 3, above.

Currently, the synthesis of linker (Scheme 17) afforded compound 5, referred to as intermediate Linker-5, which was characterized by ¹H NMR and HRMS. The first NH₂-L-3′-O-azidomethyl-dNTP we focused on is the synthesis of NH₂-L-3′-O-azidomethyl-dUTP (Scheme 18). Currently, the synthesis of NH₂-L-3′-O-azidomethyl-dUTP was completed to the step 3 and the intermediate “dUTP-FL-3” was obtained. The synthetic plans of other L-3′-O-azidomethy-dNTP-FL intermediates are also attached below (Schemes 19-21).

In view of the compatibility of fluorophore attached dNTP with 9° N DNA polymerase, we also designed the synthetic routes of NH₂-(L)3′-O—N₃-7-deaza-dGTP (Scheme 22) and NH₂-(L) 3′-O—N₃-7-deaza-dATP (Scheme 23). The starting materials of nucleotide bases (6-chloro-7-deazaguanine and 6-chloro-7-deaza-7-iodopurine) and 2-deoxy sugar (1-chloro-3,5-di-O-p-toluoyl-2-L-deoxyribofuranose) are commercially available.

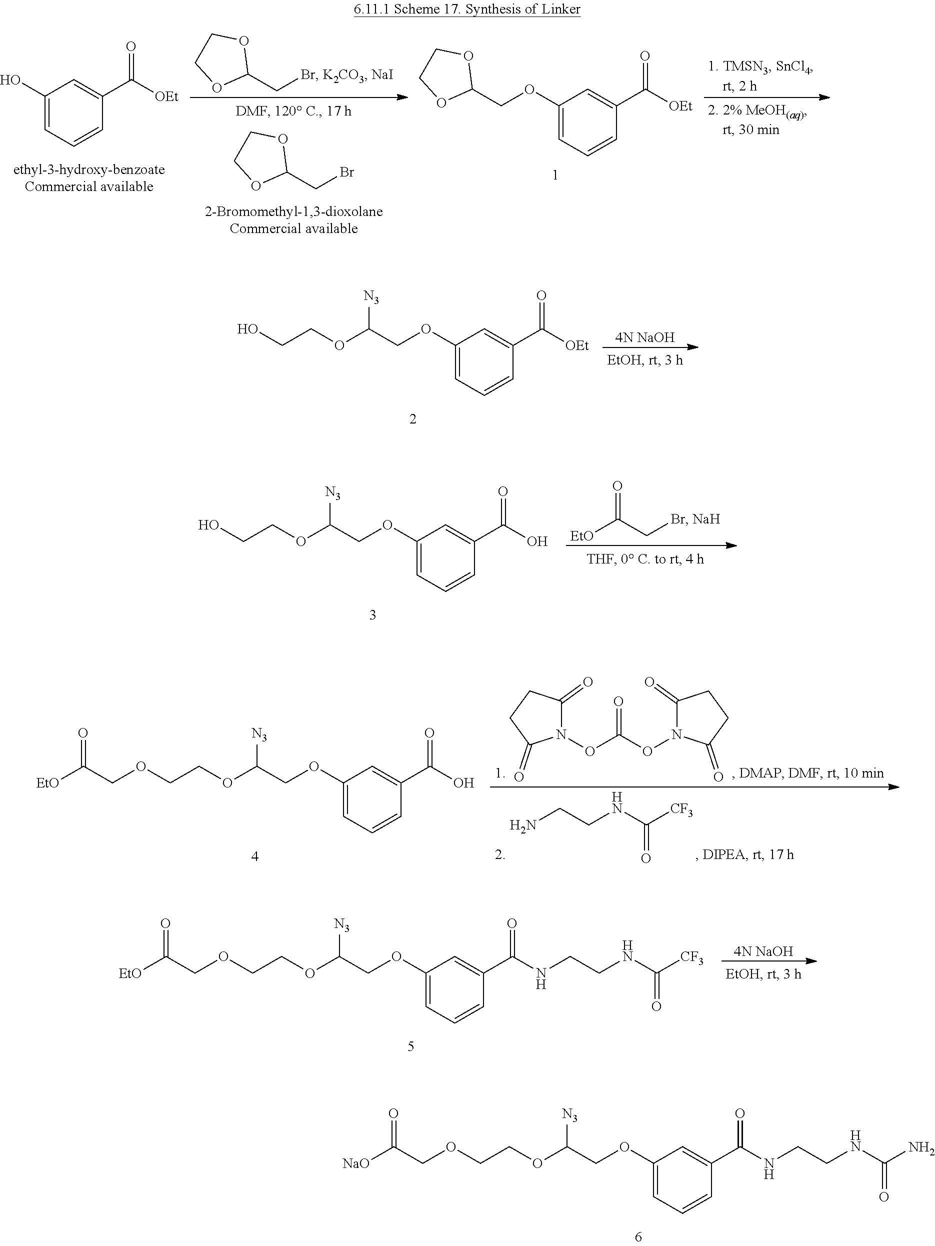

### Synthesis of Linker-1

To a solution of Ethyl-3-hydroxybenzoate (3.32 g, 20 mmol) in anhydrous DMF (8 ML) was added potassium carbonate (5.53 g, 40 mmol), sodium iodide (1.2 g, 0.4 mmol) and 2-bromomethyl-1,3-dioxolane (8.3 mL, 80 mmol) at room temperature. The mixture was heated to 120° C. and stirred overnight. The mixture was warmed to room temperature and evaporated under reduced pressure. The residue was diluted with CH₂Cl₂ (250 mL) and washed with water. The aqueous layer was washed with CH₂Cl₂ twice (2×50 mL). The combined organic layer was dried with MgSO₄, filtered and concentrated. The residue was purified by column chromatography (hexanes-EtOAc, 19:1 to 2:1) to obtain Linker-1 (4.6 g, 91%). R_{f} 0.54 (100% CH₂Cl₂); ¹H NMR (400 MHz, CDCL₃): δ 7.67 (dt, 1H, 1=1.1, 7.1 Hz, ArH), 7.61 (dd, 1H, J=1.5, 2.6 Hz, ArH), 7.35 (t, 1H, J=7.8 Hz, ArH), 7.15 (ddd, 1H, J=1.0, 2.7, 8.6 Hz, ArH), 5.32 (t, 1H, J=4.0 Hz, CH), 4.38 (q, 2H, J=7.2 Hz, OCH₂CH₃), 4.12-3.96 (m, 6H, OCH₂CH₂O, ArOCH₂), 1.40 (t, 3H, J_{gem}=7.2 Hz, OCH₂CH₃); HRMS

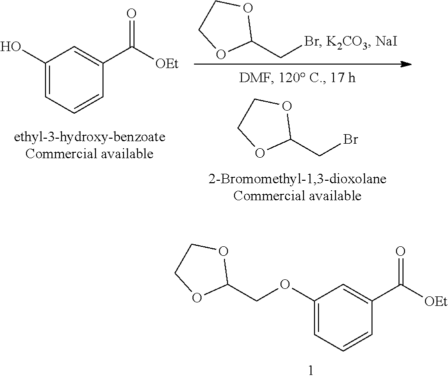

### Synthesis of Linker-2

To a solution of Linker-1 (2.70 g, 10.7 mmol) in azidotrimethylsilane (1.55 mL, 11.8 mmol) was added tin (IV) chloride (80 uL) at room temperature. The mixture was stirred at room temperature for 2 hours, then 2% aqueous methanol (10 mL) was added. The mixture was stirred at room temperature for 30 min, then concentrated under reduced pressure. The residue was co-evaporated with EtOH (2×30 mL) and the resulting residue was purified by column chromatography (hexanes-EtOAc, 2:1 to 1:1) to obtain Linker-2 (1.42 g, 45%). R_{f} 0.32 (2:1 hexanes-EtOAc); ¹H NMR (400 MHz, CDCl₃): δ 7.70 (dt, 1H, J=1.1, 7.6 Hz, ArH), 7.61 (dd, 1H, J=1.4, 2.5 Hz, ArH), 7.38 (t, 1H, J=7.8 Hz, ArH), 7.15 (ddd, 1H, J=0.9, 2.5, 8.1 Hz, ArH), 4.90 (t, 1H, J=5.3 Hz, CHN₃), 4.40 (q, 2H, J=7.2 Hz, OCH₂CH₃), 4.25 (dd, 1H, J=5.3, 10.2 Hz, ArOCH₂), 4.17 (dd, 1H, J=5.3, 10.2 Hz, ArOCH₂), 4.06-4.00 (m, 1H, OCH₂), 3.91-3.73 (m, 3H, OCH₂CH₂OH), 1.40 (t, 3H, J_{gem}=7.2 Hz, OCH₂CH₃); HRMS (ESI) m/z [M−H]⁻ calcd for C₁₅H₁₈N₃O₇ 352.1236. Found 352.1165

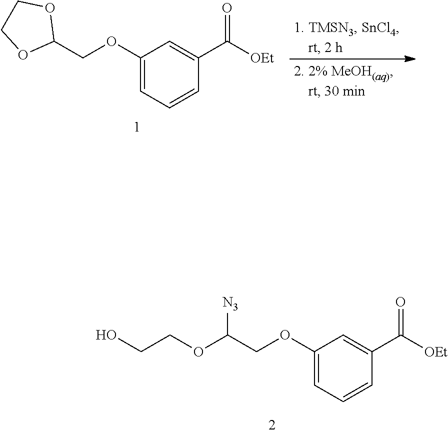 [SMILES: CCOC(=O)c1cccc(OCC2OCCO2)c1 ; "1. TMSN3, SnCl4,    rt, 2 h" ; CCOC(=O)c1cccc(OCC(N=[N+]=[N-])OCCO)c1]

### Synthesis of Linker-3

To a solution of Linker-2 (1.42 g, 4.8 mmol) in EtOH (5 mL) was added 4N NaOH (5 mL) at room temperature. The mixture was stirred at room temperature for 3 hours, then the mixture was concentrated under reduced pressure and acidified by 2N HCl (20 mL) and extracted with CH₂Cl₂ twice (2×50 mL). The combined organic layer was dried over MgSO₄ and filtered and concentrated to obtain Linker-3 (1.21 g, 94%). R_{f} 0.41 (19:1 EtOAc-CH₃OH); ¹H NMR (400 MHz, CDCL₃): δ 7.78 (dt, 1H, 1=1.1, 7.6 Hz, ArH), 7.67 (dd, 1H, J=1.4, 2.5 Hz, ArH), 7.43 (t, 1H, J=7.8 Hz, ArH), 7.21 (ddd, 1H, J=0.9, 2.5, 8.1 Hz, ArH), 4.92 (t, 1H, 0.1=5.2 Hz, CHN₃), 4.26 (dd, 1H, J=5.2, 10.1 Hz, ArOCH₂), 4.17 (dd, 1H, J=5.2, 10.1 Hz, ArOCH₂), 4.08-4.01 (m, 1H, OCH₂), 3.89-3.77 (m, 3H, OCH₂CH₂OH); HRMS (ESI) m/z [M−H]⁻ calcd for C₁₁H₁₂N₃O₅ 266,0867. Found 266.0794

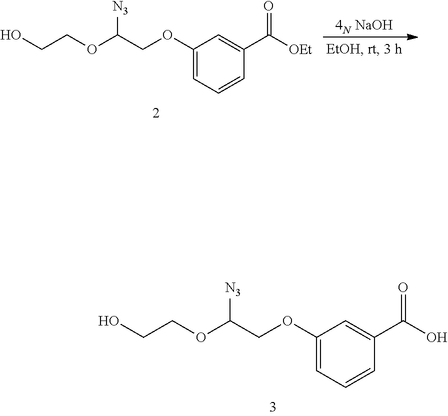

### Synthesis of Linker-4

To a solution of Linker-3 (320 mg, 1.57 mmol) in THF (5 mL) was added 60% NaH (188 mg, 4.71 mmol) at 0° C. The mixture was stirred at 0° C. for 10 min, then mixture was added ethyl 2-bromoacetate (382 μL, 3.45 mmol). The mixture was warmed to room temperature and stirred for 4 hours. After stirring 4 hours, cold water (50 mL) was poured into reaction mixture and the resulting mixture was extracted with CH₂Cl₂ (50 mL). The organic layer was discarded and the aqueous layer was acidified by addition of 2N HCl (25 mL). The resulting mixture was extracted with CH₂Cl₂ (2×50 mL) and the collecting organic layers were dried over Na₂SO₄ and filtered and concentrated. The resulting residue was purified by column chromatography (98:2, CH₂Cl₂—CH₃OH) to obtain the Linker-4 (120 mg, 22%) as light yellowish oil. R_{f} 0.67 (1:1:0.5 EtOAc-Hexanes-AcOH); ¹H NMR (400 MHz, CDCl₃): δ 7.75 (app dt, 1H, J=1.1, 7.5 Hz, ArH), 7.64 (dd, 1H, J=1.4, 2.5 Hz, ArH), 7.39 (t, 1H, J=7.8 Hz, ArH), 7.19 (ddd, 1H, J=0.8, 2.6, 8.2 Hz, ArH), 4.96 (app t, 1H, J=5.0 Hz, CHN₃), 4.28-4.12 (m, 6H, ArOCH₂, OCH₂CH₃, OCH₂C═O), 4.05 (app dt, 1H, J=4.1, 11.4 Hz, OCH₂), 3.97-3.87 (m, 1H, OCH₂), 3.82 (appt, 2H, J=4.8 Hz, OCH), 1.29 (t, 3H, J=7.2 Hz, OCH₂CH₃) HRMS (ESI) m/z [M−H]⁻ calcd for C₁₅H₁₈N₃O₇ 352.1236. Found 352.1165.

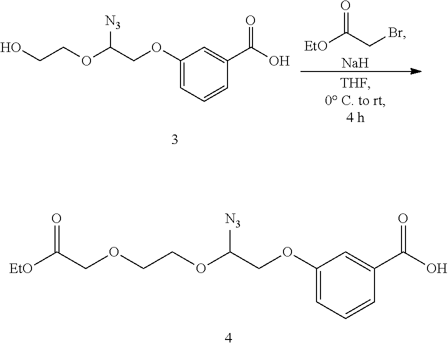

### Synthesis of Linker-5

To a solution of Linker-4 (120 mg, 0.339 mmol) in DMF (1 mL) was added DSC (104 mg, 0.407 mmol) and DMAP (49.8 mg, 0.407 mmol) at room temperature. The mixture was stirred at room temperature for 10 min, then mixture was added N-(2-aminoethyl)-2,2,2-trifluoroacetamide (78.5 mg, 0.407 mmol) and DIPEA (142 μL, 0.815 mmol) at room temperature. The mixture was stirred at room temperature for overnight, and quenched with 1N Na₂HPO₄ solution (25 mL). The mixture was extracted with CH₂Cl₂ until no product were observed in aqueous layer. The combined organic layers were dried over Na₂SO₄ and filtered and concentrated. The resulting residue was purified by column chromatography (1:1, Hexanes-EtOAc) to obtain Linker-5 (42 mg, 25%) as colorless oil. R_{f} 0.62 (1:1:0.5 EtOAc-Hexanes-AcOH); ¹H NMR (400 MHz, CDCl₃): δ 8.25 (bs, 1H, NH), 7.42-7.30 (m, 4H, ArH, NH), 7.07 (ddd, 1H, J=0.9, 2.5, 8.0 Hz, ArH), 4.90 (app t, 1H, J=4.9 Hz, CHN₃), 4.25-4.07 (m, 6H, ArOCH₂, OCH₂CH₃, OCH₂C═O), 4.05-3.99 (m, 1H, OCH₂), 3.89-3.82 (m, 1H, OCH₂), 3.79 (appt, 2H, J=4.2 Hz, OCH₂), 3.70-3.62 (m, 2H, NCH₂CH₂N), 3.61-3.54 (m, 2H, NCH₂CH₂N), 1.28 (t, 3H, J=7.2 Hz, OCH₂CH₃)

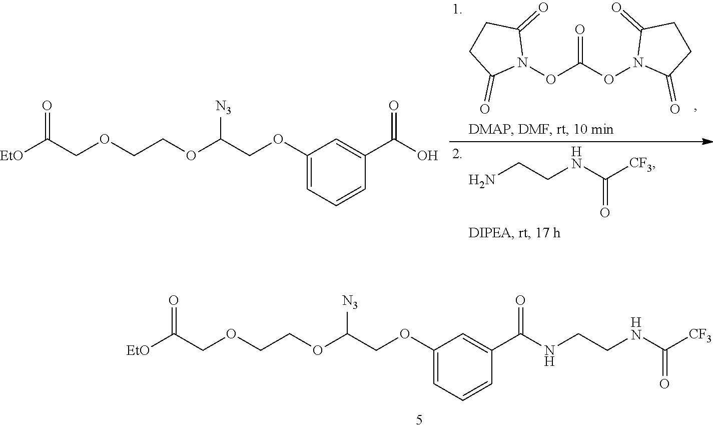 [SMILES: CCOC(=O)COCCOC(COc1cccc(C(=O)O)c1)N=[N+]=[N-] ; O=C(ON1C(=O)CCC1=O)ON1C(=O)CCC1=O ; NCCNC(=O)C(F)(F)F ; CCOC(=O)COCCOC(COc1cccc(C(=O)NCCNC(=O)C(F)(F)F)c1)N=[N+]=[N-]]

### 6.11.2. Synthesis of Intermediate NH₂-(L) 3′-O-Azidomethyl-dNTP

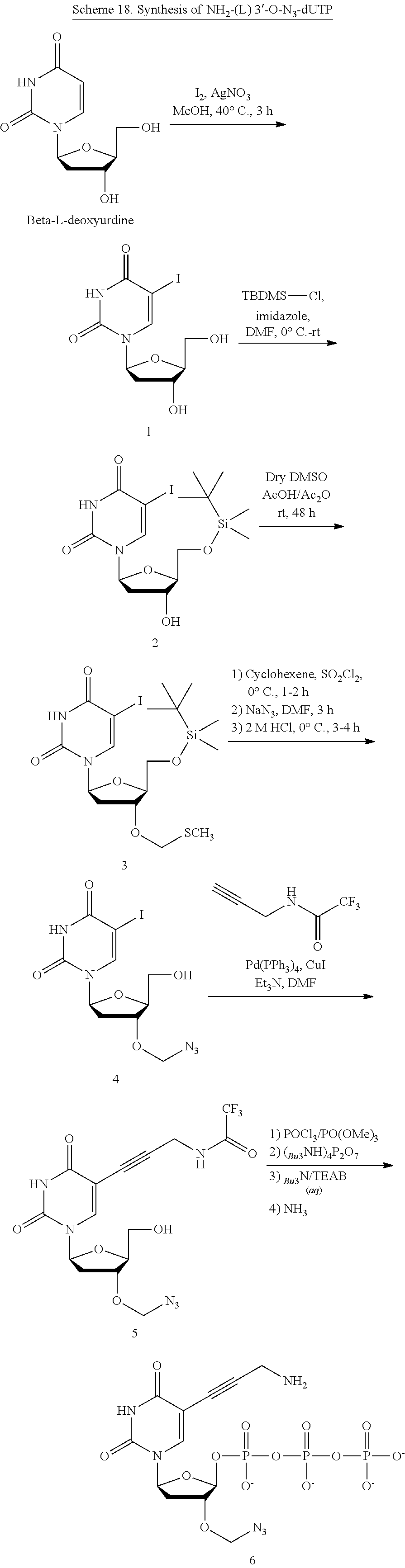

### Experimental Procedure:

Synthesis of dUTP-FL-1: Suspended Beta-L-deoxyuridine (2.4 g, 10.51 mmol, 1.0 eq.) in methanol (30 mL) and stirred for 10 min. Later added Iodine (8.0 g, 31.55 mmol, 3 eq) and Silver nitrate (5.3 g, 31.55 mmol, 3 eq) and stirred the reaction mixture for 3 hr at 40° C. After the completion of the reaction, the reaction mixture was filtered and the filtrate was subjected to column chromatography on silica gel using MeOH/CH₂Cl₂ 1:19 to 1:9 to obtain compound dUTP-FL-1 as a white solid (1.8 g, 48%). R_{f} 0.65 (9:1 DCM-MeOH), ¹HNMR (500 MHz, DMSO-d₆ δ 11.63 (s, 1H), 8.36 (s, 1H), 6.05 (t, J=6.5 Hz, 1H), 4.24-4.12 (m, 2H), 3.79-3.69 (m, 2H), 3.61-3.46 (m, 2H), 2.15-1.96 (m, 2H).

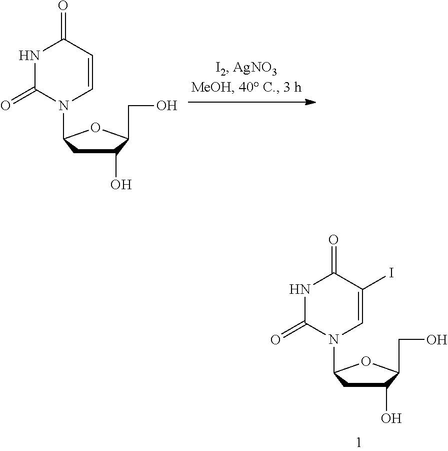 [SMILES: O=c1ccn([C@@H]2C[C@H](O)[C@H](CO)O2)c(=O)[nH]1 ; O=c1[nH]c(=O)n([C@@H]2C[C@H](O)[C@H](CO)O2)cc1I]

Synthesis of dUTP-FL-2: Compound 1 (400 g, 1.12 mmol, 1.0 eq.) was solubilized in anhydrous DMF (20 mL). To it, imidazole (114.3 mg, 1.68 mmol, 1.5 eq.) and tert-butyldimethylsilyl chloride (185.6 mg, 1.23 mmol, 1.1 eq.) were added at 0° C. under nitrogen. The reaction mixture was stirred for 12 h at room temperature. Then add cold water and extracted with EtOAc (3×50 mL). The combined organic layers were dried on anhydrous Na₂SO₄, concentrated, and the resulting residue was subjected to column chromatography using 2-5% MeOH in CH₂Cl₂ as eluent to afford compound dUTP-FL-2 as a foam type white solid (348 mg, 63%). R_{f} 0.45 (19:1 DCM-MeOH)¹HNMR (500 MHz,) δ 8.38 (s, 1H), 8.10 (s, J=4.0 Hz, 1H), 6.29 (dd, J=8.1, 5.6 Hz, 1H), 4.47 (dd, J=5.4, 3.1 Hz, 1H), 4.08 (q, J=2.3 Hz, 1H), 3.86 (ddd, J=40.5, 11.4, 2.5 Hz, 2H), 2.41 (ddd, J=13.4, 5.6, 2.1 Hz, 1H), 2.09 (ddd, J=13.6, 8.1, 5.7 Hz, 2H), 1.98 (d, J=3.5 Hz, 1H), 0.92 (s, J=2.9 Hz, 9H), 0.15 (s, 3H)-0.13 (s, 3H).

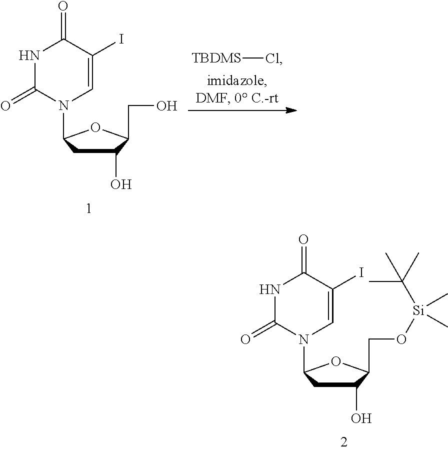

Synthesis of dUTP-FL-3: To a stirred solution of 2 (108 mg; 0.23 mmol) in DMSO (8 ml), acetic acid (4 ml) and acetic anhydride (12 ml) were added. The reaction mixture was stirred at room temperature for 48 h. A saturated NaHCO₃ solution (50 ml) was added at 0° C. and stirred for 30 min, and the aqueous layer was extracted with EtOAc (3×100 ml). The combined organic extract was dried over Na₂SO₄ and concentrated. The crude product was purified by flash column chromatography (ethyl acetate/hexane, 1:1 to 7:3) to afford dUTP-FL-3 as a foam-type white solid (106 mg, 87%)). R_{f} 0.85 (1:1 Hexane-EtOAc) ¹HNMR (500 MHz,) δ 8.86 (s), 8.09 (s), 7.27 (s), 6.23 (dd, J=8.3, 5.5 Hz), 5.42-5.25 (m), 4.36 (d, J=5.9 Hz), 4.17 (d, J=2.1 Hz), 3.87 (ddd, J=56.2, 11.5, 2.3 Hz), 2.54 (ddd, J=13.6, 5.5, 1.7 Hz), 2.12 (s), 2.04 (ddd, J=14.0, 8.3, 5.9 Hz), 1.36-1.21 (m, 1H), 0.95 (s, 9H), 0.17 (s, 3H), 0.16 (s, 3H).

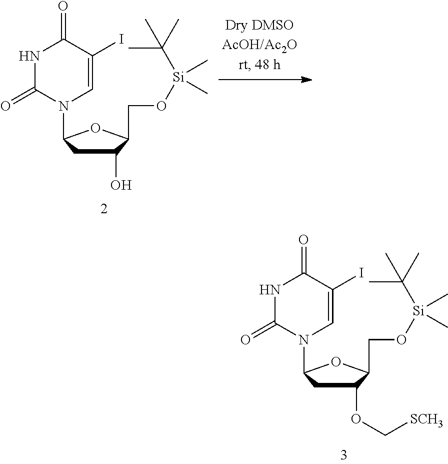 [SMILES: CC(C)(C)[Si](C)(C)OC[C@@H]1O[C@H](n2cc(I)c(=O)[nH]c2=O)C[C@@H]1O ; CSCO[C@H]1C[C@@H](n2cc(I)c(=O)[nH]c2=O)O[C@H]1CO[Si](C)(C)C(C)(C)C]

### 6.12. Example 12. Design of Synthesis of (L) 3′-O-Azidomethyl-7-Deaza-dNTP-Label

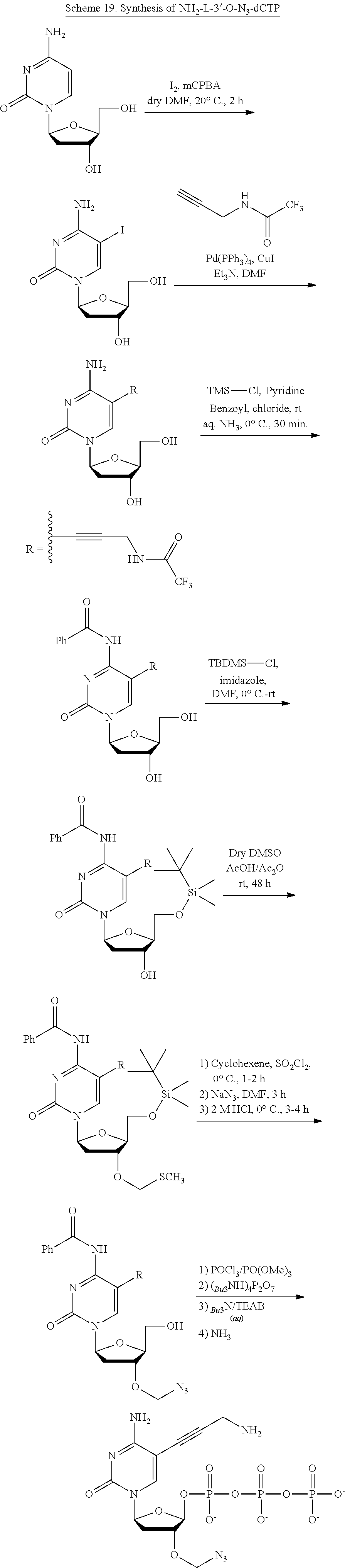

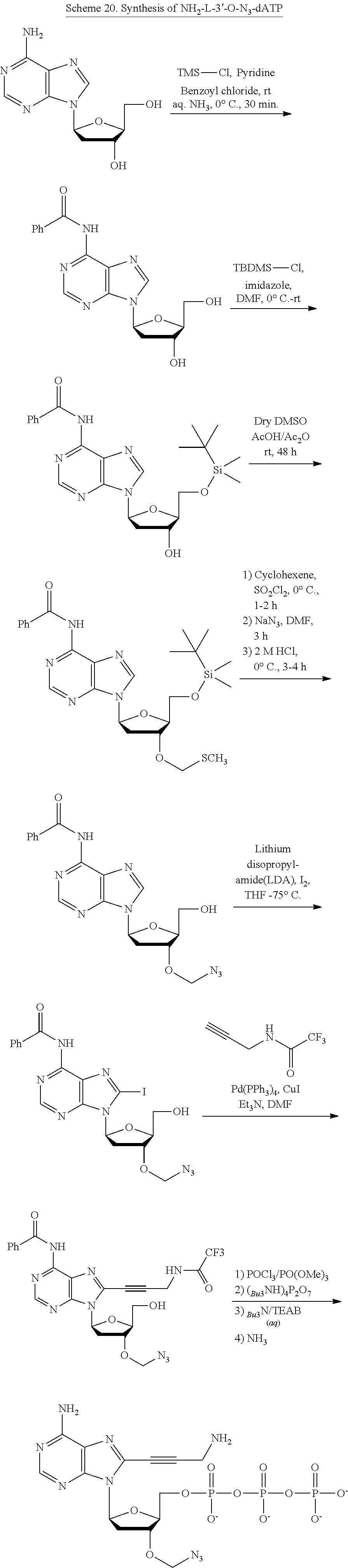

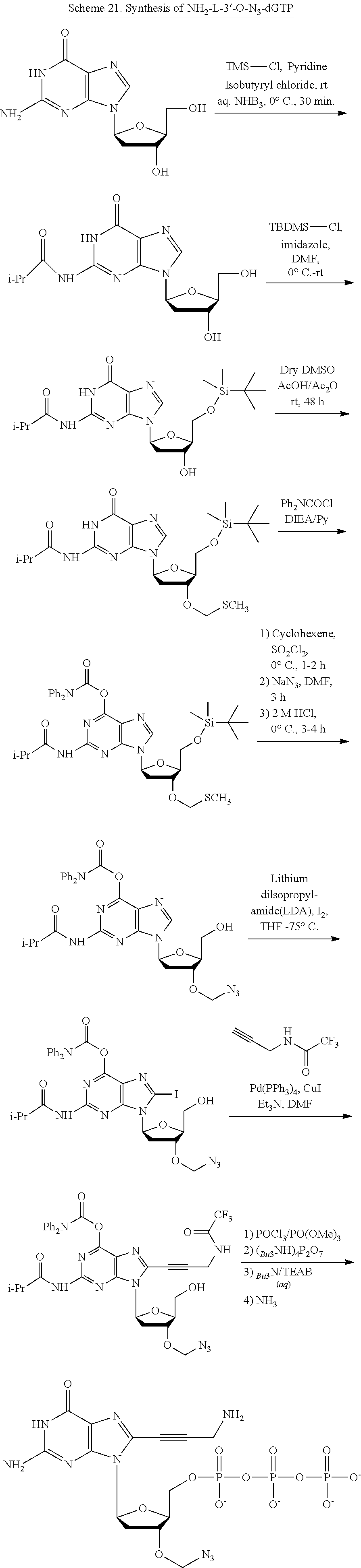

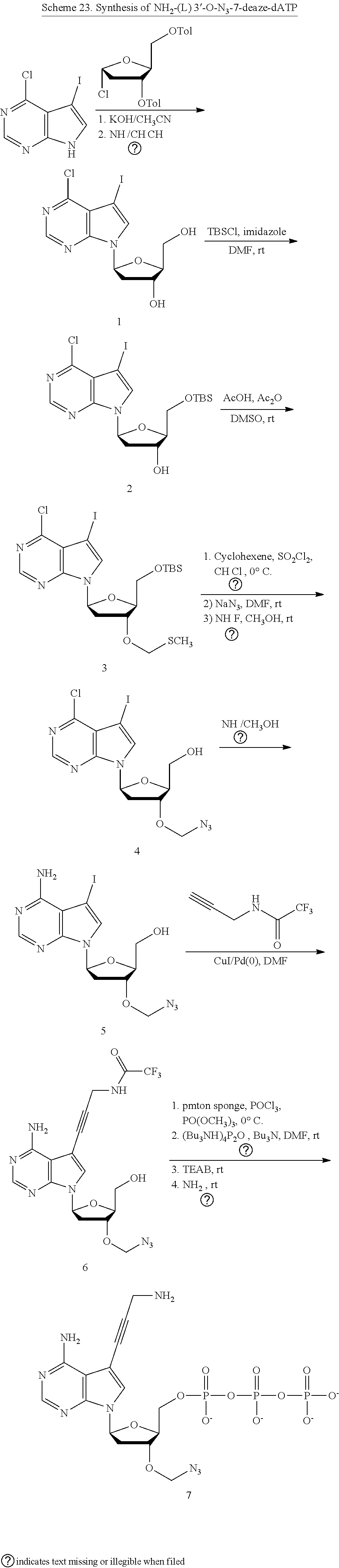

### Synthetic Protocol of NH₂-(L) 3′-O—N₃-7-Deaza-dATP

To a solution of 4-chloro-5-iodo-7H-pyrrolo[2.3-d]pyrimidine (1.0 g, 3.58 mmol) in CH₃CH (60 mL), powdered KOH (85%, 0.5 g, 7.57 mmol) and TDA-1 (0.075 mL, 0.24 mmol) were added at room temperature. After stirring for 10 min, halogenose (1.7 g, 4.37 mmol) was introduced and the stirring was continued for another 10 min Insoluble material was filtered off and washed several times with hot acetone. The combined filtrates were evaporated to dryness. The residue was applied onto flash chromatography (silical gel, column 5×15 cm, elution with petroleum ether-EtOAc, 4:1). The combined fractions containing the product were evaporated to yield 4-chloro-7-[2-deoxy-3,5-di-O-(4-methylbenzoyl-β-L-erythro-pentofuranosyl)-5-iodo-7H-pyrrolo[2,3-d]pyrimidine as a colorless solid (2.02 g, 89%). A solution of intermediate (1.8 g, 2.85 mmol) in NH₃/CH₃OH (saturated at 0° C., 145 mL) was stirred at room temperature for 24 hours and the solvent was evaporated. Flash chromatography (silica gel, column 4×16 cm, CH₂Cl₂/CH₃OH 9:1) yielded compound 7-deaza-dA-1 as colorless solid (0.45 g, 40%. R_{f} 0.45 (CH₂Cl₂/CH₃OH, 9:1). To a stirred mixture of 7-deaza-dA-1 (1.00 g, 2.83 mmol) and imidazole (462 mg, 6.79 mmol) in anhydrous DMF (14.0 mL), tert-butyldimethylsilyl chloride (TBDMSCl) (510 mg, 3.28 mmol) was added. The reaction mixture was stirred at room temperature for 20 h. After evaporation, the residue was purified by flash column chromatography (CH₂Cl₂—CH₃OH, 20:1) to afford 7-deaza-dA-2 as white solid (1.18 g, 89%). To a stirred solution of compound 7-deaza-dA-2 in DMSO, acetic acid and acetic anhydride are added. The reaction mixture is stirred at room temperature for 48 h. A saturated NaHCO₃ solution is added and the aqueous layer is extracted with CH₂Cl₂. The combined organic extract is washed with saturated NaHCO₃ solution and dried over Na₂SO₄. After concentration, the residue is purified by flash column chromatography (hexane/ethyl acetate, 1:1 to 1:4) to afford compound 7-deaza-dA-3. To a stirred solution of compound 7-deaza-dA-3 in dry CH₂Cl₂ under nitrogen, cyclohexene, and SO₂Cl₂ are added. The reaction mixture is stirred at 0° C. for 2 h. The solvent is first removed under reduced pressure and then under a high-vacuum pump for 10 min. The residue is dissolved in dry DMF and reacted with NaN₃ at room temperature for 3 h. The reaction mixture is dispersed in distilled water (50 ml) and extracted with CH₂Cl₂ (3×50 ml). The combined organic layer is dried over Na₂SO₄ and concentrated under reduced pressure. The residue is dissolved in MeOH and stirred with NH₄F at room temperature for 24 h. The solvent is removed under reduced pressure. The reaction mixture is concentrated under reduced pressure and partitioned between H₂O and CH₂Cl₂. The organic layer is separated and dried over Na₂SO₄. After concentration, the crude product is purified by flash column chromatography (ethyl acetate/methanol, 100:0 to 98:2) to afford compound 7-deaza-dA-4. Compound 7-deaza-dA-4 was dissolved in 7M ammonia in methanol solution. The solution was stirred at 115-120° C. for 15 h. After evaporation, the residue was purified by flash column chromatography (CH₂Cl₂—CH₃OH, 20:1) to afford 7-deaza-dA-5. To a solution of 7-deaza-dA-5 in anhydrous DMF, tetrakis(triphenylphosphine)palladium(0) and CuI were added. The reaction mixture was stirred at room temperature for 10 min. Then N-propargyltrifluoroacetamide and Et₃N were added into the above reaction mixture. The reaction was stirred at room temperature for 1.5 h with exclusion of iar and light. After evaporation, the residue was dissolved in ethyl acetate. The mixture was washed with saturated aqueous NaHCO₃, NaCl and dried over anhydrous Na₂SO₄. After evaporation, the residue was purified by flash column chromatography (CH₂Cl₂—CH₃OH, 10:1) to afford 7-deaza-dA-6. Compound 7-deaza-dA-6 and proton sponge are dried in a vacuum desiccator over P₂O₅ overnight before dissolving in trimethyl phosphate. Then freshly distilled POCl₃ is added dropwise at 0° C. and the mixture is stirred at 0° C. for 2 h. Subsequently, a well-vortexed mixture of tributylammonium pyrophosphate and tributylamine in anhydrous DMF is added in one portion at room temperature and stirred for 30 min. Triethyl ammonium bicarbonate solution (TEAB) (0.1 M; pH 8.0) is then added and the mixture is stirred for 1 h at room temperature. Then concentrated NH₄OH is added and stirred overnight at room temperature. The resulting mixture is concentrated under vacuum and the residue is diluted with 5 ml of water. The crude mixture is then purified with anion exchange chromatography on DEAE-Sephadex A-25 at 4° C. using a gradient of TEAB (pH 8.0; 0.1-1.0 M). The crude product is further purified by reverse-phase HPLC to afford (L) 3′-O-azidomethyl-7-deaza-dATP (compound 7-deaza-dA-7).

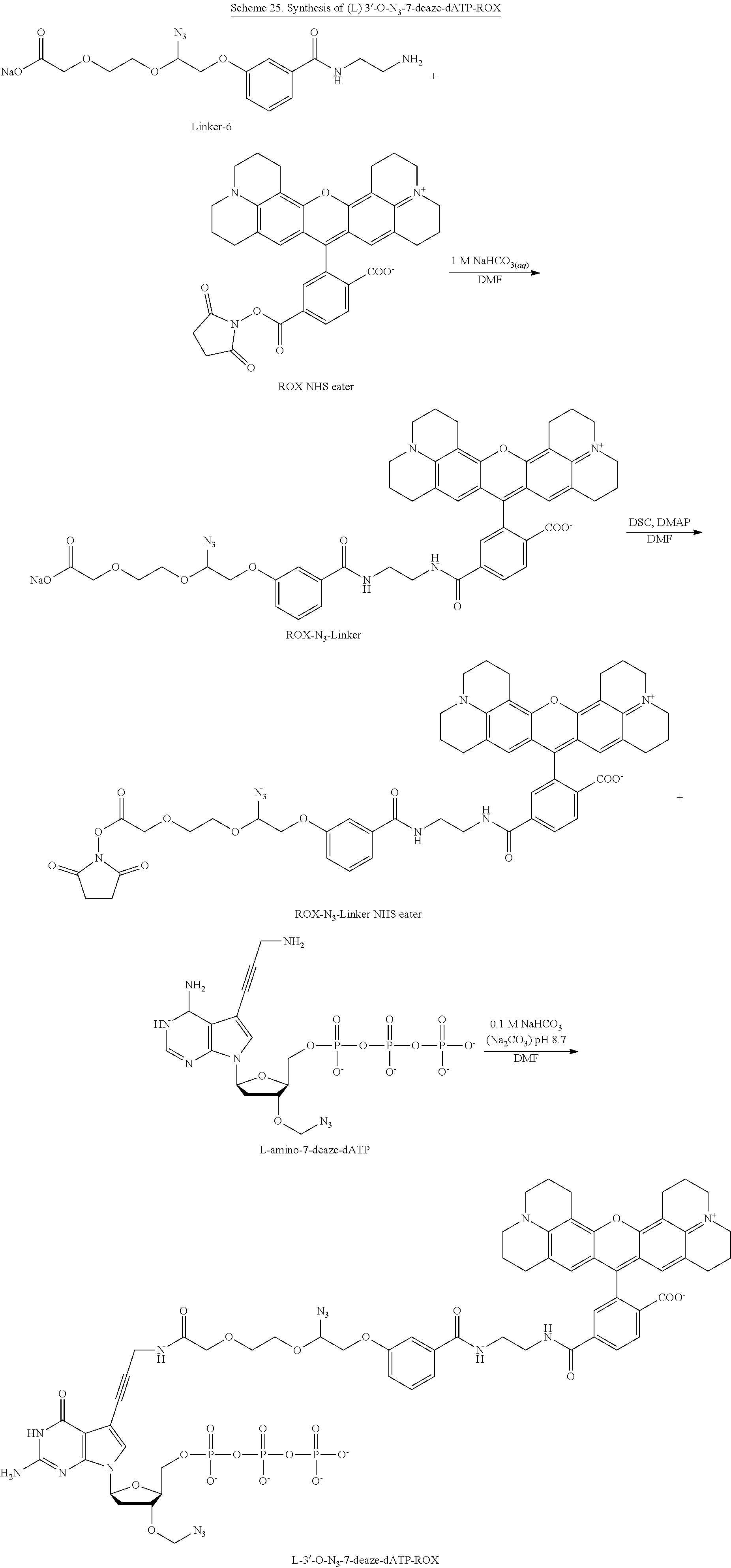

Azido-ROX compound (ROX-N₃-Linker). (2-{2-[3-(2-Amino-ethylcarbamoyl)-phenoxy]-1-azido-ethoxy}-ethoxy)-acetic acid Linker-6 (7.0 mg, 0.019 mmol) prepared according to the literature (Milton J, Ruediger S, Liu X (2006) US Patent Appl US20060160081A1) is dissolved in DMF (300 μl) and 1 M NaHCO₃ aqueous solution (100 μl). A solution of ROX NHS (N-hydroxysuccinimide) ester (Invitrogen) (0.013 mmol) in DMF (400 μl) is added slowly to the above reaction mixture and then stirred at room temperature for 5 h with exclusion of light. The crude product is purified on a preparative silica gel TLC plate (CHCl₃/CH₃OH, 1:4).

L-3′-O-azidomethyl-7-deaza-dATP-ROX compound (L-3′-O—N₃-7-deaza-dATP-ROX). To a stirred solution of ROX-N₃-Linker in dry DMF (2 ml), DSC (N,N′-disuccinimidyl carbonate) (3.4 mg, 13.2 μmol) and DMAP (4-dimethylaminopyridine) (1.6 mg, 13.2 μmol) is added. The reaction mixture is stirred at room temperature for 2 h. TLC indicated that ROX-N₃-Linker is completely converted to compound ROX-N₃-Linker NHS ester, which is directly used to couple with L-amino-7-deaza-dATP (13 μmol) in NaHCO₃/Na₂CO₃ buffer (pH 8.7, 0.1 M) (300 μl). The reaction mixture is stirred at room temperature for 3 h with exclusion of light. The reaction mixture is purified by a preparative silica gel TLC plate (CH₃OH/CH₂Cl₂, 1:1). The crude product is further purified on reverse-phase HPLC to afford L-3′-O-azidomethyl-dATP-ROX (L-3′-O—N₃-dATP-ROX).

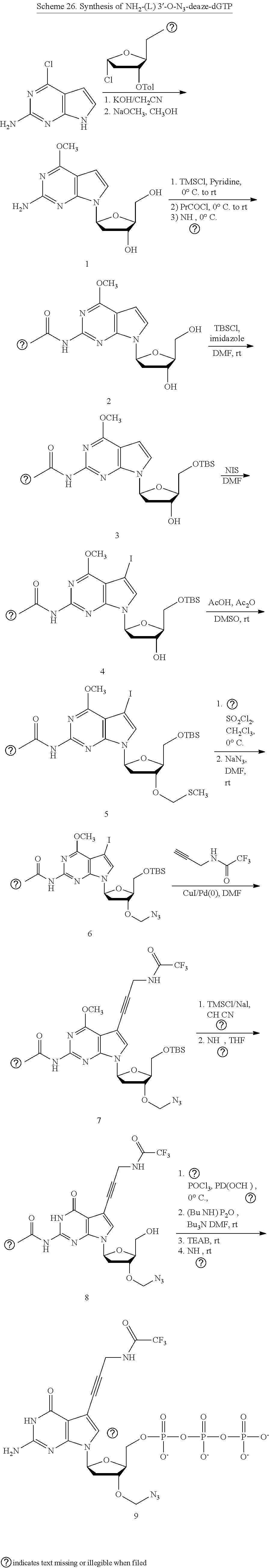

Synthetic protocol of NH₂-(L) 3′-O—N₃-7-deaza-dGTP To a suspension of powdered KOH (85%, 1.15 g, 17.42 mmol) and TDA-1 (0.2 mL, 0.63 mmol) in CH₃CN (60 mL), 2-amino-4-chloro-7H-pyrrolo[2.3-d]pyrimidine (842 mg, 4.99 mmol) was added at room temperature. After stirring for 5 min, halogenose (2.53 g, 6.51 mmol) was introduced within 15 min, and the stirring was continued for 30 min. Insoluble material was filtered off and washed several times with CH₃CN. The combined filtrates were evaporated to dryness. The residue was applied onto flash chromatography (silical gel, column 6×12 cm, elution with CH₂Cl₂). The combined fractions containing the product were evaporated to yield a colorless solid (2.21 g, 85%). A solution of intermediate (1.04 g, 2.00 mmol) in 0.5M NaOCH₃/CH₃OH (60 mL) was stirred under reflux for 3 hours. The mixture was neutralized with AcOH and evaporated. The residue was applied onto flash chromatography (silica gel, CH₂Cl₂/CH₃OH, 95:5) yielded compound 7-deaza-dG-1 as colorless solid (400 mg, 71%). To a stirred solution of the 7-deoxy-dG-1 is co-evaporated with anhydrous pyridine (3×4 mL) and then dissolved in anhydrous pyridine (2 mL). The resulting solution is protected from moisture (drying tube), purged with argon and placed on ice. To the ice cold solution, TMS-Cl (4.51 mL, 58.4 mmol, 8.2 eq.) is added dropwise via a syringe. The ice bath is then removed and the mixture is stirred for 2 hours. The solution is cooled on ice and isobutyric chloride (0.29 mL, 15.69 mmol, 2.2 eq.) is added dropwise via a syringe and the ice bath is removed. After stirring for another 2 hours at room temperature, the reaction is placed again on ice and ice cold water (20 mL) is slowly added, followed after 15 minutes by concentrated ammonia solution (1.5 mL) to get a final 2.5 M concentration of ammonia. The mixture is kept on ice for 30 minutes, and then evaporated to dryness. The residue is co-evaporated with toluene (3×5 mL) to remove traces of water, resuspended in MeOH and filtered to remove the precipitate. The filtrate is then concentrated, dissolved in a small amount of MeOH, absorbed on silica gel and purified by column chromatography (DCM/MeOH 95:5 to 91:9 (v/v)) to give compound 7-deaza-dG-2. Compound 7-deaza-dG-2 is co-evaporated three times with dry pyridine, dried under high vacuum, and dissolved in 2 cm³ dry N,N-dimethylformamide in an ice bath. Imidazole (419 mg, 6.15 mmol) is added and the mixture is stirred for 15 min at 0° C. and for 15 min at room temperature. Then, 241 mg tert-butyldimethylsilyl chloride (1.59 mmol) is added and the solution is stirred at 60° C. for another 2 h. The mixture is diluted with dichloromethane, washed with brine, dried over sodium sulfate, and evaporated. The crude product is purified by column chromatography on silica gel (methanol:dichloromethane 0:100-2:98) as white foam (compound 7-deaza-dG-3). To a vigorously stirred solution of 7-deaza-dG-3 in anhydrous DMF was added NIS. The reaction mixture was stirred at room temperature for 22 h, and then most solvent was removed under vacuum. Diethyl ether and saturated NaHCO₃ were added. The organic layer was washed with saturated NaCl, and dried over Na₂SO₄. After evaporation, the residue was purified by flash column chromatography to afford 7-deaza-dG-4. To a stirred solution of compound 7-deaza-dG-4 in DMSO (12 ml), acetic acid (5.5 ml) and acetic anhydride (17.6 ml) are added. The reaction mixture is stirred at room temperature for 48 h. A saturated NaHCO₃ solution (100 ml) is added and the aqueous layer is extracted with CH₂Cl₂ (3×100 ml). The combined organic extract is washed with saturated NaHCO₃ solution (100 ml) and dried over Na₂SO₄. After concentration, the residue is purified by flash column chromatography to afford compound 7-deaza-dG-5. To a stirred solution of compound 7-deaza-dG-5 in dry CH₂Cl₂ under nitrogen, cyclohexene, and SO₂Cl₂ are added. The reaction mixture is stirred at 0° C. for 2 h. The solvent is first removed under reduced pressure and then under a high-vacuum pump for 10 min. The residue is dissolved in dry DMF and reacted with NaN₃ at room temperature for 3 h. The reaction mixture is dispersed in distilled water (50 ml) and extracted with CH₂Cl₂ (3×50 ml). The combined organic layer is dried over Na₂SO₄ and concentrated under reduced pressure. The residue is purified by flash column chromatography (ethyl acetate/methanol, 100:0 to 98:2) to afford compound 7-deaza-dG-6. To a solution of 7-deaza-dG-6 in anhydrous DMF, tetrakis(triphenylphosphine)palladium(0) and CuI were added. The reaction mixture was stirred at room temperature for 10 min. Then N-propargyltrifluoroacetamide and Et₃N were added into the above reaction mixture. The reaction was stirred at room temperature for 1.5 h with exclusion of iar and light. After evaporation, the residue was dissolved in ethyl acetate. The mixture was washed with saturated aqueous NaHCO₃, NaCl and dried over anhydrous Na₂SO₄. After evaporation, the residue was purified by flash column chromatography to 7-deaza-dG-7. To a stirred solution of 7-deaza-dG-7 in anhydrous CH₃CN were added NaI and chlorotrimethylsilane. The reaction was stirred at room temperature for 1 h and then at 50° C. for 12 h. The solvent was evaporated, and the residue was dissolved in THF. Tetrabutylammonium fluoride (TBAF) in THF solution was added, and the reaction was stirred at room temperature for 1 h. The solvent was evaporated, and the residue was dissolved in EtOAc. The solution was washed with saturated aqueous NaCl and drived over anhydrous Na₂SO₄. After evaporation of the solvent, the residue was purified by flash column chromatography (EtOAc-CH₃OH) to afford 7-deaza-dG-8. Compound 7-deaza-dG-8 and proton sponge are dried in a vacuum desiccator over P₂O₅ overnight before dissolving in trimethyl phosphate. Then freshly distilled POCl₃ is added dropwise at 0° C. and the mixture is stirred at 0° C. for 2 h. Subsequently, a well-vortexed mixture of tributylammonium pyrophosphate and tributylamine in anhydrous DMF is added in one portion at room temperature and stirred for 30 min. Triethyl ammonium bicarbonate solution (TEAB) (0.1 M; pH 8.0) is then added and the mixture is stirred for 1 h at room temperature. Then concentrated NH₄OH is added and stirred overnight at room temperature. The resulting mixture is concentrated under vacuum and the residue is diluted with 5 ml of water. The crude mixture is then purified with anion exchange chromatography on DEAE-Sephadex A-25 at 4° C. using a gradient of TEAB (pH 8.0; 0.1-1.0 M). The crude product is further purified by reverse-phase HPLC to afford (L) 3′-O-azidomethyl-7-deaza-dATP (compound 7-deaza-dG-9).

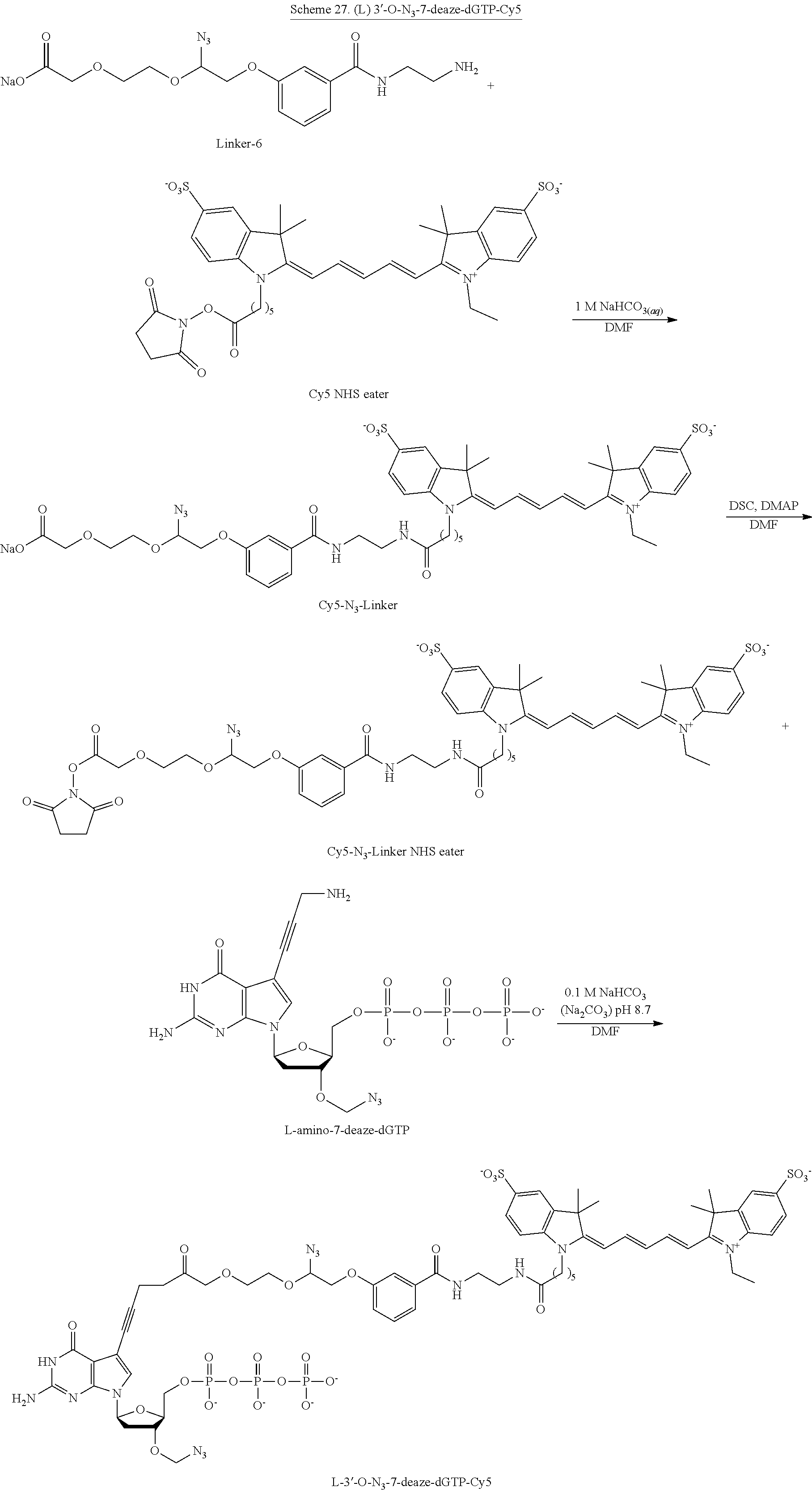

Azido-Cy5 compound (Cy5-N₃-Linker). (2-{2-[3-(2-Amino-ethylcarbamoyl)-phenoxy]-1-azido-ethoxy}-ethoxy)-acetic acid Linker-6 (7.0 mg, 0.019 mmol) prepared according to the literature (Milton J, Ruediger S, Liu X (2006) US Patent Appl US20060160081A1) is dissolved in DMF (300 μl) and 1 M NaHCO₃ aqueous solution (100 μl). A solution of Cy5 NHS (N-hydroxysuccinimide) ester (Invitrogen) (0.013 mmol) in DMF (400 μl) is added slowly to the above reaction mixture and then stirred at room temperature for 5 h with exclusion of light. The crude product is purified on a preparative silica gel TLC plate (CHCl₃/CH₃OH, 1:4).

L-3′-O-azidomethyl-7-deaza-dGTP-Cy5 compound (L-3′-O—N₃-7-deaza-dGTP-Cy5). To a stirred solution of Cy5-N₃-Linker in dry DMF (2 ml), DSC (N,N′-disuccinimidyl carbonate) (3.4 mg, 13.2 μmol) and DMAP (4-dimethylaminopyridine) (1.6 mg, 13.2 μmol) is added. The reaction mixture is stirred at room temperature for 2 h. TLC indicated that Cy5-N₃-Linker is completely converted to compound Cy5-N₃-Linker NHS ester, which is directly used to couple with L-amino-7-deaza-dGTP (13 μmol) in NaHCO₃/Na₂CO₃ buffer (pH 8.7, 0.1 M) (300 μl). The reaction mixture is stirred at room temperature for 3 h with exclusion of light. The reaction mixture is purified by a preparative silica gel TLC plate (CH₃OH/CH₂Cl₂, 1:1). The crude product is further purified on reverse-phase HPLC to afford L-3′-O-azidomethyl-dGTP-Cy5 (L-3′-O—N₃-dGTP-Cy5).

### 6.13. Example 13. Chemical Synthesis of (D)-Form 9° N DNA Polymerase Via Solid Phase Peptide Synthesis and Native Chemical Ligation

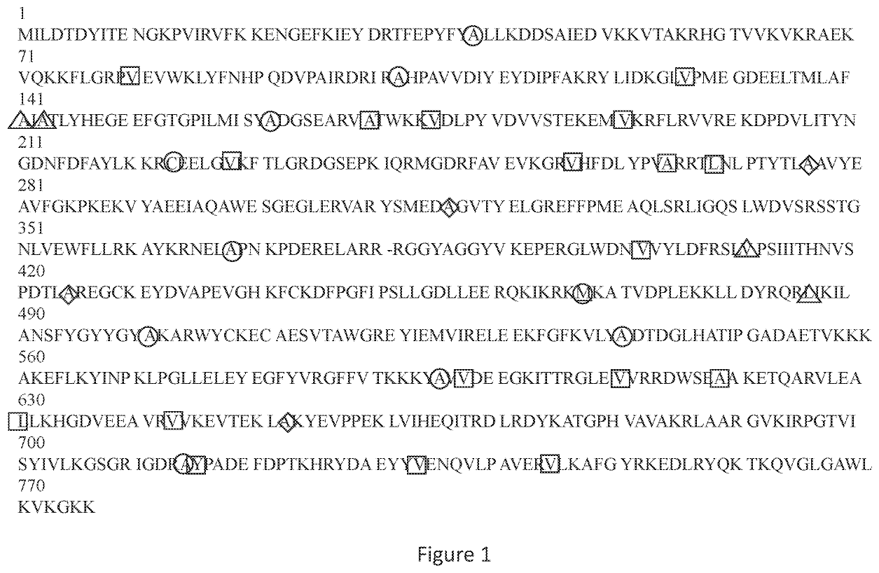

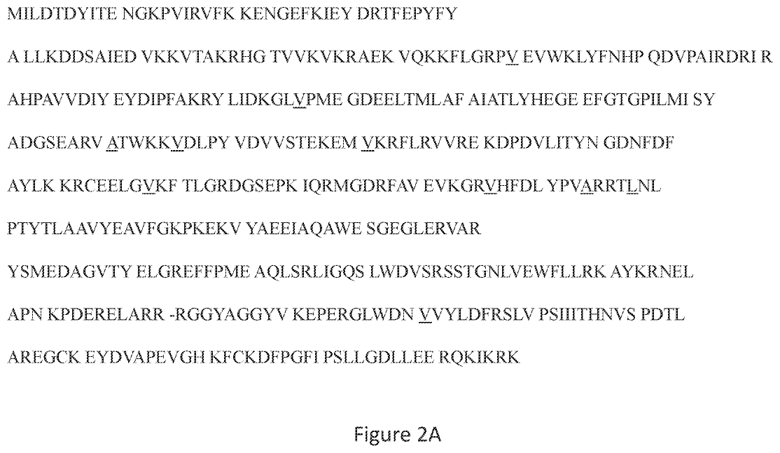

FIG. 1 provides the amino-acid sequence of (D)-form mutant 9° N DNA polymerase (candidate #1-1), where all the amino acids are D-form amino acids. Diamond=mutation introduced for NCL; Circle=NCL site (start of the ‘second’ strand); Square=potential substitution sites for isoleucines; Triangle=mutation introduced for modified nucleotides.

| | Amino acid positions | |
|---|---|---|
| | (in reference to SEQ ID NO: 1) | modifications |
| Modification for | E276, K317, N424, S651 | E276A, K317G, N424A, S651A |
| NCL (native | | |
| chemical ligation) | | |
| sites | | |
| NCL (native | A40, A102, A163, C223, A368, | |
| chemical ligation) | M467, A500, A539, A595, A714 | |
| sites | | |
| Isoleucine for | I80, I127, I171, I176, I191, I228, | I80V, I127V, I171A, I176V, |
| substitution | I256, I264, I268, I400, I597, I610, | I191V, I228V, I256V, I264A, |
| | I618, I630, I642, I715, I733, I744 | I268L, I400V, I597V, I610V, |
| | | I618A, I630L, I642V, I715Y, |
| | | I733V, I744V |
| Modification for | D141, E143, Y409, A485 | D141A, E143A, Y409V, A485L |
| interaction with | D141, E143, L408, Y409, P410, | D141A, E143A, L408A, Y409A, |
| modified | A485, T514, I521 | P4101, A485V, T514S, I521L |
| nucleotides | M129, 130-131, D141, E143, | M129L, 130-131D, D141A, |
| | L408, Y409, P410, A485, T514, | E143A, L408A, Y409A, P4101, |
| | I521 | A485V, T514S, I521L |

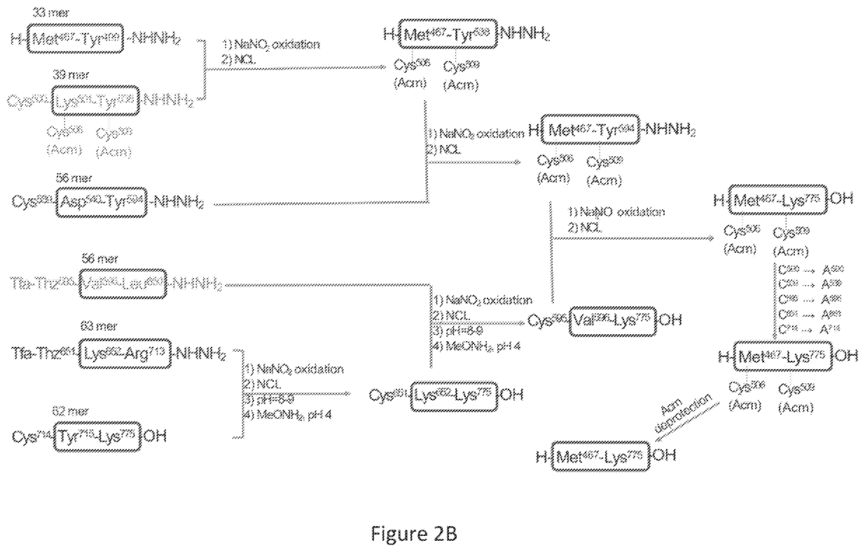

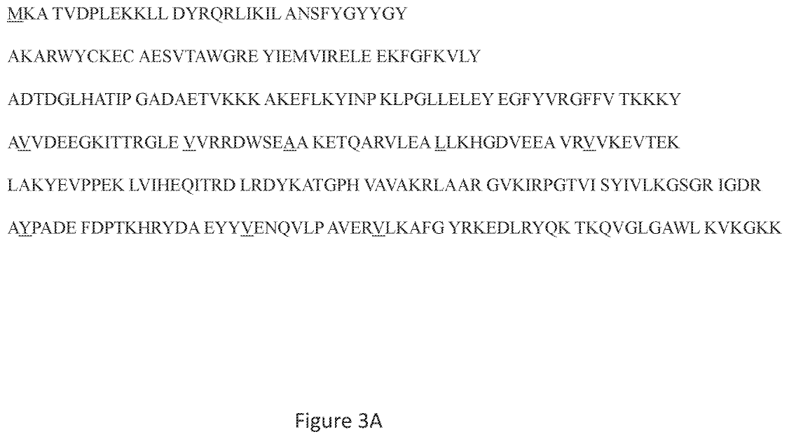

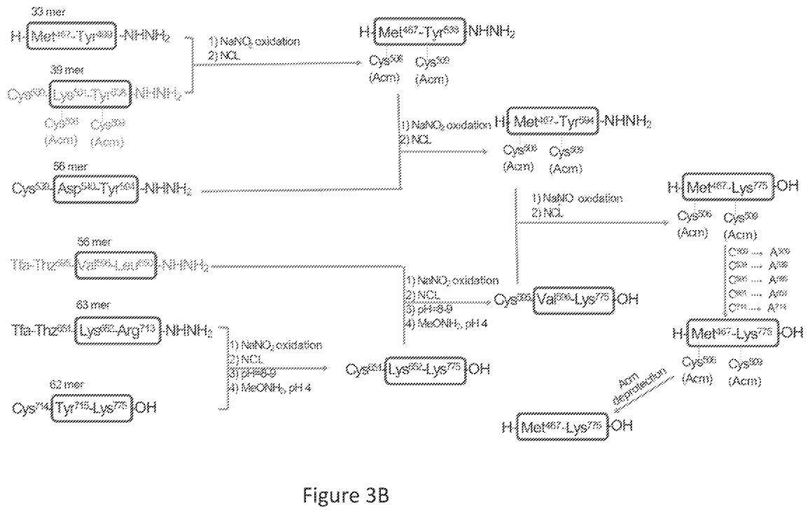

The 9° N DNA polymerase was split into two fragments (a 466-aa 9° N—N fragment and a 310-aa 9° N—C fragment) at the split site between K466 and M467. The synthesis of 466-aa 9° N—N fragment was designed as nine synthetic peptides (D-9° N—N-1 to D-9° N—N-9) (FIG. 2A) via solid phase peptide synthesis which ligate at the certain cysteine residue as shown in FIG. 2B. Similarly, the 310-aa 9° N—C fragment was split into six synthetic peptides (FIG. 3A), which are synthesized via solid phase peptide synthesis, and then ligate at the certain cysteine residues as shown in FIG. 3D.

### 6.13.1. Experimental Methods

Materials. 2-Chlorotrityl chloride resin (loading=0.98 mmol g⁻¹) was purchased from Purepep. Fmoc-D-amino acids, D-4-thiazolidinecarboxylic acid, hydrazine hydrate, ethyl cyanoglyoxylate-2-oxime (Oxyma), N,N′-diisopropylcarbodiimide (DIC), trifuoroacetic acid, N,N-dimethylformamide (DMF), dichloromethane, Piperidine, thioanisole, triisopropylsilane, 1,2-ethanedithiol, and trifuoroacetic acid for peptide synthesis were purchased commercially from Chempep, Sigma-aldrich, Alfa aesar, TCI, etc. The reagents for NCL reaction, i.e, guanidine hydrochloride (Gn·HCl), Na₂HPO₄ 12H₂O, NaH₂PO₄-2H₂O, sodium nitrite (NaNO₂), Sodium hydroxide (NaOH), hydrochloric acid sodium 2-mercaptoethanesulfonate, 4-Mercaptophenylacetic acid (MPAA), Tris(2-carboxyethyl)phosphine hydrochloride (TCEP-HCl), DL-1,4-dithiothreitol (DTT), 2,2′-azobis[2-(2-imidazolin-2-yl) propane]dihydrochloride (VA-044), Glutathione (reduced form), and palladium chloride (PdCl₂) were purchased commercially from Sigma-aldrich, Alfa aesar, TCI, Duksan, etc.

Fmoc-based SPPS. All peptides were synthesized by Fmoc-based SPPS on the Liberty Blue automated microwave peptide synthesizer (CEM) and PurePep® Chorus automated peptide synthesizer. All the peptide hydrazides were synthesized on hydrazine-2-chlorotrityl chloride resin. For each peptide hydrazide, the first residue was attached to the hydrazine-2-chlorotrityl chloride resin by a double coupling method using 5 equiv. amino acid, 10 equiv. DIC, and 5 equiv. Oxymapure. All resins were swelled in DMF for 30 min before coupling. The Fmoc groups of assembled amino acids were removed by treatment with 20% piperidine and 0.1 M Oxyma in DMF at 85° C. Coupling of amino acids except Fmoc-Cys(Trt)-OH and Fmoc-His(Trt)-OH was carried out at 85° C. using 5 equiv. amino acid, 5 equiv. Oxymapure and 10 equiv. DIC for 2 min. The coupling reactions for Fmoc-Cys(Trt)-OH and Fmoc-His(Trt)-OH were carried out at 50° C. for 10 min to avoid side reactions at high temperature. Trifluoroacetyl thiazolidine-4-carboxylic acid-OH was coupled using 5 equiv. Oxymapure and 10 equiv. DIC at room temperature overnight. Double coupling strategy was used for the peptides beyond 20 amino acids. After the completion of peptide chain assembly, peptides were cleaved from resin using H₂O/thioanisole/triisopropylsilane/1,2-ethanedithiol/trifluoroacetic acid (0.5/0.5/0.5/0.25/8.25) (vol/vol). The cleavage reaction took 2.5 h under agitation at 27° C. Cold ether was added to precipitate the crude peptide. After centrifugation, the supernatant was discarded and the precipitates were washed twice with ether. The crude peptides were dissolved in CH₃CN/H₂O, analyzed by RP-HPLC and purified by semi-preparative HPLC. Collected peptide fractions were analyzed by electrospray ionization mass spectrometry (ESI-MS).

Native Chemical Ligation (NCL). The C-terminal peptide hydrazide segment was dissolved in acidified ligation buffer (aqueous solution of 6M Gn·HCl and 0.1 M NaH₂PO₄, pH 3.0). The mixture was cooled in an ice-salt bath (−15° C.), and 10 equiv. NaNO₂ in acidified ligation buffer (pH 3.0) was added. The activation reaction system was kept in an ice-salt bath under stirring for 20 min, after which 40 equiv. MPAA in ligation buffer and 1 equiv. N-terminal cysteine peptide were added, and the pH of the solution was adjusted to 6.6-6.8 at room temperature. After overnight reaction, 150 mM TCEP in ligation buffer (pH adjusted to 7.0) was added to dilute the system twice and the reaction system was kept at room temperature for 30 min with stirring. Finally, the ligation product was analyzed by HPLC and purified by semi-preparative HPLC. Purified ligation fractions were analyzed by ESI-MS.

### Tfa-D-Thz-OH Synthesis:

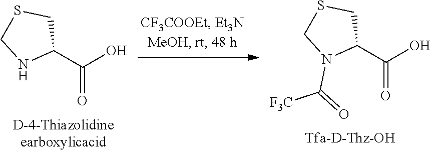 [SMILES: O=C(O)[C@H]1CSCN1 ; O=C(O)[C@H]1CSCN1C(=O)C(F)(F)F]

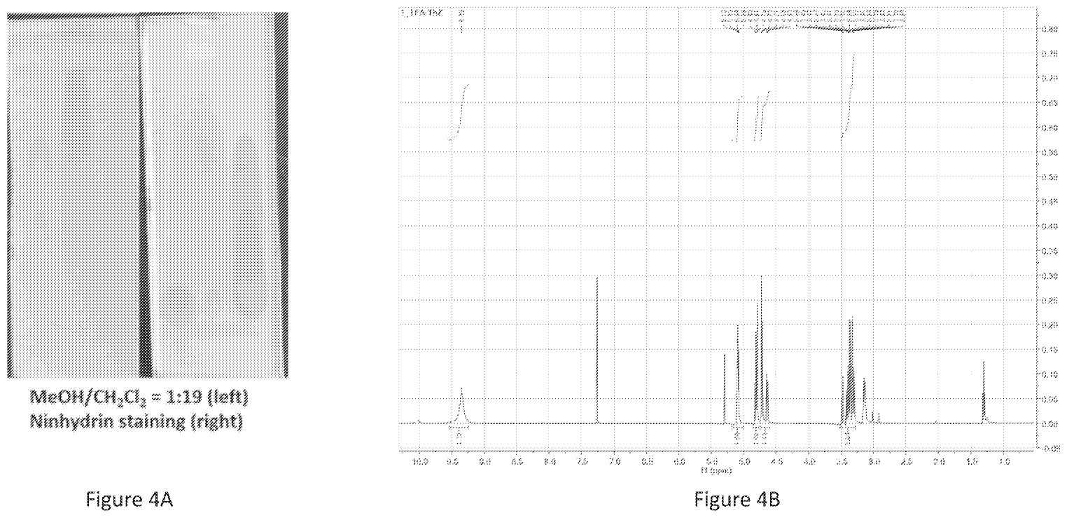

To a solution of D-4-thiazolidinecarboxylic acid (1 g, 7.509 mmol) suspended in MeOH (40 mL) was added triethylamine (2.62 mL, 18.772 mmol) and the suspension was stirred for 10 min. Ethyltrifluroacetate (0.98 mL, 8.2599 mmol) was then added drop-wise and the mixture was stirred for 48 h at RT. Then the mixture was concentrated and subjected to silicagel chromatography using MeOH/Dichloromethane (1:5) give Tfa-(D)-Thz-OH, i.e. (S)-3-(2,2,2-trifluoroacetyl)thiazolidine-4-carboxylic acid) as a pale-yellow oil (0.9 g, 3.92 mmol, 52.3%) (FIG. 4A). ¹H NMR (500 MHz, CDCl₃): δ (ppm) 9.35 (s, 1H), 5.09 (dd, J=6.9, 4.2 Hz, 1H), 4.86-4.77 (m, 1H, rotamers), 4.75-4.61 (m, 1H, rotamers), 3.52-3.28 (m, 2H, rotamers) (FIG. 4B).

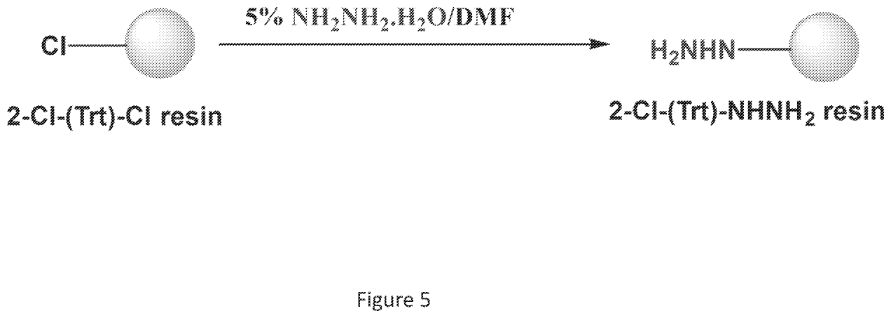

### Fmoc-Based SPPS (FIG. 5):

1) Weigh 102 mg of 2-Cl-(Trt)-Cl resin (0.98 mmol g⁻¹) into a peptide synthesis vessel.
2) Add 5 ml of DMF to the resin from the top of the reaction vessel, gently agitate it for 10 s and then drain it.
3) Repeat Step 2 two more times.
4) Add 5 ml of DCM to the resin, gently agitate it for 10 s and then drain it.
5) Repeat Step 4 two more times.
6) Repeat Step 2 three more times.
7) Swell the resin in 4 ml of 50% (vol/vol) DMF/DCM for 30 min, and then drain it.
8) Add 4 ml of 5% (vol/vol) NH₂NH₂·H₂O to the resin for hydrazination. Gently agitate the mixture for 30 min in a constant-temperature shaker at 30° C. and then drain the solution by vacuum filtration.
9) Add 4 ml of DMF to the resin, gently agitate it for 10 s and then drain it.
10) Repeat Step 8.
11) Wash the resin by repeating Steps 2-6 twice.
12) Add 4 ml of 5% (vol/vol) MeOH/DMF to the resin, gently agitate it for 10 min and then drain it to unreacted sites on the resin to be capped.
13) Repeat Steps 2-6 to wash the resin thoroughly.
14) Add 4 ml of DCM to the resin, agitate it gently for 10 s and then drain it. Directly use the resin for the next coupling step.
15) For storage, dry the resin under high vacuum for 2 h and store dried 2-Cl-(Trt)-NHNH₂ resin at −20° C. under argon gas for up to 2 weeks.

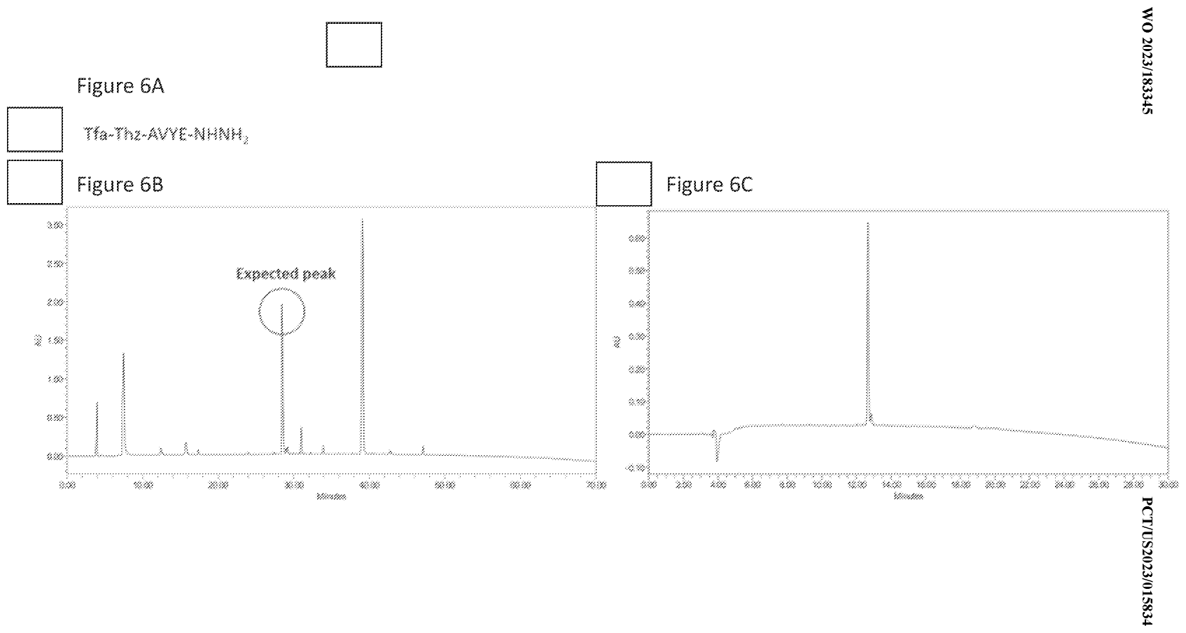

Solid Phase Peptide Synthesis of D-9° N—N-6@5-mer: Tfa-Thz-AVYE-NHNH₂ (FIG. 6A). D-9° N—N-6@5-mer was synthesized on PurePep® Chorus automated peptide synthesizer by following the conditions mentioned in experimental methods. D-9° N—N-6@5-mer synthesized by the process was analyzed by HPLC chromatogram and the results are provided in FIGS. 6B-6C.

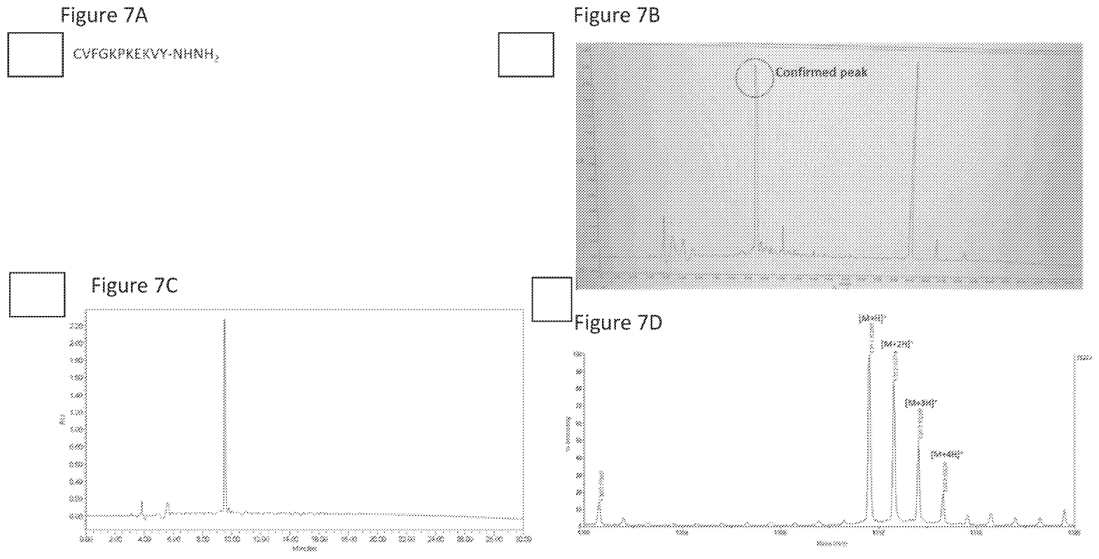

Solid Phase Peptide Synthesis of D-9° N—N-6@11-mer: CVFGKPKEKVY-NHNH₂: D-9° N—N-6@11-mer was synthesized on PurePep® Chorus automated peptide synthesizer by following the conditions mentioned in experimental methods. D-9° N—N-6@11-mer synthesized by the process was analyzed by HPLC chromatogram and the results are provided in FIGS. 7B-7D.

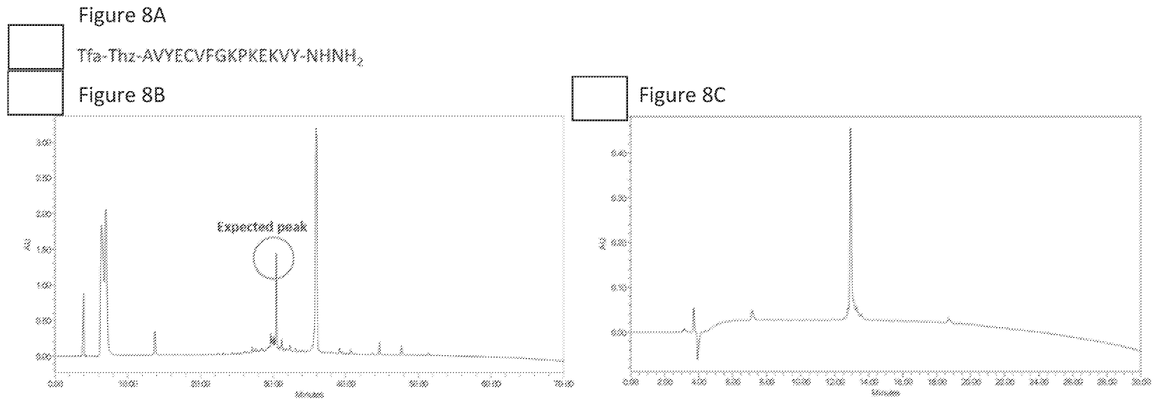

Solid Phase Peptide Synthesis of D-9° N—N-6@24-mer: CEEIAQAWE SGEGLERVAR YSMED-NHNH₂ (SEQ ID NO: 13) (FIG. 8A). D-9° N—N-6@24-mer was synthesized on PurePep® Chorus automated peptide synthesizer by following the conditions mentioned in experimental methods. D-9° N—N-6@24-mer synthesized by the process was analyzed by HPLC chromatogram and the results are provided in FIGS. 8B-8C.

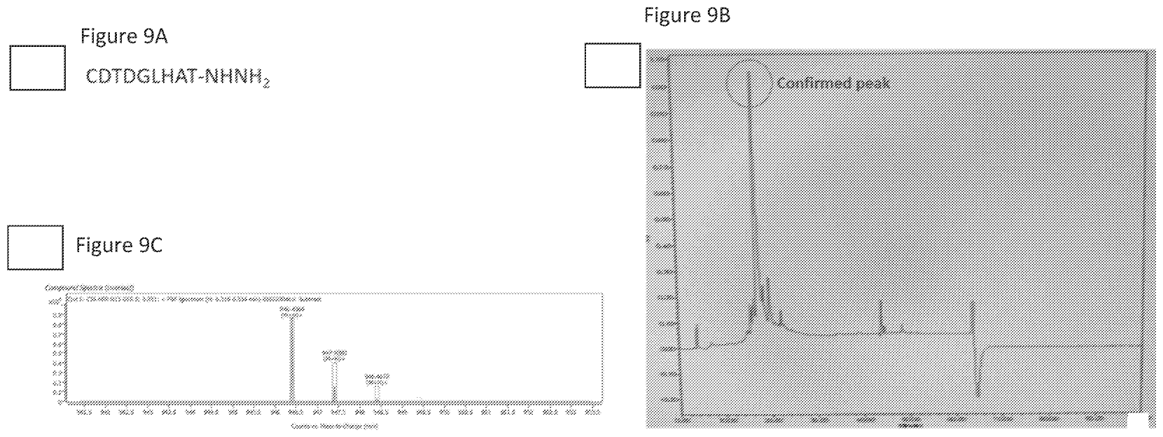

Solid Phase Peptide Synthesis of D-9° N—C-3@9-mer: CDTDGLHAT-NHNH₂(SEQ ID NO: 14) (FIG. 9A) D-9° N—C-3@9-mer was synthesized on the Liberty Blue 2.0 automated microwave peptide synthesizer (CEM) by following the conditions mentioned in experimental methods. D-9° N—C-3@9-mer synthesized by the process was analyzed by HPLC chromatogram and the results are provided in FIGS. 9B-9C.

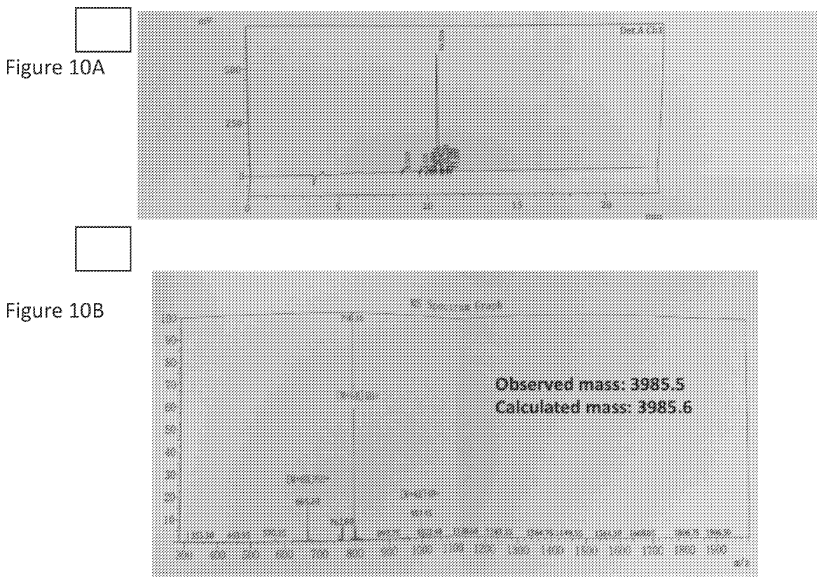

Solid Phase Peptide Synthesis of Customized D-9° N—C-1@33-mer: MKATVDPLEK KLLDYRQRLI KILANSFYGYYGY-NHNH₂. D-9° N—C-1@33-mer synthesized by the process was analyzed by HPLC chromatogram and the results are provided in FIGS. 10A-10B.

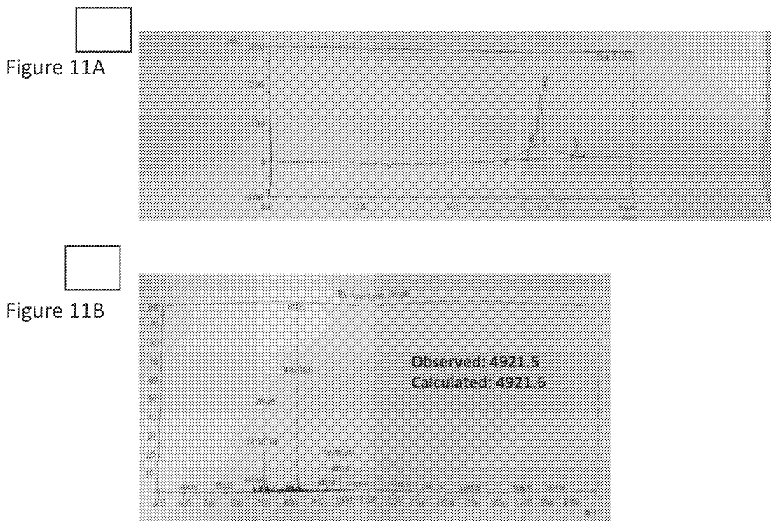

Solid Phase Peptide Synthesis of Customized D-9° N—C-2@39-mer: CKARWY-C(Acm)-KE-C(Acm) AESVTAWGRE YIEMVIRELE EKFGFKVLY-NHNH₂. D-9° N—C-2@39-mer synthesized by the process was analyzed by HPLC chromatogram and the results are provided in FIGS. 1A-11B.

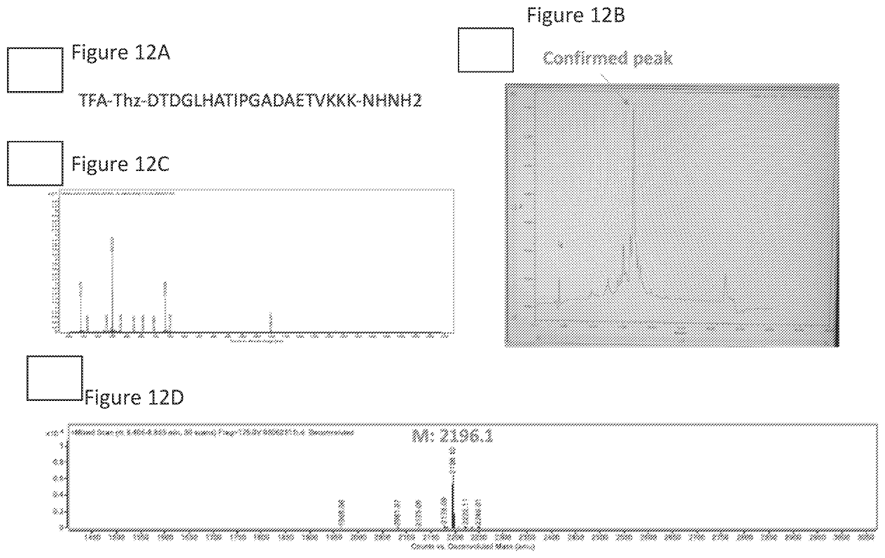

Solid phase peptide synthesis of D-9° N—C-3@21-mer: TFA-Thz-DTDGLHATIPGADAETVKKK-NHNH₂ (SEQ ID NO: 15). D-9° N—C-3@21-mer was synthesized on the Liberty Blue 2.0 automated microwave peptide synthesizer (CEM) by following the conditions mentioned in experimental methods. The product was observed as the TFA-deprotected D-9° N—C-3@21-mer as provided in FIGS. 12B and 12C.

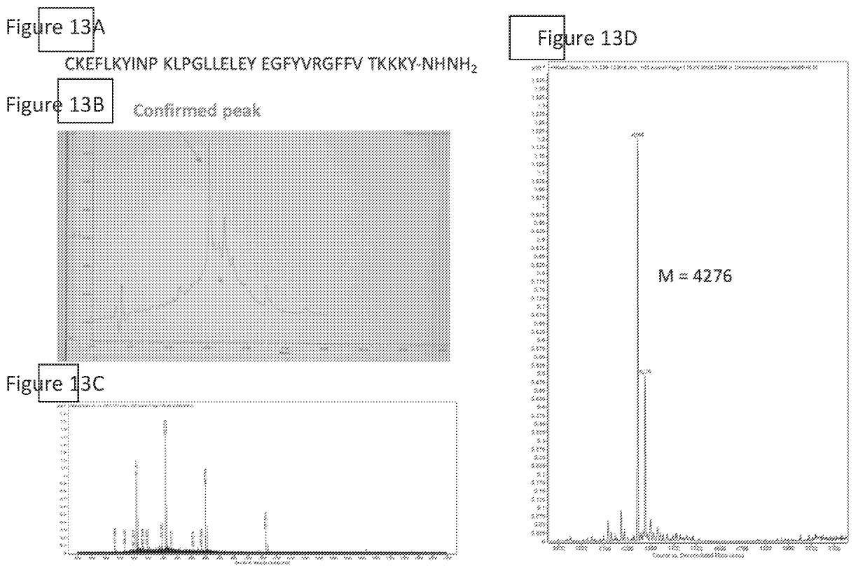

Solid phase peptide synthesis of D-9° N—C-3@Cys35-mer: CKEFLKYINP KLPGLLELEY EGFYVRGFFV TKKKY-NHNH₂. (SEQ ID NO: 16) D-9° N—C-3@Cys35-mer was synthesized on the Liberty Blue 2.0 automated microwave peptide synthesizer (CEM) by following the conditions mentioned in experimental methods. The product was observed as expected mass. as provided in FIGS. 13B-13D.

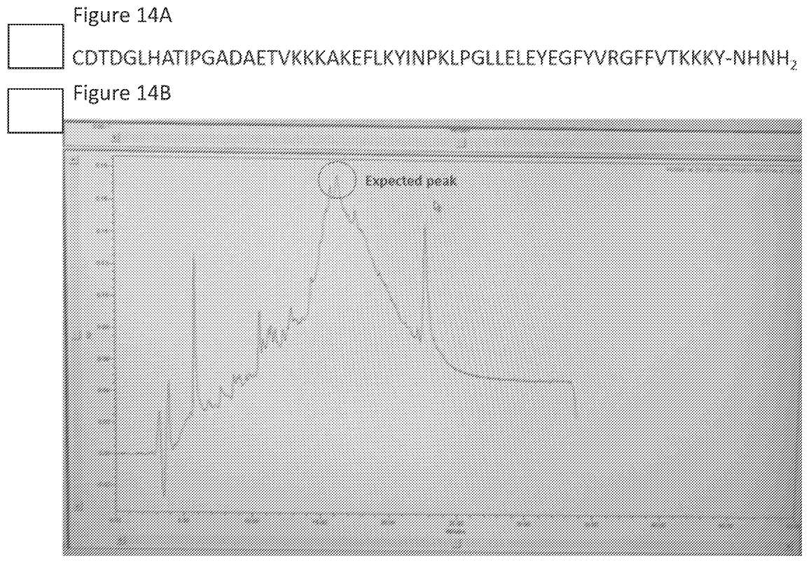

Solid phase peptide synthesis of D-9° N—C-3@56-mer: CDTDGLHATIPGADAETVKKKAKEFLKYINPKLPGLLELEYEGFYVRGFFVTKKKY-NHNH₂. (SEQ ID NO: 17) (FIG. 14A) D-9° N—C-3@56-mer was synthesized on the Liberty Blue 2.0 automated microwave peptide synthesizer (CEM) by following the conditions mentioned in experimental methods. The product was observed as expected mass. as provided in FIG. 14B.

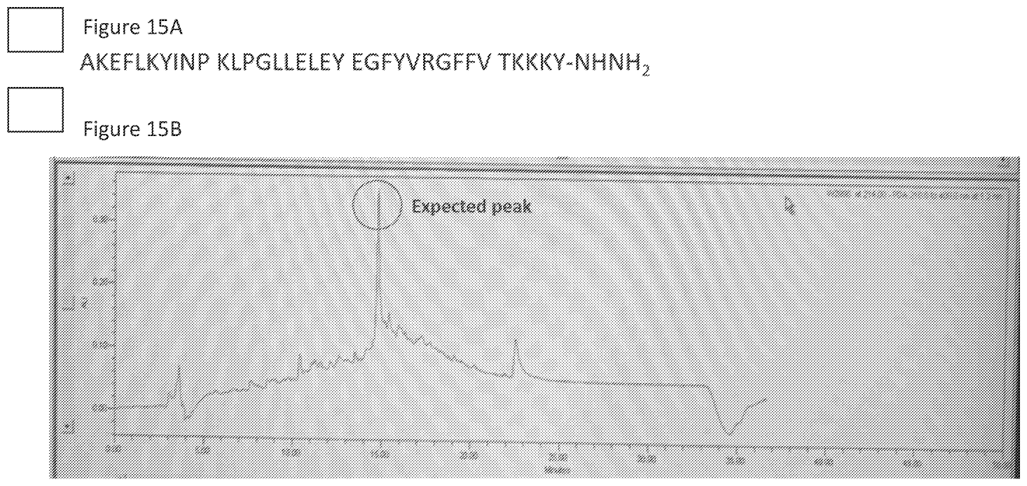

D-9° N—C-3@35-mer: AKEFLKYINP KLPGLLELEY EGFYVRGFFV TKKKY-NHNH₂. (SEQ ID NO: 18) (FIG. 15A) D-9° N—C-3@35-mer was synthesized on the Liberty Blue 2.0 automated microwave peptide synthesizer (CEM) by following the conditions mentioned in experimental methods. The product was observed as expected mass. as provided in FIG. 15B.

Native Chemical Ligation (NCL): Reagent Preparation: NaOH, 1 M and 6 M. Dissolve 40 mg or 240 mg of NaOH in 1 ml of deionized H2O. HCl, 6 M. Mix 1 ml of 12 M HCl with 1 ml of deionized H₂O. Two different phosphate solutions (0.1 M) containing 6 M Gn·HCl (pH 3.0-3.1 and pH 5.7-6.0, respectively). For a 10-ml solution, mix 156 mg of NaH₂PO₄·2H₂O and 5.74 g of Gn·HCl into a 10-ml volumetric flask and adjust it to the final volume with deionized H₂O. Adjust the pH to 3.0-3.1 with 6 M NaOH and 6 M HCl. Filter the solution by using a 13 mm×0.22 μm microporous membrane filter. The filtered solution can be stored at 4° C. for at least 1 month. Na₂HPO₄ 12H₂O (0.1 M) containing 6 M Gn·HCl (pH 5.7-6.0) also prepared as same manner. NaNO₂, 0.5 M. Dissolve 17 mg of NaNO₂ in 0.5 ml of deionized H₂O. TCEP, 0.1 M. Dissolve 58 mg of TCEP·HCl into 1 ml of 0.2 M phosphate solution (pH 3.0) containing 6 M guanidine hydrochloride (Gn·HCl). Adjust the pH to 6.0-7.0, and filter the solution by using a 13 mm×0.22 μm microporous membrane filter. VA-044, 0.1 M. Weight 9.7 mg of VA-044 into a 2-ml Eppendorf reaction tube and add 0.3 ml of 0.2 M phosphate solution (pH 6.9-7.0) containing 6 M Gn·HCl. Completely dissolve VA-044 by using a vortex and an ultrasonic cleaning bath.

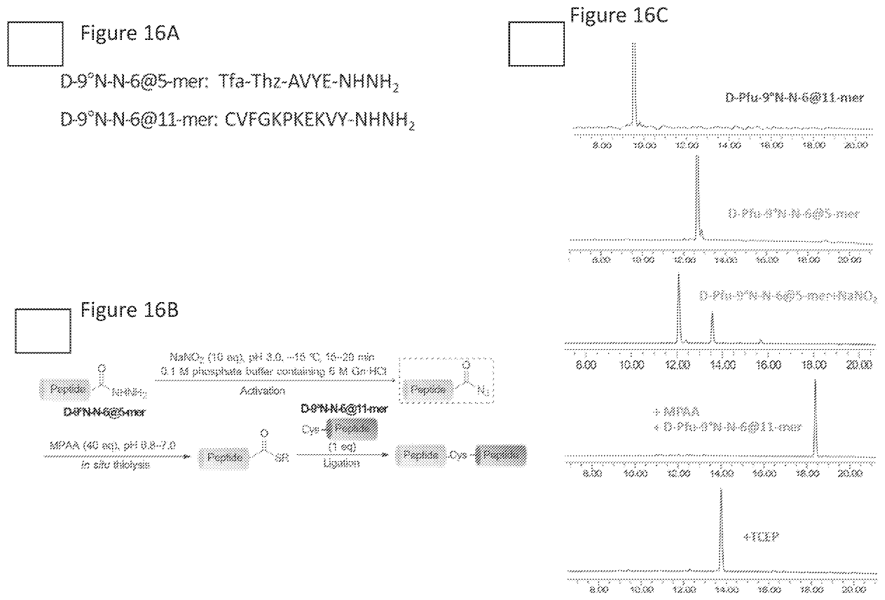

Synthesis of D-9° N—N-6@16-mer: Tfa-Thz-AVYE(CVFGKPKEKVY-NHNH₂(SEQ ID NO: 19 and SEQ ID NO: 20) (FIG. 16A). The process for the preparation of D-9° N—N-6@16-mer is illustrated in FIG. 16B and the HPLC chromatogram analysis of the D-9° N—N-6@16-mer is provided in FIG. 16C.

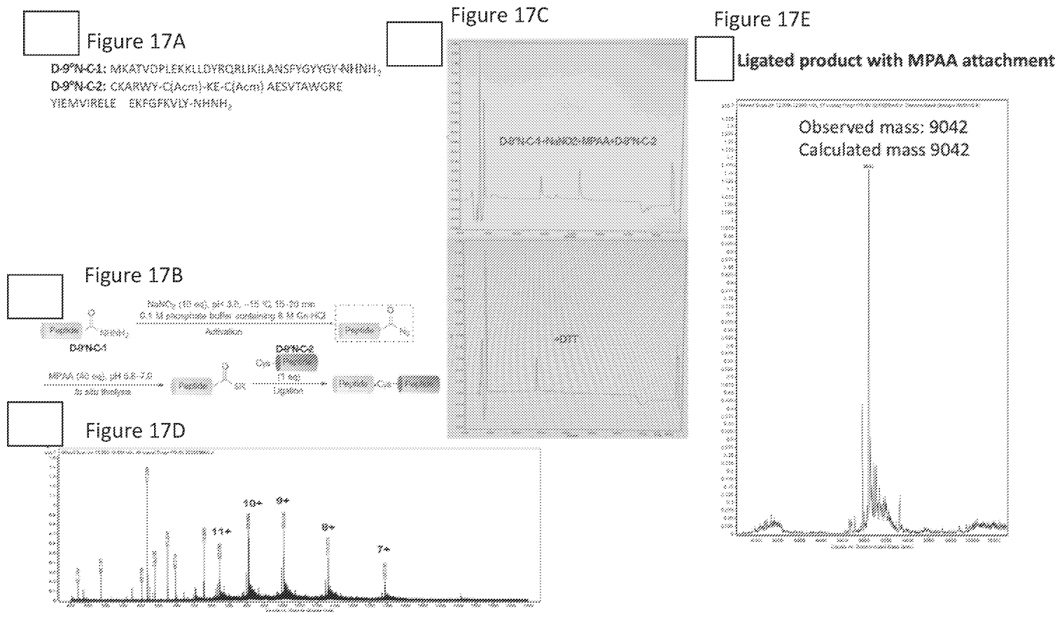

Synthesis of D-9° N—C-7@72-mer: MKATVDPLEKKLLDYRQRLIKILANSFYGYYGYCKARWY-C(Acm)-KE-C(Acm) AESVTAWGRE YIEMVIRELE EKFGFKVLY-NHNH₂ (SEQ ID NO: 21, 22, 23) (FIG. 17A). The process for the preparation of D-9° N—C-7@72-mer is illustrated in FIG. 17B. FIG. 17C is the analytical HPLC chromatogram of the peaks transformation in progressing the NCL reaction (λ=214 nm). FIG. 17D is ESI-MS of D-9° N—C-7@72-mer with MPAA attachment and FIG. 17E shows MPAA-attached ligated.

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### 8. SEQUENCES

| SEQ ID NO | Sequence |
|---|---|
| SEQ ID NO: 1 | MILDTDYITENGKPVIRVFKKENGEFKIEYDRTFEPYFYALLKDDSAIEDVKKVTAKRHG |
| (9º N | TVVKVKRAEKVQKKFLGRPIEVWKLYFNHPQDVPAIRDRIRAHPAVVDIYEYDIPFAKRY |
| polymerase) | LIDKGLIPMEGDEELTMLAFDIETLYHEGEEFGTGPILMISYADGSEARVITWKKIDLPY |
| | VDVVSTEKEMIKRFLRVVREKDPDVLITYNGDNFDFAYLKKRCEELGIKFTLGRDGSEPK |
| | IQRMGDRFAVEVKGRIHFDLYPVIRRTINLPTYTLEAVYEAVFGKPKEKVYAEEIAQAWE |
| | SGEGLERVARYSMEDAKVTYELGREFFPMEAQLSRLIGQSLWDVSRSSTGNLVEWFLLRK |
| | AYKRNELAPNKPDERELARRRGGYAGGYVKEPERGLWDNIVYLDFRSLYPSIHITHNVSP |
| | DTLNREGCKEYDVAPEVGHKFCKDFPGFIPSLLGDLLEERQKIKRKMKATVDPLEKKLLD |
| | YRQRAIKILANSFYGYYGYAKARWYCKECAESVTAWGREYIEMVIRELEEKFGFKVLYAD |
| | TDGLHATIPGADAETVKKKAKEFLKYINPKLPGLLELEYEGFYVRGFFVTKKKYAVIDEE |
| | GKITTRGLEIVRRDWSEIAKETQARVLEAILKHGDVEEAVRIVKEVTEKLSKYEVPPEKL |
| | VIHEQITRDLRDYKATGPHVAVAKRLAARGVKIRPGTVISYIVLKGSGRIGDRAIPADEF |
| | DPTKHRYDAEYYIENQVLPAVERILKAFGYRKEDLRYQKTKQVGLGAWLKVKGKK |
| SEQ ID NO: 2 | MILDTDYITENGKPVIRVFKKENGEFKIEYDRTFEPYFYALLKDDSAIEDVKKVTAKRHG |
| (Amino-acid | TVVKVKRAEKVQKKFLGRPIEVWKLYFNHPQDVPAIRDRIRAHPAVVDIYEYDIPFAKRY |
| sequence of (D)- | LIDKGLIPMEGDEELTMLAFAIATLYHEGEEFGTGPILMISYADGSEARVITWKKIDLPY |
| form mutant | VDVVSTEKEMIKRFLRVVREKDPDVLITYNGDNFDFAYLKKRCEELGIKFTLGRDGSEPK |
| 9º N DNA | IQRMGDRFAVEVKGRIHFDLYPVIRRTINLPTYTLAAVYEAVFGKPKEKVYAEEIAQAWE |
| polymerase | SGEGLERVARYSMEDAGVTYELGREFFPMEAQLSRLIGQSLWDVSRSSTGNLVEWELLRK |
| (candidate #1) | AYKRNELAPNKPDERELARRRGGYAGGYVKEPERGLWDNIVYLDERSLVPSIHITHNVSP |
| | DTLAREGCKEYDVAPEVGHKFCKDFPGFIPSLLGDLLEERQKIKRKMKATVDPLEKKLLD |
| | YRQRLIKILANSFYGYYGYAKARWYCKECAESVTAWGREYIEMVIRELEEKFGFKVLYAD |
| | TDGLHATIPGADAETVKKKAKEFLKYINPKLPGLLELEYEGFYVRGFFVTKKKYAVIDEE |
| | GKITTRGLEIVRRDWSEIAKETQARVLEAILKHGDVEEAVRIVKEVTEKLAKYEVPPEKL |
| | VIHEQITRDLRDYKATGPHVAVAKRLAARGVKIRPGTVISYIVLKGSGRIGDRAIPADEF |
| | DPTKHRYDAEYYIENQVLPAVERILKAFGYRKEDLRYQKTKQVGLGAWLKVKGKK |
| SEQ ID NO: 3 | MILDTDYITENGKPVIRVFKKENGEFKIEYDRTFEPYFYALLKDDSAIEDVKKVTAKRHG |
| Amino-acid | TVVKVKRAEKVQKKFLGRPVEVWKLYFNHPQDVPAIRDRIRAHPAVVDIYEYDIPFAKRY |
| sequence of (D)- | LIDKGLVPMEGDEELTMLAFAIATLYHEGEEFGTGPILMISYADGSEARVATWKKVDLPY |
| form mutant | VDVVSTEKEMVKRFLRVVREKDPDVLITYNGDNFDFAYLKKRCEELGVKFTLGRDGSEPK |
| 9º N DNA | IQRMGDRFAVEVKGRVHFDLYPVARRTLNLPTYTLAAVYEAVFGKPKEKVYAREIAQAWE |
| polymerase | SGEGLERVARYSMEDAGVTYELGREFFPMEAQLSRLIGQSLWDVSRSSTGNLVEWFLLRK |
| (candidate #1-I) | AYKRNELAPNKPDERELARRRGGYAGGYVKEPERGLWDNVVYLDERSLVPSIIITHNVSP |
| | DTLAREGCKEYDVAPEVGHKFCKDFPGFIPSLLGDLLEERQKIKRKMKATVDPLEKKLLD |
| | YRQRLIKILANSFYGYYGYAKARWYCKECAESVTAWGREYIEMVIRELEEKFGFKVLYAD |
| | TDGLHATIPGADAETVKKKAKEFLKYINPKLPGLLELEYEGFYVRGFFVTKKKYAVVDEE |
| | GKITTRGLEVVRRDWSEAAKETQARVLEALLKHGDVEEAVRVVKEVTEKLAKYEVPPEKL |
| | VIHEQITRDLRDYKATGPHVAVAKRLAARGVKIRPGTVISYIVLKGSGRIGDRAYPADEF |
| | DPTKHRYDAEYYVENQVLPAVERVLKAFGYRKEDLRYQKTKQVGLGAWLKVKGKK |
| SEQ ID NO: 4 | MILDTDYITENGKPVIRVFKKENGEFKIEYDRTFEPYFYALLKDDSAIEDVKKVTAKRHG |
| Amino-acid | TVVKVKRAEKVQKKFLGRPIEVWKLYFNHPQDVPAIRDRIRAHPAVVDIYEYDIPFAKRY |
| sequence of (D)- | LIDKGLIPMEGDEELTMLAFAIATLYHEGEEFGTGPILMISYADGSEARVITWKKIDLPY |
| form mutant | VDVVSTEKEMIKRFLRVVREKDPDVLITYNGDNFDFAYLKKRCEELGIKFTLGRDGSEPK |
| 9º N DNA | IQRMGDRFAVEVKGRIHFDLYPVIRRTINLPTYTLAAVYEAVFGKPKEKVYAEEIAQAWE |
| polymerase | SGEGLERVARYSMEDAGVTYELGREFFPMEAQLSRLIGQSLWDVSRSSTGNLVEWELLRK |
| (candidate #2) | AYKRNELAPNKPDERELARRRGGYAGGYVKEPERGLWDNIVYLDFRSAAISIIITHNVSP |
| | DTLAREGCKEYDVAPEVGHKFCKDFPGFIPSLLGDLLEERQKIKRKMKATVDPLEKKLLD |
| | YRQRVIKILANSFYGYYGYAKARWYCKECAESVSAWGREYLEMVIRELEEKFGFKVLYAD |
| | TDGLHATIPGADAETVKKKAKEFLKYINPKLPGLLELEYEGFYVRGFFVTKKKYAVIDEE |
| | GKITTRGLEIVRRDWSEIAKETQARVLEAILKHGDVEEAVRIVKEVTEKLAKYEVPPEKL |
| | VIHEQITRDLRDYKATGPHVAVAKRLAARGVKIRPGTVISYIVLKGSGRIGDRAIPADEF |
| | DPTKHRYDAEYYIENQVLPAVERILKAFGYRKEDLRYQKTKQVGLGAWLKVKGKK |
| SEQ ID NO: 5 | MILDTDYITENGKPVIRVFKKENGEFKIEYDRTFEPYFYALLKDDSAIEDVKKVTAKRHG |
| Amino-acid | TVVKVKRAEKVQKKFLGRPVEVWKLYFNHPQDVPAIRDRIRAHPAVVDIYEYDIPFAKRY |
| sequence of (D)- | LIDKGLVPMEGDEELTMLAFAIATLYHEGEEFGTGPILMISYADGSEARVATWKKVDLPY |
| form mutant | VDVVSTEKEMVKRFLRVVREKDPDVLITYNGDNFDFAYLKKRCEELGVKFTLGRDGSEPK |
| 9º N DNA | IQRMGDRFAVEVKGRVHFDLYPVARRTLNLPTYTLAAVYEAVFGKPKEKVYAEEIAQAWE |
| polymerase | SGEGLERVARYSMEDAGVTYELGREFFPMEAQLSRLIGQSLWDVSRSSTGNLVEWFLLRK |
| (candidate #2-1) | AYKRNELAPNKPDERELARRRGGYAGGYVKEPERGLWDNVVYLDFRSAAISIIITHNVSP |
| | DTLAREGCKEYDVAPEVGHKFCKDFPGFIPSLLGDLLEERQKIKRKMKATVDPLEKKLLD |
| | YRQRVIKILANSFYGYYGYAKARWYCKECAESVSAWGREYLEMVIRELEEKFGFKVLYAD |
| | TDGLHATIPGADAETVKKKAKEFLKYINPKLPGLLELEYEGFYVRGFFVTKKKYAVVDEE |
| | GKITTRGLEVVRRDWSEAAKETQARVLEALLKHGDVEEAVRVVKEVTEKLAKYEVPPEKL |
| | VIHEQITRDLRDYKATGPHVAVAKRLAARGVKIRPGTVISYIVLKGSGRIGDRAYPADEF |
| | DPTKHRYDAEYYVENQVLPAVERVLKAFGYRKEDLRYQKTKQVGLGAWLKVKGKK |
| SEQ ID NO: 6 | MILDTDYITENGKPVIRVFKKENGEFKIEYDRTFEPYFYALLKDDSAIEDVKKVTAKRHG |
| Amino-acid | TVVKVKRAEKVQKKFLGRPIEVWKLYFNHPQDVPAIRDRIRAHPAVVDIYEYDIPFAKRY |
| sequence of (D)- | LIDKGLIPLEDGDEELTMLAFAIATLYHEGEEFGTGPILMISYADGSEARVITWKKIDLP |
| form mutant | YVDVVSTEKEMIKRFLRVVREKDPDVLITYNGDNFDFAYLKKRCEELGIKFTLGRDGSEP |
| 9º N DNA | KIQRMGDRFAVEVKGRIHFDLYPVIRRTINLPTYTLAAVYEAVFGKPKEKVYAEEIAQAW |
| polymerase | ESGEGLERVARYSMEDAGVTYELGREFFPMEAQLSRLIGQSLWDVSRSSTGNLVEWFLLR |
| (candidate #3) | KAYKRNELAPNKPDERELARRRGGYAGGYVKEPERGLWDNIVYLDFRSAAISIIITHNVS |
| | PDTLAREGCKEYDVAPEVGHKFCKDFPGFIPSLLGDLLEERQKIKRKMKATVDPLEKKLL |
| | DYRQRVIKILANSFYGYYGYAKARWYCKECAESVSAWGREYLEMVIRELEEKFGFKVLYA |
| | DTDGLHATIPGADAETVKKKAKEFLKYINPKLPGLLELEYEGFYVRGFFVTKKKYAVIDE |
| | EGKITTRGLEIVRRDWSEIAKETQARVLEAILKHGDVEEAVRIVKEVTEKLAKYEVPPEK |
| | LVIHEQITRDLRDYKATGPHVAVAKRLAARGVKIRPGTVISYIVLKGSGRIGDRAIPADE |
| | FDPTKHRYDAEYYIENQVLPAVERILKAFGYRKEDLRYQKTKQVGLGAWLKVKGKK |
| SEQ ID NO: 7 | MILDTDYITENGKPVIRVFKKENGEFKIEYDRTFEPYFYALLKDDSAIEDVKKVTAKRHG |
| Amino-acid | TVVKVKRAEKVQKKFLGRPVEVWKLYFNHPQDVPAIRDRIRAHPAVVDIYEYDIPFAKRY |
| sequence of (D)- | LIDKGLVPLEDGDEELTMLAFAIATLYHEGEEFGTGPILMISYADGSEARVATWKKVDLP |
| form mutant | YVDVVSTEKEMVKRFLRVVREKDPDVLITYNGDNFDFAYLKKRCEELGVKFTLGRDGSEP |
| 9º N DNA | KIQRMGDRFAVEVKGRVHFDLYPVARRTLNLPTYTLAAVYEAVFGKPKEKVYAEEIAQAW |
| polymerase | ESGEGLERVARYSMEDAGVTYELGREFFPMEAQLSRLIGQSLWDVSRSSTGNLVEWFLLR |
| (candidate #3-I) | KAYKRNELAPNKPDERELARRRGGYAGGYVKEPERGLWDNVVYLDFRSAAISHIITHNVS |
| | PDTLAREGCKEYDVAPEVGHKFCKDFPGFIPSLLGDLLEERQKIKRKMKATVDPLEKKLL |
| | DYRQRVIKILANSFYGYYGYAKARWYCKECAESVSAWGREYLEMVIRELEEKFGFKVLYA |
| | DTDGLHATIPGADAETVKKKAKEFLKYINPKLPGLLELEYEGFYVRGFFVTKKKYAVVDE |
| | EGKITTRGLEVVRRDWSEAAKETQARVLEALLKHGDVEEAVRVVKEVTEKLAKYEVPPEK |
| | LVIHEQITRDLRDYKATGPHVAVAKRLAARGVKIRPGTVISYIVLKGSGRIGDRAYPADE |
| | FDPTKHRYDAEYYVENQVLPAVERVLKAFGYRKEDLRYQKTKQVGLGAWLKVKGKK |
| SEQ ID NO: 8 | MILDTDYITENGKPVIRVFKKENGEFKIEYDRTFEPYFYALLKDDSAIEDVKKVTAKRHG |
| | TVVKVKRAEKVQKKFLGRPVEVWKLYFNHPQDVPAIRDRIRAHPAVVDIYEYDIPFAKRY |
| | LIDKGLVPMEGDEELTMLAFAIATLYHEGEEFGTGPILMISYADGSEARVATWKKVDLPY |
| | VDVVSTEKEMVKRFLRVVREKDPDVLITYNGDNFDFAYLKKRCEELGVKFTLGRDGSEPK |
| | IQRMGDRFAVEVKGRVHFDLYPVARRTLNLPTYTLAAVYEAVFGKPKEKVYABEIAQAWE |
| | SGEGLERVARYSMEDAGVTYELGREFFPMEAQLSRLIGQSLWDVSRSSTGNLVEWELLRK |
| | AYKRNELAPNKPDERELARRRGGYAGGYVKEPERGLWDNVVYLDFRSLVPSIHITHNVSP |
| | DTLAREGCKEYDVAPEVGHKFCKDFPGFIPSLLGDLLEERQKIKRKMKATVDPLEKKLLD |
| | YRQRLIKILANSFYGYYGYAKARWYCKECAESVTAWGREYIEMVIRELEEKFGFKVLYAD |
| | TDGLHATIPGADAETVKKKAKEFLKYINPKLPGLLELEYEGFYVRGFFVTKKKYAVVDEE |
| | GKITTRGLEVVRRDWSEAAKETQARVLEALLKHGDVEEAVRVVKEVTEKLAKYEVPPEKL |
| | VIHEQITRDLRDYKATGPHVAVAKRLAARGVKIRPGTVISYIVLKGSGRIGDRAYPADEF |
| | DPTKHRYDAEYYVENQVLPAVERVLKAFGYRKEDLRYQKTKQVGLGAWLKVKGKK |
| SEQ ID NO: 9 | MILDTDYITENGKPVIRVFKKENGEFKIEYDRTFEPYFYALLKDDSAIEDVKKVTAKRHG |
| | TVVKVKRAEKVQKKFLGRPVEVWKLYFNHPQDVPAIRDRIRAHPAVVDIYEYDIPFAKRY |
| | LIDKGLVPMEGDEELTMLAFAIATLYHEGEEFGTGPILMISYADGSEARVATWKKVDLPY |
| | VDVVSTEKEMVKRFLRVVREKDPDVLITYNGDNFDFAYLKKRCEELGVKFTLGRDGSEPK |
| | IQRMGDRFAVEVKGRVHFDLYPVARRTLNLPTYTLAAVYEAVFGKPKEKVYABEIAQAWE |
| | SGEGLERVARYSMEDAGVTYELGREFFPMEAQLSRLIGQSLWDVSRSSTGNLVEWELLRK |
| | AYKRNELAPNKPDERELARRRGGYAGGYVKEPERGLWDNVVYLDERSLVPSIHITHNVSP |
| | DTLAREGCKEYDVAPEVGHKFCKDFPGFIPSLLGDLLEERQKIKRK |
| SEQ ID NO: 10 | MKATVDPLEKKLLDYRQRLIKILANSFYGYYGYAKARWYCKECAESVTAWGREYIEMVIR |
| | ELEEKFGFKVLYADTDGLHATIPGADAETVKKKAKEFLKYINPKLPGLLELEYEGFYVRG |
| | FFVTKKKYAVVDEEGKITTRGLEVVRRDWSEAAKETQARVLEALLKHGDVEEAVRVVKEV |
| | TEKLAKYEVPPEKLVIHEQITRDLRDYKATGPHVAVAKRLAARGVKIRPGTVISYIVLKG |
| | SGRIGDRAYPADEFDPTKHRYDAEYYVENQVLPAVERVLKAFGYRKEDLRYQKTKQVGLG |
| | AWLKVKGKK |
| SEQ ID NO: 11 | AVYE |
| SEQ ID NO: 12 | CVFGKPKEKVY |
| SEQ ID NO: 13 | AVYECVFGKPKEKVY |
| SEQ ID NO: 14 | CDTDGLHAT |
| SEQ ID NO: 15 | DTDGLHATIPGADAETVKKK |
| SEQ ID NO: 16 | CKEFLKYINP KLPGLLELEY EGFYVRGFFV TKKKY |
| SEQ ID NO: 17 | CDTDGLHATIPGADAETVKKKAKEFLKYINPKLPGLLELEYEGFYVRGFFVTKKKY |
| SEQ ID NO: 18 | AKEFLKYINP KLPGLLELEY EGFYVRGFFV TKKKY |
| SEQ ID NO: 19 | AVYE |
| SEQ ID NO: 20 | CVFGKPKEKVY |
| SEQ ID NO: 21 | MKATVDPLEKKLLDYRQRLIKILANSFYGYYGY |
| SEQ ID NO: 22 | CKARWY |
| SEQ ID NO: 23 | AESVTAWGRE YIEMVIRELE EKFGFKVLY |

1. A nucleotide comprising:
a. a pentose sugar comprising a cyclic form of a (3R,4S)-3,4,5-trihydroxypentanal or a (4R)-4,5-dihydroxypentanal;
wherein the H of the 5-hydroxyl group of the pentose sugar is substituted by one or more phosphate group; and
wherein the H of the 3-hydroxyl group of the (3R,4S)-3.4.5-trihydroxypentanal is optionally substituted by a cleavable protecting group;
b. a nitrogenous base linked to the 2 position of the pentose sugar, and
c. optionally a cleavable label comprising a cleavable linker and a label;
wherein at least one of a cleavable protecting group and a cleavable label is present.

2. The nucleotide of claim 0, wherein the cleavable label is present and is linked to the nitrogenous base, the 3′ O, or the 5′ phosphate group.

3. The nucleotide of claim 0, having a structure according to Formula I: 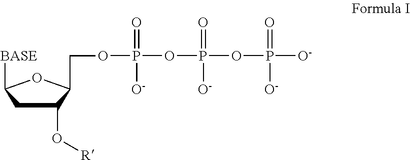
wherein Base is a nitrogenous base, and R′ is a cleavable protecting group.

4. The nucleotide of claim 0, having a structure according to Formula II 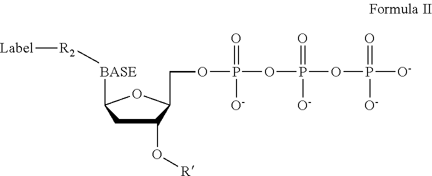
wherein Base is a nitrogenous base; R′ is a cleavable protecting group or H; R₂-Label is a cleavable label comprising a cleavable linker R₂ and a label.

5. The nucleotide of claim 0, having a structure according to Formula III: 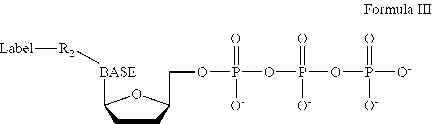
wherein Base is a nitrogenous base; R₂-Label is a cleavable label comprising a cleavable linker R₂ and a label.

6. The nucleotide of claim 1 wherein the label is selected from the group consisting of:

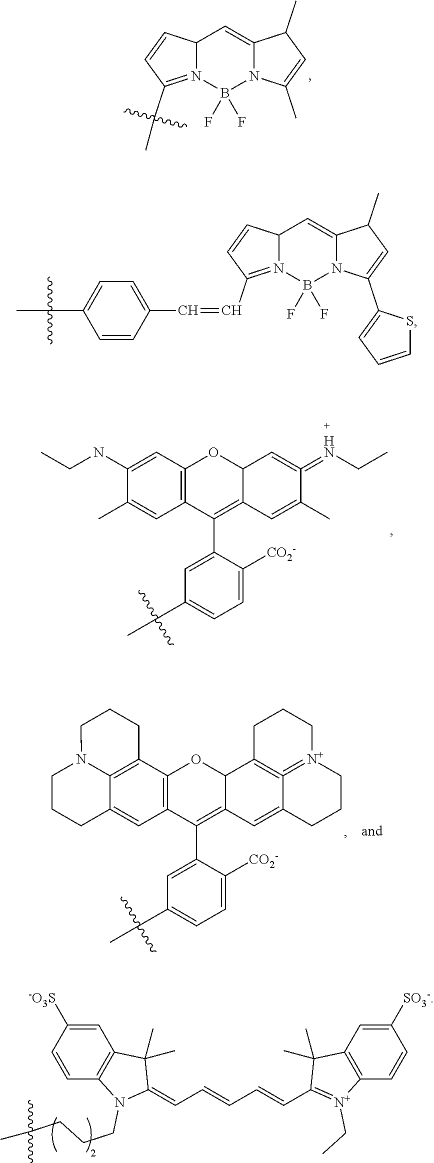

7. The nucleotide of claim 1, wherein the cleavable protecting group is selected from an allyl, a dimethyl disulfide, a nitrobenzyl, and an azido protecting group.

8. The nucleotide of claim 7, wherein the cleavable protecting group is selected from:

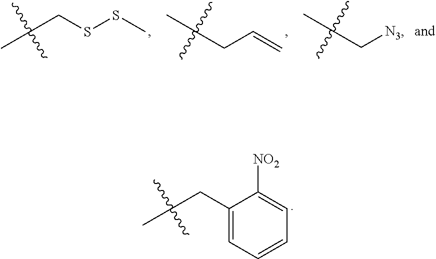

9. The nucleotide of claim 1, wherein the cleavable linker is photocleavable, is cleaved by contact with water-soluble phosphines, or is cleaved by water-soluble transition metal-containing catalysts.

10. The nucleotide of claim 9, wherein the cleavable linker comprises an allyl or an azido group.

11. The nucleotide of claim Error!Reference source not found., wherein the cleavable linker comprises:

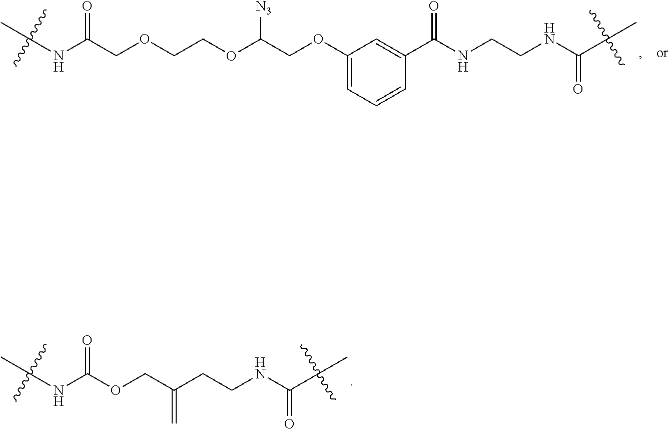 [SMILES: *NC(=O)COCCOC(COc1cccc(C(=O)NCCNC(*)=O)c1)N=[N+]=[N-] ; *NC(=O)OCC(=C)CCNC(*)=O]

12. The nucleotide of claim 3, selected from: 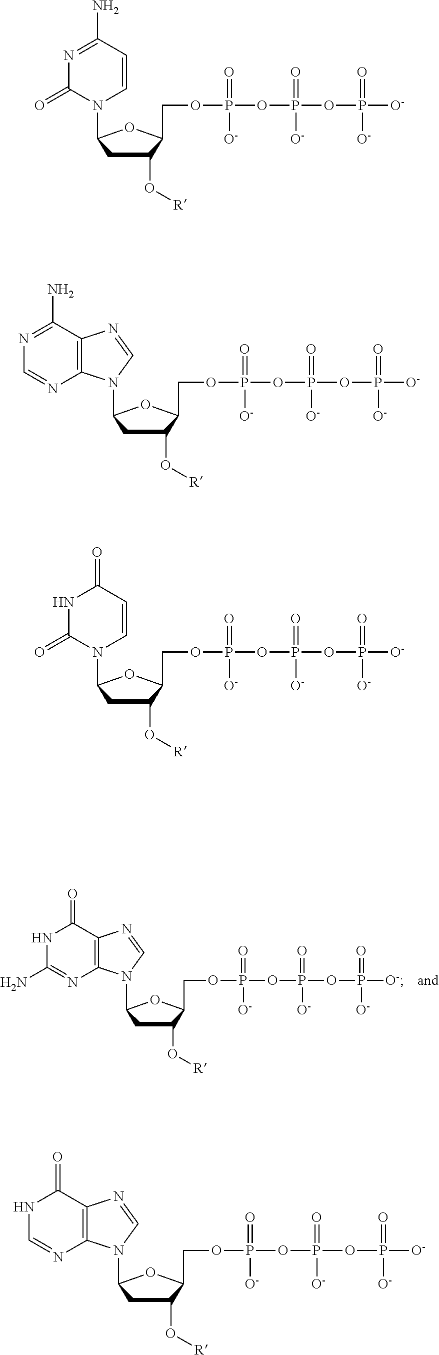
wherein the R′ is selected from:

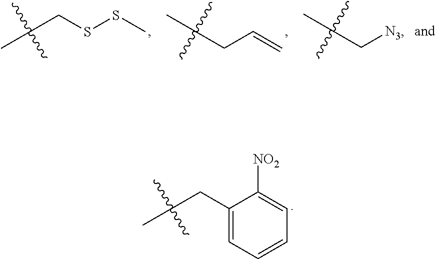

13. The nucleotide of claim 4, selected from: 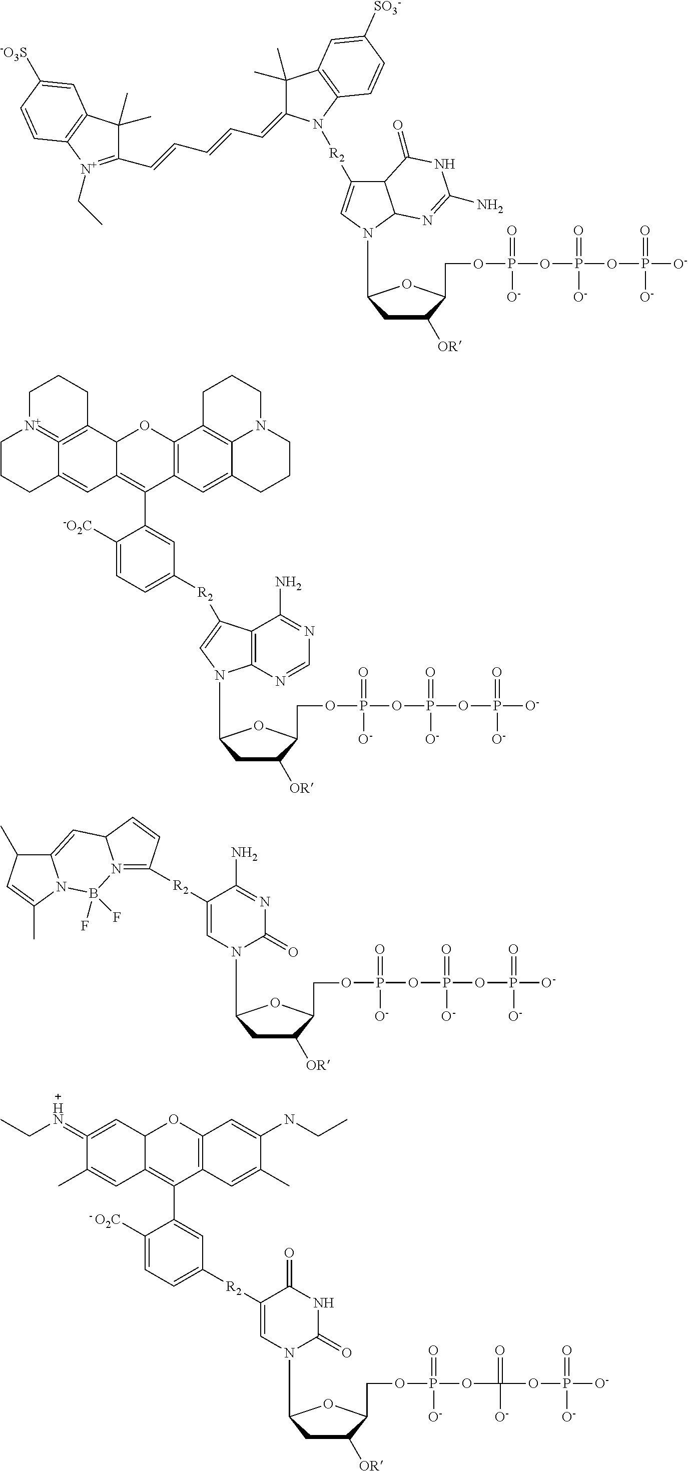 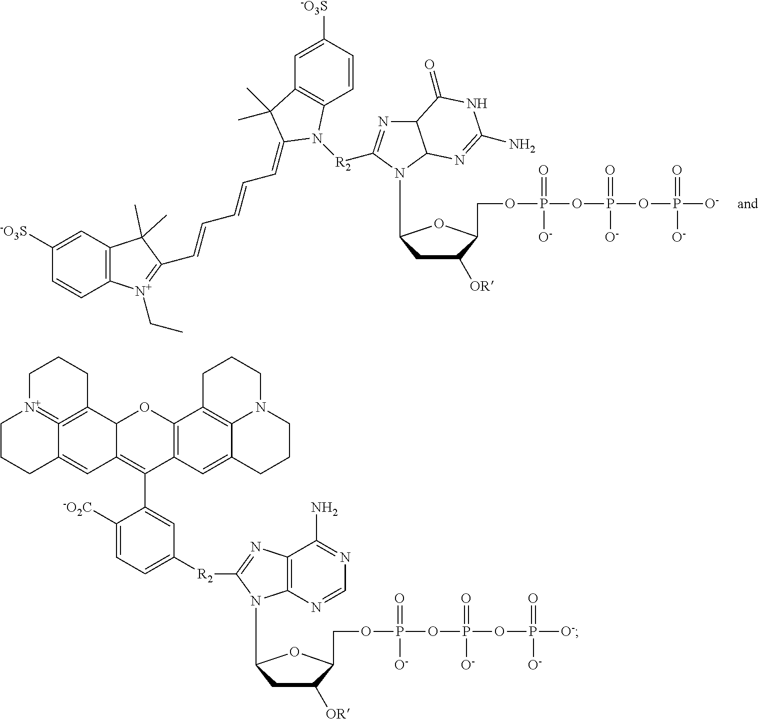
wherein the R′ is selected from:
R₂ comprises: H,

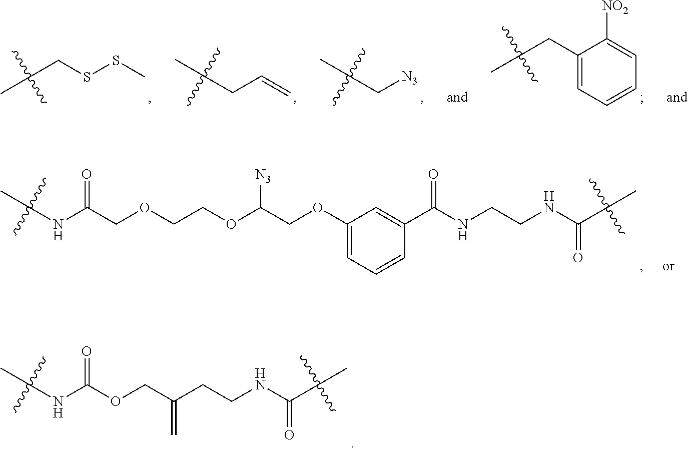

14. The nucleotide of claim 5, selected from: 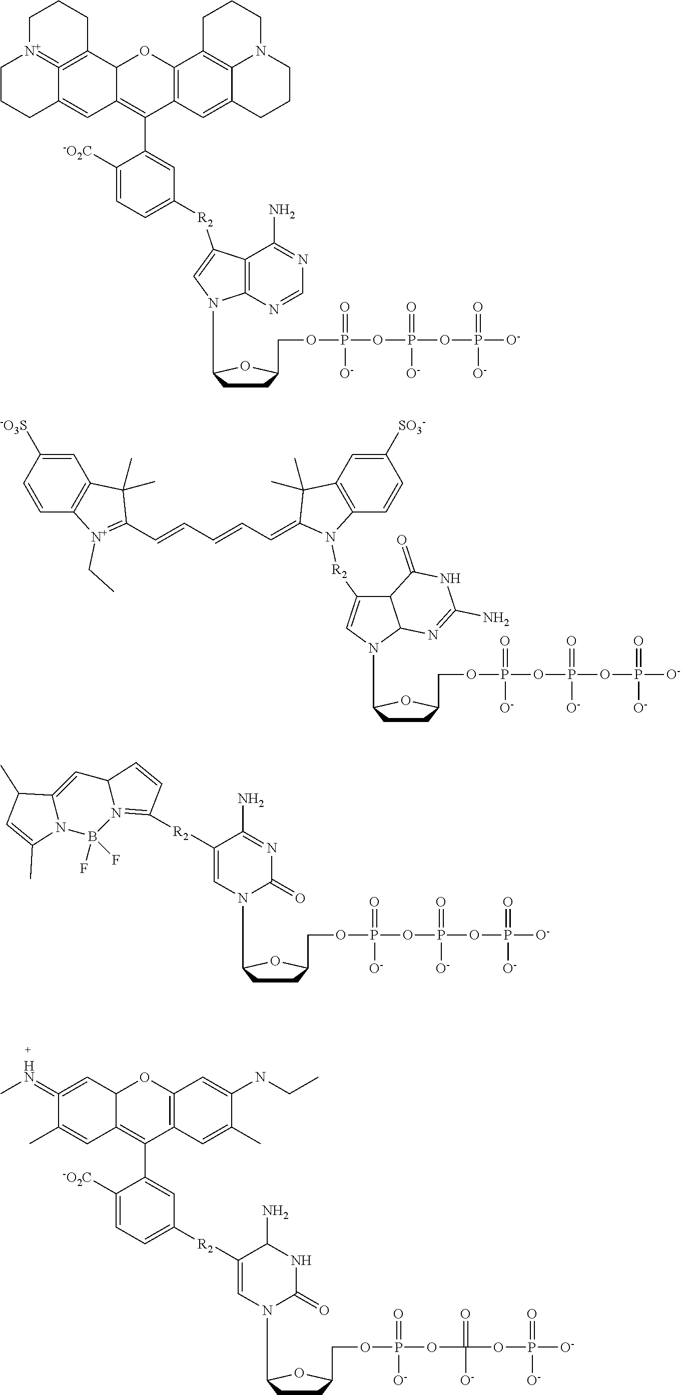 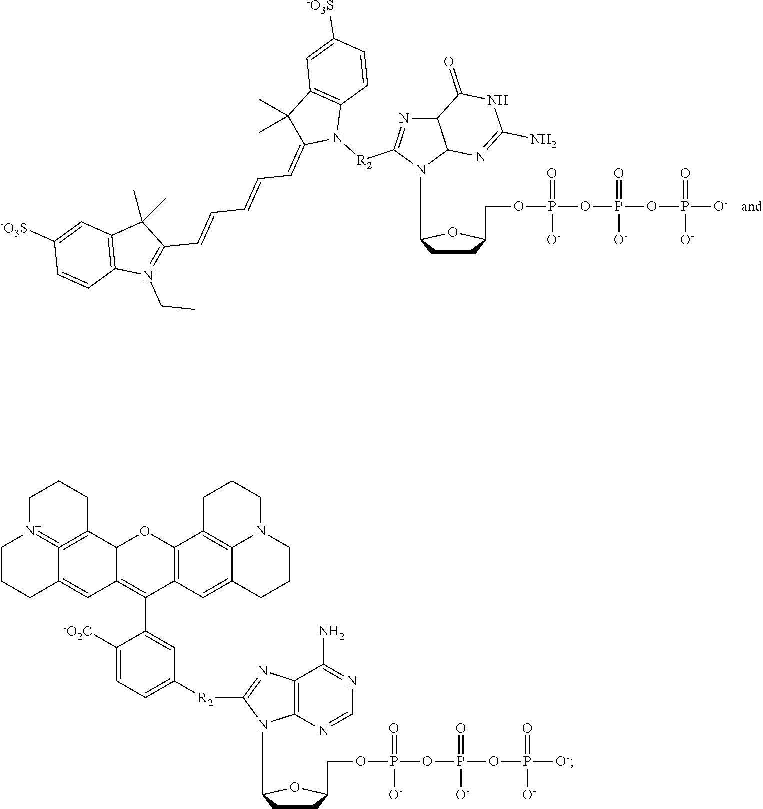
wherein R₂ comprises:

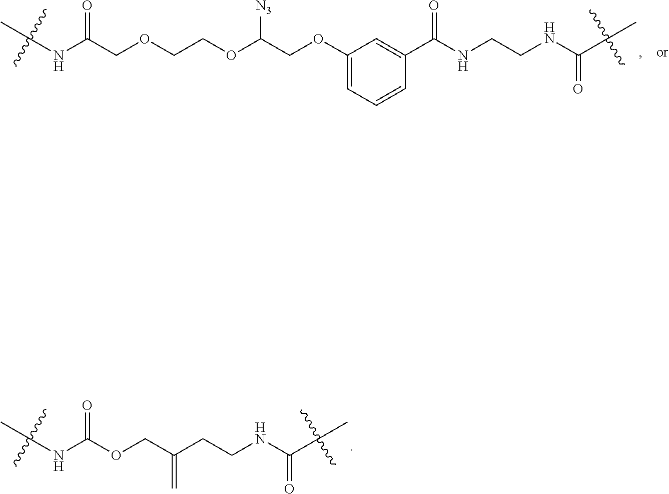 [SMILES: *NC(=O)COCCOC(COc1cccc(C(=O)NCCNC(*)=O)c1)N=[N+]=[N-] ; *NC(=O)OCC(=C)CCNC(*)=O]

15. The nucleotide of claim 4, selected from: 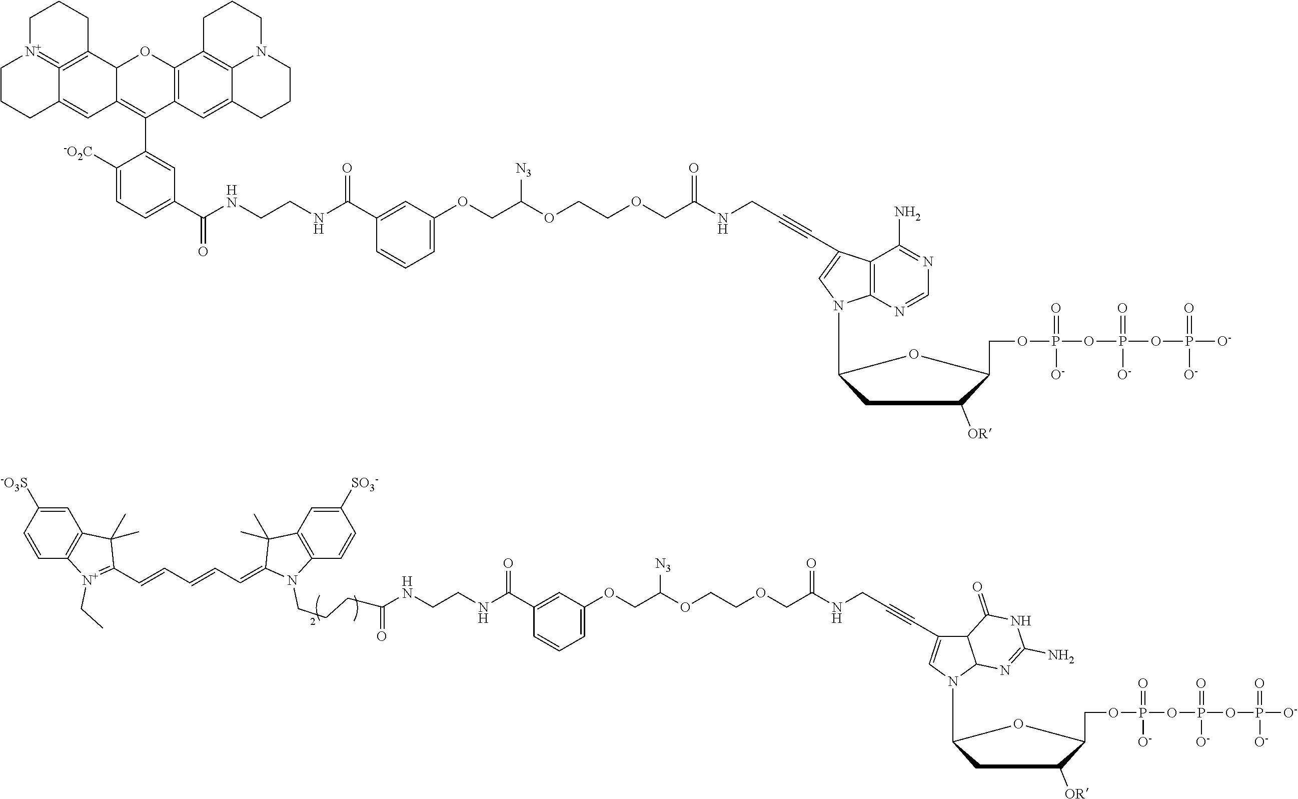 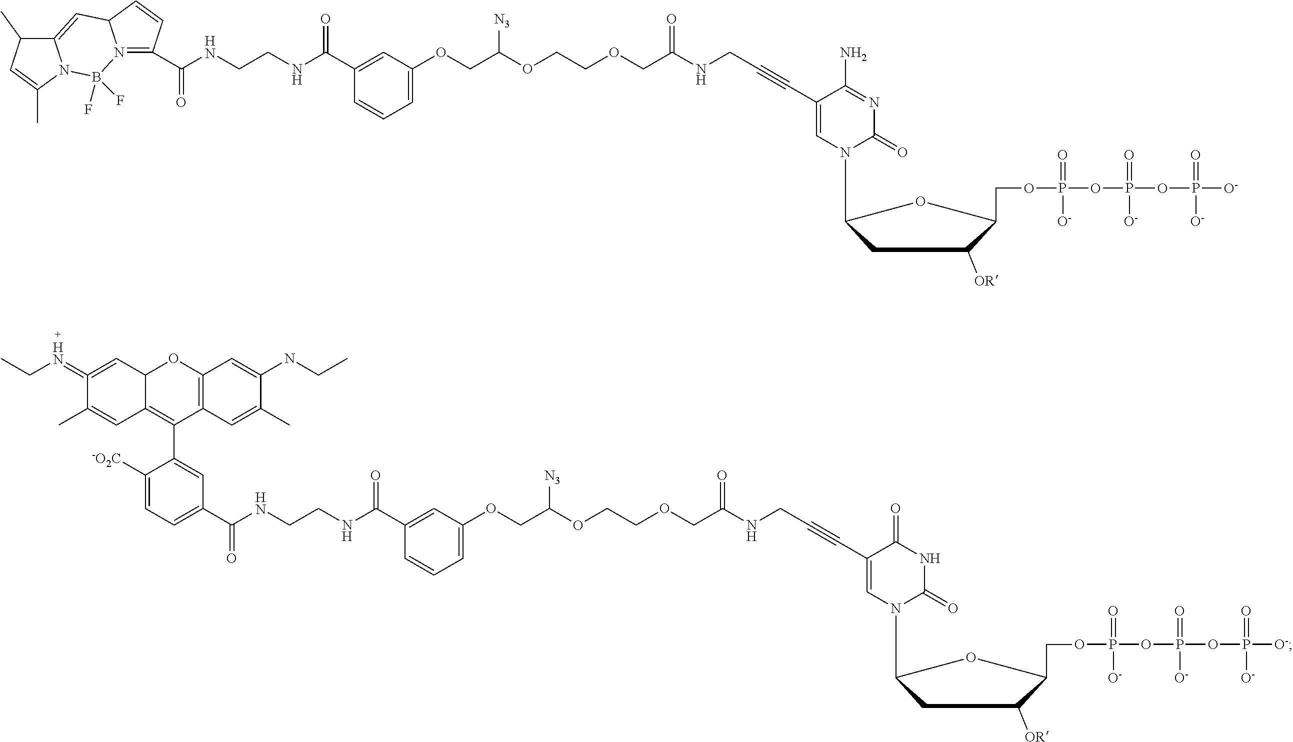 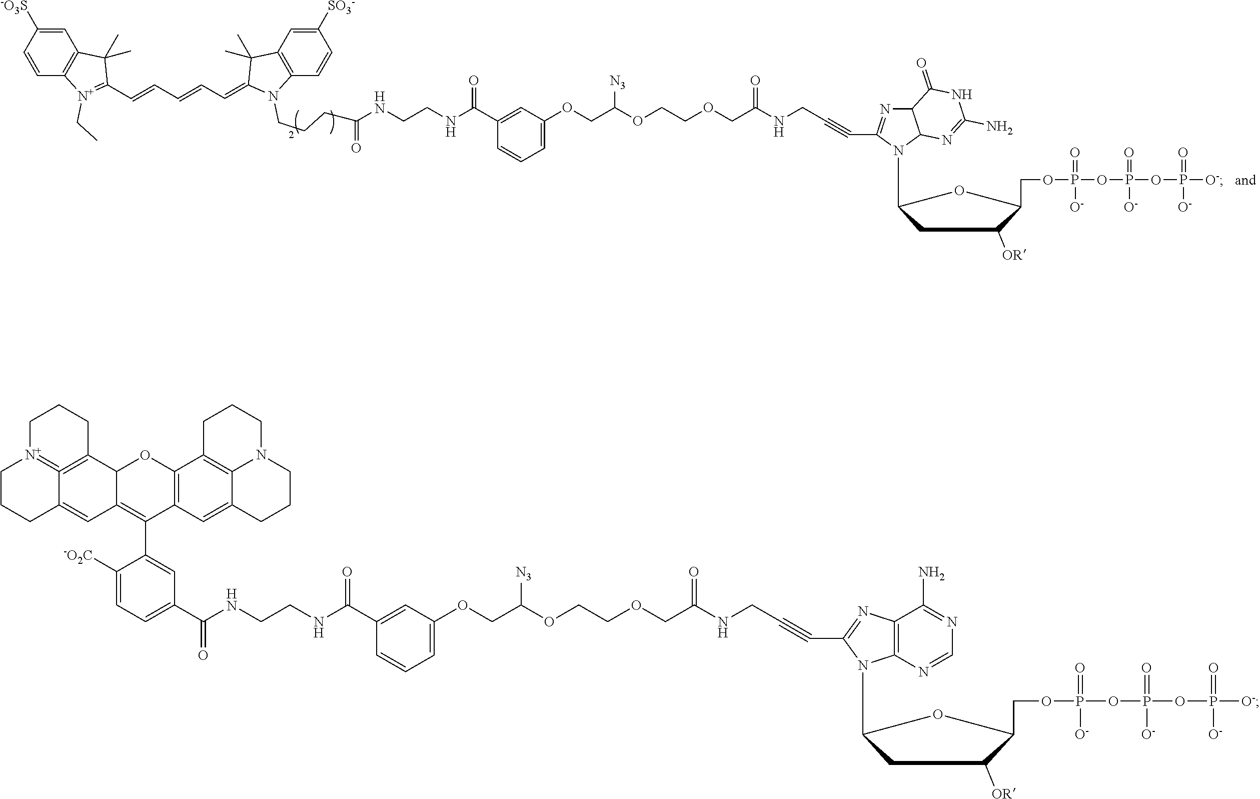
wherein the R′ is selected from:
H,

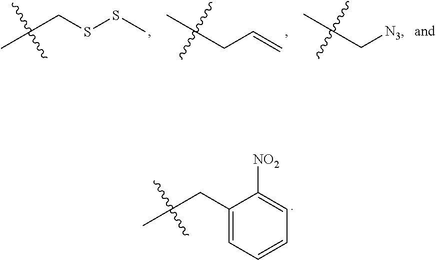

16. The nucleotide of claim 5, selected from: 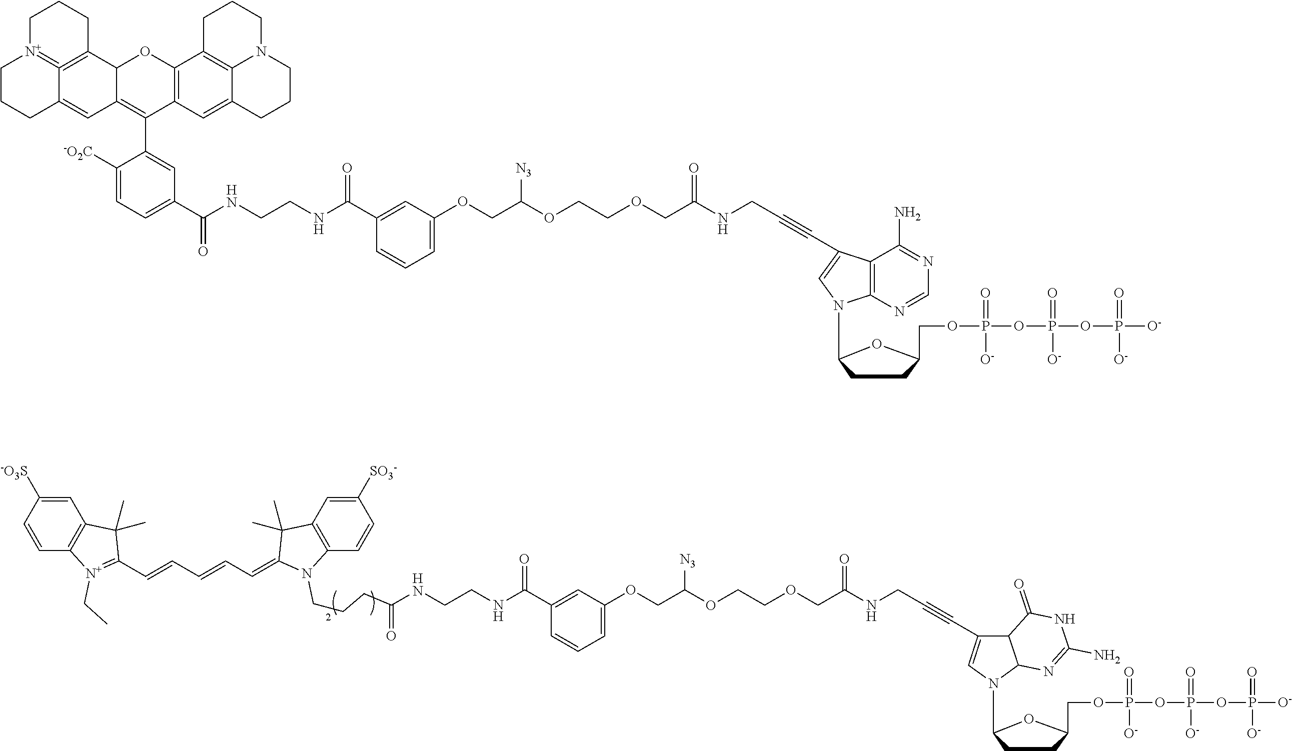 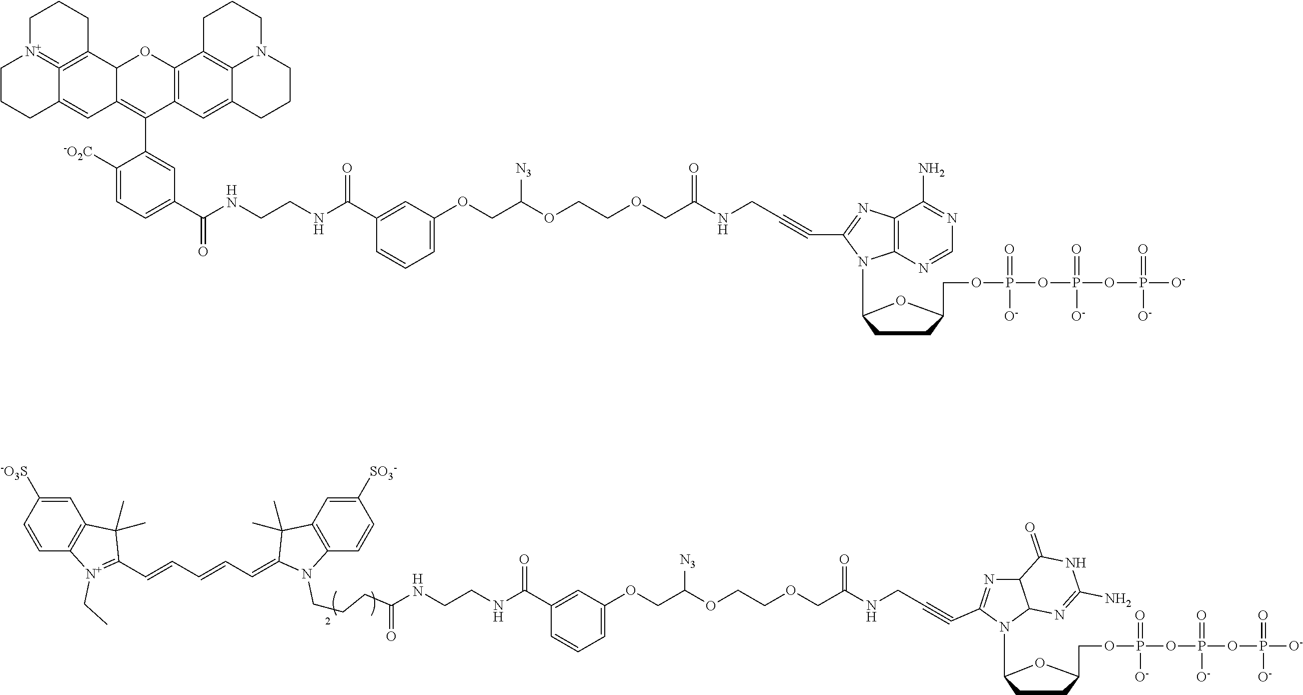 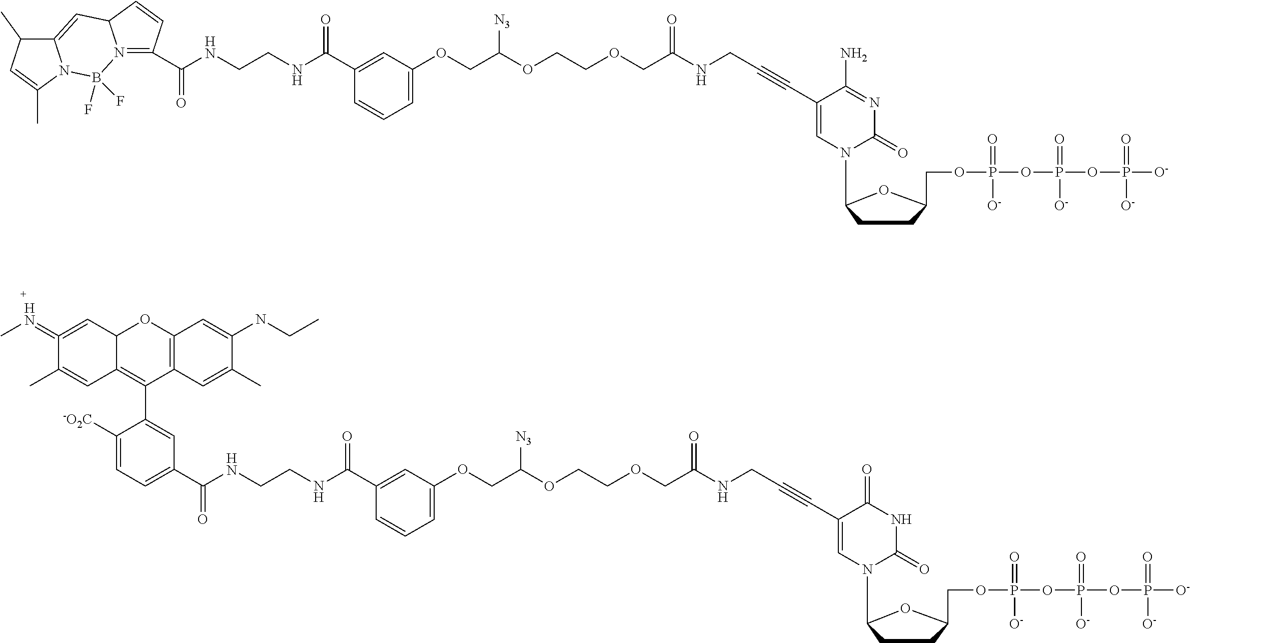 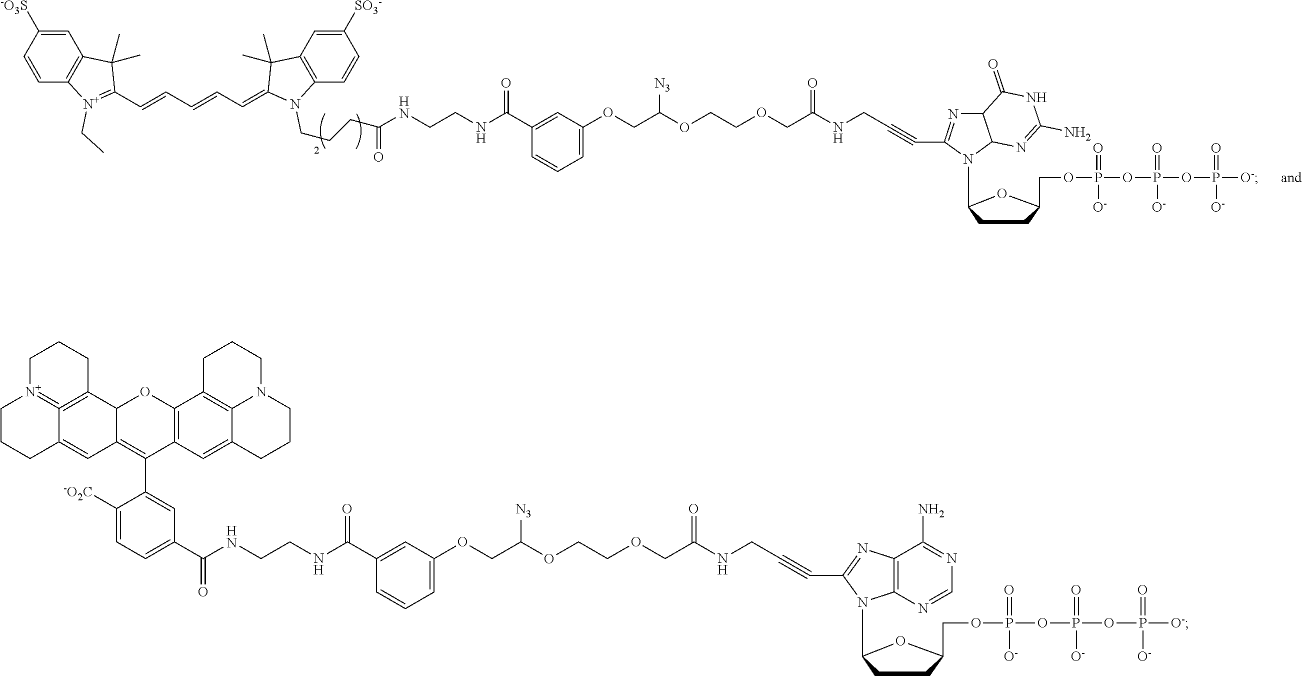
wherein the R′ is selected from:
H,

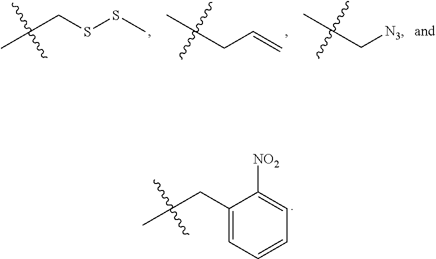

17. A mirror-image nucleic acid polymerase comprising a sequence having at least 90% sequence identity to SEQ ID NO: 1, wherein the polymerase comprises D-form amino acids.

18. The polymerase of claim 17, wherein the polymerase consists of D-form amino acids.

19-20. (canceled)

21. The polymerase of claim 17, comprising one or more modifications at one or more amino acid sites selected from E276, K317, N424, and S651 compared to SEQ ID NO: 1.

22-38. (canceled)

39. A method of replicating an L-polynucleotide, comprising the step of: incubating a mixture comprising (i) the L-polynucleotide, (ii) an L-primer, (iii) L-dNTP, (iv) the polymerase of claim 1, and (v) a buffer, thereby inducing replication of the L-polymerase.

40-43. (canceled)

44. A method of sequencing an L-polynucleotide, comprising the cycle of:
a. incubating a mixture comprising (i) the L-polynucleotide, (ii) an L-primer, (iii) L-3′-O—R′-dNTP-R₂-Label, (iv) the polymerase of claim 17, and (v) a buffer, thereby obtaining a replication product;
b. detecting a signal from the L-3′-O—R′-dNTP-R₂-Label incorporated into the replication product; and
c. inducing cleavage of the R′ group and R₂ group of the L-3′-O—R′-dNTP-R₂-Label incorporated into the replication product.

45-49. (canceled)

50. A method of sequencing an L-polynucleotide, comprising the steps of:
d. incubating a mixture comprising (i) the L-polynucleotide, (ii) an L-primer, (iii) L-dNTP, (iv) an L-ddNTP-R₂-Label or L-3′-O—R′-dNTP-R₂-Label, (v) the polymerase of claim 17, and (vi) a buffer, thereby obtaining a replication product;
e. separating the replication product; and
f. detecting a signal from the L-ddNTP-R₂-Label or L-3′-O—R′-dNTP-R₂-Label incorporated into the replication product.

51-60. (canceled)

61. A method of sequencing an L-polynucleotide, comprising the cycle of:
a. incubating a mixture comprising (i) the L-polynucleotide, (ii) an L-primer, (iii) L-3′-O—R′-dNTP, (iv) L-ddNTPs-R₂-Label or L-dNTPs-R₂-Label, (v) the polymerase of claim 17, and (vi) a buffer, thereby obtaining a replication product;
b. detecting a signal from the L-ddNTP-R₂-Label or L-3′-O—R′-dNTP-R₂-Label incorporated into the replication product; and
c. inducing cleavage of i) the R′ group of the L-3′-O—R′-dNTP and R₂ group of L-ddNTPs-R₂-Label incorporated into the replication product; or ii) the R′ group of the L-3′-O—R′-dNTP, R′ and R₂ group of the L-3′-O—R′-dNTP-R₂-Label incorporated into the replication product.

62-72. (canceled)

73. A kit for replication of an L-polynucleotide, comprising (i) the polymerase of claim 17 and (ii) optionally, a buffer.

74-86. (canceled)


SEQUENCE LISTING (35 sequences)

SEQ ID NO: 1 - protein, 775 aa, Thermococcus sp.
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 2 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLAAVYEAVFGKPKEKVYAEEIAQAWESGEGLERVARYSMEDAGVTYELGREFFPMEAQLSRLIGQSLWDVSRSSTGNLVEWFLLRKAYKRNELAPNKPDERELARRRGGYAGGYVKEPERGLWDNIVYLDFRSLVPSIIITHNVSPDTLAREGCKEYDVAPEVGHKFCKDFPGFIPSLLGDLLEERQKIKRKMKATVDPLEKKLLDYRQRLIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLAKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 3 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPVEVWKLYFNHPQDVPAIRDRIRAHPAVVDIYEYDIPFAKRYLIDKGLVPMEGDEELTMLAFAIATLYHEGEEFGTGPILMISYADGSEARVATWKKVDLPYVDVVSTEKEMVKRFLRVVREKDPDVLITYNGDNFDFAYLKKRCEELGVKFTLGRDGSEPKIQRMGDRFAVEVKGRVHFDLYPVARRTLNLPTYTLAAVYEAVFGKPKEKVYAEEIAQAWESGEGLERVARYSMEDAGVTYELGREFFPMEAQLSRLIGQSLWDVSRSSTGNLVEWFLLRKAYKRNELAPNKPDERELARRRGGYAGGYVKEPERGLWDNVVYLDFRSLVPSIIITHNVSPDTLAREGCKEYDVAPEVGHKFCKDFPGFIPSLLGDLLEERQKIKRKMKATVDPLEKKLLDYRQRLIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVVDEEGKITTRGLEVVRRDWSEAAKETQARVLEALLKHGDVEEAVRVVKEVTEKLAKYEVPPEKLVIHEQITRDLRDYKATGPHVAVAKRLAARGVKIRPGTVISYIVLKGSGRIGDRAYPADEFDPTKHRYDAEYYVENQVLPAVERVLKAFGYRKEDLRYQKTKQVGLGAWLKVKGKK

SEQ ID NO: 4 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLAAVYEAVFGKPKEKVYAEEIAQAWESGEGLERVARYSMEDAGVTYELGREFFPMEAQLSRLIGQSLWDVSRSSTGNLVEWFLLRKAYKRNELAPNKPDERELARRRGGYAGGYVKEPERGLWDNIVYLDFRSAAISIIITHNVSPDTLAREGCKEYDVAPEVGHKFCKDFPGFIPSLLGDLLEERQKIKRKMKATVDPLEKKLLDYRQRVIKILANSFYGYYGYAKARWYCKECAESVSAWGREYLEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLAKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 5 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPVEVWKLYFNHPQDVPAIRDRIRAHPAVVDIYEYDIPFAKRYLIDKGLVPMEGDEELTMLAFAIATLYHEGEEFGTGPILMISYADGSEARVATWKKVDLPYVDVVSTEKEMVKRFLRVVREKDPDVLITYNGDNFDFAYLKKRCEELGVKFTLGRDGSEPKIQRMGDRFAVEVKGRVHFDLYPVARRTLNLPTYTLAAVYEAVFGKPKEKVYAEEIAQAWESGEGLERVARYSMEDAGVTYELGREFFPMEAQLSRLIGQSLWDVSRSSTGNLVEWFLLRKAYKRNELAPNKPDERELARRRGGYAGGYVKEPERGLWDNVVYLDFRSAAISIIITHNVSPDTLAREGCKEYDVAPEVGHKFCKDFPGFIPSLLGDLLEERQKIKRKMKATVDPLEKKLLDYRQRVIKILANSFYGYYGYAKARWYCKECAESVSAWGREYLEMVIRELEEKFGFKVLYADTDGLHATIPGADAETVKKKAKEFLKYINPKLPGLLELEYEGFYVRGFFVTKKKYAVVDEEGKITTRGLEVVRRDWSEAAKETQARVLEALLKHGDVEEAVRVVKEVTEKLAKYEVPPEKLVIHEQITRDLRDYKATGPHVAVAKRLAARGVKIRPGTVISYIVLKGSGRIGDRAYPADEFDPTKHRYDAEYYVENQVLPAVERVLKAFGYRKEDLRYQKTKQVGLGAWLKVKGKK

SEQ ID NO: 6 - protein, 776 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPLEDGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLAAVYEAVFGKPKEKVYAEEIAQAWESGEGLERVARYSMEDAGVTYELGREFFPMEAQLSRLIGQSLWDVSRSSTGNLVEWFLLRKAYKRNELAPNKPDERELARRRGGYAGGYVKEPERGLWDNIVYLDFRSAAISIIITHNVSPDTLAREGCKEYDVAPEVGHKFCKDFPGFIPSLLGDLLEERQKIKRKMKATVDPLEKKLLDYRQRVIKILANSFYGYYGYAKARWYCKECAESVSAWGREYLEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLAKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 7 - protein, 776 aa, synthetic construct
MILDTDYITENGKPVIRVFKKENGEFKIEYDRTFEPYFYALLKDDSAIEDVKKVTAKRHGTVVKVKRAEKVQKKFLGRPVEVWKLYFNHPQDVPAIRDRIRAHPAVVDIYEYDIPFAKRYLIDKGLVPLEDGDEELTMLAFAIATLYHEGEEFGTGPILMISYADGSEARVATWKKVDLPYVDVVSTEKEMVKRFLRVVREKDPDVLITYNGDNFDFAYLKKRCEELGVKFTLGRDGSEPKIQRMGDRFAVEVKGRVHFDLYPVARRTLNLPTYTLAAVYEAVFGKPKEKVYAEEIAQAWESGEGLERVARYSMEDAGVTYELGREFFPMEAQLSRLIGQSLWDVSRSSTGNLVEWFLLRKAYKRNELAPNKPDERELARRRGGYAGGYVKEPERGLWDNVVYLDFRSAAISIIITHNVSPDTLAREGCKEYDVAPEVGHKFCKDFPGFIPSLLGDLLEERQKIKRKMKATVDPLEKKLLDYRQRVIKILANSFYGYYGYAKARWYCKECAESVSAWGREYLEMVIRELEEKFGFKVLYADTDGLHATIPGADAETVKKKAKEFLKYINPKLPGLLELEYEGFYVRGFFVTKKKYAVVDEEGKITTRGLEVVRRDWSEAAKETQARVLEALLKHGDVEEAVRVVKEVTEKLAKYEVPPEKLVIHEQITRDLRDYKATGPHVAVAKRLAARGVKIRPGTVISYIVLKGSGRIGDRAYPADEFDPTKHRYDAEYYVENQVLPAVERVLKAFGYRKEDLRYQKTKQVGLGAWLKVKGKK

SEQ ID NO: 8 - protein, 775 aa, synthetic construct; identical to SEQ ID NO: 3

SEQ ID NO: 9 - protein, 466 aa, synthetic construct
MILDTDYITENGKPVIRVFKKENGEFKIEYDRTFEPYFYALLKDDSAIEDVKKVTAKRHGTVVKVKRAEKVQKKFLGRPVEVWKLYFNHPQDVPAIRDRIRAHPAVVDIYEYDIPFAKRYLIDKGLVPMEGDEELTMLAFAIATLYHEGEEFGTGPILMISYADGSEARVATWKKVDLPYVDVVSTEKEMVKRFLRVVREKDPDVLITYNGDNFDFAYLKKRCEELGVKFTLGRDGSEPKIQRMGDRFAVEVKGRVHFDLYPVARRTLNLPTYTLAAVYEAVFGKPKEKVYAEEIAQAWESGEGLERVARYSMEDAGVTYELGREFFPMEAQLSRLIGQSLWDVSRSSTGNLVEWFLLRKAYKRNELAPNKPDERELARRRGGYAGGYVKEPERGLWDNVVYLDFRSLVPSIIITHNVSPDTLAREGCKEYDVAPEVGHKFCKDFPGFIPSLLGDLLEERQKIKRK

SEQ ID NO: 10 - protein, 309 aa, synthetic construct
MKATVDPLEKKLLDYRQRLIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVVDEEGKITTRGLEVVRRDWSEAAKETQARVLEALLKHGDVEEAVRVVKEVTEKLAKYEVPPEKLVIHEQITRDLRDYKATGPHVAVAKRLAARGVKIRPGTVISYIVLKGSGRIGDRAYPADEFDPTKHRYDAEYYVENQVLPAVERVLKAFGYRKEDLRYQKTKQVGLGAWLKVKGKK

SEQ ID NO: 11 - protein, 4 aa, synthetic construct
AVYE

SEQ ID NO: 12 - protein, 11 aa, synthetic construct
CVFGKPKEKVY

SEQ ID NO: 13 - protein, 15 aa, synthetic construct
  SITE 1: note = Trifluoroacetic acid thiazolidine alanine
  SITE 15: note = Tyrosine hydrazine
AVYECVFGKPKEKVY

SEQ ID NO: 14 - protein, 9 aa, synthetic construct
  SITE 9: note = Threonine hydrazine
CDTDGLHAT

SEQ ID NO: 15 - protein, 20 aa, synthetic construct
  SITE 1: note = Trifluoroacetic acid thiazolidine aspartic acid
  SITE 20: note = Lysine hydrazine
DTDGLHATIPGADAETVKKK

SEQ ID NO: 16 - protein, 35 aa, synthetic construct
  SITE 35: note = Tyrosine hydrazine
CKEFLKYINPKLPGLLELEYEGFYVRGFFVTKKKY

SEQ ID NO: 17 - protein, 56 aa, synthetic construct
  SITE 56: note = Tyrosine hydrazine
CDTDGLHATIPGADAETVKKKAKEFLKYINPKLPGLLELEYEGFYVRGFFVTKKKY

SEQ ID NO: 18 - protein, 35 aa, synthetic construct
  SITE 35: note = Tyrosine hydrazine
AKEFLKYINPKLPGLLELEYEGFYVRGFFVTKKKY

SEQ ID NO: 19 - protein, 4 aa, synthetic construct
  SITE 1: note = Trifluoroacetic acid thiazolidine alanine
  SITE 4: note = Glutamic acid hydrazine
AVYE

SEQ ID NO: 20 - protein, 11 aa, synthetic construct
  SITE 11: note = Tyrosine hydrazine
CVFGKPKEKVY

SEQ ID NO: 21 - protein, 33 aa, synthetic construct
  SITE 33: note = Tyrosine hydrazine
MKATVDPLEKKLLDYRQRLIKILANSFYGYYGY

SEQ ID NO: 22 - protein, 10 aa, synthetic construct
  SITE 7: note = Cysteine acetamidomethyl
  SITE 10: note = Cysteine acetamidomethyl
CKARWYCKEC

SEQ ID NO: 23 - protein, 29 aa, synthetic construct
  SITE 29: note = Tyrosine hydrazine
AESVTAWGREYIEMVIRELEEKFGFKVLY

SEQ ID NO: 24 - protein, 24 aa, synthetic construct
  SITE 24: note = Aspartic acid hydrazine
CEEIAQAWESGEGLERVARYSMED

SEQ ID NO: 25 - protein, 15 aa, synthetic construct
AVYECVFGKPKEKVY

SEQ ID NO: 26 - protein, 9 aa, synthetic construct
CDTDGLHAT

SEQ ID NO: 27 - protein, 20 aa, synthetic construct
DTDGLHATIPGADAETVKKK

SEQ ID NO: 28 - protein, 35 aa, synthetic construct; identical to SEQ ID NO: 16

SEQ ID NO: 29 - protein, 56 aa, synthetic construct; identical to SEQ ID NO: 17

SEQ ID NO: 30 - protein, 35 aa, synthetic construct; identical to SEQ ID NO: 18

SEQ ID NO: 31 - protein, 33 aa, synthetic construct; identical to SEQ ID NO: 21

SEQ ID NO: 32 - protein, 6 aa, synthetic construct
CKARWY

SEQ ID NO: 33 - protein, 29 aa, synthetic construct
AESVTAWGREYIEMVIRELEEKFGFKVLY

SEQ ID NO: 34 - protein, 39 aa, synthetic construct
  SITE 7: note = Cysteine acetamidomethyl
  SITE 10: note = Cysteine acetamidomethyl
  SITE 39: note = Tyrosine hydrazine
CKARWYCKECAESVTAWGREYIEMVIRELEEKFGFKVLY

SEQ ID NO: 35 - protein, 72 aa, synthetic construct
  SITE 40: note = Cysteine acetamidomethyl
  SITE 43: note = Cysteine acetamidomethyl
  SITE 72: note = Tyrosine hydrazine
MKATVDPLEKKLLDYRQRLIKILANSFYGYYGYCKARWYCKECAESVTAWGREYIEMVIRELEEKFGFKVLY